(19) United States
(12) Patent Application Publication
(10) Pub. No.: US 2023/0373960 A1
(43) Pub. Date: Nov. 23, 2023

(54) INDOLINE COMPOUNDS AND DERIVATIVES AS EGFR INHIBITORS

(71) Applicant: BeiGene, Ltd., Grand Kayman (KY)

(72) Inventors: Bailin LEI, Beijing (CN); Huaqing LIU, Beijing (CN); Songzhe HAN, Beijing (CN); Zhiwei WANG, Beijing (CN)

(21) Appl. No.: 18/027,665

(22) PCT Filed: Sep. 22, 2021
(86) PCT No.: PCT/CN2021/119536

§ 371 Date: Mar. 22, 2023

(30) Foreign Application Priority Data
Sep. 22, 2020 (WO) PCT/CN220/116906
Aug. 23, 2021 (WO) PCT/CN2021/113968

(51) Int. Cl. C07D 401/14; C07D 405/14; C07D 471/10; C07D 487/04; C07D 519/00; A61P 35/00

(52) U.S. Cl. CPC C07D 401/14; C07D 405/14; C07D 471/10; C07D 487/04; C07D 519/00; A61P 35/00

(57) ABSTRACT

Disclosed herein are novel pharmaceutically active compounds and their preparation methods. The compounds inhibit mutant EGFR and are useful in the treatment of oncological diseases.

### FIELD OF THE INVENTION

Disclosed herein are novel pharmaceutically active compounds and their preparation methods. The compounds inhibit mutant EGFR and are useful in the treatment of oncological diseases.

### BACKGROUND OF THE INVENTION

Epidermal growth factor receptor (EGFR) that belongs to the ErbB family is a transmembrane receptor tyrosine kinase (RTK), which plays a fundamentally key role in cell proliferation, differentiation, and motility (Y. Yarden, et al., Nat. Rev. Mol. Cell Biol. 2001; 2:127-137). Homo- or heterodimerization of EGFR and other ErbB family members activates cytoplasmic tyrosine kinase domains to initiate intracellular signaling. Overexpression or activating mutations of EGFR are associated with the development of many types of cancers, such as pancreatic cancer, breast cancer, glioblastoma multiforme, head and neck cancer, and non-small cell lung cancer (Yewale C., et al. Biomaterials. 2013, 34 (34): 8690-8707). The activating mutations in the EGFR tyrosine kinase domain (L858R mutation and exon-19 deletion) have been identified as oncogenic drivers for NSCLC (Konduri, K., et al. Cancer Discovery 2016, 6 (6), 601-611). The first-generation EGFR tyrosine kinase inhibitors (EGFR-TKIs) gefitinib and erlotinib have approved for NSCLC patients with EGFR activation mutations (M. Maemondo, N. Engl. J. Med. 362 (2010) 2380-2388). Although most patients with EGFR mutant NSCLC respond to these therapies, patients typically develop resistance after an average of one year on treatment. There are several mechanisms of acquired resistance to gefitinib and erlotinib, including a secondary threonine 790 to methionine 790 mutation (T790M), which is also called “gatekeeper” T790M mutation (Xu Y., et al. Cancer Biol Ther. 2010, 9 (8): 572-582). Therefore, the second-generation EGFR-TKIs afatinib and the third-generation EGFR-TKIs osimertinib (AZD9291) were developed as irreversible EGFR inhibitors that bind to Cys797 for the treatment of patients with T790M mutation. In particular, osimertinib that largely spares WT EGFR has achieved a greater clinical response rate in NSCLC patients with EGFR T790M. However, several recent studies have reported a tertiary Cys797 to Ser797 (C797S) point mutation with osimertinib clinical therapy (Thress K S, et al. Nat. Med. 2015, 21 (6): 560-562). There is a need for drugs which can overcome EGFR (C797S) resistance obstacle in non-small cell lung cancer (NSCLC).

The present application provides novel EGFR-TKI to inhibit oncogenic EGFR harboring all the current resistance mutations, L858R, T790M and C797S.

### SUMMARY OF THE INVENTION

One objective of the present invention is to provide compounds and derivatives which are selective tyrosine kinase inhibitors of mutant EGFR, their use as therapeutically active substances, especially as agents for the treatment of oncological diseases and their preparation methods.

Aspect 1. A compound of Formula (I):

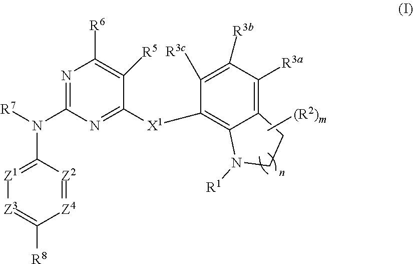

or an N-oxide thereof or a pharmaceutically acceptable salt thereof or a stereoisomer thereof or prodrug thereof, wherein:
X¹ is a single bond, NR⁴, O, S, S(O), S(O)₂ or CH₂;
Z¹ is N or CR⁹, Z² is N or CR¹⁰, Z³ is N or CR¹¹, Z⁴ is N or CR¹²;
R¹ is —S(O)R^{1a}, —S(O)₂R^{1a}, —C(O)R^{1a}, —P(O)R₁ₐR_{1b} or

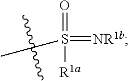

R^{1a} and R^{1b} are each independently H, —C₁₋₈alkyl, —C₂₋₈alkenyl, —C₂₋₈alkynyl, cycloalkyl, heterocyclyl, aryl, heteroaryl, —CN, —OR^{1d}, —CH₂CONR^{1d}R^{1e}, —CH₂CH₂CONR^{1d}R^{1e}, —CH₂CH₂CH₂CONR^{1d}R^{1e}, —NR^{1d}R^{1e}, —CH₂NR^{1d}R^{1e}, —CH₂CH₂NR^{1d}R^{1e}, —CH₂CH₂CH₂NR^{1d}R^{1e} or —NR^{1d}COR^{1e}, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1f};
R^{1d} and R^{1e} are each independently hydrogen, —C₁₋₈alkyl, -haloC₁₋₈alkyl, —C₂₋₈alkenyl, —C₂₋₈alkynyl, cycloalkyl, heterocyclyl, aryl or heteroaryl; or
R^{1d} and R^{1e} together with the atom(s) to which they are attached, form a 3- to 12-membered ring, said ring comprising 0, 1 or 2 additional heteroatom(s) independently selected from the group consisting of nitrogen, oxygen or optionally oxidized sulfur as ring member(s), said ring is optionally substituted with at least one substituent R^{1f};
R^{1f}, at each of its occurrence, is independently hydrogen, halogen, —C₁₋₈alkyl, -haloC₁₋₈alkyl, —C₂₋₈alkenyl, —C₂₋₈alkynyl, cycloalkyl, halocycloalkyl, heterocyclyl, aryl, heteroaryl, oxo (═O), —CN, —OR^{1g}, —COR^{1g}, —CO₂R^{1g}, —CONR^{1g}R^{1h}, —NR^{1g}R^{1h}, —NR^{1g}COR^{1h} or —NR^{1g}CO₂R^{1h}; or
two R^{1f} together with the atom(s) to which they are attached, form a 3- to 12-membered ring, said ring comprising 0, 1 or 2 additional heteroatom(s) independently selected from the group consisting of nitrogen, oxygen or optionally oxidized sulfur as ring member(s), said ring is optionally substituted with at least one substituent hydrogen, halogen, hydroxyl, —C₁₋₈alkyl, -haloC₁₋₈alkyl, —C₂₋₈alkenyl, —C₂₋₈alkynyl, cycloalkyl, heterocyclyl, aryl, heteroaryl, oxo or —CN;
R^{1g} and R^{1h} are each independently hydrogen, halogen, hydroxyl, —C₁₋₈alkyl, -haloC₁₋₈alkyl, —C₂₋₈alkenyl, —C₂₋₈alkynyl, cycloalkyl, heterocyclyl, aryl or heteroaryl;
R², R^{3a}, R^{3b} and R^{3c} are each hydrogen, halogen, —C₁₋₈alkyl, cycloalkyl, heterocyclyl, aryl, heteroaryl, —CN, oxo (═O), —OR^{2a}, —COR^{2a}, —CO₂R^{2a}, —CONR^{2a}R^{2b}, —NR^{2a}R^{2b}, —NR^{2a}COR^{2b} or —NR^{2a}CO₂R^{2b}, wherein each of —C₁₋₈alkyl, cycloalkyl, heterocyclyl, aryl or heteroaryl is optionally substituted with at least one substituent R^{2c}; or
(R² and R^{3a}) together with the atoms to which they are attached, form a 3- to 12-membered ring, said ring comprising 0, 1 or 2 additional heteroatom(s) independently selected from the group consisting of nitrogen, oxygen or optionally oxidized sulfur as ring member(s), said ring is optionally substituted with at least one substituent R^{2c}; or
when m≥2, two germinal R² together with the atom to which they are attached, form a 3-to 12-membered spiro ring, or two R² together with the atoms to which they are attached, form a 3- to 12-membered ring, said ring comprising 0, 1 or 2 additional heteroatom(s) independently selected from the group consisting of nitrogen, oxygen or optionally oxidized sulfur as ring member(s), said ring is optionally substituted with at least one substituent R^{2c}; or
(R^{3a} and R^{3b}) or (R^{3b} and R^{3c}) together with the atoms to which they are attached, form a 3-to 12-membered ring, said ring comprising 0, 1 or 2 additional heteroatom(s) independently selected from the group consisting of nitrogen, oxygen or optionally oxidized sulfur as ring member(s), said ring is optionally substituted with at least one substituent R^{2c};
R^{2a} and R^{2b} are each independently hydrogen, —C₁₋₈alkyl, —C₂₋₈alkenyl, —C₂₋₈alkynyl, C₁₋₈alkoxy —C₁₋₈alkyl-, cycloalkyl, heterocyclyl, aryl or heteroaryl, wherein each of —C₁₋₈alkyl, -C₂₋₈alkenyl, —C₂₋₈alkynyl, C₁₋₈alkoxy-C₁₋₈alkyl-, cycloalkyl, heterocyclyl, aryl or heteroaryl is optionally substituted with at least one substituent R^{2c};
R^{2c}, at each of its occurrence, is independently halogen, amino, —C₁₋₈alkyl, —C₂₋₈alkenyl, —C₂₋₈alkynyl, cycloalkyl, heterocyclyl, aryl, heteroaryl, oxo (═O), —CN, —OR^{2d}, —COR^{2d}, —CO₂R^{2d}, —CONR^{2d}R^{2e}, —NR^{2d}R^{2e}, —NR^{2d}COR^{2e}, or —NR^{2d}CO₂R^{2e};
R^{2d} and R^{2e}, are each independently hydrogen, —C₁₋₈alkyl, -haloC₁₋₈alkyl, —C₂₋₈alkenyl, —C₂₋₈alkynyl, cycloalkyl, heterocyclyl, aryl or heteroaryl;
R⁴ and R⁷ are each independently hydrogen, —C₁₋₈alkyl, —C₂₋₈alkenyl, —C₂₋₈alkynyl or cycloalkyl, wherein each of said —C₁₋₈alkyl, —C₂₋₈alkenyl, —C₂₋₈alkynyl or cycloalkyl is optionally substituted with at least one substituent R^{4a};
R^{4a} is independently hydrogen, halogen, —C₁₋₈alkyl, -haloC₁₋₈alkyl, —C₂₋₈alkenyl, —C₂₋₈alkynyl, cycloalkyl, —CN or —OR^{4b};
R^{4b} is hydrogen, —C₁₋₈alkyl, -haloC₁₋₈alkyl, C₁₋₈alkoxy-C₁₋₈alkyl- or —C₃₋₆cycloalkyl;
R⁵ and R⁶ are independently hydrogen, halogen, —C₁₋₈alkyl, —C₂₋₈alkenyl, —C₂₋₈alkynyl, cycloalkyl, heterocyclyl, aryl, heteroaryl, —CN, —OR^{5a}, —COR^{5a}, —CO₂R^{5a}, —CONR^{5a}R^{5b}, —NR^{5a}R^{5b} or —NR^{5a}COR^{5b}, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{5c};
R^{5a} are R^{5b} are each independently hydrogen, —C₁₋₈alkyl, —C₂₋₈alkenyl, —C₂₋₈alkynyl, C₁₋₈alkoxy C₁₋₈alkyl-, cycloalkyl, heterocyclyl, aryl or heteroaryl, wherein each of said —C₁₋₈alkyl, —C₂₋₈alkenyl, —C₂₋₈alkynyl, C₁₋₈alkoxy-C₁₋₈alkyl-, cycloalkyl, heterocyclyl, aryl or heteroaryl is optionally substituted with at least one substituent R^{5c};
R^{5c}, at each occurrence, is independently halogen, —C₁₋₈alkyl, -haloC₁₋₈alkyl, —C₂₋₈alkenyl, —C₂₋₈alkynyl, cycloalkyl, heterocyclyl, aryl or heteroaryl;
or R⁵ and R⁶, together with the atoms to which they are attached, form a 3- to 12-membered ring, said ring comprising 0, 1 or 2 additional heteroatom(s) independently selected from the group consisting of nitrogen, oxygen, sulfur or optionally oxidized sulfur as ring member(s), said ring is optionally substituted with at least one substituent R⁵⁶;
R⁵⁶ is hydrogen, halogen, —C₁₋₈alkyl, -haloC₁₋₈alkyl, —C₂₋₈alkenyl, —C₂₋₈alkynyl, —C₃₋₆cycloalkyl, 3- to 8-membered heterocyclyl, —C₆₋₁₂aryl, 3- to 8-membered heteroaryl, C₁₋₈alkoxy —C₁₋₈alkyl-, oxo (═O), —CN, —OR^{56a}, —COR^{56a}, —CO₂R^{56a}, —CONR^{56a}R^{56b}, —NR^{56a}R^{56b} or —NR^{56a}COR^{56b};
R^{56a} and R^{56b} are each independently hydrogen, —C₁₋₈alkyl, -haloC₁₋₈alkyl, —C₂₋₈alkenyl, —C₂₋₈alkynyl, C₁₋₈alkoxy-C₁₋₈alkyl-, cycloalkyl, heterocyclyl, aryl or heteroaryl;
R⁸ is halogen, —C₁₋₈alkyl, —C₂₋₈alkenyl, —C₂₋₈alkynyl, cycloalkyl, heterocyclyl, aryl, heteroaryl, —OR^{8a} or —NR^{8a}R^{8b}, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{8c}.

R^{8a} and R^{8b} are each independently hydrogen, —C₁₋₈alkyl, —C₂₋₈alkenyl, —C₂₋₈alkynyl, C₁₋₈alkoxy —C₁₋₈alkyl-, cycloalkyl, heterocyclyl, aryl or heteroaryl, wherein each of said —C₁₋₈alkyl, —C₂₋₈alkenyl, —C₂₋₈alkynyl, C₁₋₈alkoxy-C₁₋₈alkyl-, cycloalkyl, heterocyclyl, aryl or heteroaryl is optionally substituted with at least one substituent R^{8d}; or
R^{8a} and R^{8b}, together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen or optionally oxidized sulfur as ring member(s), said ring is optionally substituted with at least one substituent R^{8d};
R^{8c} is independently hydrogen, halogen, —C₁₋₈alkyl, —C₂₋₈alkenyl, —C₂₋₈alkynyl, cycloalkyl, heterocyclyl, aryl, heteroaryl, oxo (═O), —CN, —OR^{8f}, —COR^{8f}, —CO₂R^{8f}, —CONR^{8f}R^{8g}, —NR^{8f}R^{8g} or —NR^{8f}COR^{8g}, wherein each of said —C₁₋₈alkyl, —C₂₋₈alkenyl, —C₂₋₈alkynyl, cycloalkyl, heterocyclyl, aryl or heteroaryl is optionally substituted with at least one R^{e}; or
two R^{8c} together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen or optionally oxidized sulfur as ring member(s), said ring is optionally substituted with at least one R^{8e};
R^{8d} and R^{8e} are each independently hydrogen, halogen, —C₁₋₈alkyl, —C₂₋₈alkenyl, —C₂₋₈alkynyl, cycloalkyl, heterocyclyl, aryl, heteroaryl, oxo (═O), —CN, —OR^{8h}, —COR^{8h}, —CO₂R^{8h}, —CONR^{8h}R⁸ⁱ, —NR^{8h}R⁸ⁱ or —NR^{8h}COR⁸ⁱ, wherein each of said —C₁₋₈alkyl, —C₂₋₈alkenyl, —C₂₋₈alkynyl, cycloalkyl, heterocyclyl, aryl or heteroaryl is optionally substituted with at least one halogen, —C₁₋₈alkyl, —C₂₋₈alkenyl, —C₂₋₈alkynyl, cycloalkyl, heterocyclyl, aryl, heteroaryl, hydroxyl, —C₁₋₈alkoxy or C₁₋₈alkoxy-C₁₋₈alkyl-; or
two R^{8e} together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen or optionally oxidized sulfur as ring member(s), said ring is optionally substituted with at least one halogen, —C₁₋₈alkyl, —C₂₋₈alkenyl, —C₂₋₈alkynyl, cycloalkyl, heterocyclyl, aryl, heteroaryl, hydroxyl, —C₁₋₈alkoxy or C₁₋₈alkoxy-C₁₋₈alkyl-;
R^{8f}, R^{8g}, R^{8h} and R⁸ⁱ are each independently hydrogen, —C₁₋₈alkyl, -haloC₁₋₈alkyl, C₁₋₈alkoxy —C₁₋₈alkyl-, —C₂₋₈alkenyl, —C₂₋₈alkynyl, cycloalkyl, heterocyclyl, aryl or heteroaryl;
R⁹, R¹⁰, R¹¹ and R¹² are each independently hydrogen, halogen, —C₁₋₈alkyl, —C₂₋₈alkenyl, —C₂₋₈alkynyl, cycloalkyl, heterocyclyl, —C₆₋₁₂aryl, heteroaryl, —CN, —OR^{9d}, —CH₂CONR^{9d}R^{9e}, —CH₂CH₂CONR^{9d}R^{9e}, —CH₂CH₂CH₂CONR^{9d}R^{9e}, —NR^{9d}R^{9e}, —CH₂NR^{9d}R^{9e}, —CH₂CH₂NR^{9d}R^{9e}, —CH₂CH₂CH₂NR^{9d}R^{9e} or —NR^{9d}COR^{9e}, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{9f}; or
(R⁹ and R¹¹) or (R¹⁰ and R¹²) together with the atom(s) to which they are attached, form a 3-to 12-membered ring, said ring comprising 0, 1 or 2 additional heteroatom(s) independently selected from the group consisting of nitrogen, oxygen or optionally oxidized sulfur as ring member(s), said ring is optionally substituted with at least one substituent R^{9f};
R^{9d} and R^{9e} are each independently hydrogen, —C₁₋₈alkyl, —C₂₋₈alkenyl, —C₂₋₈alkynyl, cycloalkyl, heterocyclyl, aryl, or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one halogen, —C₁₋₈alkyl, -haloC₁₋₈alkyl, —C₂₋₈alkenyl, —C₂₋₈alkynyl, cycloalkyl, heterocyclyl, aryl, heteroaryl, hydroxyl, —C₁₋₈alkoxy or C₁₋₈alkoxy-C₁₋₈alkyl-; or
R^{9d} and R^{9e} together with the atom(s) to which they are attached, form a 3- to 12-membered ring, said ring comprising 0, 1 or 2 additional heteroatom(s) independently selected from the group consisting of nitrogen, oxygen or optionally oxidized sulfur as ring member(s), said ring is optionally substituted with at least one substituent R^{9f};
R^{9f}, at each of its occurrence, is independently hydrogen, halogen, -haloC₁₋₈alkyl, —C₁₋₈alkyl, —C₂₋₈alkenyl, —C₂₋₈alkynyl, cycloalkyl, aryl, heteroaryl, oxo (═O), —CN, —OR^{9g}, —COR^{9g}, —CO₂R^{9g}, —CONR^{9g}R^{9h}, —NR^{9g}R^{9h}, —NR^{9g}COR^{9h} or —NR^{9g}CO₂R^{9h}; or
two R⁹ together with the atom(s) to which they are attached, form a 3- to 12-membered ring, said ring comprising 0, 1 or 2 additional heteroatom(s) independently selected from the group consisting of nitrogen, oxygen or optionally oxidized sulfur as ring member(s), said ring is optionally substituted with at least one halogen, —C₁₋₈alkyl, -haloC₁₋₈alkyl, —C₂₋₈alkenyl, —C₂₋₈alkynyl, cycloalkyl, heterocyclyl, aryl, heteroaryl, hydroxyl, —C₁₋₈alkoxy or C₁₋₈alkoxy-C₁₋₈alkyl-;
R^{9g} and R^{9h} are each independently hydrogen, halogen, hydroxyl, —C₁₋₈alkyl, -haloC₁₋₈alkyl, —C₂₋₈alkenyl, —C₂₋₈alkynyl, cycloalkyl, heterocyclyl, aryl or heteroaryl;
n is 0, 1, 2 or 3;
m is 0, 1, 2, 3 or 4.

In some embodiments, Z¹ is CR⁹, Z² is CR¹⁰, Z³ is CR¹¹, Z⁴ is CR¹². In some embodiments, Z¹, Z², Z³ and Z⁴ are each CH. In some embodiments, Z¹ is N, Z² is CR¹⁰, Z³ is CR¹¹, Z⁴ is CR¹². In some embodiments, Z¹ is N, and Z², Z³ and Z⁴ are each CH.

Aspect 2. The compound of Aspect 1, wherein
R¹ is —S(O)₂R^{1a}, —C(O)R^{1a} or

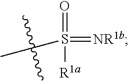

R^{1a} and R^{1b} are each H, methyl, ethyl, propyl, butyl, pentyl, hexyl, heptyl, octyl, cyclopropyl, cyclobutyl, cyclopentyl, cyclohexyl, cycloheptyl, cyclooctyl, azacyclopropanyl, azacyclobutanyl, tetrahydropyrrole, piperidinyl, piperazinyl, morphinyl, epoxyethyl, epoxybutanyl, oxacyclopentanyl, tetrahydropyran, phenyl, pyrrolyl, imidazolyl, pyrazolyl, thiazolyl, thienyl, oxazolyl, pyridinyl, indolyl, quinolinyl, isoquinolinyl, benzimidazolyl, benzothiazolyl, benzopyrazolyl, —CH₂CONR^{1d}R^{1e}, —CH₂CH₂CONR^{1d}R^{1e}, —CH₂CH₂CH₂CONR^{1d}R^{1e}, —NR^{1d}R^{1e}, —CH₂NR^{1d}R^{1e}, —CH₂CH₂NR^{1d}R^{1e} or —CH₂CH₂CH₂NR^{1d}R^{1e}, wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, azacyclopropanyl, azacyclobutanyl, tetrahydropyrrolyl, piperidinyl, piperazinyl, morphinyl, epoxyethyl, epoxybutanyl, oxacyclopentanyl, tetrahydropyran, phenyl, pyrrolyl, imidazolyl, pyrazolyl, thiazolyl, thienyl, oxazolyl, pyridinyl, indolyl, quinolinyl, isoquinolinyl, benzimidazolyl, benzothiazolyl or benzopyrazolyl is optionally substituted with at least one substituent R^{1f};
R^{1d} and R^{1e} are each independently hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, azacyclopropanyl, azacyclobutanyl, tetrahydropyrrolyl, piperidinyl, piperazinyl, morphinyl, epoxyethyl, epoxybutanyl, oxacyclopentanyl, tetrahydropyran, phenyl, pyrrolyl, imidazolyl, pyrazolyl, thiazolyl, thienyl, oxazolyl, pyridinyl, indolyl, quinolinyl, isoquinolinyl, benzimidazolyl, benzothiazolyl or benzopyrazolyl; or
R^{1f}, at each of its occurrence, is independently hydrogen, —F, —Cl, —Br, —I, hydroxyl, methyl, ethyl, propyl, butyl, pentyl, hexyl, heptyl, octyl, cyclopropyl, cyclobutyl, cyclopentyl, cyclohexyl, cycloheptyl, cyclooctyl, azacyclopropanyl, azacyclobutanyl, tetrahydropyrrolyl, piperidinyl, piperazinyl, morphinyl, epoxyethyl, epoxybutanyl, oxacyclopentanyl, tetrahydropyran, phenyl, pyrrolyl, imidazolyl, pyrazolyl, thiazolyl, thienyl, oxazolyl, pyridinyl, indolyl, quinolinyl, isoquinolinyl, benzimidazolyl, benzothiazolyl, benzopyrazolyl or —CN, or
two R^{1f} together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen or optionally oxidized sulfur as ring member(s), said ring is optionally substituted with at least one substituent hydrogen, F, Cl, Br, I, hydroxyl, methyl, ethyl, propyl, butyl, pentyl, hexyl, heptyl, octyl, cyclopropyl, cyclobutyl, cyclopentyl, cyclohexyl, cycloheptyl, cyclooctyl, azacyclopropanyl, azacyclobutanyl, tetrahydropyrrolyl, piperidinyl, piperazinyl, morphinyl, epoxyethyl, epoxybutanyl, oxacyclopentanyl, tetrahydropyran, phenyl, pyrrolyl, imidazolyl, pyrazolyl, thiazolyl, thienyl, oxazolyl, pyridinyl, indolyl, quinolinyl, isoquinolinyl, benzimidazolyl, benzothiazolyl, benzopyrazolyl, oxo or —CN;
R^{1g} and R^{1h} are each independently hydrogen, F, Cl, Br, I, hydroxyl, methyl, ethyl, propyl, butyl, pentyl, hexyl, heptyl, octyl, cyclopropyl, cyclobutyl, cyclopentyl, cyclohexyl, cycloheptyl, cyclooctyl, azacyclopropanyl, azacyclobutanyl, tetrahydropyrrolyl, piperidinyl, piperazinyl, morphinyl, epoxyethyl, epoxy butane, oxacyclopentanyl, tetrahydropyran, phenyl, pyrrolyl, imidazolyl, pyrazolyl, thiazolyl, thienyl, oxazolyl, pyridinyl, indolyl, quinolinyl, isoquinolinyl, benzimidazolyl, benzothiazolyl or benzopyrazolyl.

Aspect 3. The compound of Aspect 1 wherein
R¹ is —S(O)₂R^{1a}, —C(O)R^{1a} or

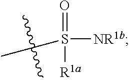

R^{1a} is —CH₃, —C₂H₅, —C₃H₇, —C₄H₉, —C₅H₁₁,_-cyclopropyl, -tert-butyl,

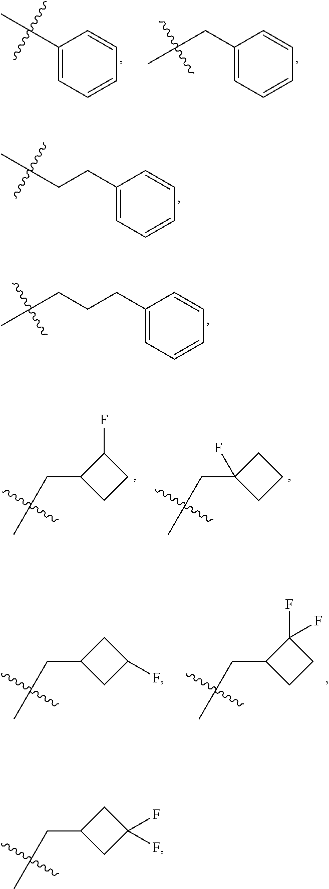

—CH₂F, —CHF₂, —CF₃, —N(CH₃)₂, —NHCH₃, —CH₂N(CH₃)₂, —CH₂CH₂N(CH₃)₂ or —CH₂CH₂CH₂N(CH₃)₂.

Aspect 4. The compound of Aspect 1, wherein
R² and R^{3a}, R^{3b} and R^{3c}, at each of their occurrences, are hydrogen, —F, —Cl, —Br, —I, —C₁₋₈alkyl, C₃₋₈cycloalkyl, —CN, oxo (═O), —OR^{2a} or —COR^{2a}, wherein each of —C₁₋₈alkyl, C₃₋₈cycloalkyl, 3- to 8-membered heterocyclyl, C₆-C₁₂aryl or 5- to 12-membered heteroaryl is optionally substituted with at least one substituent R^{2c},
(R² and R^{3a}), two R² or (R^{3a} and R^{3b}) or (R^{3b} and R^{3c}) together with the atom(s) to which they are attached, form a 3- to 8-membered ring, said ring comprising 0, 1 or 2 additional heteroatom(s) independently selected from the group consisting of nitrogen, oxygen or optionally oxidized sulfur as ring member(s), said ring is optionally substituted with at least one substituent R^{2c};
R^{2a} is independently hydrogen, —C₁₋₈alkyl, —C₂₋₈alkenyl, —C₂₋₈alkynyl, C₁₋₈alkoxy-C₁₋₈alkyl-, C₃₋₈cycloalkyl, 3- to 8-membered heterocyclyl, C₆₋₁₂aryl or 5- to 12-membered heteroaryl, wherein each of —C₁₋₈alkyl, —C₂₋₈alkenyl, —C₂₋₈alkynyl, C₁₋₈alkoxy-C₁₋₈alkyl- or C₃₋₈cycloalkyl is optionally substituted with at least one substituent R^{2c};
R^{2c}, at each of its occurrence, is independently —F, —Cl, —Br, —I, hydroxyl, —NH₂, —CH₃, —C₂H₅, —C₃H₇, —C₄H₉, —C₅H₁₁, —C₆H₁₃, —C₇H₁₅, —C₈H₁₇, phenyl, oxo (═O), —CN, —OCH₃, —OC₂H₅, —OC₃H₇, —OC₄H₉, —OC₅H₁₁, —OC₆H₁₃, —OC₇H₁₅, —OC₈H₁₇, —COCH₃, —COC₂H₅, —COC₃H₇, —COC₄H₉, —COC₅H₁₁, —COC₆H₁₃, —COC₇H₁₅, —COC₈H₁₇, —CO₂CH₃, —CO₂C₂H₅, —CO₂C₃H₇, —CO₂C₄H₉, —CO₂C₅H₁₁, —CO₂C₆H₁₃, —CO₂C₇H₁₅ or —CO₂C₈H₁₇.

Aspect 5. The compound of Aspect 1, wherein
R² is hydrogen, —F, —Cl, —Br, —I, —CH₃, —C₂H₅, —C₃H₇, —C₄H₉, —C₅H₁₁, —C₆H₁₃, —C₇H₁₅, —C₈H₁₇, —CH₂OH, —CH₂CH₂OH, —CH₂CH₂CH₂OH, or oxo; or
when m≥2, two germinal R² together with the atom to which they are attached, form a 3-, 4-, 5- or 6-membered spiro ring, said ring comprising 0, 1 or 2 additional heteroatom(s) independently selected from the group consisting of nitrogen, oxygen or optionally oxidized sulfur as ring member(s).

Aspect 6. The compound of Aspect 1, wherein
R² is hydrogen, F, Cl, Br, I, —CH₃, —C₂H₅, —C₃H₇, —C₄H₉, —C₅H₁₁, —CH₂OH, —CH₂CH₂OH, —CH₂CH₂CH₂OH or oxo; or
when m≥2, two germinal R² together with the atom to which they are attached, form a spiro cyclopropyl or spiro cyclobutyl.

Aspect 7. The compound of Aspect 1, wherein
R^{3a}, R^{3b} and R^{3c} are each hydrogen, F, Cl, Br, I, -methyl, ethyl, propyl, butyl, pentyl, hexyl, heptyl, octyl, cyclopropyl, cyclobutyl, cyclopentyl, cyclohexyl, cycloheptyl, cyclooctyl, —CN, —OR^{2a}, —COR^{2a} or —CO₂R^{2a}, wherein each of methyl, ethyl, propyl, butyl, pentyl, hexyl, heptyl, octyl, cyclopropyl, cyclobutyl, cyclopentyl, cyclohexyl, cycloheptyl or cyclooctyl is optionally substituted with at least one substituent R^{2c},
R^{2a} is each independently hydrogen, —C₁₋₈alkyl, —C₂₋₈alkenyl, —C₂₋₈alkynyl, C₁₋₈alkoxy-C₁₋₈alkyl- or C₃₋₈cycloalkyl, wherein each of —C₁₋₈alkyl, —C₂₋₈alkenyl, —C₂₋₈alkynyl, C₁₋₈alkoxy-C₁₋₈alkyl- or C₃₋₈cycloalkyl is optionally substituted with at least one substituent R^{2c};
R^{2c}, at each of its occurrence, is independently hydroxyl, halogen, —C₁₋₈alkyl, —C₂₋₈alkenyl, —C₂₋₈alkynyl or —C₃₋₈cycloalkyl.

Aspect 8. The compound of Aspect 1, wherein
R^{3a}, R^{3b} and R^{3c} are each —H, —F, —Cl, —Br, —I, hydroxyl, amino, —CH₃, —C₂H₅, —C₃H₇, —C₄H₉, —C₅H₁₁, —C₆H₁₃, —C₇H₁₅, —C₈H₁₇, phenyl, oxo (═O), —CN, —OCH₃, —OC₂H₅, —OC₃H₇, —OC₄H₉, —OC₅H₁₁, —OC₆H₁₃, —OC₇H₁₅, —OC₈H₁₇, —COCH₃, —COC₂H₅, —COC₃H₇, —COC₄H₉, —COC₅H₁₁, —COC₆H₁₃, —COC₇H₁₅, —COC₈H₁₇, —CO₂CH₃, —CO₂C₂H₅, —CO₂C₃H₇, —CO₂C₄H₉, —CO₂C₅H₁₁, —CO₂C₆H₁₃, —CO₂C₇H₁₅ or —CO₂C₈H₁₇.

Aspect 9. The compound of Aspect 1, wherein

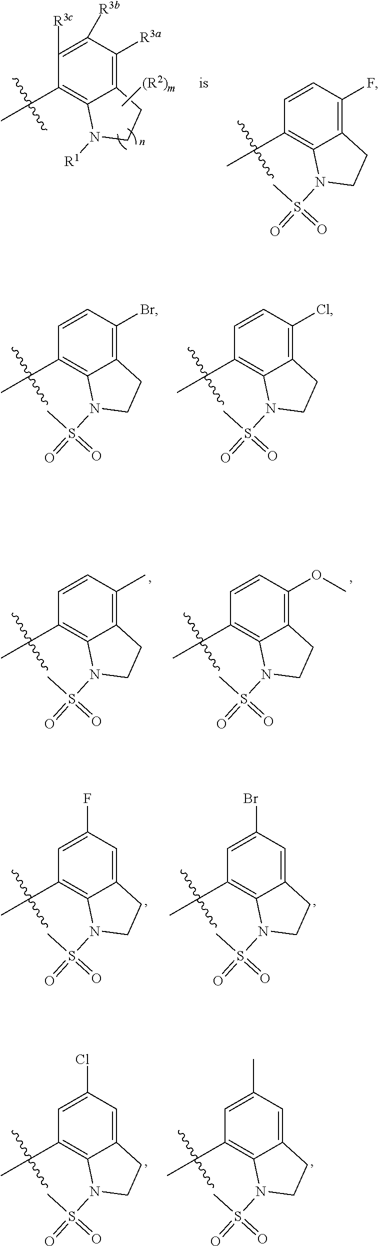

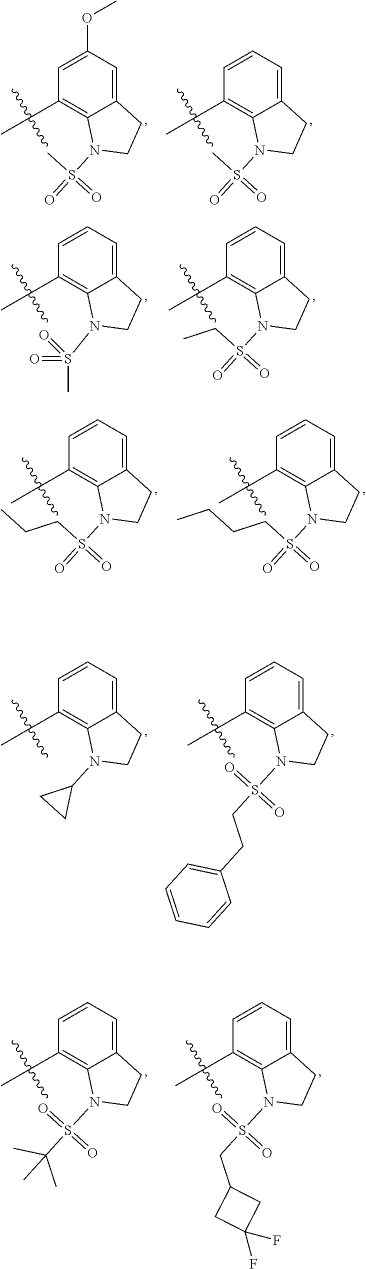

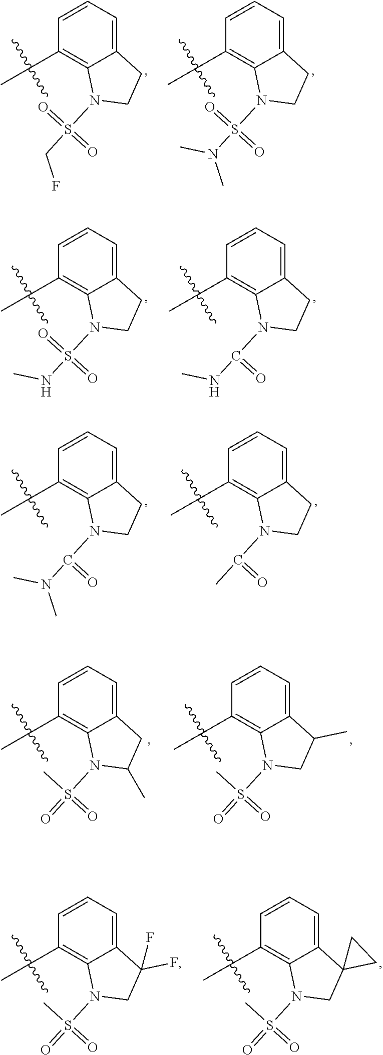

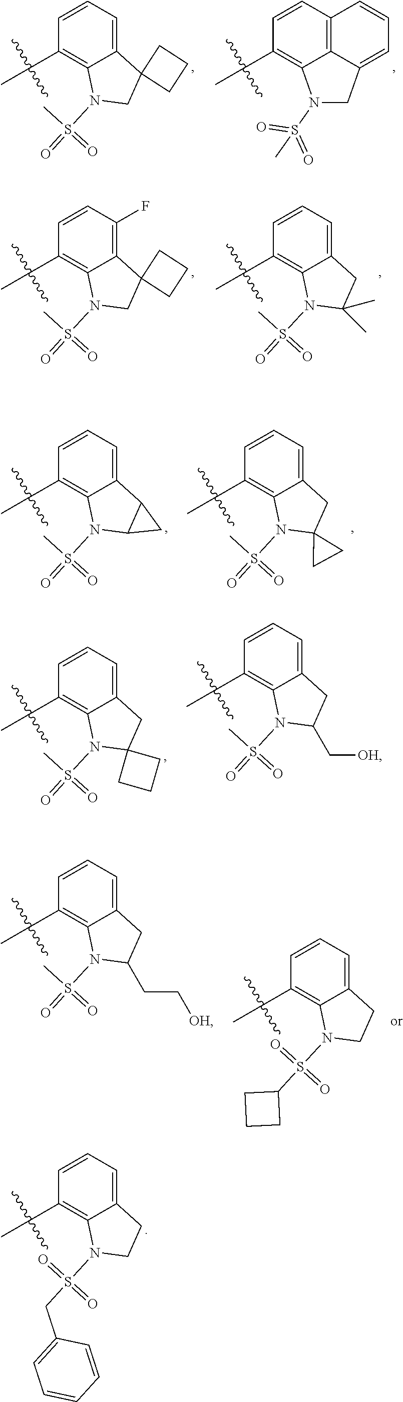

Aspect 10. The compound of Aspect 1, wherein
R⁴ and R⁷ are each independently —H, —CH₃, —C₂H₅, —C₃H₇, —C₄H₉, —C₅H₁₁, —C₆H₁₃, —C₇H₁₅ or —C₈H₁₇.

Aspect 11. The compound of Aspect 1, wherein
R⁵ and R⁶ are independently is hydrogen, —F, —Cl, —Br, —I, hydroxyl, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, —CN, —OR^{5a} or —NR^{8a}R^{8b}, wherein each of said methyl, ethyl, propyl, butyl, pentyl, hexyl, heptyl, octyl, cyclopropyl, cyclobutyl, cyclopentyl, cyclohexyl, cycloheptyl or cyclooctyl is optionally substituted with at least one substituent R^{5c};
R^{5a} are R^{5b} are each independently hydrogen, —C₁₋₈alkyl, —C₂₋₈alkenyl, —C₂₋₈alkynyl, C₁₋₈alkoxy-C₁₋₈alkyl- or —C₃₋₆cycloalkyl, wherein each of said —C₁₋₈alkyl, —C₂₋₈alkenyl, —C₂₋₈alkynyl, C₁₋₈alkoxy-C₁₋₈alkyl- or —C₃₋₆cycloalkyl is optionally substituted with at least one substituent R^{5c}.

R^{5c}, at each of its occurrence, is independently —F, —Cl, —Br, —I, hydroxyl, methyl, ethyl, propyl, butyl, pentyl, hexyl, heptyl or octyl.

Aspect 12. The compound of Aspect 1, wherein
R⁵ and R⁶ are independently is —H, —F, —Cl, —Br, —I, —CH₃, —C₂H₅, —C₃H₇, —C₄H₉, —C₅H₁₁,

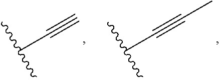

—OCH₃, —OC₂H₅, —OC₃H₇, —OC₄H₉, —OC₅H₁₁, —CH₂F, —CHF₂, —CF₃, —CN, —NH₂, —NHCH₃, —NHC₂H₅ or —N(CH₃)₂.

Aspect 13. The compound of Aspect 1, wherein
R⁵ and R⁶, together with the atom(s) to which they are attached, form a 4-, 5-, 6- or 7-membered ring, said ring comprising 0, 1 or 2 additional heteroatom(s) independently selected from the group consisting of nitrogen, oxygen or optionally oxidized sulfur as ring member(s), said ring is optionally substituted with at least one substituent R⁵⁶;
R⁵⁶ is —H, —F, —Cl, —Br, —I, —CH₃, —C₂H₅, —C₃H₇, —C₄H₉, —CH₁₁, —C₆H₁₃, —C₇H₁₅, —C₈H₁₇, —C₂₋₈alkenyl, —C₂₋₈alkynyl, —CN, —OR^{56a}, —COR^{56a} or —CO₂R^{56a}, wherein each of said —CH₃, —C₂H₅, —C₃H₇, —C₄H₉, —CH₁₁, —C₆H₁₃, —C₇H₁₅, —C₈H₁₇, —C₂₋₈alkenyl or —C₂₋₈alkynyl is optionally substituted with at least one halogen.

Aspect 14. The compound of Aspect 1, wherein

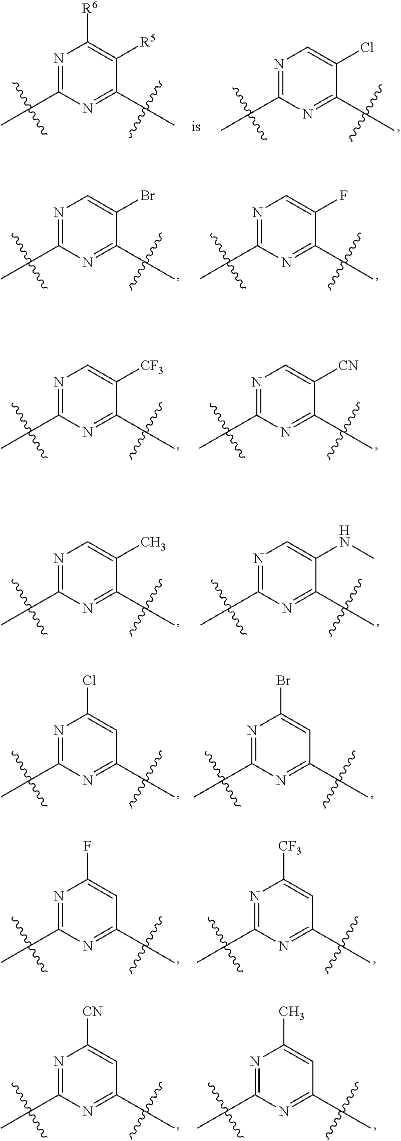

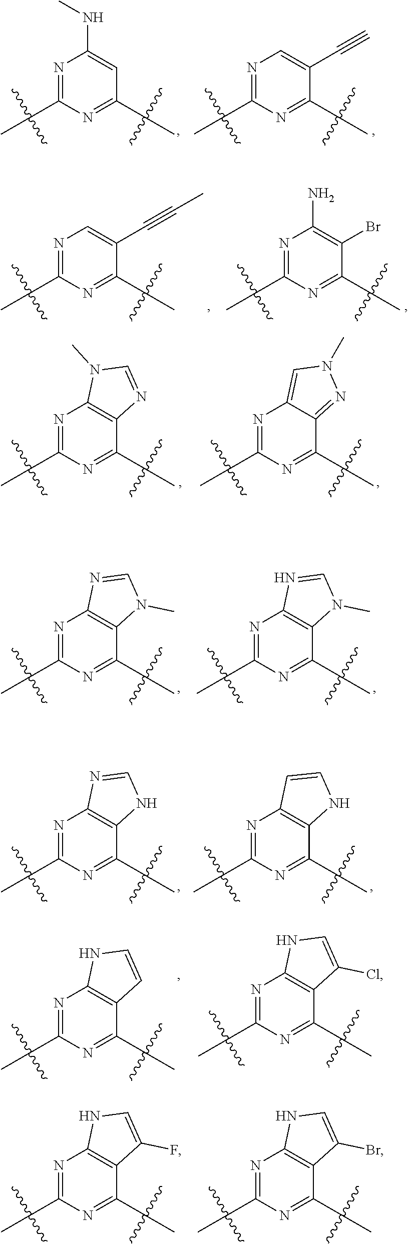

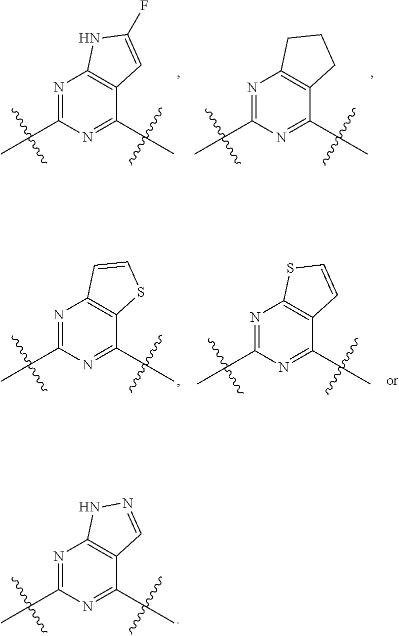 [SMILES: *c1nc(*)c2cc(F)[nH]c2n1 ; *c1nc(*)c2c(n1)CCC2 ; *c1nc(*)c2sccc2n1 ; *c1nc(*)c2ccsc2n1 ; *c1nc(*)c2cn[nH]c2n1]

Aspect 15. The compound of Aspect 1, wherein

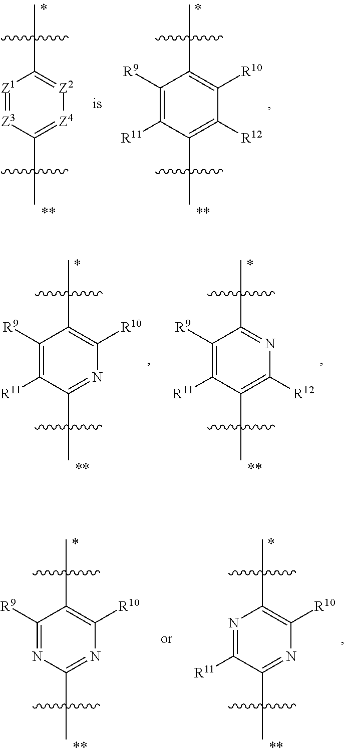

wherein * refers to the position linked to the —N(R⁷)— moiety, and ** refers to the position linked to R⁸.

Aspect 16. The compound of Aspect 1 or 15, wherein
R⁹, R¹⁰, R¹¹ and R¹² are each independently hydrogen, halogen, —C₁₋₈alkyl, —C₂₋₈alkenyl, 3-to 8-membered heterocyclyl, —CN or —OR^{9d}, wherein each of said —C₁₋₈alkyl, —C₂₋₈alkenyl, —C₂₋₈alkynyl, —C₃₋₈cycloalkyl, 3- to 8-membered heterocyclyl, —C₆₋₁₂aryl or 5- to 8-membered heteroaryl is optionally substituted with at least one substituent R^{9f}; or
(R⁹ and R¹¹) or (R¹⁰ and R¹²) or (R¹⁴ and R¹⁵) together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen or optionally oxidized sulfur as ring member(s), said ring is optionally substituted with at least one substituent R^{9f};
R^{9d} and R^{9e} are each independently —H, —CH₃, —C₂H₅, —C₃H₇, —C₄H₉, —C₅H₁₁, —C₆H₁₃, —C₇H₁₅ or —C₈H₁₇; or
R^{9d} and R^{9e} together with the atom(s) to which they are attached, form a 3- to 12-membered ring, said ring comprising 0, 1 or 2 additional heteroatom(s) independently selected from the group consisting of nitrogen, oxygen or optionally oxidized sulfur as ring member(s), said ring is optionally substituted with at least one substituent R^{9f}; R^{9f}, at each of its occurrence, is independently hydrogen, halogen, hydroxyl, —C₁₋₈alkyl, —C₂₋₈alkenyl, —C₂₋₈alkynyl, —C₃₋₈cycloalkyl, 3- to 8-membered heterocyclyl, —C₆₋₁₂aryl, 5- to 8-membered heteroaryl, oxo (═O), —CN, —OR⁹⁹, —COR⁹⁹, —CO₂R^{9g}, —CONR^{9g}R^{9h}, —NR^{9g}R^{9h}, —NR^{9g}COR^{9h} or —NR^{9g}CO₂R^{9h}.

R^{9g} and R^{9h} are each independently —H, —F, —Cl, —Br, —I, —CH₃, —C₂H₅, —C₃H₇, —C₄H₉, —C₅H₁₁, —C₆H₁₃, —C₇H₁₅, —C₈H₁₇ or —OH.

Aspect 17. The compound of Aspect 1 or 15, wherein
R⁹, R¹⁰, R¹¹ and R¹² are each independently —H, —F, —Cl, —Br, —I, —CH₃, —C₂H₅, —C₃H₇, —C₄H₉, —C₅H₁₁, —C₆H₁₃, —C₇H₁₅, —C₈H₁₇, —CN, —OCH₃, —OC₂H₅, —OC₃H₇, —OC₄H₉, —OC₅H₁₁, —OC₆H₁₃, —OC₇H₁₅ or —OC₈H₁₇; or
(R⁹ and R¹¹) or (R¹⁰ and R¹²) together with the atom(s) to which they are attached, form a 5-, 6- or 7-membered ring, said ring comprising 0, 1 or 2 additional heteroatom(s) independently selected from the group consisting of nitrogen or oxygen, said ring is optionally substituted with at least one substituent R^{9f};
R^{9f}, at each of its occurrence, is independently —H, —F, —Cl, —Br, —I, —CH₃, —C₂H₅, —C₃H₇, —C₄H₉, —C₅H₁₁, —C₆H₁₃, —C₇H₁₅, —C₈H₁₇, —CN, —OCH₃, —OC₂H₅, —OC₃H₇, —OC₄H₉, —OC₅H₁₁, —OC₆H₁₃, —OC₇H₁₅ or —OC₈H₁₇; or
two R^{9f} together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen or optionally oxidized sulfur as ring member(s), said ring is optionally substituted with at least one —F, —Cl, —Br, —I, —CH₃, —C₂H₅, —C₃H₇, —C₄H₉, —C₅H₁₁, —C₆H₁₃, —C₇H₁₅, —C₈H₁₇, —CN, —OCH₃, —OC₂H₅, —OC₃H₇, —OC₄H₉, —OC₅H₁₁, —OC₆H₁₃, —OC₇H₁₅ or —OC₈H₁₇.

Aspect 18. The compound of Aspect 1 or 15, wherein

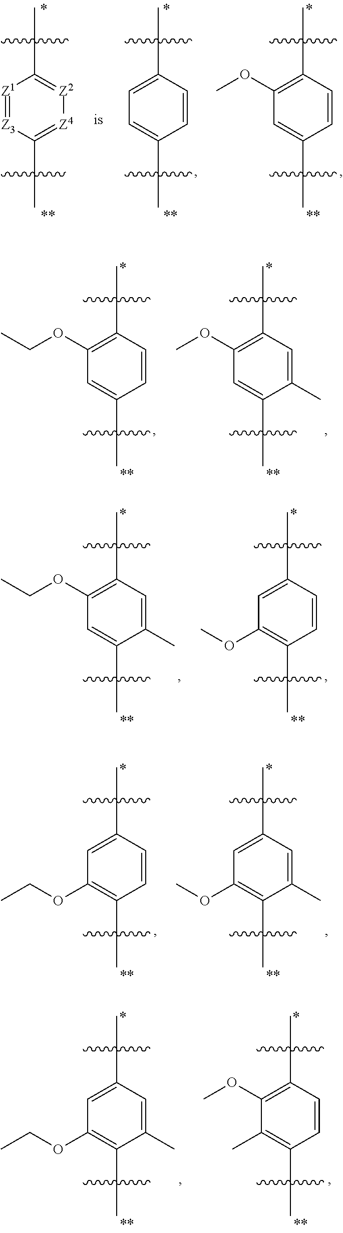

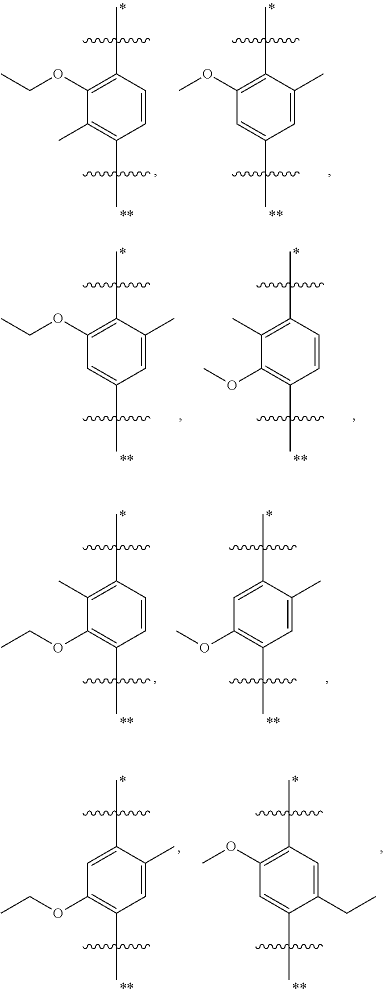

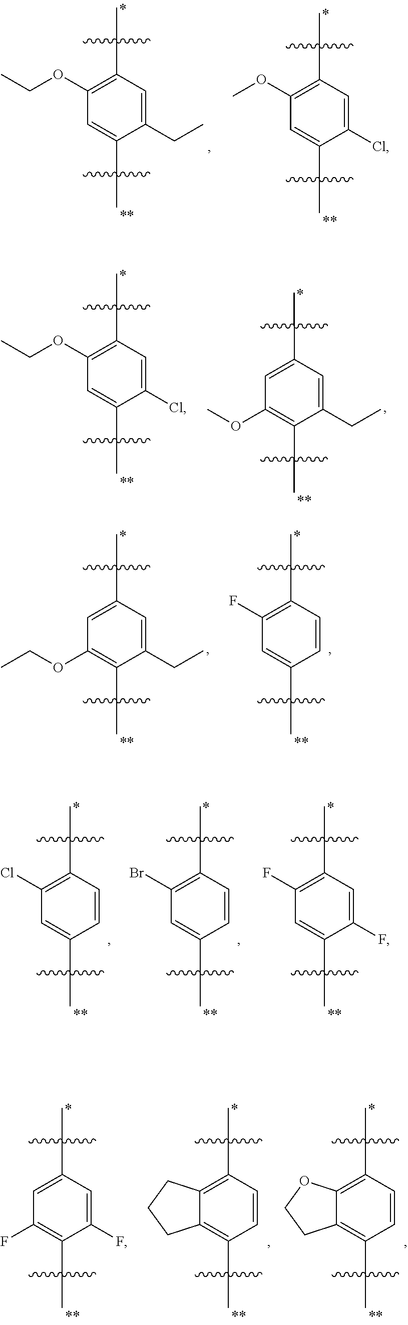

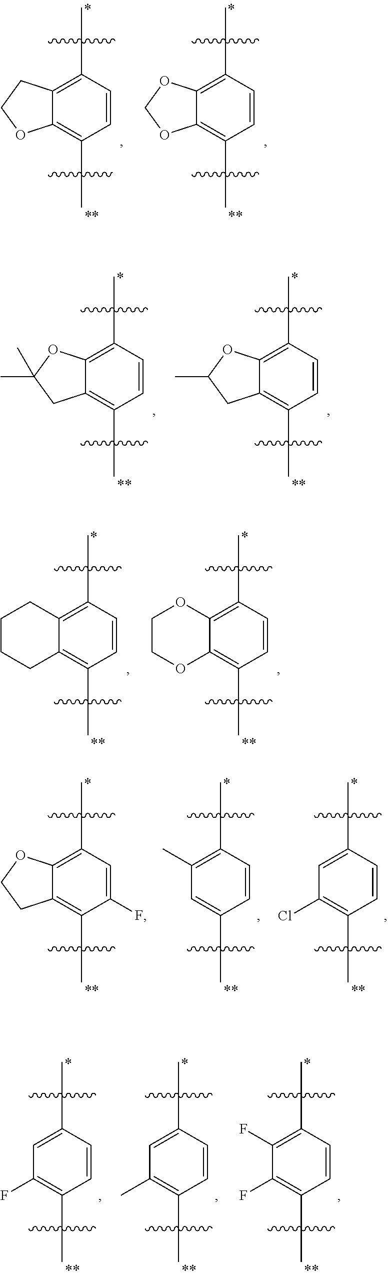

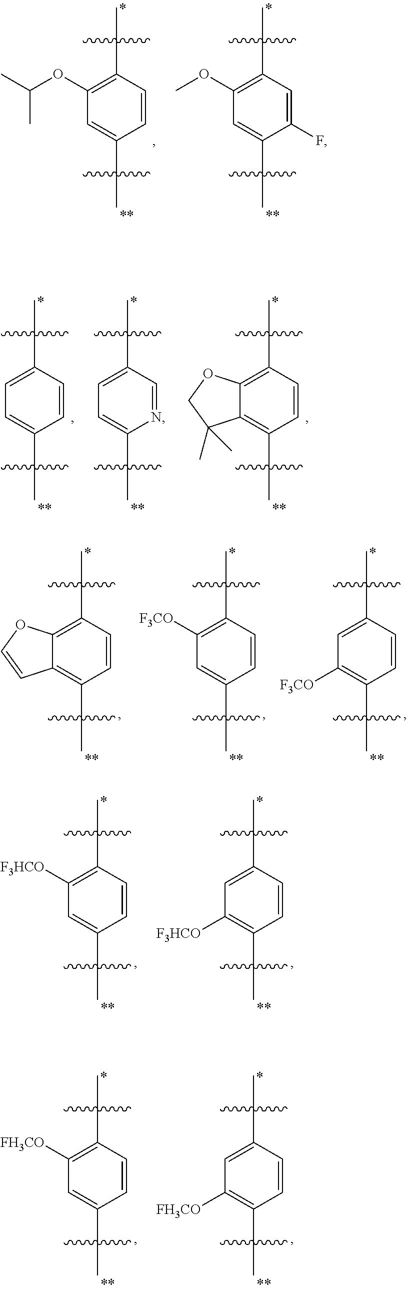

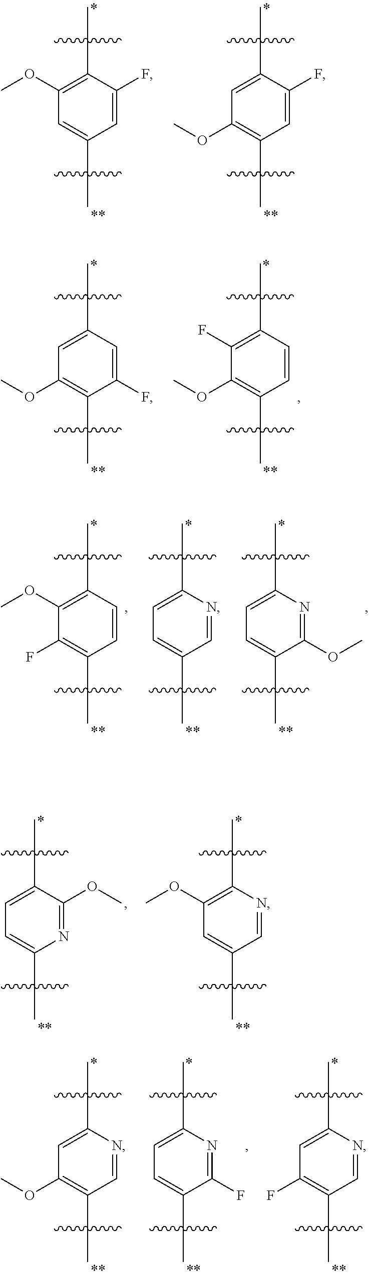

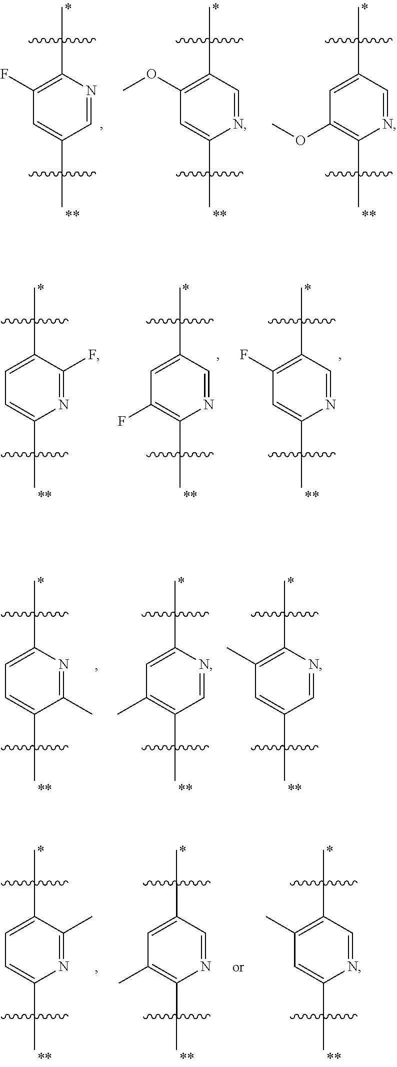

wherein refers to the position linked to the —N(R⁷)— moiety, and refers to the position linked to R⁸.

Aspect 19. The compound of Aspect 1, wherein
R⁸ is —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 4- to 8-membered monocyclic heterocyclyl comprising 1 or 2 nitrogen atoms as the ring member(s), spiro heterocyclyl selected from the group consisting of azaspiro[5.5]undecanyl, diazaspiro[5.5]undecanyl, azaspiro[4.5]decanyl, diazaspiro[4.5]decanyl, azaspiro[3.5]nonanyl, diazaspiro[3.5]nonanyl, azaspiro[4.4]nonanyl, diazaspiro[4.4]nonanyl, azaspiro[3.4]octanyl, diazaspiro[3.4]octanyl, azaspiro[3.3]heptanyl or diazaspiro[3.3]heptanyl (Preferably 3,9-diazaspiro[5.5]undecan-9-yl, 2,7-diazaspiro[3.5]nonan-7-yl, 2,8-diazaspiro[4.5]decan-8yl or 2,6-diazaspiro[3.3]heptan-6-yl), bridged heterocyclyl selected from the group consisting of azabicyclo[2.2.1]heptanyl, diazabicyclo[2.2.1]heptanyl, azabicyclo[3.1.1]heptanyl, diazabicyclo[3.1.1]heptanyl, azabicyclo[2.2.2]octanyl, diazabicyclo[2.2.2]octanyl, azabicyclo[3.2.1]octanyl or diazabicyclo[3.2.1]octanyl (Preferably 2-azabicyclo[2.2.1]heptan-2-yl, 6-azabicyclo[3.1.1]heptan-3-yl, 2-azabicyclo[2.2.2]octan-5-yl, 3-azabicyclo[3.2.1]octan-8-yl), 5-to 8-membered heteroaryl, —OR^{8a} or —NR^{8a}R^{8b}, wherein each of said methyl, ethyl, propyl, butyl, pentyl, hexyl, heptyl, octyl, cyclobutyl, cyclopentyl, cyclohexyl, cycloheptyl, cyclooctyl, 4- to 8-membered monocyclic heterocyclyl, spiro heterocyclyl, or heteroaryl is optionally substituted with at least one substituent R^{8c};
R^{8a} and R^{8b} are each independently hydrogen, methyl, ethyl, propyl, butyl, pentyl, hexyl, heptyl, octyl, cyclobutyl, cyclopentyl, cyclohexyl, 4- to 8-membered heterocyclyl, 5- to 8-membered heteroaryl, C₁₋₈alkoxy-C₁₋₈alkyl-, phenyl or 5- to 8-membered heteroaryl, wherein each of said methyl, ethyl, propyl, butyl, pentyl, hexyl, heptyl, octyl, cyclobutyl, cyclopentyl, cyclohexyl, 4- to 8-membered heterocyclyl, 5- to 8-membered heteroaryl, C₁₋₈alkoxy-C₁₋₈alkyl-, phenyl or 5- to 8-membered heteroaryl is optionally substituted with at least one substituent R^{8d}; or
R^{8a} and R^{8b} together with the atom(s) to which they are attached, form a 3-, 4-, 5-, 6-, 7- or 8-membered ring, said ring comprising 0, 1 or 2 additional heteroatom(s) independently selected from the group consisting of nitrogen or oxygen as ring member(s), said ring is optionally substituted with at least one substituent R^{8d};
R^{8c} is independently hydrogen, —F, —Cl, —Br, —I, methyl, ethyl, propyl, butyl, pentyl, hexyl, heptyl, octyl, cyclobutyl, cyclopentyl, cyclohexyl, phenyl —C₂₋₈alkenyl, —C₂₋₈alkynyl, 3- to 12-membered heterocyclyl, 5- to 8-membered heteroaryl, oxo (═O), —CN, —OCH₃, —OC₂H₅, —OC₃H₇, —OC₄H₉, —OC₅H₁₁, —OC₆H₁₃, —OC₇H₁₅, —OC₈H₁₇, —COCH₃, —COC₂H₅, —COC₃H₇, —COC₄H₉, —COC₅H₁₁, —COC₆H₁₃, —COC₇H₁₅, —COC₈H₁₇, —CONR^{8f}R^{8g}, —NR^{8f}R^{8g} or —NR^{8f} COR^{8g}, wherein each of said methyl, ethyl, propyl, butyl, pentyl, hexyl, heptyl, octyl, cyclobutyl, cyclopentyl, cyclohexyl, phenyl —C₂₋₈alkenyl, —C₂₋₈alkynyl, 3- to 12-membered heterocyclyl, 5- to 8-membered heteroaryl is optionally substituted with at least one R^{8e}; or
two R^{8c} together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen or optionally oxidized sulfur as ring member(s), said ring is optionally substituted with at least one R^{8e};
R^{8d}, and R^{8e} are each independently hydrogen, —F, —Cl, —Br, —I, methyl, ethyl, propyl, butyl, pentyl, hexyl, heptyl, octyl, cyclobutyl, cyclopentyl, cyclohexyl, —C₂₋₈alkenyl, —C₂₋₈alkynyl, 3- to 8-membered heterocyclyl, phenyl, 5- to 8-membered heteroaryl, oxo (═O), —CN, —OH, —OCH₃, —OC₂H₅, —OC₃H₇, —OC₄H₉, —OC₅H₁₁, —OC₆H₁₃, —OC₇H₁₅, —OC₈H₁₇, —COCH₃, —COC₂H₅, —COC₃H₇, —COC₄H₉, —COC₅H₁₁, —COC₆H₁₃, —COC₇H₁₅, —COC₈H₁₇, —CO₂CH₃, —CO₂C₂H₅, —CO₂C₃H₇, —CO₂C₄H₉, —CO₂C₅H₁₁, —CO₂C₆H₁₃, —CO₂C₇H₁₅, —CO₂C₈H₁₇, —CONR^{8h}R⁸ⁱ, —NR^{8h}R⁸ⁱ or —NR^{8h}COR⁸ⁱ, wherein each of said methyl, ethyl, propyl, butyl, pentyl, hexyl, heptyl, octyl, cyclobutyl, cyclopentyl, cyclohexyl, —C₂₋₈alkenyl, —C₂₋₈alkynyl, 3- to 8-membered heterocyclyl, phenyl, 5- to 8-membered heteroaryl is optionally substituted with at least one —F, —Cl, —Br, —I, methyl, ethyl, propyl, butyl, pentyl, hexyl, heptyl, octyl, cyclobutyl, cyclopentyl, cyclohexyl, —C₂₋₈alkenyl, —C₂₋₈alkynyl, 3- to 8-membered heterocyclyl, phenyl, 5- to 8-membered heteroaryl, phenyl, hydroxyl, —OCH₃, —OC₂H₅, —OC₃H₇, —OC₄H₉, —OC₅H₁₁, —OC₆H₁₃, —OC₇H₁₅, —OC₈H₁₇ or C₁₋₈alkoxy-C₁₋₈alkyl-;
R^{8f}, R^{8g}, R^{8h} and R⁸ⁱ are each independently hydrogen, methyl, ethyl, propyl, butyl, pentyl, hexyl, heptyl, octyl, cyclobutyl, cyclopentyl, cyclohexyl, C₁₋₈alkoxy-C₁₋₈alkyl-, —C₂₋₈alkenyl, —C₂₋₈alkynyl, 3- to 8-membered heterocyclyl, phenyl or 5- to 8-membered heteroaryl.

Aspect 20. The compound of Aspect 1, wherein
R⁸ is F, Cl, Br, methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl, pyrazolyl, —OR^{8a} or —NR^{8a}R^{8b}, wherein each of said methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl, pyrazolyl is optionally substituted with at least one substituent R^{8c};
R^{8a} and R^{8b} are each independently methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl or pyrazolyl, wherein each of said methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl or pyrazolyl is optionally substituted with at least one substituent R^{8d}; or
R^{8a} and R^{8b} together with the atom(s) to which they are attached, form a 4-, 5-, 6-, 7- or 8-membered ring, said ring comprising 0, 1 or 2 additional heteroatom(s) independently selected from the group consisting of nitrogen or oxygen as ring member(s), said ring is optionally substituted with at least one substituent R^{8d};
R^{8c} is independently hydrogen, F, Cl, Br, methyl, ethyl, propyl, butyl, pentyl, oxo (═O), azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl, pyrazolyl, —CN, —OR^{8f} or —NR^{8f}R⁸⁹, wherein each of said methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl, pyrazolyl is optionally substituted with at least one R^{8e}; or
two R^{8c} together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen or optionally oxidized sulfur as ring member(s), said ring is optionally substituted with at least one R^{8e};
R^{8d} and R^{8e} are each independently hydrogen, F, Cl, Br, methyl, ethyl, propyl, butyl, pentyl, oxo (═O), azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl, pyrazolyl, —CN, —OR^{8h} or —NR^{8h}R⁸ⁱ, wherein each of said methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl, pyrazolyl is optionally substituted with at least one F, Cl, Br, methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl or pyrazolyl; or
two R^{8e} together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen or optionally oxidized sulfur as ring member(s), said ring is optionally substituted with at least one F, Cl, Br, methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl, pyrazolyl or —CN;
R^{8f}, R^{8g}, R^{8h} and R⁸ⁱ are each independently hydrogen, methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl or pyrazolyl.

Aspect 21. The compound of Aspect 1, wherein R⁸ is

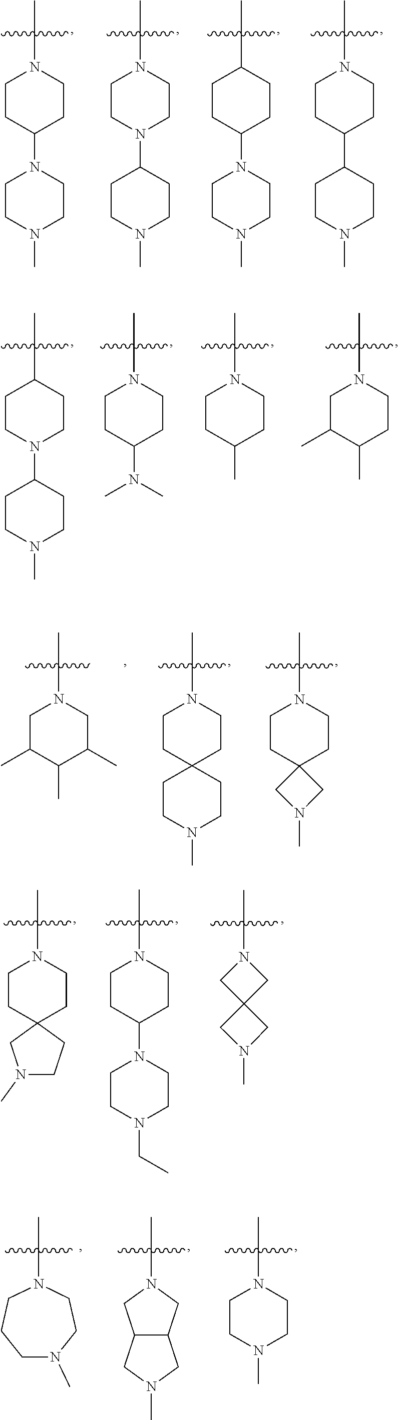

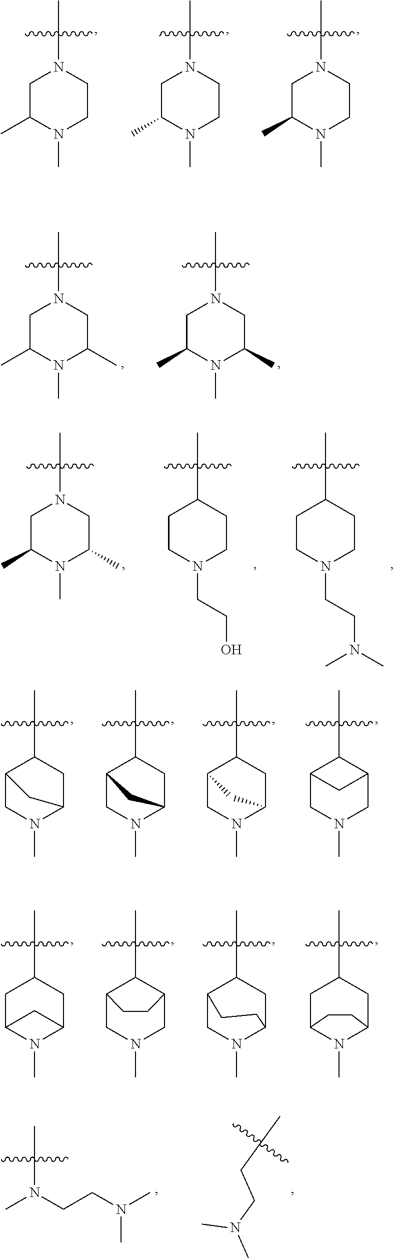

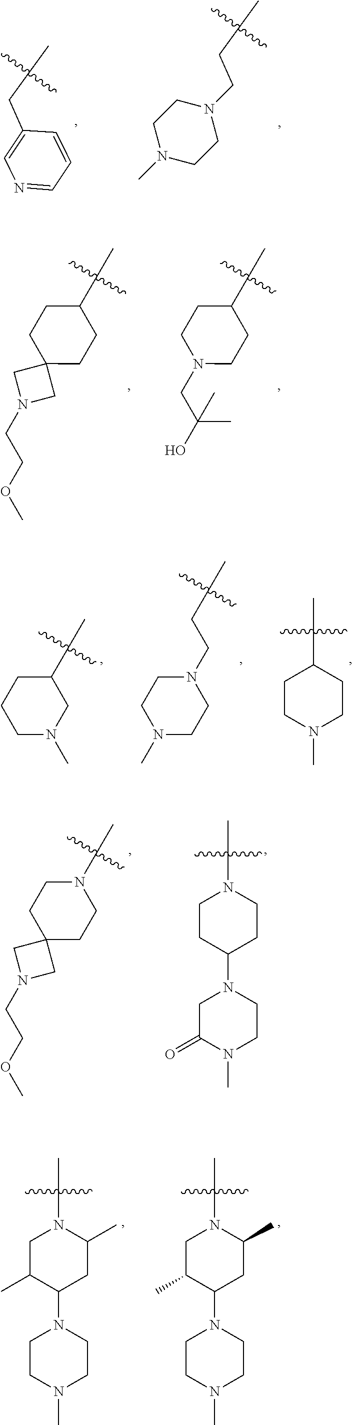

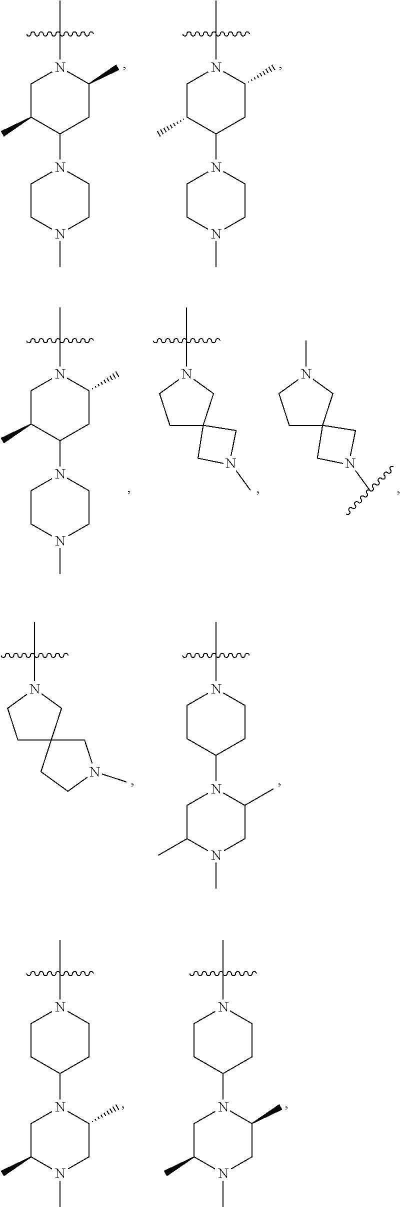

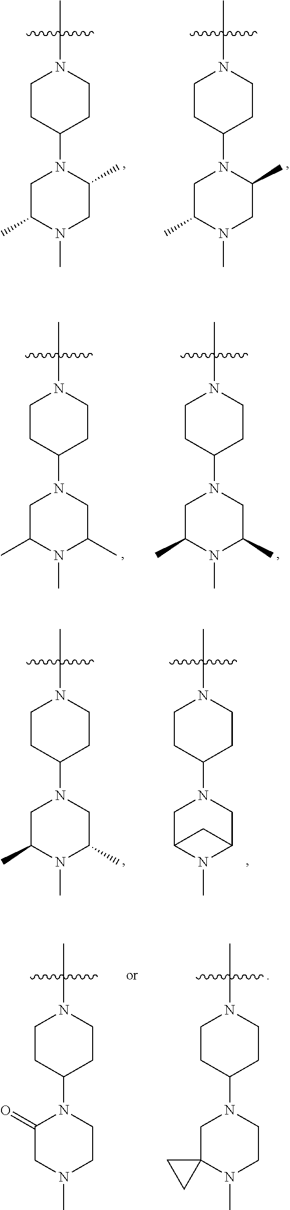

Aspect 22. The compound of Aspect 1, the compound is

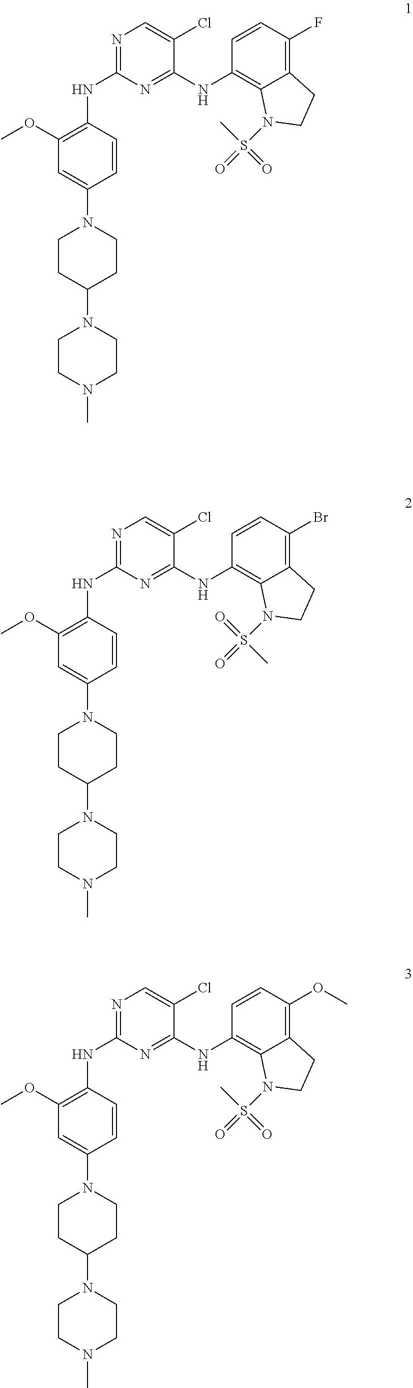

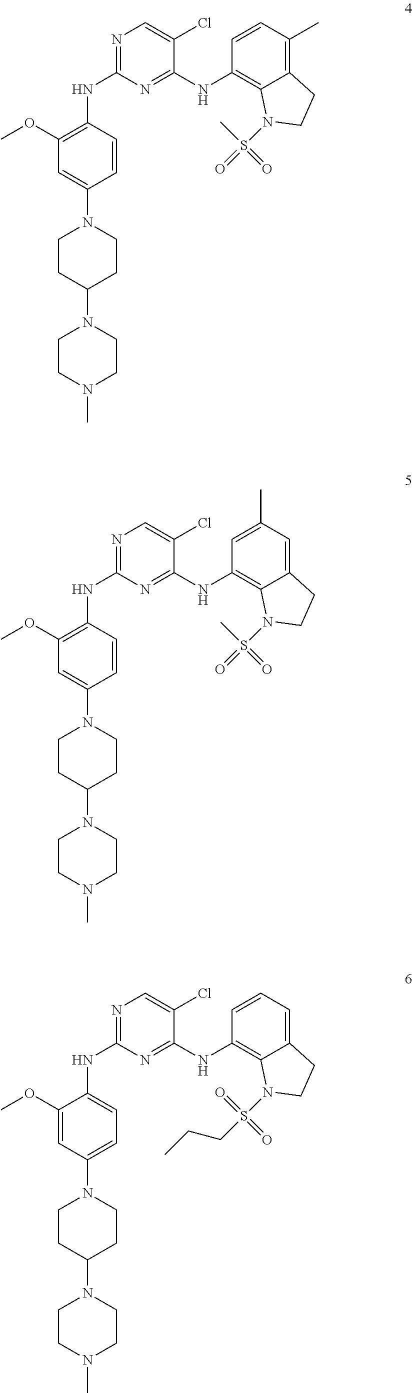

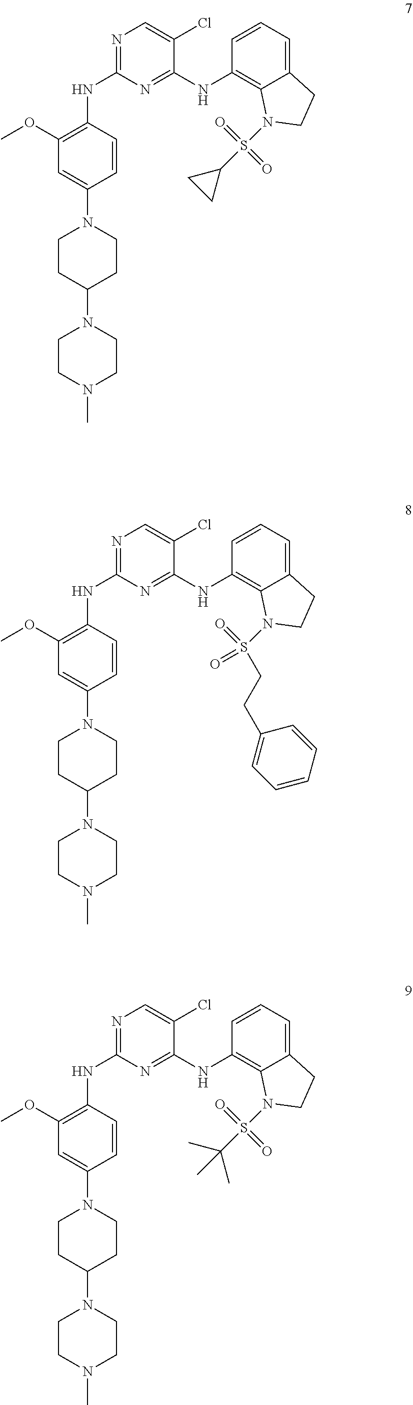 [SMILES: COc1cc(N2CCC(N3CCN(C)CC3)CC2)ccc1Nc1ncc(Cl)c(Nc2cccc3c2N(S(=O)(=O)C2CC2)CC3)n1 ; COc1cc(N2CCC(N3CCN(C)CC3)CC2)ccc1Nc1ncc(Cl)c(Nc2cccc3c2N(S(=O)(=O)CCc2ccccc2)CC3)n1 ; COc1cc(N2CCC(N3CCN(C)CC3)CC2)ccc1Nc1ncc(Cl)c(Nc2cccc3c2N(S(=O)(=O)C(C)(C)C)CC3)n1]

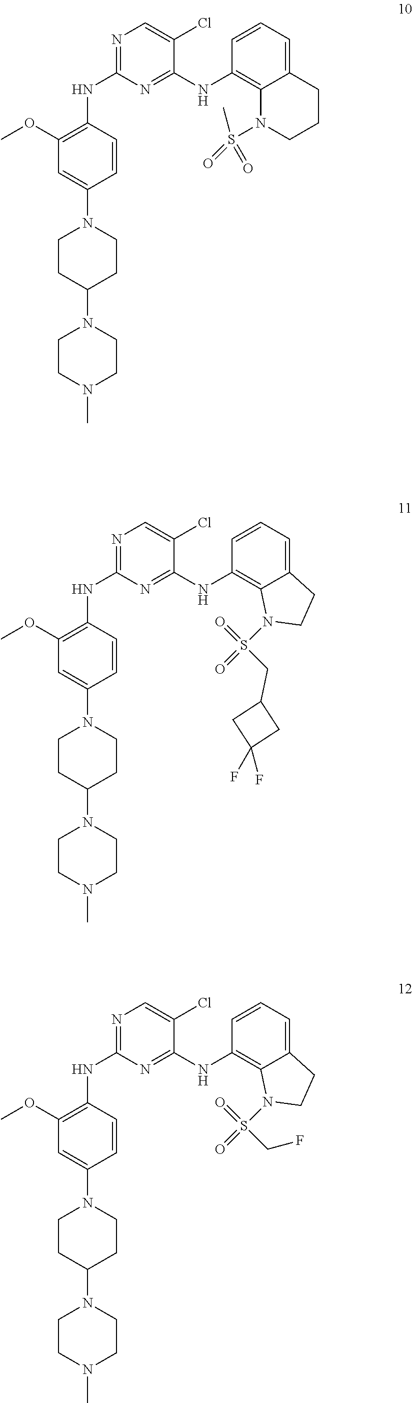 [SMILES: COc1cc(N2CCC(N3CCN(C)CC3)CC2)ccc1Nc1ncc(Cl)c(Nc2cccc3c2N(S(C)(=O)=O)CCC3)n1 ; COc1cc(N2CCC(N3CCN(C)CC3)CC2)ccc1Nc1ncc(Cl)c(Nc2cccc3c2N(S(=O)(=O)CC2CC(F)(F)C2)CC3)n1 ; COc1cc(N2CCC(N3CCN(C)CC3)CC2)ccc1Nc1ncc(Cl)c(Nc2cccc3c2N(S(=O)(=O)CF)CC3)n1]

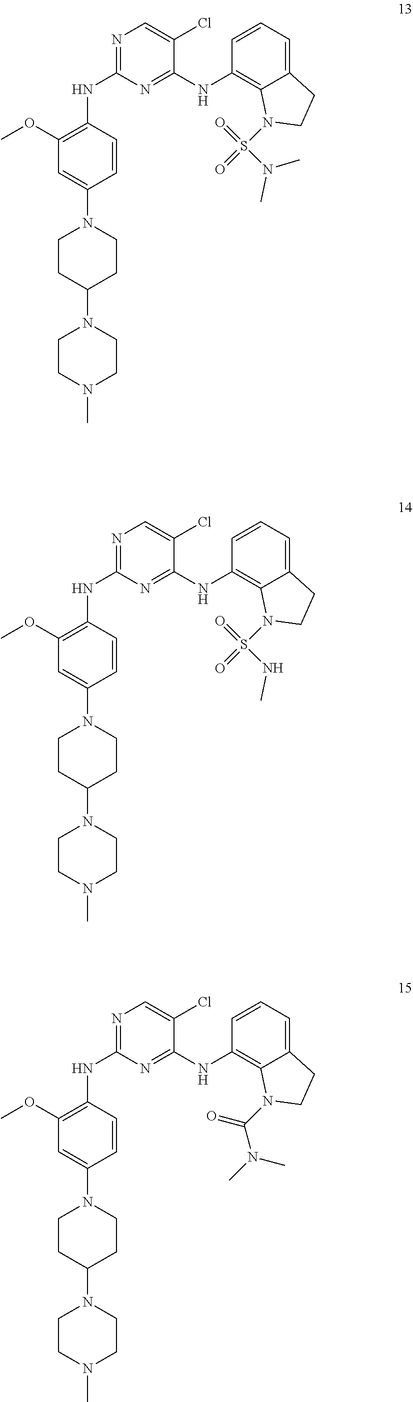 [SMILES: COc1cc(N2CCC(N3CCN(C)CC3)CC2)ccc1Nc1ncc(Cl)c(Nc2cccc3c2N(S(=O)(=O)N(C)C)CC3)n1 ; CNS(=O)(=O)N1CCc2cccc(Nc3nc(Nc4ccc(N5CCC(N6CCN(C)CC6)CC5)cc4OC)ncc3Cl)c21 ; COc1cc(N2CCC(N3CCN(C)CC3)CC2)ccc1Nc1ncc(Cl)c(Nc2cccc3c2N(C(=O)N(C)C)CC3)n1]

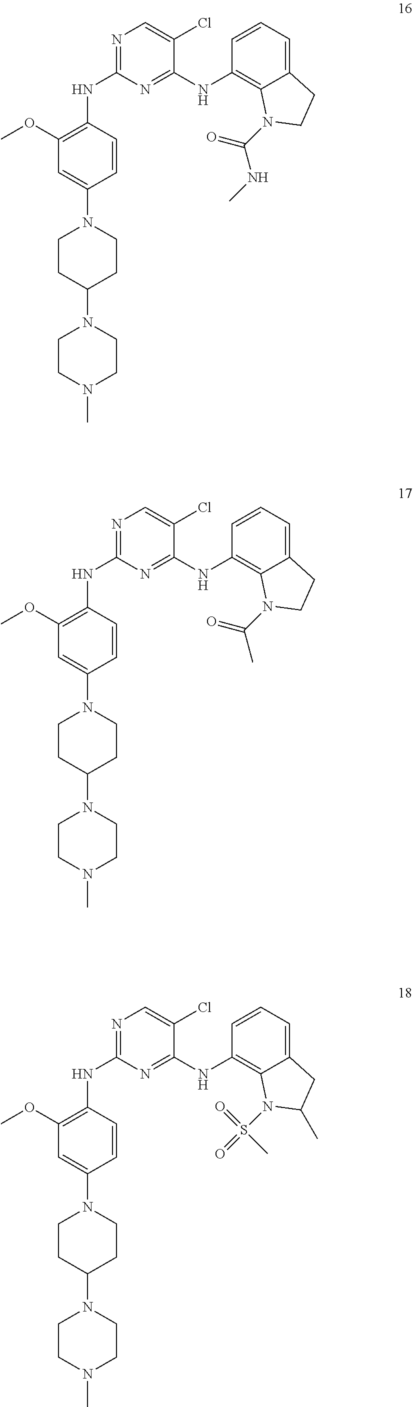

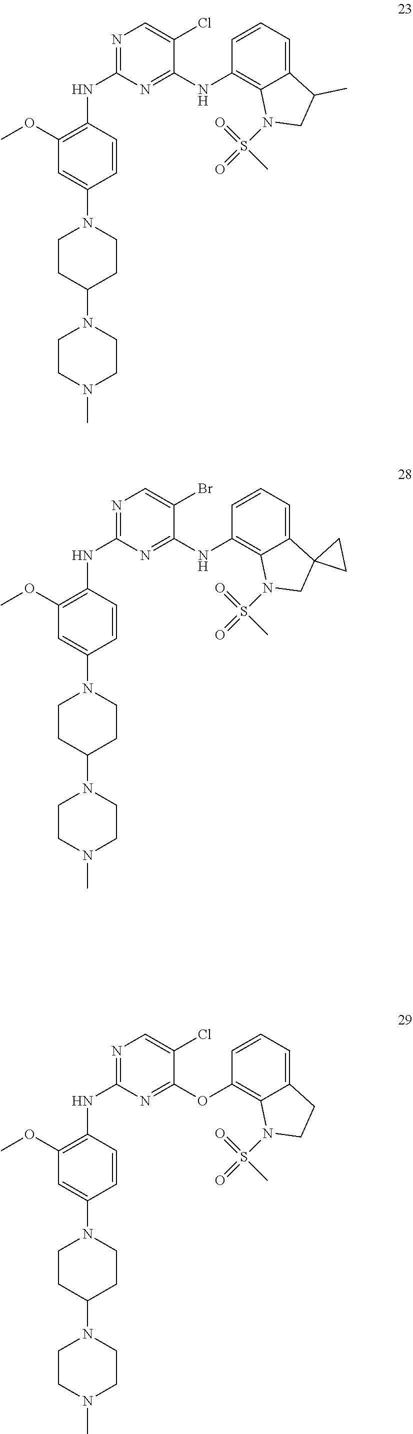

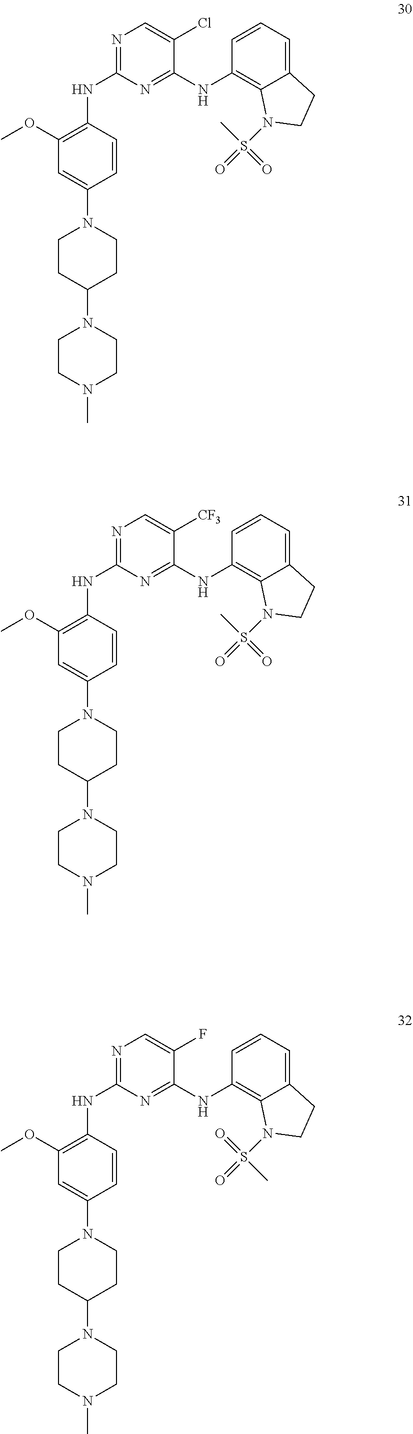 [SMILES: COc1cc(N2CCC(N3CCN(C)CC3)CC2)ccc1Nc1ncc(Cl)c(Nc2cccc3c2N(S(C)(=O)=O)CC3)n1 ; COc1cc(N2CCC(N3CCN(C)CC3)CC2)ccc1Nc1ncc(C(F)(F)F)c(Nc2cccc3c2N(S(C)(=O)=O)CC3)n1 ; COc1cc(N2CCC(N3CCN(C)CC3)CC2)ccc1Nc1ncc(F)c(Nc2cccc3c2N(S(C)(=O)=O)CC3)n1]

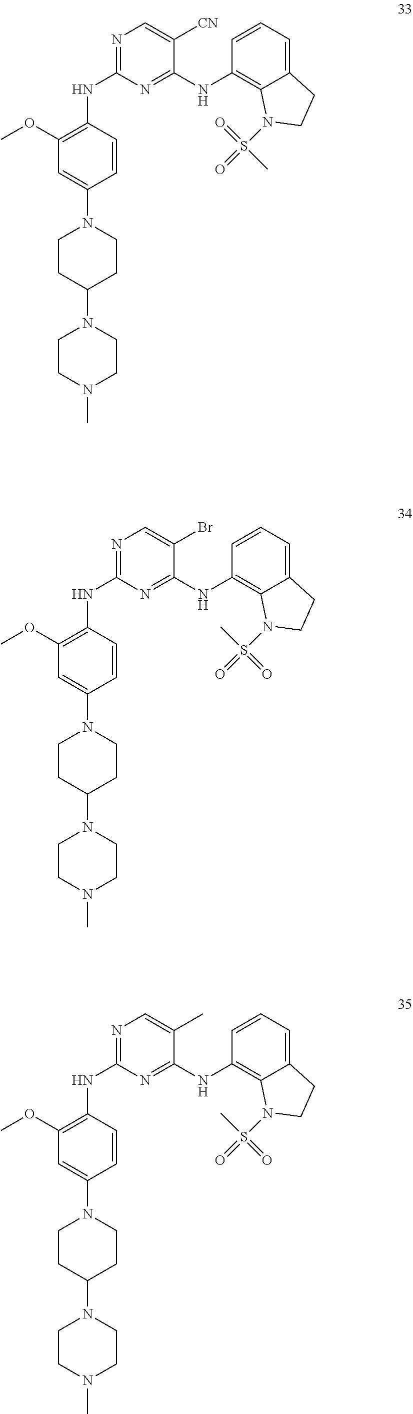 [SMILES: COc1cc(N2CCC(N3CCN(C)CC3)CC2)ccc1Nc1ncc(C#N)c(Nc2cccc3c2N(S(C)(=O)=O)CC3)n1 ; COc1cc(N2CCC(N3CCN(C)CC3)CC2)ccc1Nc1ncc(Br)c(Nc2cccc3c2N(S(C)(=O)=O)CC3)n1 ; COc1cc(N2CCC(N3CCN(C)CC3)CC2)ccc1Nc1ncc(C)c(Nc2cccc3c2N(S(C)(=O)=O)CC3)n1]

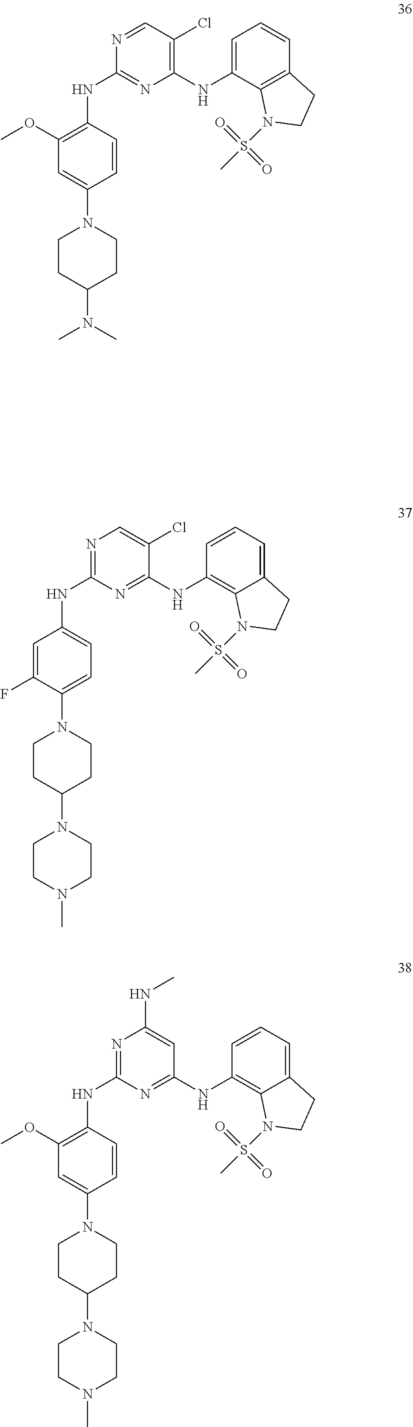 [SMILES: COc1cc(N2CCC(N(C)C)CC2)ccc1Nc1ncc(Cl)c(Nc2cccc3c2N(S(C)(=O)=O)CC3)n1 ; CN1CCN(C2CCN(c3ccc(Nc4ncc(Cl)c(Nc5cccc6c5N(S(C)(=O)=O)CC6)n4)cc3F)CC2)CC1 ; CNc1cc(Nc2cccc3c2N(S(C)(=O)=O)CC3)nc(Nc2ccc(N3CCC(N4CCN(C)CC4)CC3)cc2OC)n1]

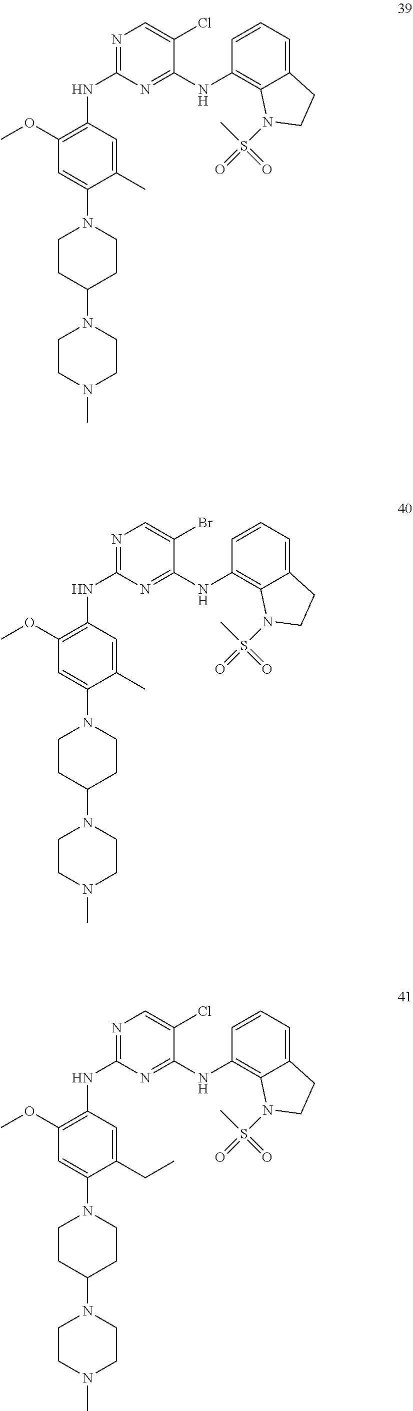 [SMILES: COc1cc(N2CCC(N3CCN(C)CC3)CC2)c(C)cc1Nc1ncc(Cl)c(Nc2cccc3c2N(S(C)(=O)=O)CC3)n1 ; COc1cc(N2CCC(N3CCN(C)CC3)CC2)c(C)cc1Nc1ncc(Br)c(Nc2cccc3c2N(S(C)(=O)=O)CC3)n1 ; CCc1cc(Nc2ncc(Cl)c(Nc3cccc4c3N(S(C)(=O)=O)CC4)n2)c(OC)cc1N1CCC(N2CCN(C)CC2)CC1]

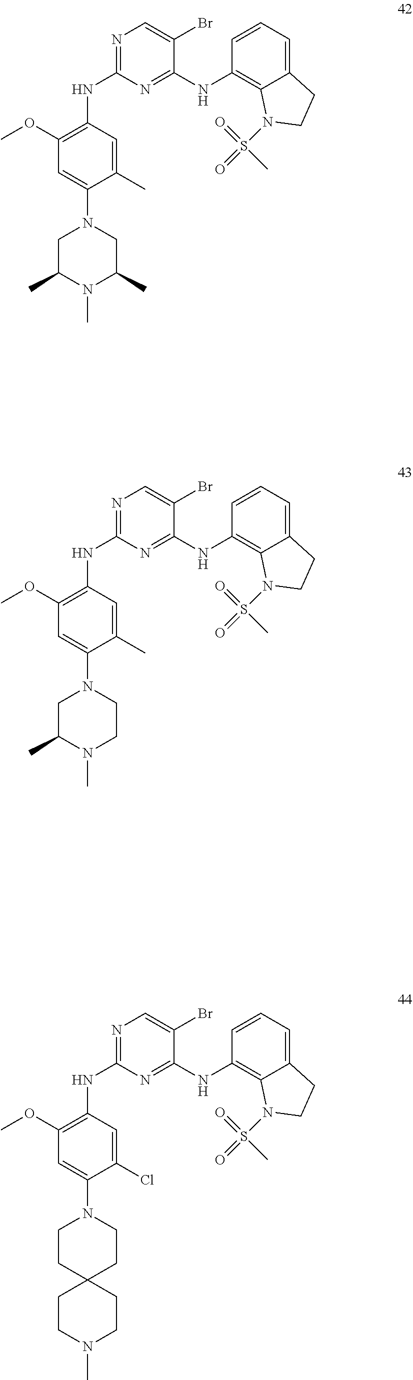 [SMILES: COc1cc(N2C[C@@H](C)N(C)[C@@H](C)C2)c(C)cc1Nc1ncc(Br)c(Nc2cccc3c2N(S(C)(=O)=O)CC3)n1 ; COc1cc(N2CCN(C)[C@@H](C)C2)c(C)cc1Nc1ncc(Br)c(Nc2cccc3c2N(S(C)(=O)=O)CC3)n1 ; COc1cc(N2CCC3(CCN(C)CC3)CC2)c(Cl)cc1Nc1ncc(Br)c(Nc2cccc3c2N(S(C)(=O)=O)CC3)n1]

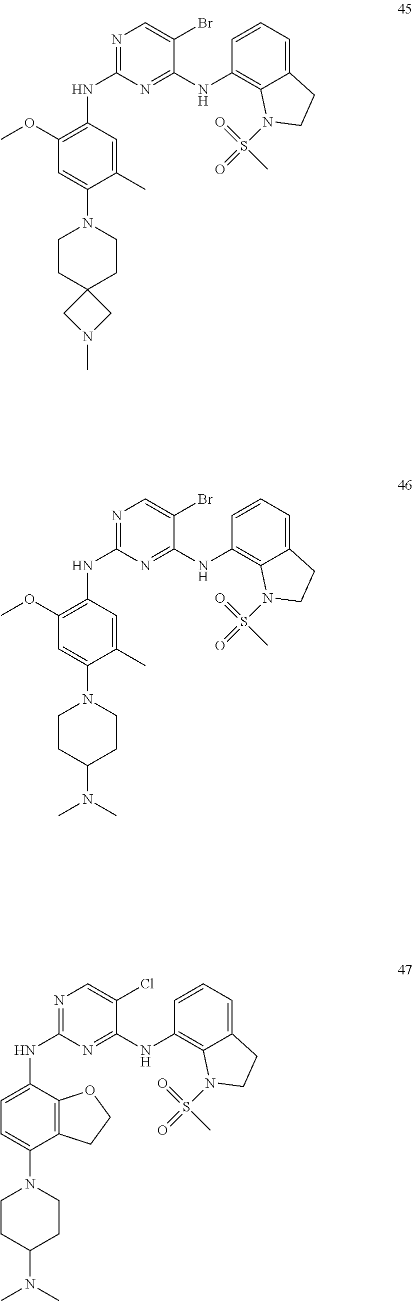 [SMILES: COc1cc(N2CCC3(CC2)CN(C)C3)c(C)cc1Nc1ncc(Br)c(Nc2cccc3c2N(S(C)(=O)=O)CC3)n1 ; COc1cc(N2CCC(N(C)C)CC2)c(C)cc1Nc1ncc(Br)c(Nc2cccc3c2N(S(C)(=O)=O)CC3)n1 ; CN(C)C1CCN(c2ccc(Nc3ncc(Cl)c(Nc4cccc5c4N(S(C)(=O)=O)CC5)n3)c3c2CCO3)CC1]

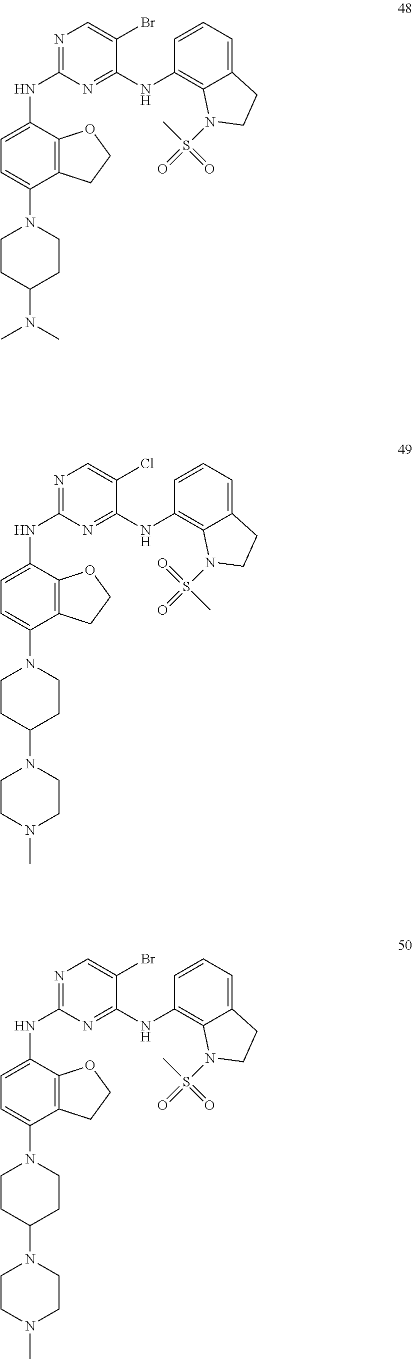 [SMILES: CN(C)C1CCN(c2ccc(Nc3ncc(Br)c(Nc4cccc5c4N(S(C)(=O)=O)CC5)n3)c3c2CCO3)CC1 ; CN1CCN(C2CCN(c3ccc(Nc4ncc(Cl)c(Nc5cccc6c5N(S(C)(=O)=O)CC6)n4)c4c3CCO4)CC2)CC1 ; CN1CCN(C2CCN(c3ccc(Nc4ncc(Br)c(Nc5cccc6c5N(S(C)(=O)=O)CC6)n4)c4c3CCO4)CC2)CC1]

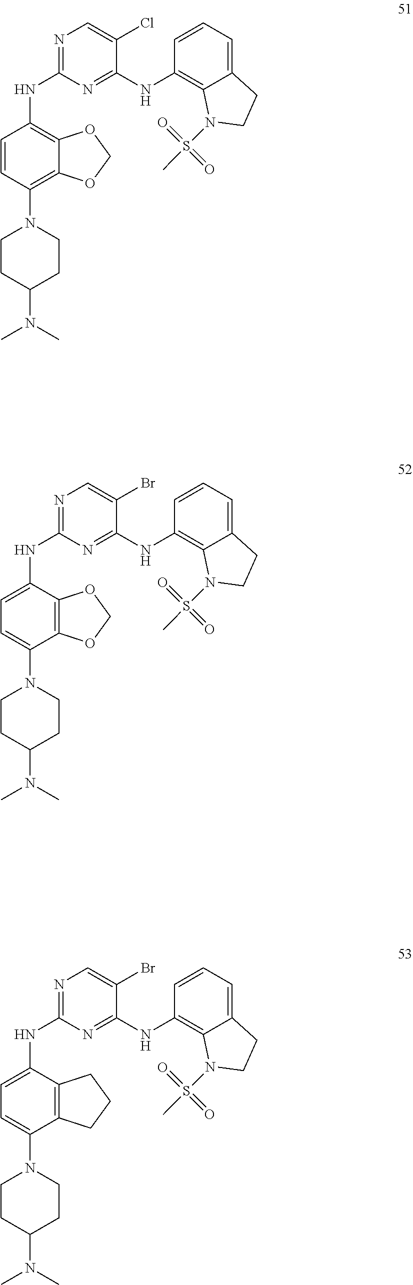 [SMILES: CN(C)C1CCN(c2ccc(Nc3ncc(Cl)c(Nc4cccc5c4N(S(C)(=O)=O)CC5)n3)c3c2OCO3)CC1 ; CN(C)C1CCN(c2ccc(Nc3ncc(Br)c(Nc4cccc5c4N(S(C)(=O)=O)CC5)n3)c3c2OCO3)CC1 ; CN(C)C1CCN(c2ccc(Nc3ncc(Br)c(Nc4cccc5c4N(S(C)(=O)=O)CC5)n3)c3c2CCC3)CC1]

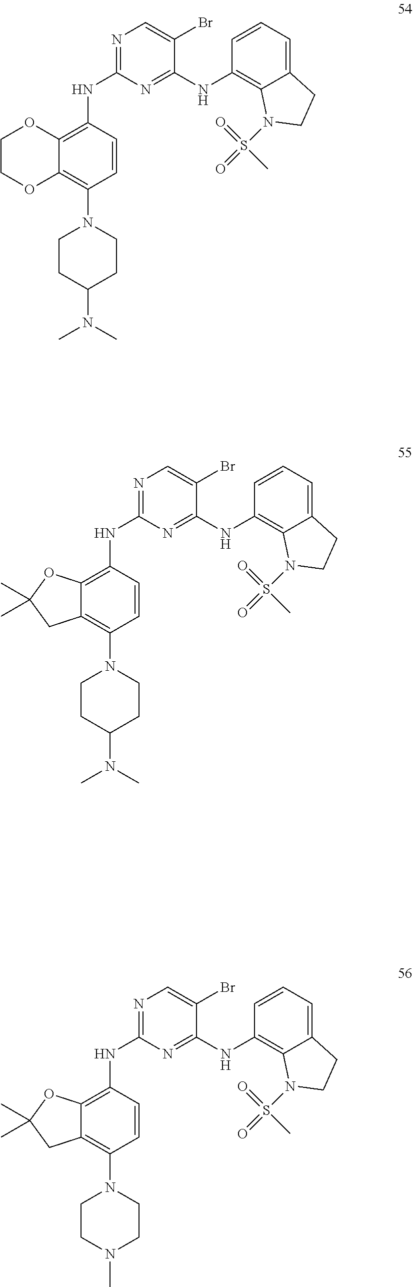 [SMILES: CN(C)C1CCN(c2ccc(Nc3ncc(Br)c(Nc4cccc5c4N(S(C)(=O)=O)CC5)n3)c3c2OCCO3)CC1 ; CN(C)C1CCN(c2ccc(Nc3ncc(Br)c(Nc4cccc5c4N(S(C)(=O)=O)CC5)n3)c3c2CC(C)(C)O3)CC1 ; CN1CCN(c2ccc(Nc3ncc(Br)c(Nc4cccc5c4N(S(C)(=O)=O)CC5)n3)c3c2CC(C)(C)O3)CC1]

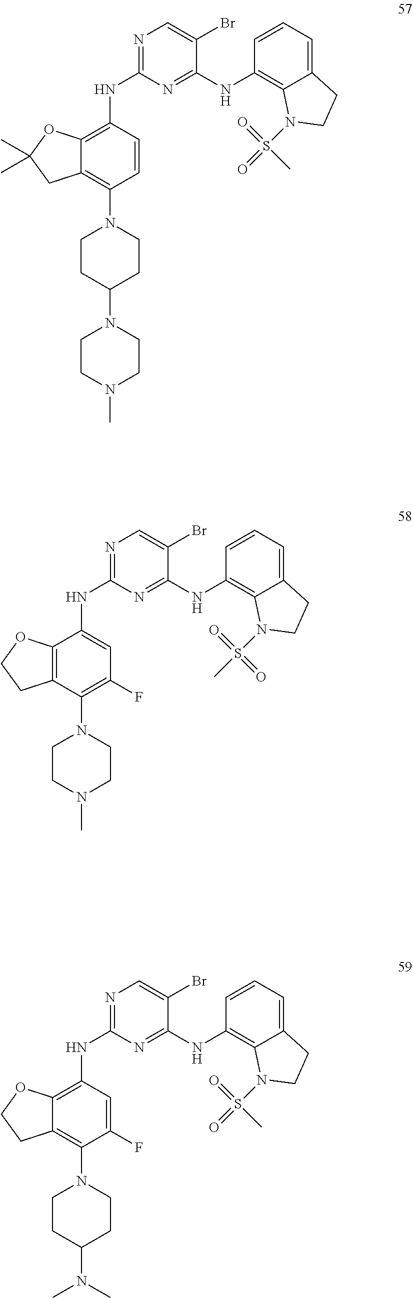 [SMILES: CN1CCN(C2CCN(c3ccc(Nc4ncc(Br)c(Nc5cccc6c5N(S(C)(=O)=O)CC6)n4)c4c3CC(C)(C)O4)CC2)CC1 ; CN1CCN(c2c(F)cc(Nc3ncc(Br)c(Nc4cccc5c4N(S(C)(=O)=O)CC5)n3)c3c2CCO3)CC1 ; CN(C)C1CCN(c2c(F)cc(Nc3ncc(Br)c(Nc4cccc5c4N(S(C)(=O)=O)CC5)n3)c3c2CCO3)CC1]

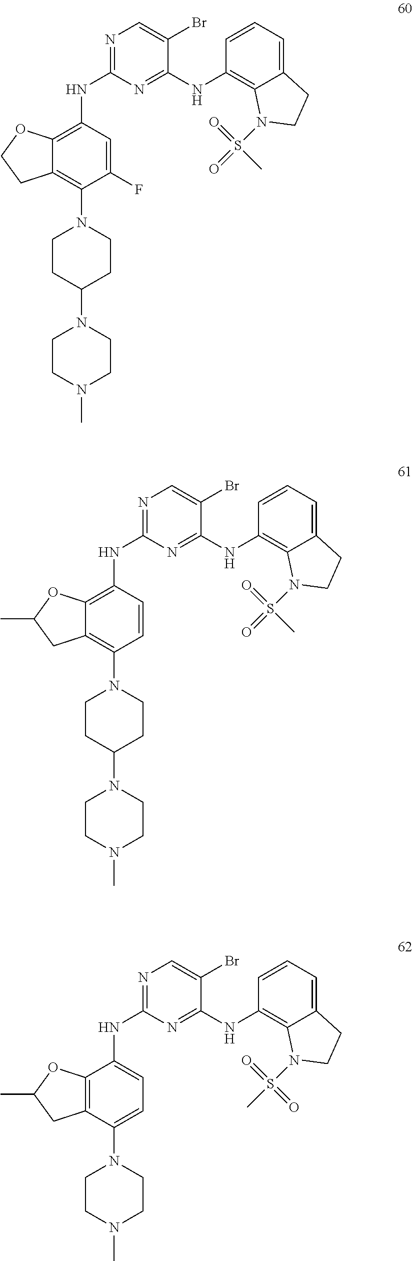 [SMILES: CN1CCN(C2CCN(c3c(F)cc(Nc4ncc(Br)c(Nc5cccc6c5N(S(C)(=O)=O)CC6)n4)c4c3CCO4)CC2)CC1 ; CC1Cc2c(N3CCC(N4CCN(C)CC4)CC3)ccc(Nc3ncc(Br)c(Nc4cccc5c4N(S(C)(=O)=O)CC5)n3)c2O1 ; CC1Cc2c(N3CCN(C)CC3)ccc(Nc3ncc(Br)c(Nc4cccc5c4N(S(C)(=O)=O)CC5)n3)c2O1]

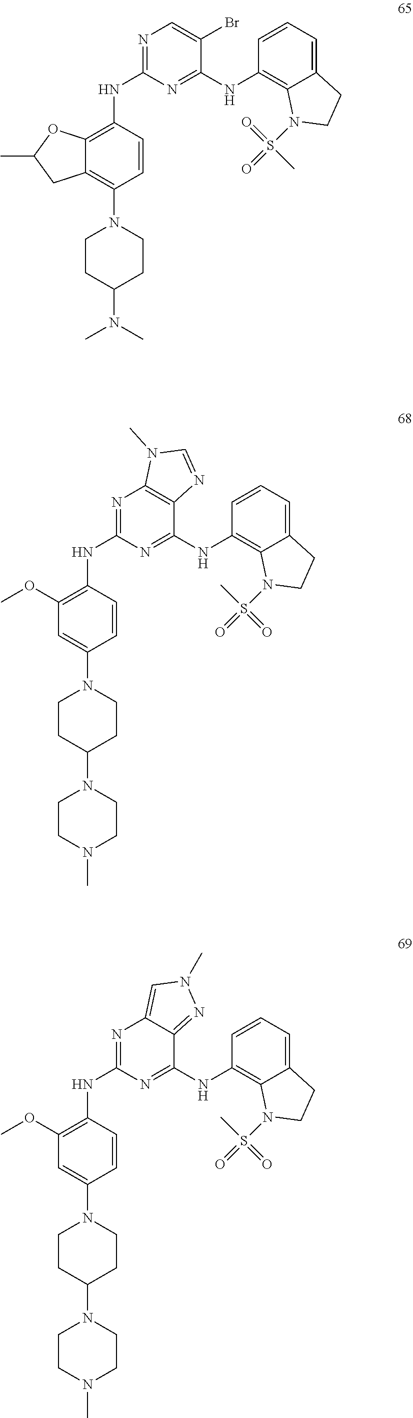 [SMILES: CC1Cc2c(N3CCC(N(C)C)CC3)ccc(Nc3ncc(Br)c(Nc4cccc5c4N(S(C)(=O)=O)CC5)n3)c2O1 ; COc1cc(N2CCC(N3CCN(C)CC3)CC2)ccc1Nc1nc(Nc2cccc3c2N(S(C)(=O)=O)CC3)c2ncn(C)c2n1 ; COc1cc(N2CCC(N3CCN(C)CC3)CC2)ccc1Nc1nc(Nc2cccc3c2N(S(C)(=O)=O)CC3)c2nn(C)cc2n1]

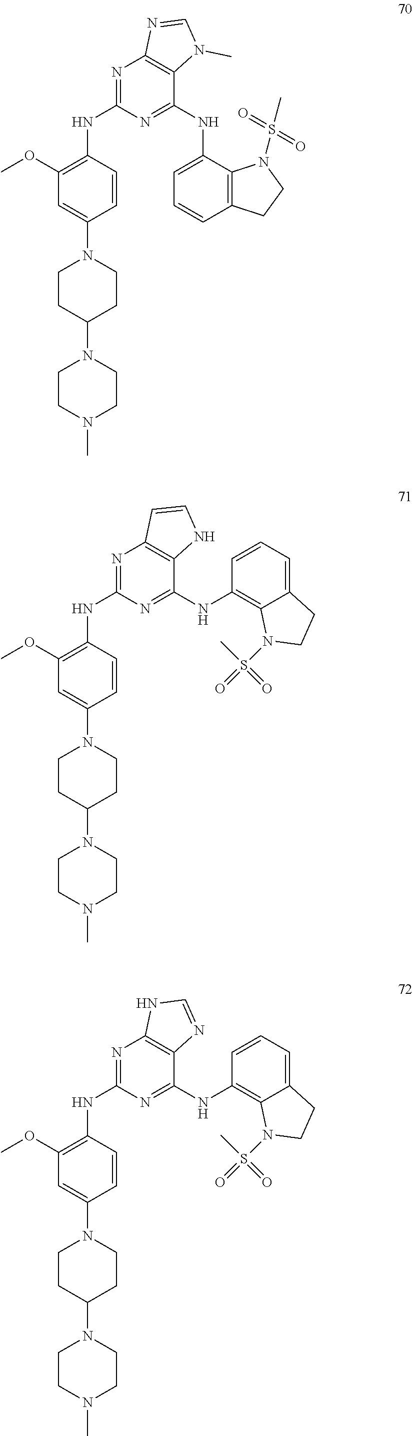 [SMILES: COc1cc(N2CCC(N3CCN(C)CC3)CC2)ccc1Nc1nc(Nc2cccc3c2N(S(C)(=O)=O)CC3)c2c(ncn2C)n1 ; COc1cc(N2CCC(N3CCN(C)CC3)CC2)ccc1Nc1nc(Nc2cccc3c2N(S(C)(=O)=O)CC3)c2[nH]ccc2n1 ; COc1cc(N2CCC(N3CCN(C)CC3)CC2)ccc1Nc1nc(Nc2cccc3c2N(S(C)(=O)=O)CC3)c2nc[nH]c2n1]

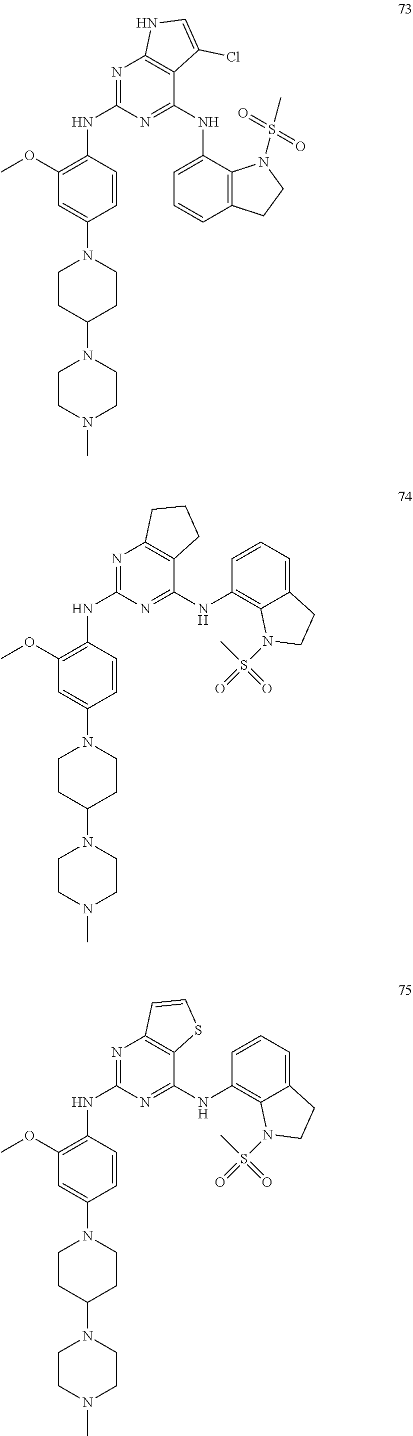 [SMILES: COc1cc(N2CCC(N3CCN(C)CC3)CC2)ccc1Nc1nc(Nc2cccc3c2N(S(C)(=O)=O)CC3)c2c(Cl)c[nH]c2n1 ; COc1cc(N2CCC(N3CCN(C)CC3)CC2)ccc1Nc1nc2c(c(Nc3cccc4c3N(S(C)(=O)=O)CC4)n1)CCC2 ; COc1cc(N2CCC(N3CCN(C)CC3)CC2)ccc1Nc1nc(Nc2cccc3c2N(S(C)(=O)=O)CC3)c2sccc2n1]

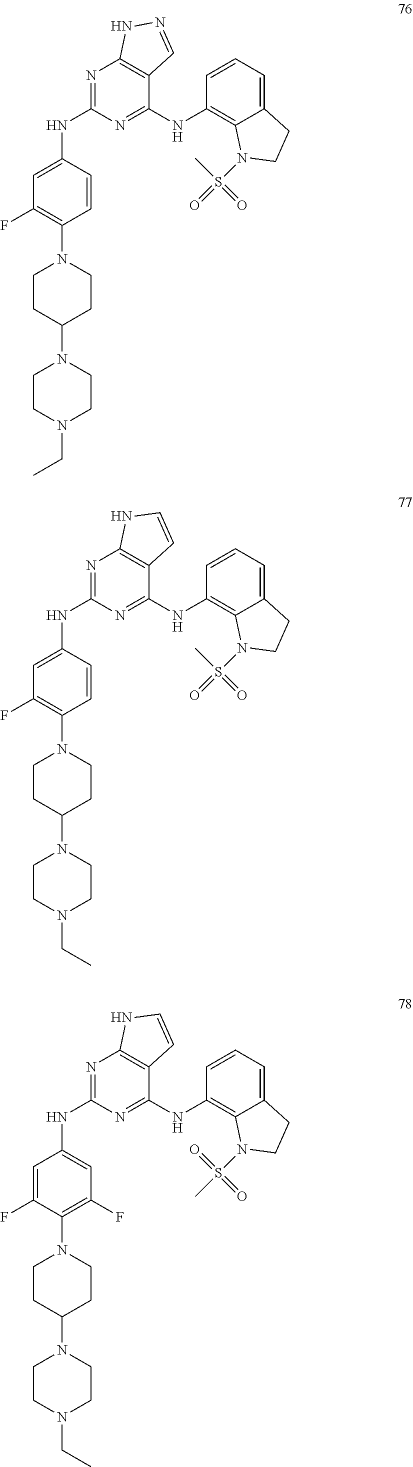 [SMILES: CCN1CCN(C2CCN(c3ccc(Nc4nc(Nc5cccc6c5N(S(C)(=O)=O)CC6)c5cn[nH]c5n4)cc3F)CC2)CC1 ; CCN1CCN(C2CCN(c3ccc(Nc4nc(Nc5cccc6c5N(S(C)(=O)=O)CC6)c5cc[nH]c5n4)cc3F)CC2)CC1 ; CCN1CCN(C2CCN(c3c(F)cc(Nc4nc(Nc5cccc6c5N(S(C)(=O)=O)CC6)c5cc[nH]c5n4)cc3F)CC2)CC1]

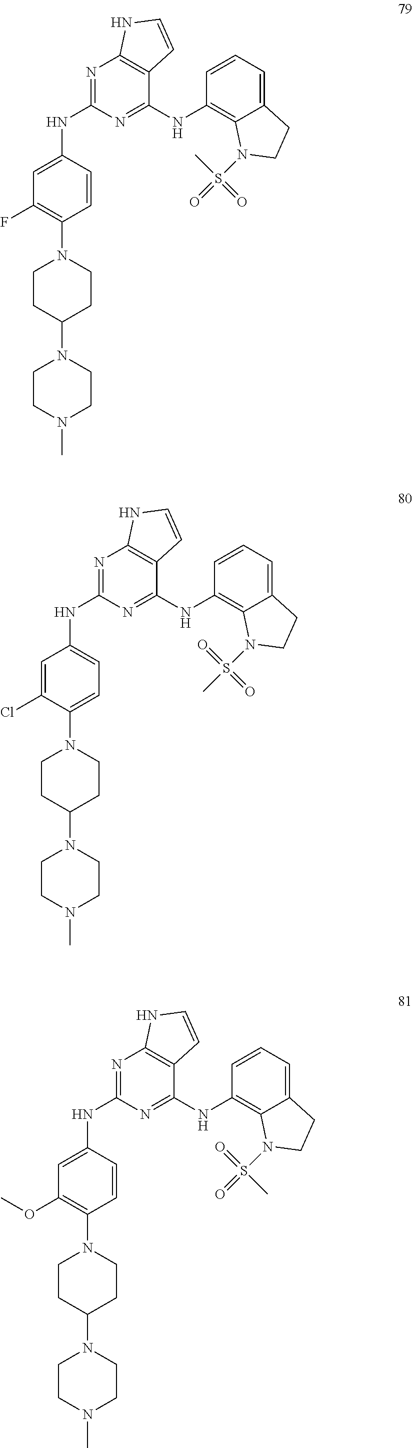 [SMILES: CN1CCN(C2CCN(c3ccc(Nc4nc(Nc5cccc6c5N(S(C)(=O)=O)CC6)c5cc[nH]c5n4)cc3F)CC2)CC1 ; CN1CCN(C2CCN(c3ccc(Nc4nc(Nc5cccc6c5N(S(C)(=O)=O)CC6)c5cc[nH]c5n4)cc3Cl)CC2)CC1 ; COc1cc(Nc2nc(Nc3cccc4c3N(S(C)(=O)=O)CC4)c3cc[nH]c3n2)ccc1N1CCC(N2CCN(C)CC2)CC1]

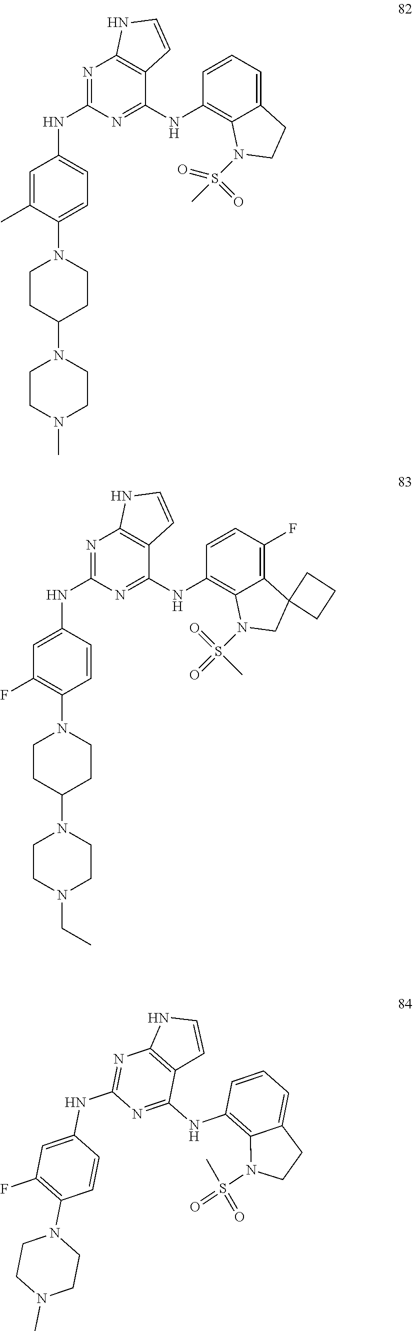 [SMILES: Cc1cc(Nc2nc(Nc3cccc4c3N(S(C)(=O)=O)CC4)c3cc[nH]c3n2)ccc1N1CCC(N2CCN(C)CC2)CC1 ; CCN1CCN(C2CCN(c3ccc(Nc4nc(Nc5ccc(F)c6c5N(S(C)(=O)=O)CC65CCC5)c5cc[nH]c5n4)cc3F)CC2)CC1 ; CN1CCN(c2ccc(Nc3nc(Nc4cccc5c4N(S(C)(=O)=O)CC5)c4cc[nH]c4n3)cc2F)CC1]

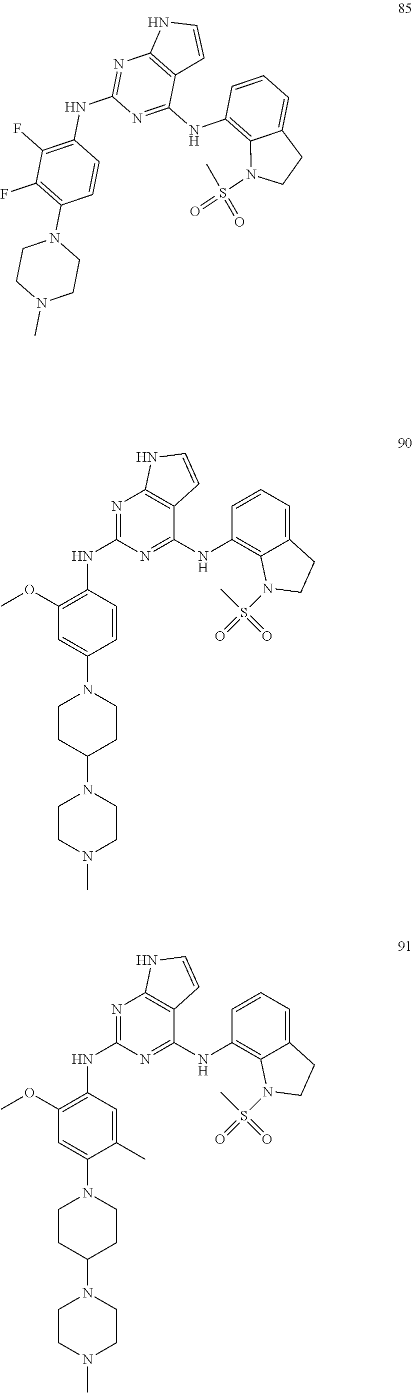 [SMILES: CN1CCN(c2ccc(Nc3nc(Nc4cccc5c4N(S(C)(=O)=O)CC5)c4cc[nH]c4n3)c(F)c2F)CC1 ; COc1cc(N2CCC(N3CCN(C)CC3)CC2)ccc1Nc1nc(Nc2cccc3c2N(S(C)(=O)=O)CC3)c2cc[nH]c2n1 ; COc1cc(N2CCC(N3CCN(C)CC3)CC2)c(C)cc1Nc1nc(Nc2cccc3c2N(S(C)(=O)=O)CC3)c2cc[nH]c2n1]

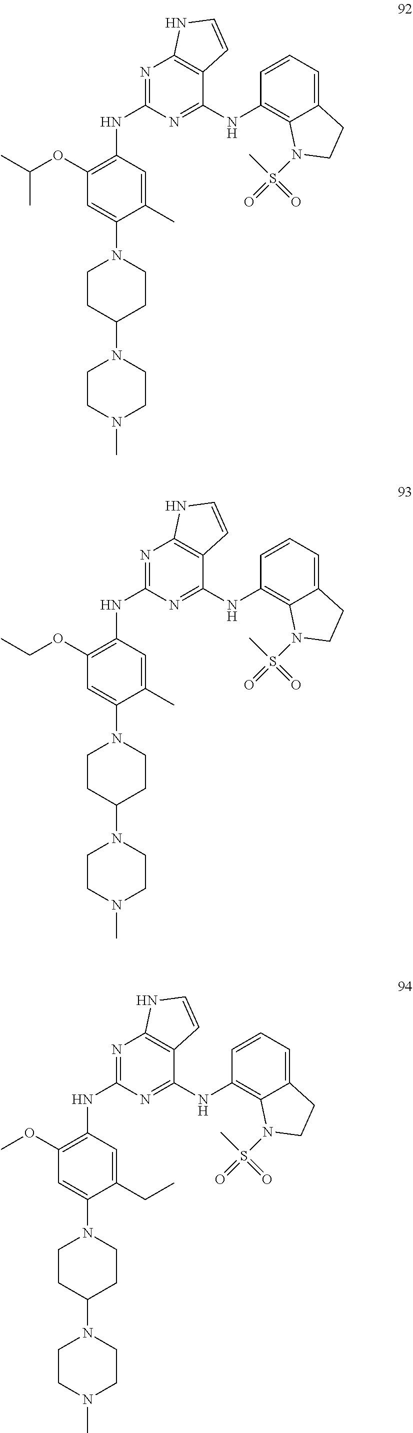 [SMILES: Cc1cc(Nc2nc(Nc3cccc4c3N(S(C)(=O)=O)CC4)c3cc[nH]c3n2)c(OC(C)C)cc1N1CCC(N2CCN(C)CC2)CC1 ; CCOc1cc(N2CCC(N3CCN(C)CC3)CC2)c(C)cc1Nc1nc(Nc2cccc3c2N(S(C)(=O)=O)CC3)c2cc[nH]c2n1 ; CCc1cc(Nc2nc(Nc3cccc4c3N(S(C)(=O)=O)CC4)c3cc[nH]c3n2)c(OC)cc1N1CCC(N2CCN(C)CC2)CC1]

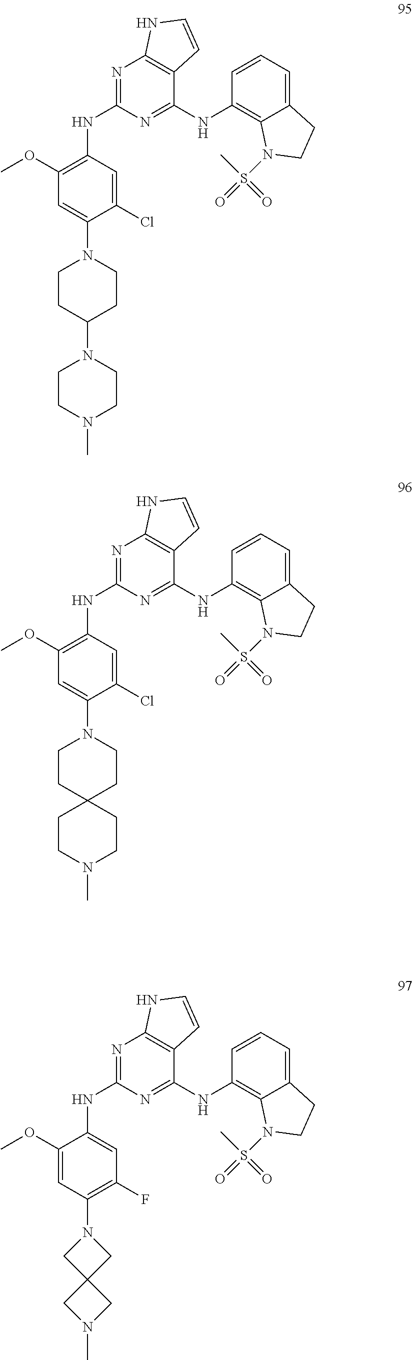 [SMILES: COc1cc(N2CCC(N3CCN(C)CC3)CC2)c(Cl)cc1Nc1nc(Nc2cccc3c2N(S(C)(=O)=O)CC3)c2cc[nH]c2n1 ; COc1cc(N2CCC3(CCN(C)CC3)CC2)c(Cl)cc1Nc1nc(Nc2cccc3c2N(S(C)(=O)=O)CC3)c2cc[nH]c2n1 ; COc1cc(N2CC3(CN(C)C3)C2)c(F)cc1Nc1nc(Nc2cccc3c2N(S(C)(=O)=O)CC3)c2cc[nH]c2n1]

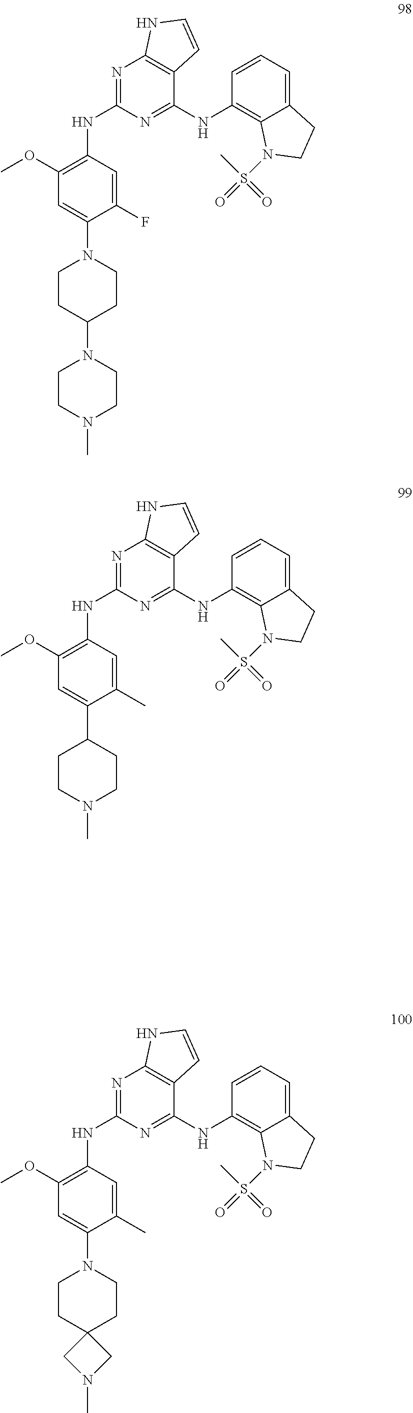 [SMILES: COc1cc(N2CCC(N3CCN(C)CC3)CC2)c(F)cc1Nc1nc(Nc2cccc3c2N(S(C)(=O)=O)CC3)c2cc[nH]c2n1 ; COc1cc(C2CCN(C)CC2)c(C)cc1Nc1nc(Nc2cccc3c2N(S(C)(=O)=O)CC3)c2cc[nH]c2n1 ; COc1cc(N2CCC3(CC2)CN(C)C3)c(C)cc1Nc1nc(Nc2cccc3c2N(S(C)(=O)=O)CC3)c2cc[nH]c2n1]

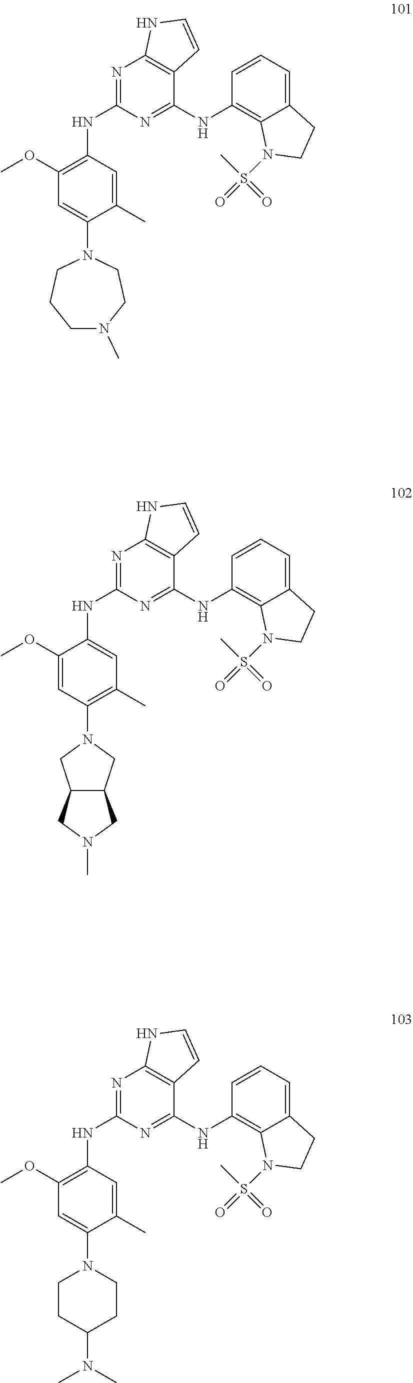 [SMILES: COc1cc(N2CCCN(C)CC2)c(C)cc1Nc1nc(Nc2cccc3c2N(S(C)(=O)=O)CC3)c2cc[nH]c2n1 ; COc1cc(N2C[C@H]3CN(C)C[C@H]3C2)c(C)cc1Nc1nc(Nc2cccc3c2N(S(C)(=O)=O)CC3)c2cc[nH]c2n1 ; COc1cc(N2CCC(N(C)C)CC2)c(C)cc1Nc1nc(Nc2cccc3c2N(S(C)(=O)=O)CC3)c2cc[nH]c2n1]

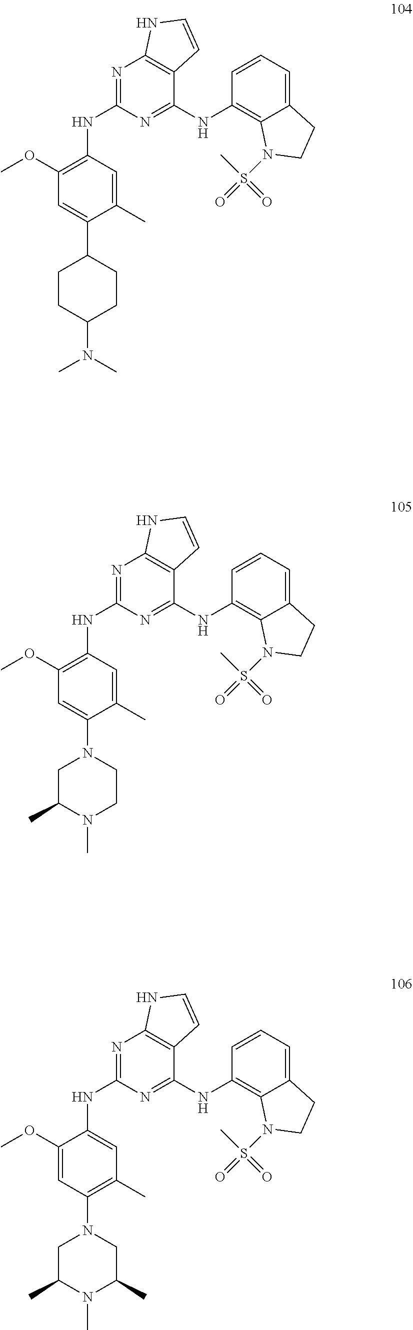 [SMILES: COc1cc(C2CCC(N(C)C)CC2)c(C)cc1Nc1nc(Nc2cccc3c2N(S(C)(=O)=O)CC3)c2cc[nH]c2n1 ; COc1cc(N2CCN(C)[C@@H](C)C2)c(C)cc1Nc1nc(Nc2cccc3c2N(S(C)(=O)=O)CC3)c2cc[nH]c2n1 ; COc1cc(N2C[C@@H](C)N(C)[C@@H](C)C2)c(C)cc1Nc1nc(Nc2cccc3c2N(S(C)(=O)=O)CC3)c2cc[nH]c2n1]

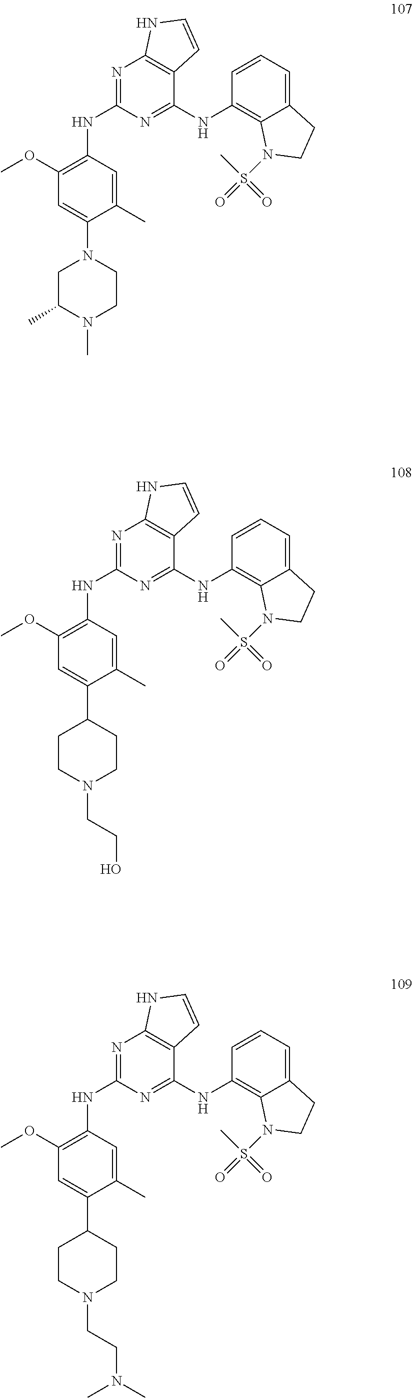 [SMILES: COc1cc(N2CCN(C)[C@H](C)C2)c(C)cc1Nc1nc(Nc2cccc3c2N(S(C)(=O)=O)CC3)c2cc[nH]c2n1 ; COc1cc(C2CCN(CCO)CC2)c(C)cc1Nc1nc(Nc2cccc3c2N(S(C)(=O)=O)CC3)c2cc[nH]c2n1 ; COc1cc(C2CCN(CCN(C)C)CC2)c(C)cc1Nc1nc(Nc2cccc3c2N(S(C)(=O)=O)CC3)c2cc[nH]c2n1]

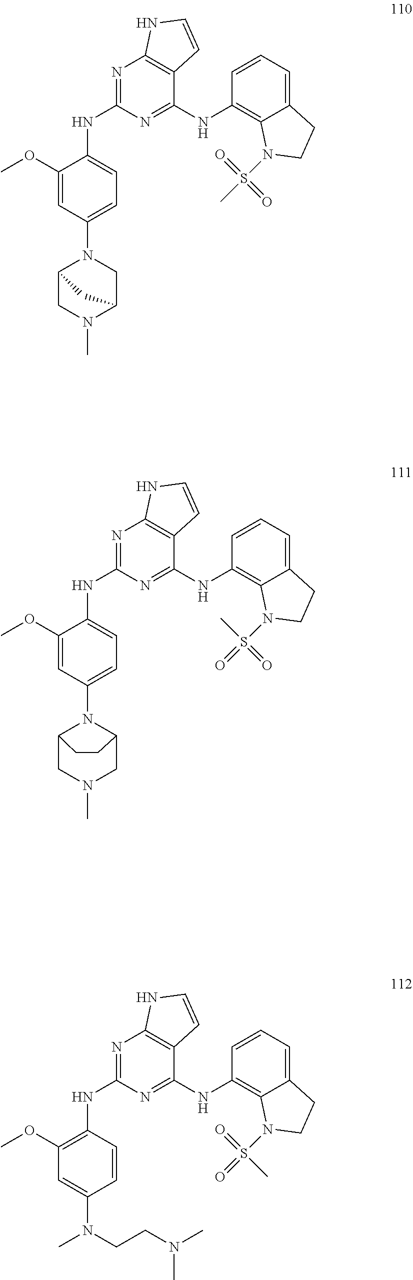 [SMILES: COc1cc(N2C[C@@H]3C[C@H]2CN3C)ccc1Nc1nc(Nc2cccc3c2N(S(C)(=O)=O)CC3)c2cc[nH]c2n1 ; COc1cc(N2C3CCC2CN(C)C3)ccc1Nc1nc(Nc2cccc3c2N(S(C)(=O)=O)CC3)c2cc[nH]c2n1 ; COc1cc(N(C)CCN(C)C)ccc1Nc1nc(Nc2cccc3c2N(S(C)(=O)=O)CC3)c2cc[nH]c2n1]

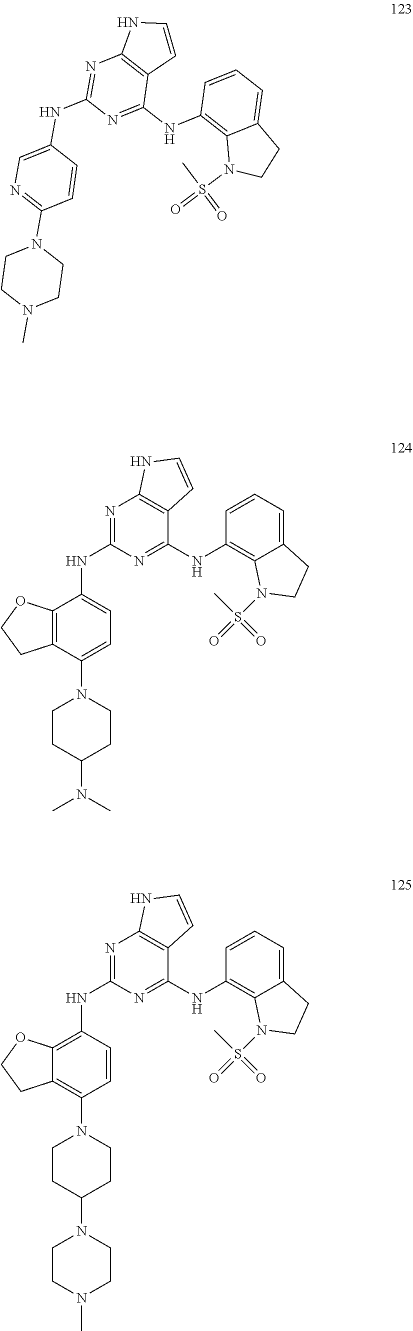 [SMILES: CN1CCN(c2ccc(Nc3nc(Nc4cccc5c4N(S(C)(=O)=O)CC5)c4cc[nH]c4n3)cn2)CC1 ; CN(C)C1CCN(c2ccc(Nc3nc(Nc4cccc5c4N(S(C)(=O)=O)CC5)c4cc[nH]c4n3)c3c2CCO3)CC1 ; CN1CCN(C2CCN(c3ccc(Nc4nc(Nc5cccc6c5N(S(C)(=O)=O)CC6)c5cc[nH]c5n4)c4c3CCO4)CC2)CC1]

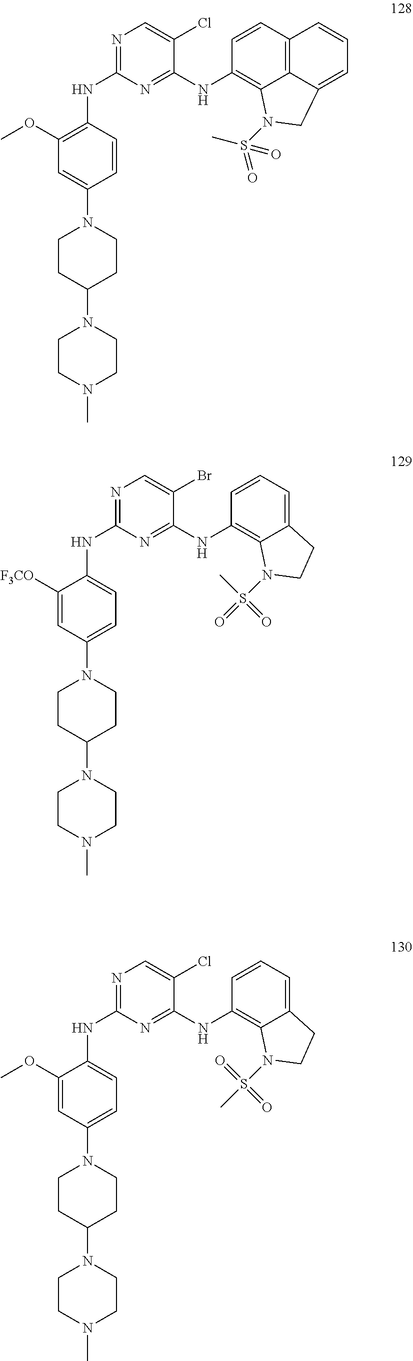 [SMILES: COc1cc(N2CCC(N3CCN(C)CC3)CC2)ccc1Nc1ncc(Cl)c(Nc2ccc3cccc4c3c2N(S(C)(=O)=O)C4)n1 ; CN1CCN(C2CCN(c3ccc(Nc4ncc(Br)c(Nc5cccc6c5N(S(C)(=O)=O)CC6)n4)c(OC(F)(F)F)c3)CC2)CC1 ; COc1cc(N2CCC(N3CCN(C)CC3)CC2)ccc1Nc1ncc(Cl)c(Nc2cccc3c2N(S(C)(=O)=O)CC3)n1]

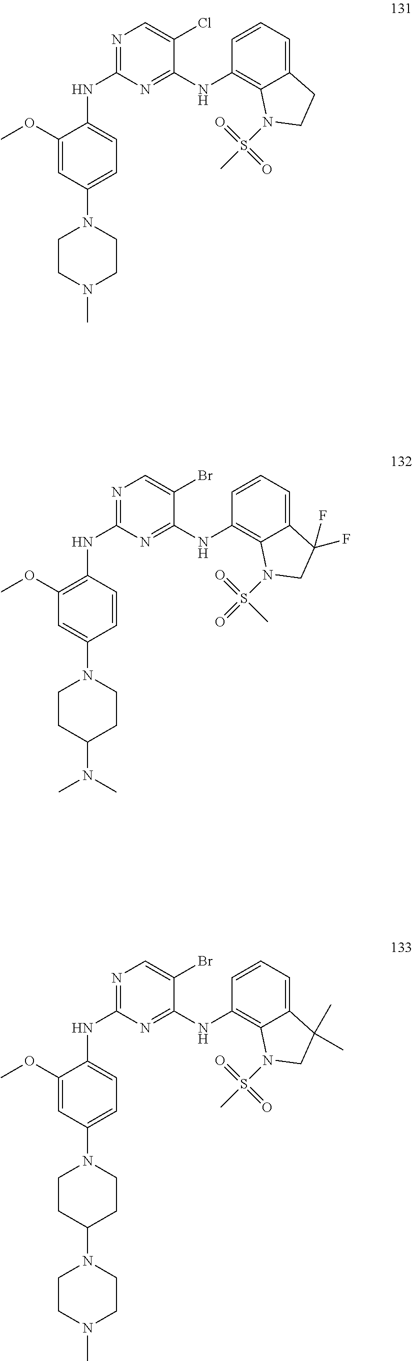

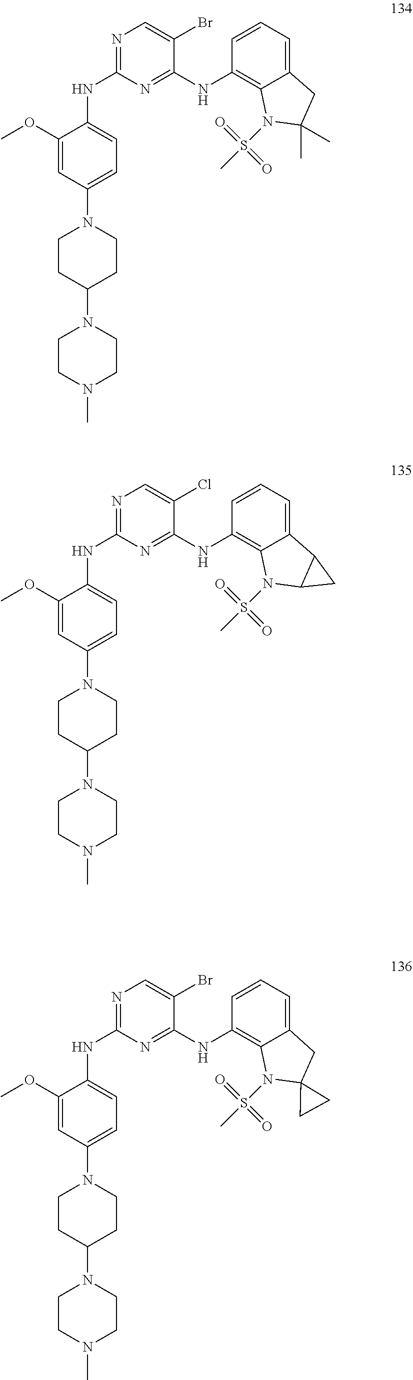

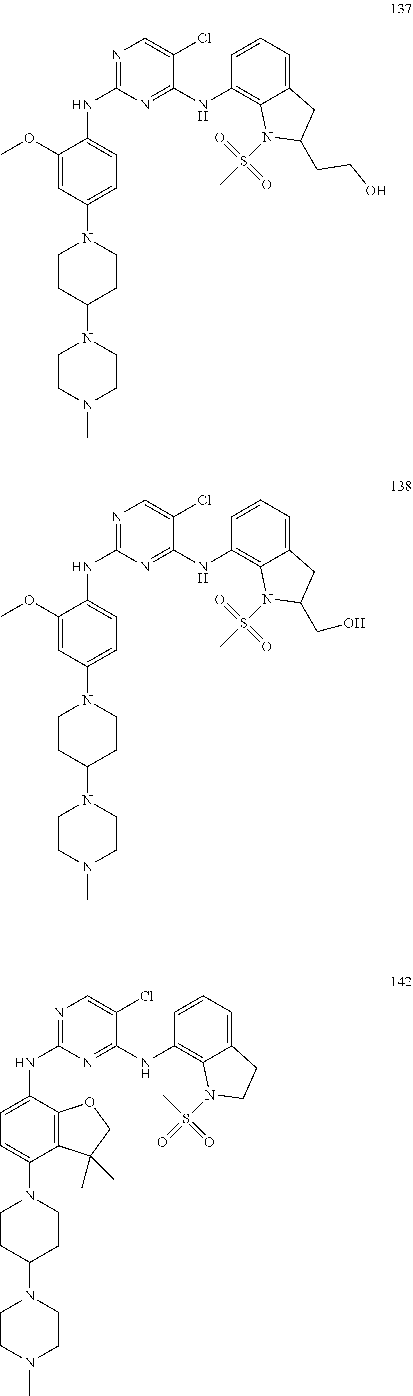 [SMILES: COc1cc(N2CCC(N3CCN(C)CC3)CC2)ccc1Nc1ncc(Cl)c(Nc2cccc3c2N(S(C)(=O)=O)C(CCO)C3)n1 ; COc1cc(N2CCC(N3CCN(C)CC3)CC2)ccc1Nc1ncc(Cl)c(Nc2cccc3c2N(S(C)(=O)=O)C(CO)C3)n1 ; CN1CCN(C2CCN(c3ccc(Nc4ncc(Cl)c(Nc5cccc6c5N(S(C)(=O)=O)CC6)n4)c4c3C(C)(C)CO4)CC2)CC1]

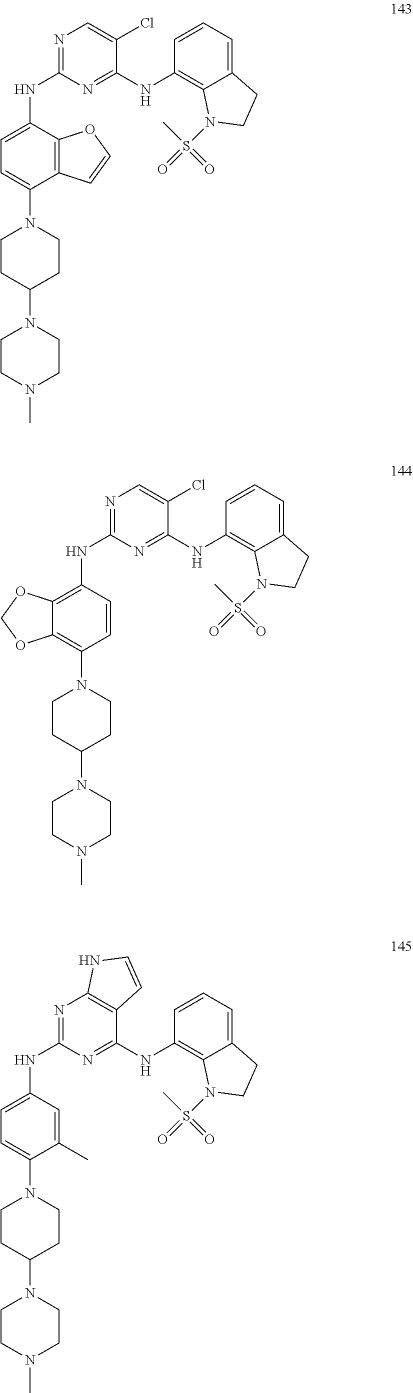 [SMILES: CN1CCN(C2CCN(c3ccc(Nc4ncc(Cl)c(Nc5cccc6c5N(S(C)(=O)=O)CC6)n4)c4occc34)CC2)CC1 ; CN1CCN(C2CCN(c3ccc(Nc4ncc(Cl)c(Nc5cccc6c5N(S(C)(=O)=O)CC6)n4)c4c3OCO4)CC2)CC1 ; Cc1cc(Nc2nc(Nc3cccc4c3N(S(C)(=O)=O)CC4)c3cc[nH]c3n2)ccc1N1CCC(N2CCN(C)CC2)CC1]

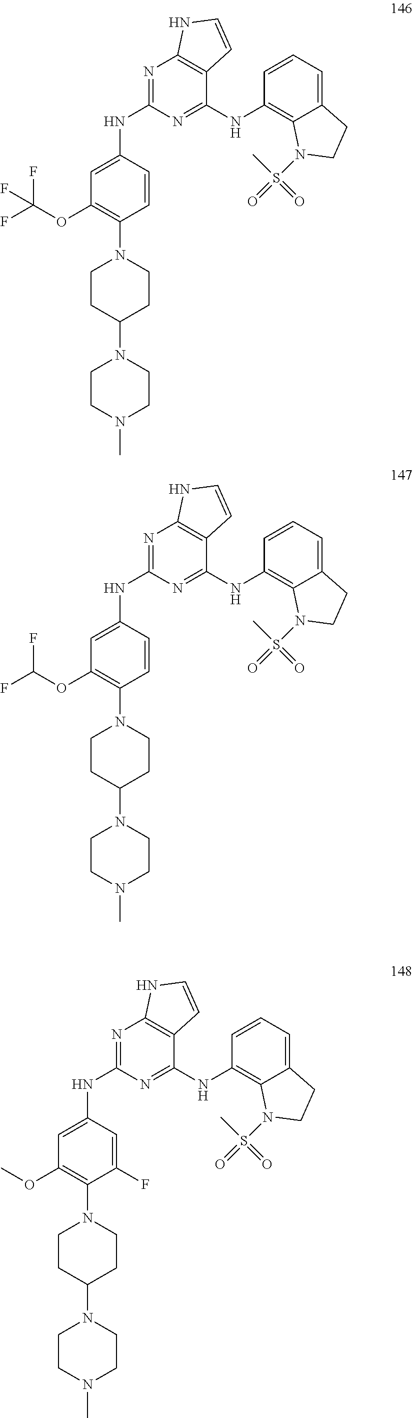 [SMILES: CN1CCN(C2CCN(c3ccc(Nc4nc(Nc5cccc6c5N(S(C)(=O)=O)CC6)c5cc[nH]c5n4)cc3OC(F)(F)F)CC2)CC1 ; CN1CCN(C2CCN(c3ccc(Nc4nc(Nc5cccc6c5N(S(C)(=O)=O)CC6)c5cc[nH]c5n4)cc3OC(F)F)CC2)CC1 ; COc1cc(Nc2nc(Nc3cccc4c3N(S(C)(=O)=O)CC4)c3cc[nH]c3n2)cc(F)c1N1CCC(N2CCN(C)CC2)CC1]

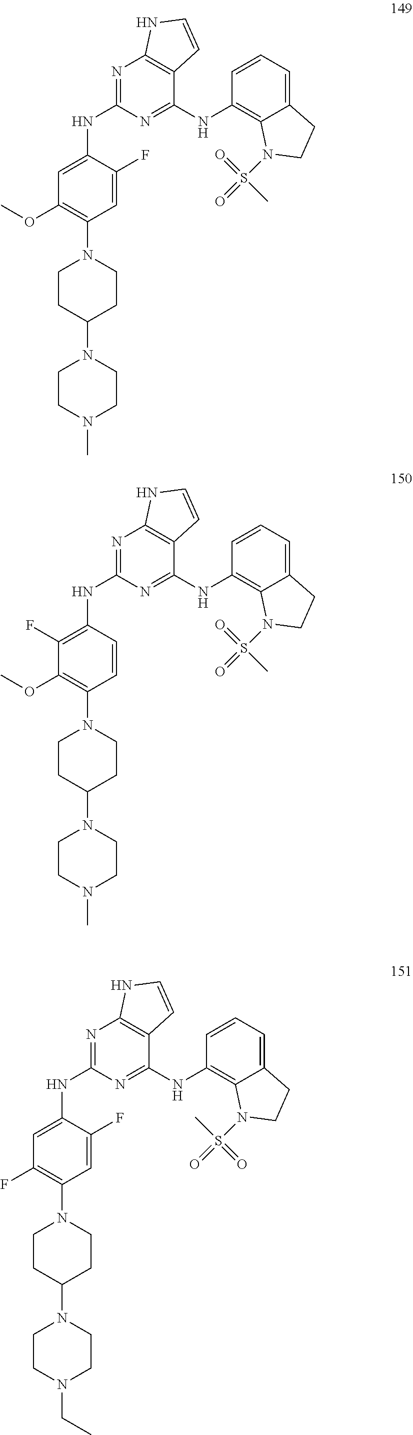 [SMILES: COc1cc(Nc2nc(Nc3cccc4c3N(S(C)(=O)=O)CC4)c3cc[nH]c3n2)c(F)cc1N1CCC(N2CCN(C)CC2)CC1 ; COc1c(N2CCC(N3CCN(C)CC3)CC2)ccc(Nc2nc(Nc3cccc4c3N(S(C)(=O)=O)CC4)c3cc[nH]c3n2)c1F ; CCN1CCN(C2CCN(c3cc(F)c(Nc4nc(Nc5cccc6c5N(S(C)(=O)=O)CC6)c5cc[nH]c5n4)cc3F)CC2)CC1]

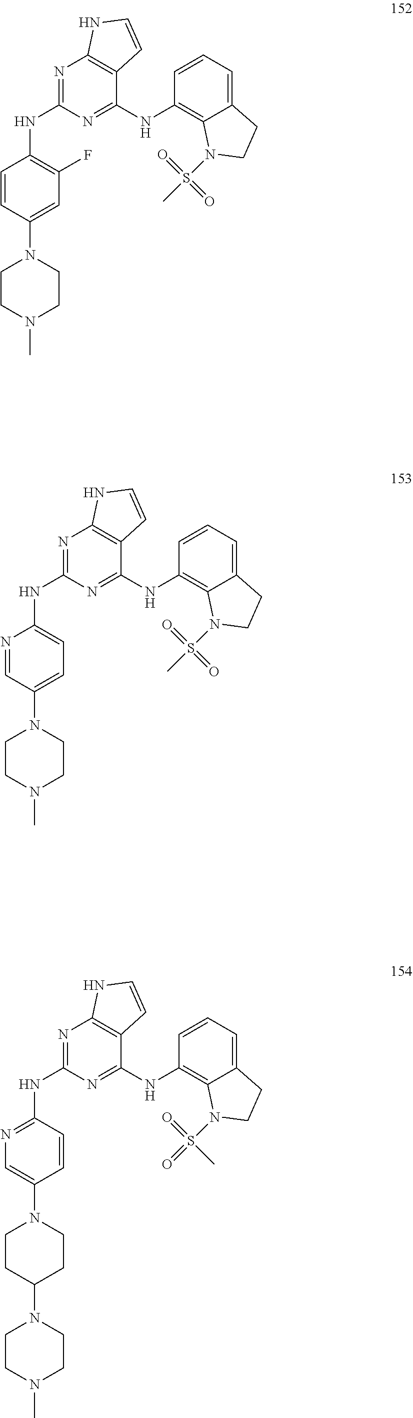 [SMILES: CN1CCN(c2ccc(Nc3nc(Nc4cccc5c4N(S(C)(=O)=O)CC5)c4cc[nH]c4n3)c(F)c2)CC1 ; CN1CCN(c2ccc(Nc3nc(Nc4cccc5c4N(S(C)(=O)=O)CC5)c4cc[nH]c4n3)nc2)CC1 ; CN1CCN(C2CCN(c3ccc(Nc4nc(Nc5cccc6c5N(S(C)(=O)=O)CC6)c5cc[nH]c5n4)nc3)CC2)CC1]

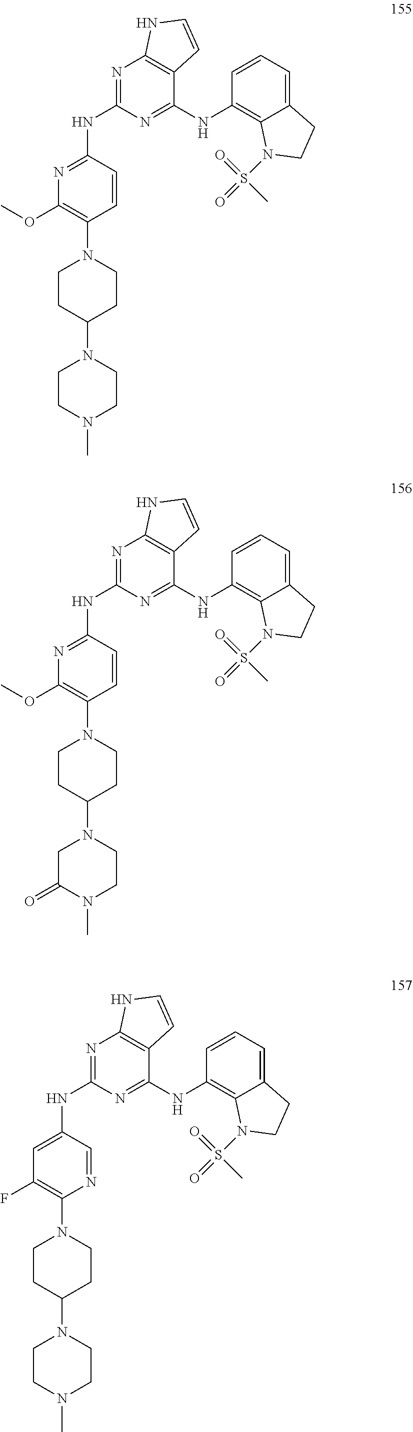 [SMILES: COc1nc(Nc2nc(Nc3cccc4c3N(S(C)(=O)=O)CC4)c3cc[nH]c3n2)ccc1N1CCC(N2CCN(C)CC2)CC1 ; COc1nc(Nc2nc(Nc3cccc4c3N(S(C)(=O)=O)CC4)c3cc[nH]c3n2)ccc1N1CCC(N2CCN(C)C(=O)C2)CC1 ; CN1CCN(C2CCN(c3ncc(Nc4nc(Nc5cccc6c5N(S(C)(=O)=O)CC6)c5cc[nH]c5n4)cc3F)CC2)CC1]

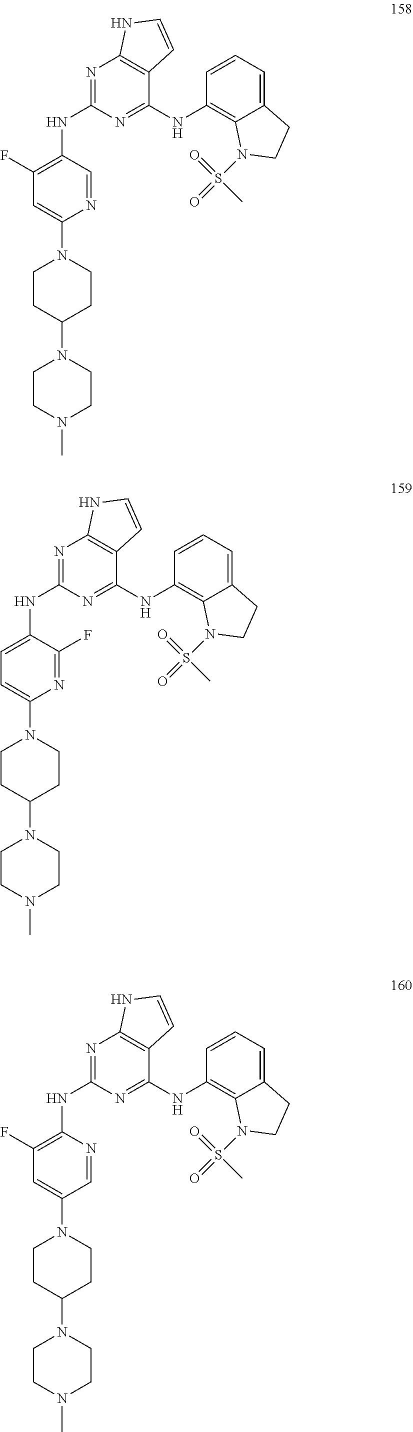 [SMILES: CN1CCN(C2CCN(c3cc(F)c(Nc4nc(Nc5cccc6c5N(S(C)(=O)=O)CC6)c5cc[nH]c5n4)cn3)CC2)CC1 ; CN1CCN(C2CCN(c3ccc(Nc4nc(Nc5cccc6c5N(S(C)(=O)=O)CC6)c5cc[nH]c5n4)c(F)n3)CC2)CC1 ; CN1CCN(C2CCN(c3cnc(Nc4nc(Nc5cccc6c5N(S(C)(=O)=O)CC6)c5cc[nH]c5n4)c(F)c3)CC2)CC1]

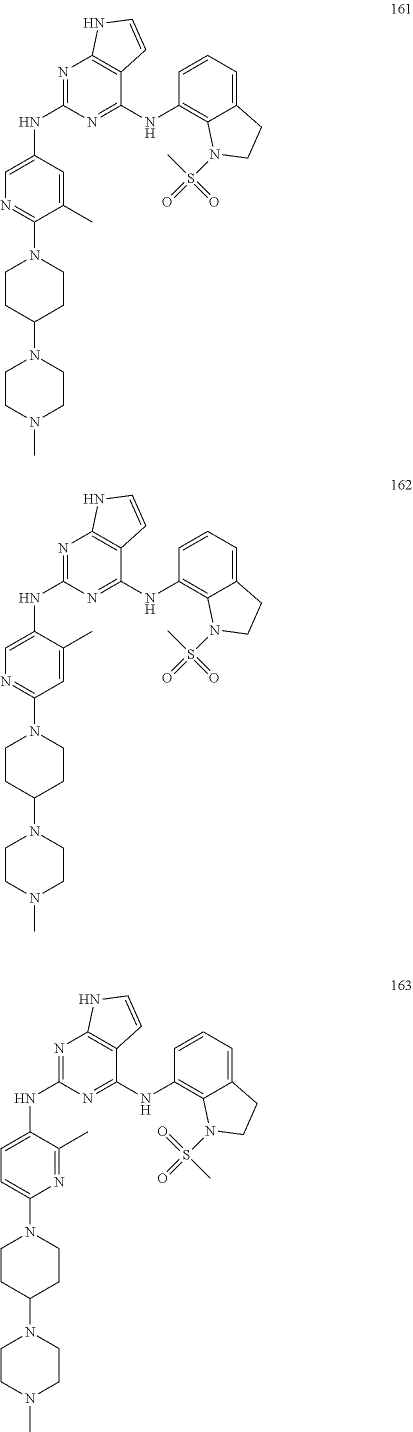 [SMILES: Cc1cc(Nc2nc(Nc3cccc4c3N(S(C)(=O)=O)CC4)c3cc[nH]c3n2)cnc1N1CCC(N2CCN(C)CC2)CC1 ; Cc1cc(N2CCC(N3CCN(C)CC3)CC2)ncc1Nc1nc(Nc2cccc3c2N(S(C)(=O)=O)CC3)c2cc[nH]c2n1 ; Cc1nc(N2CCC(N3CCN(C)CC3)CC2)ccc1Nc1nc(Nc2cccc3c2N(S(C)(=O)=O)CC3)c2cc[nH]c2n1]

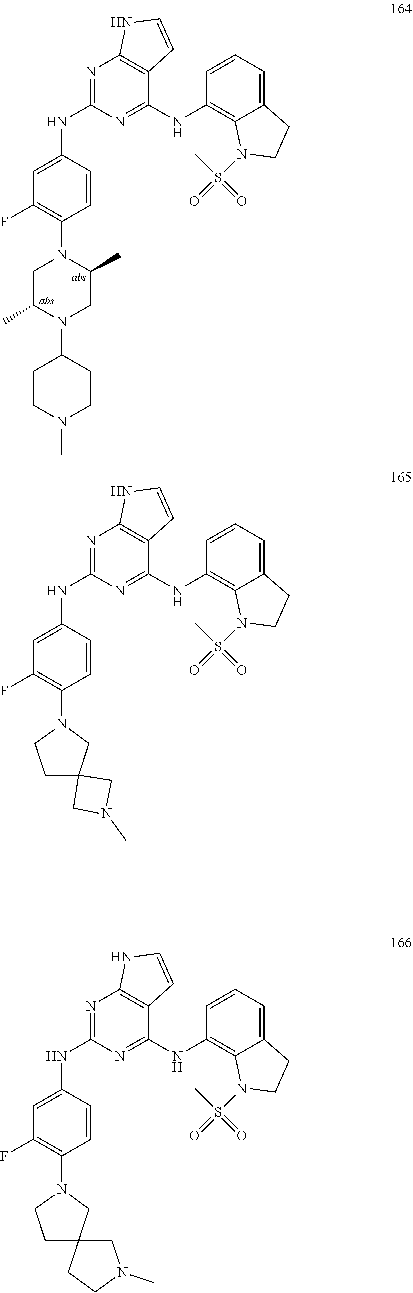 [SMILES: C[C@@H]1CN(c2ccc(Nc3nc(Nc4cccc5c4N(S(C)(=O)=O)CC5)c4cc[nH]c4n3)cc2F)[C@@H](C)CN1C1CCN(C)CC1 ; CN1CC2(CCN(c3ccc(Nc4nc(Nc5cccc6c5N(S(C)(=O)=O)CC6)c5cc[nH]c5n4)cc3F)C2)C1 ; CN1CCC2(CCN(c3ccc(Nc4nc(Nc5cccc6c5N(S(C)(=O)=O)CC6)c5cc[nH]c5n4)cc3F)C2)C1]

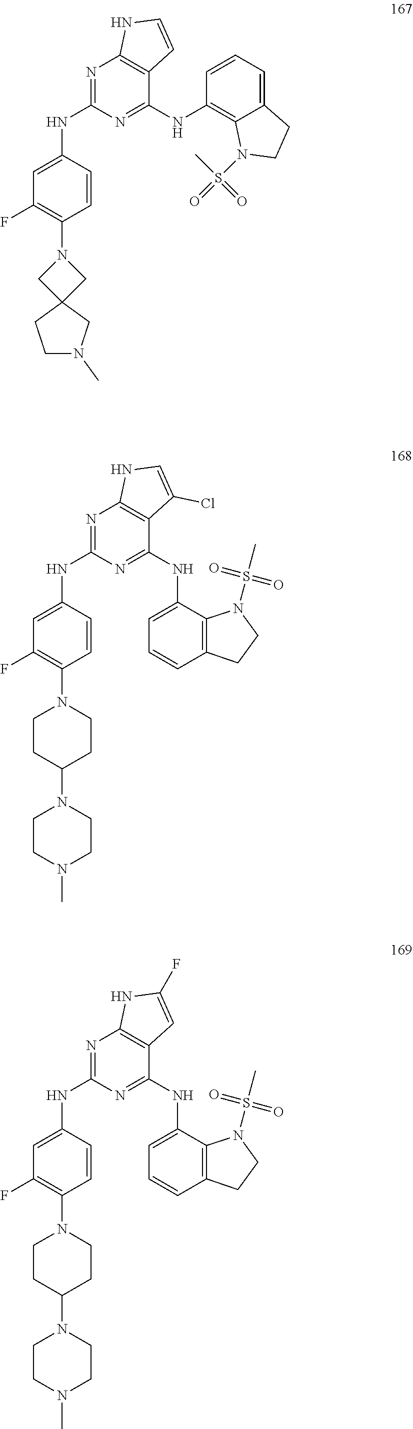 [SMILES: CN1CCC2(C1)CN(c1ccc(Nc3nc(Nc4cccc5c4N(S(C)(=O)=O)CC5)c4cc[nH]c4n3)cc1F)C2 ; CN1CCN(C2CCN(c3ccc(Nc4nc(Nc5cccc6c5N(S(C)(=O)=O)CC6)c5c(Cl)c[nH]c5n4)cc3F)CC2)CC1 ; CN1CCN(C2CCN(c3ccc(Nc4nc(Nc5cccc6c5N(S(C)(=O)=O)CC6)c5cc(F)[nH]c5n4)cc3F)CC2)CC1]

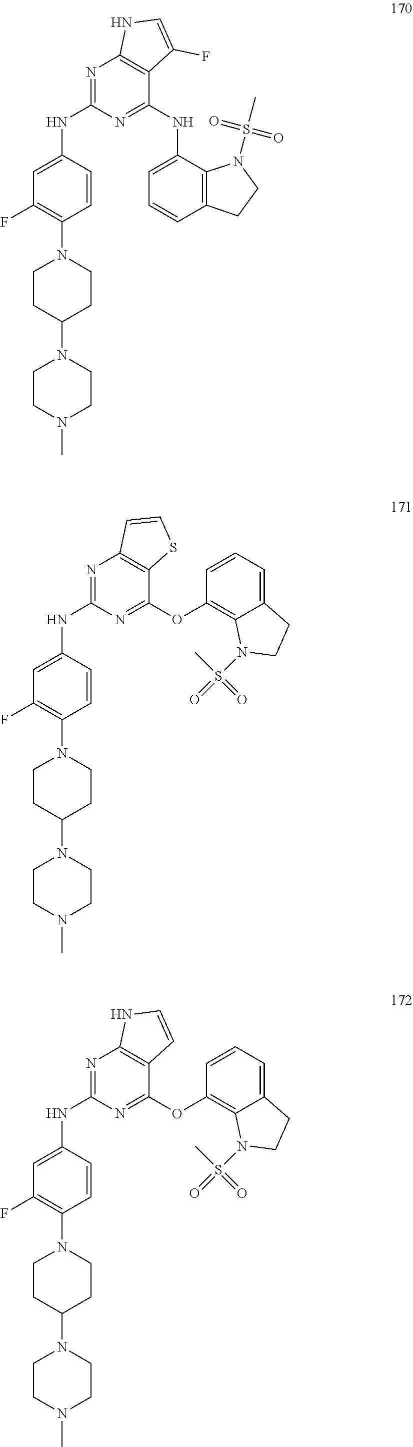 [SMILES: CN1CCN(C2CCN(c3ccc(Nc4nc(Nc5cccc6c5N(S(C)(=O)=O)CC6)c5c(F)c[nH]c5n4)cc3F)CC2)CC1 ; CN1CCN(C2CCN(c3ccc(Nc4nc(Oc5cccc6c5N(S(C)(=O)=O)CC6)c5sccc5n4)cc3F)CC2)CC1 ; CN1CCN(C2CCN(c3ccc(Nc4nc(Oc5cccc6c5N(S(C)(=O)=O)CC6)c5cc[nH]c5n4)cc3F)CC2)CC1]

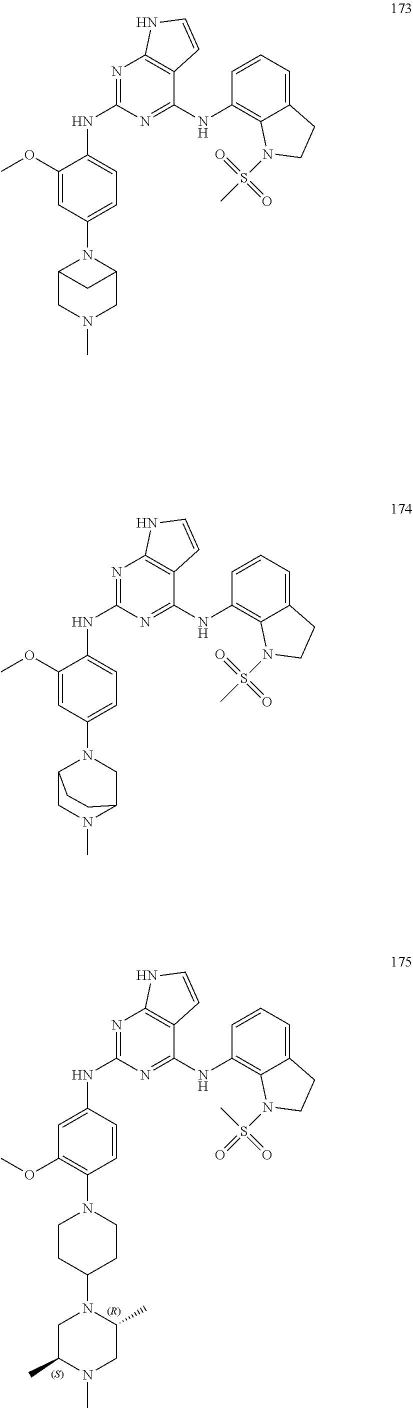

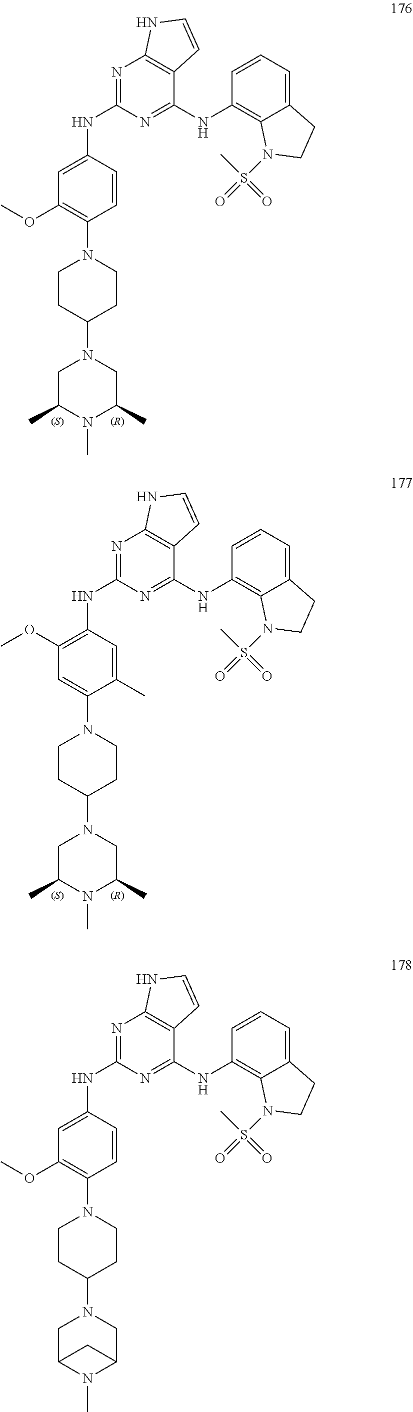

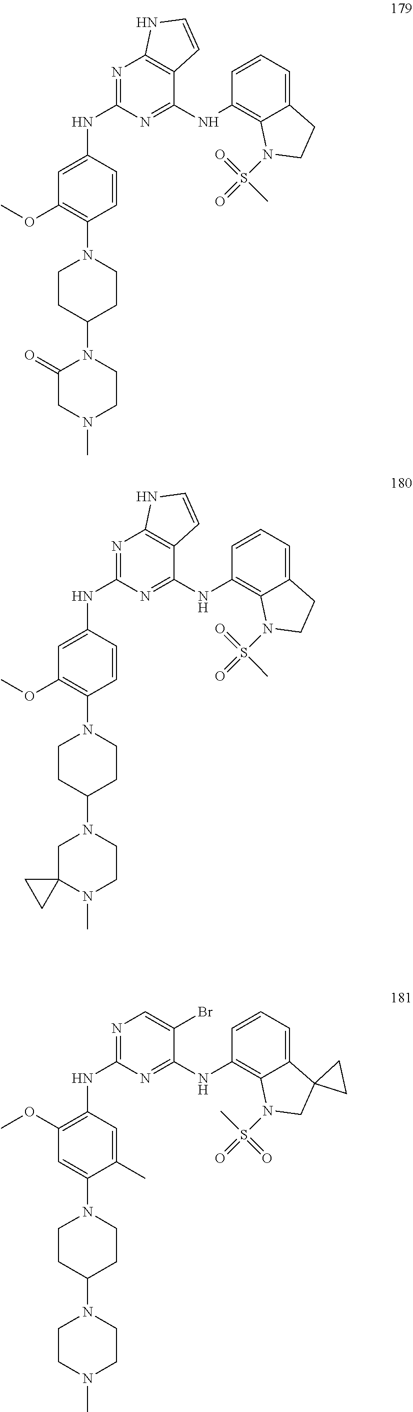 [SMILES: COc1cc(Nc2nc(Nc3cccc4c3N(S(C)(=O)=O)CC4)c3cc[nH]c3n2)ccc1N1CCC(N2CCN(C)CC2=O)CC1 ; COc1cc(Nc2nc(Nc3cccc4c3N(S(C)(=O)=O)CC4)c3cc[nH]c3n2)ccc1N1CCC(N2CCN(C)C3(CC3)C2)CC1 ; COc1cc(N2CCC(N3CCN(C)CC3)CC2)c(C)cc1Nc1ncc(Br)c(Nc2cccc3c2N(S(C)(=O)=O)CC32CC2)n1]

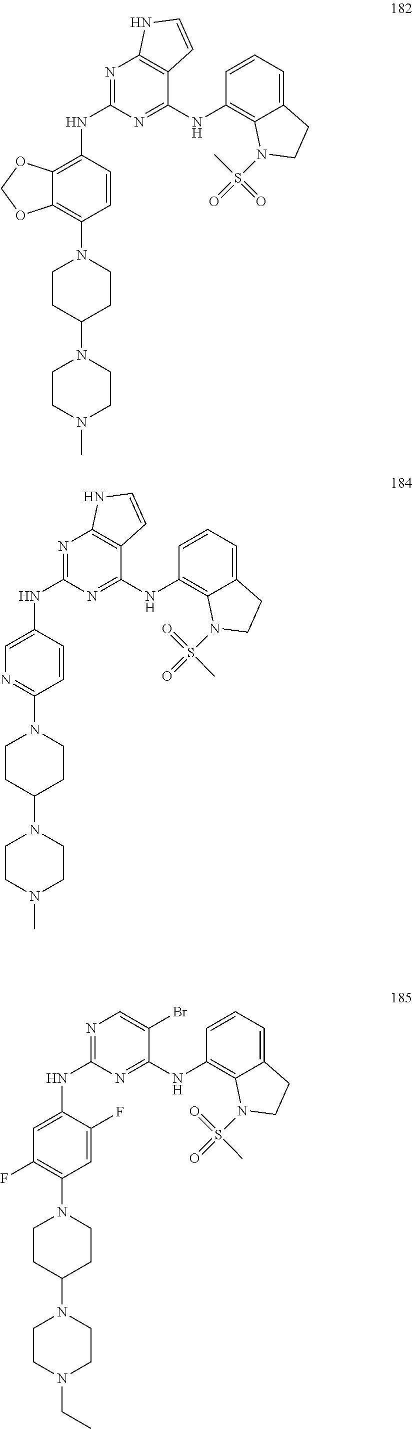

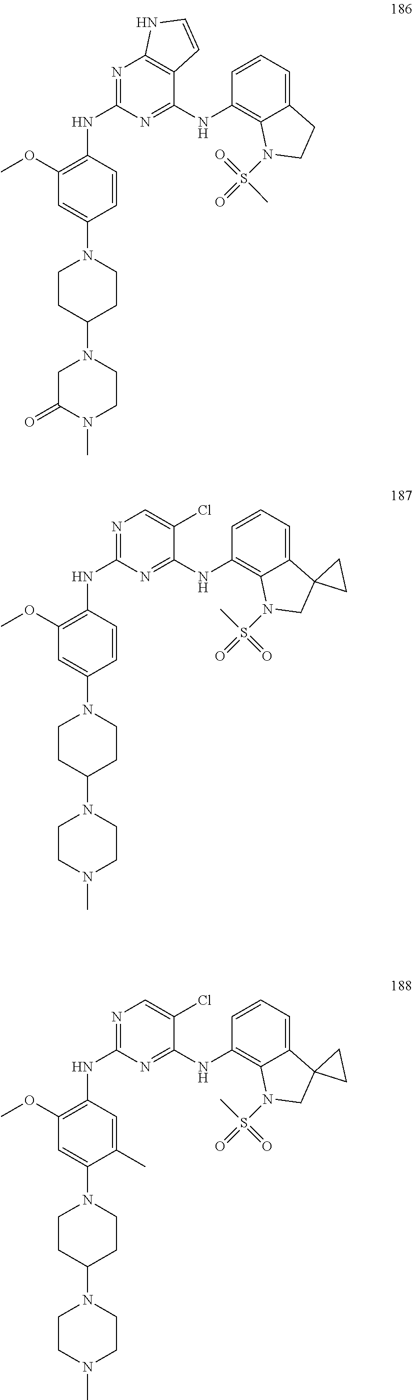 [SMILES: COc1cc(N2CCC(N3CCN(C)C(=O)C3)CC2)ccc1Nc1nc(Nc2cccc3c2N(S(C)(=O)=O)CC3)c2cc[nH]c2n1 ; COc1cc(N2CCC(N3CCN(C)CC3)CC2)ccc1Nc1ncc(Cl)c(Nc2cccc3c2N(S(C)(=O)=O)CC32CC2)n1 ; COc1cc(N2CCC(N3CCN(C)CC3)CC2)c(C)cc1Nc1ncc(Cl)c(Nc2cccc3c2N(S(C)(=O)=O)CC32CC2)n1]

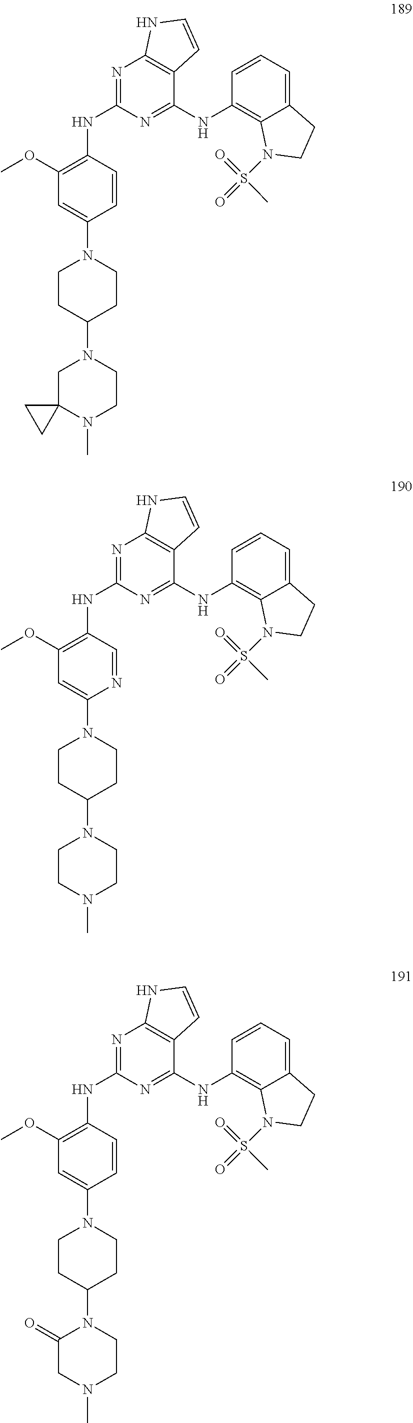 [SMILES: COc1cc(N2CCC(N3CCN(C)C4(CC4)C3)CC2)ccc1Nc1nc(Nc2cccc3c2N(S(C)(=O)=O)CC3)c2cc[nH]c2n1 ; COc1cc(N2CCC(N3CCN(C)CC3)CC2)ncc1Nc1nc(Nc2cccc3c2N(S(C)(=O)=O)CC3)c2cc[nH]c2n1 ; COc1cc(N2CCC(N3CCN(C)CC3=O)CC2)ccc1Nc1nc(Nc2cccc3c2N(S(C)(=O)=O)CC3)c2cc[nH]c2n1]

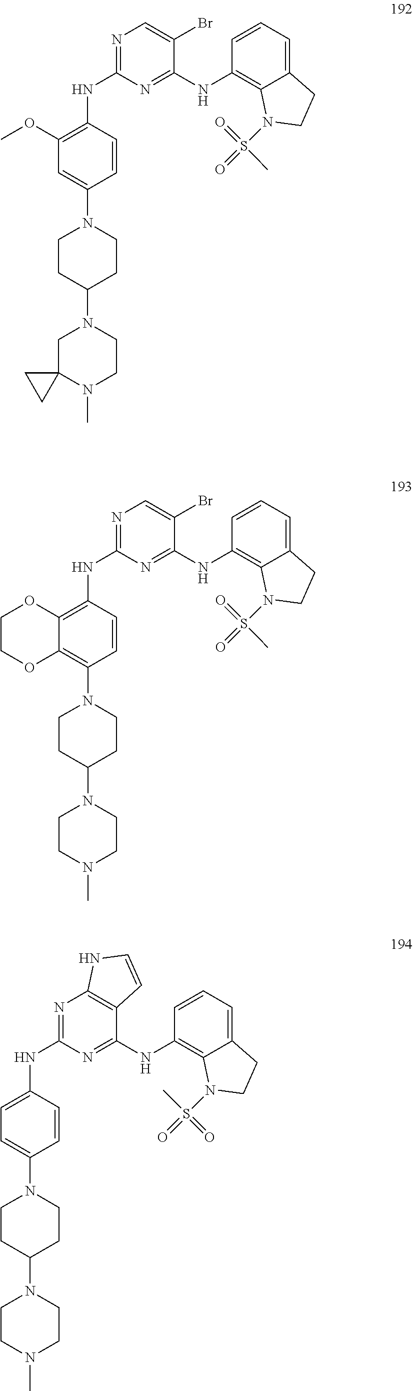 [SMILES: COc1cc(N2CCC(N3CCN(C)C4(CC4)C3)CC2)ccc1Nc1ncc(Br)c(Nc2cccc3c2N(S(C)(=O)=O)CC3)n1 ; CN1CCN(C2CCN(c3ccc(Nc4ncc(Br)c(Nc5cccc6c5N(S(C)(=O)=O)CC6)n4)c4c3OCCO4)CC2)CC1 ; CN1CCN(C2CCN(c3ccc(Nc4nc(Nc5cccc6c5N(S(C)(=O)=O)CC6)c5cc[nH]c5n4)cc3)CC2)CC1]

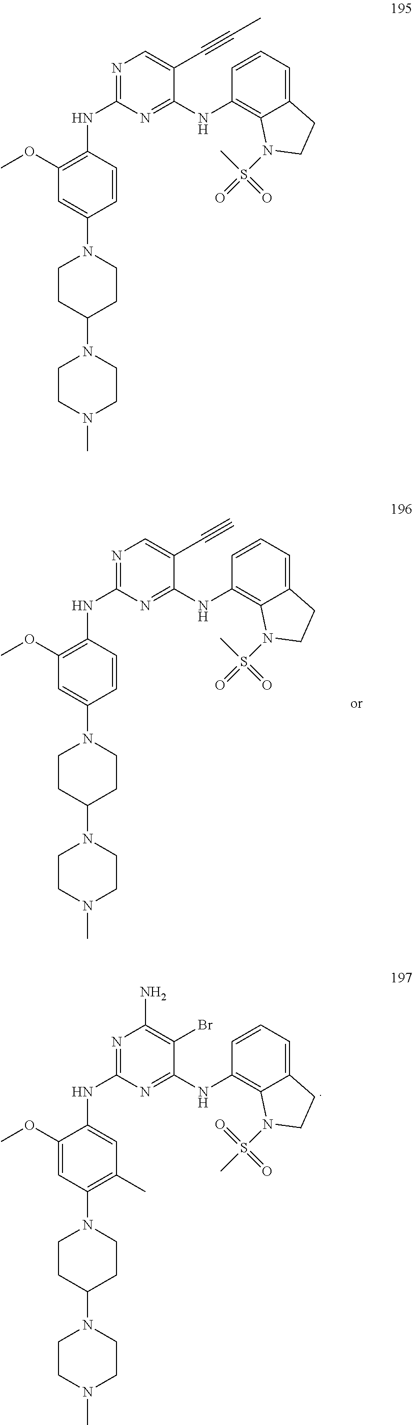 [SMILES: CC#Cc1cnc(Nc2ccc(N3CCC(N4CCN(C)CC4)CC3)cc2OC)nc1Nc1cccc2c1N(S(C)(=O)=O)CC2 ; C#Cc1cnc(Nc2ccc(N3CCC(N4CCN(C)CC4)CC3)cc2OC)nc1Nc1cccc2c1N(S(C)(=O)=O)CC2 ; COc1cc(N2CCC(N3CCN(C)CC3)CC2)c(C)cc1Nc1nc(N)c(Br)c(Nc2cccc3c2N(S(C)(=O)=O)CC3)n1]

Aspect 23. A pharmaceutical composition comprising a compound of any one of Aspect s 1-22 or a pharmaceutically acceptable salt, stereoisomer, tautomer or prodrug thereof, together with a pharmaceutically acceptable excipient.

Aspect 24. A method of treating a disease in which EGFR modulation is involved, comprising administrating a subject in need thereof an effective amount of a compound of any one of Aspects 1-22 or an N-oxide thereof or a pharmaceutically acceptable salt thereof or a stereoisomer thereof or prodrug thereof.

Aspect 25. The method of Aspect 24, wherein the disease is cancer, preferably pancreatic cancer, breast cancer, glioblastoma multiforme, head and neck cancer or non-small cell lung cancer.

Aspect 26. Use of a compound of any one of Aspects 1-22 or a pharmaceutically acceptable salt, stereoisomer, tautomer or prodrug thereof in the preparation of a medicament for treating a disease that can be affected by EGFR modulation.

Aspect 27. The use of Aspect 26, wherein the disease is cancer, preferred pancreatic cancer, breast cancer, glioblastoma multiforme, head and neck cancer or non-small cell lung cancer.

Aspect 28. Use of a compound of any one of Aspects 1-22 thereof in the preparation of PROTAC medicine for treating a disease that can be affected by EGFR modulation.

### DETAILED DESCRIPTION OF THE INVENTION

The following terms have the indicated meanings throughout the specification:

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “halogen” includes fluoro (F), chloro (Cl), bromo (Br) and iodo (I).

The term “alkenyl” includes a hydrocarbon group selected from linear and branched hydrocarbon groups comprising at least one C≡C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkynyl” includes a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further, for example, the cycloalkyl group may be selected from a monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “spiro cycloalkyl” includes a cycloalkyl as defined herein which is formed by at least two rings sharing one atom. The term “spirobicycloalkyl” refers to a bicyclic saturated carbon ring system in which the two rings are connected through just one atom.

Spirobicycloalkyl rings are taken from, but not limited to spiro[2.2]pentanyl, spiro[2.3]hexanyl, spiro[2.4]heptanyl, spiro[3.3]heptanyl, spiro[2.5]octanyl, spiro[3.4]octanyl, spiro[2.6]nonanyl, spiro[3.5]nonanyl, spiro[4.4]nonanyl, spiro[2.7]decanyl, spiro[3.6]decanyl, spiro[4.5]decanyl, spiro[3.7]undecanyl, spiro[4.6]undecanyl, spiro[5.5]undecanyl, spiro[4.7]dodecanyl, and spiro[5.6]dodecanyl.

The term “fused cycloalkyl” includes a bicyclic cycloalkyl group as defined herein which is saturated and is formed by two or more rings sharing two adjacent atoms.

The term “bridged cycloalkyl” includes a cycloalkyl as defined herein which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₋₆ cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl; and,
tricyclic ring systems such as 10 to 15 membered tricyclic ring systems wherein at least one ring is carbocyclic and aromatic, e.g., fluorenyl, anthracenyl or phenanthrenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic or tricyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

“Spiro heterocyclyl” refers to a 5- to 20-membered polycyclic heterocyclyl with rings connected through one common carbon atom (called a spiro atom), wherein said rings have one or more heteroatoms selected from the group consisting of N, O, S, SO or SO₂ heteroatoms as ring atoms, with the remaining ring atoms being C, wherein one or more rings may contain one or more double bonds, but none of the rings has a completely conjugated pi-electron system. Preferably a spiro heterocyclyl is 6- to 14-membered, and more preferably 7- to 10-membered. According to the number of common spiro atoms, a spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, and more preferably 4-membered/4-membered, 4-membered/5-membered, 4-membered/6-membered, 5-membered/5-membered, or 5-membered/6-membered mono-spiro heterocyclyl. Representative examples of spiro heterocyclyls include, but are not limited to the following groups: azaspiro[5.5]undecanyl, diazaspiro[5.5]undecanyl, azaspiro[4.5]decanyl, diazaspiro[4.5]decanyl, azaspiro[3.5]nonanyl, diazaspiro[3.5]nonanyl, azaspiro[4.4]nonanyl, diazaspiro[4.4]nonanyl, azaspiro[3.4]octanyl, diazaspiro[3.4]octanyl, azaspiro[3.3]heptanyl or diazaspiro[3.3]heptanyl, preferably 3,9-diazaspiro[5.5]undecan-9-yl, 2,7-diazaspiro[3.5]nonan-7-yl, 2,8-diazaspiro[4.5]decan-8yl or 2,6-diazaspiro[3.3]heptan-6-yl.

“Fused heterocyclyl” refers to a 5- to 20-membered polycyclic heterocyclyl group, wherein each ring in the system shares an adjacent pair of carbon atoms with another ring, wherein one or more rings may contain one or more double bonds, but none of the rings has a completely conjugated pi-electron system, and wherein said rings have one or more heteroatoms selected from the group consisting of N, O, S, SO or SO₂ heteroatoms as ring atoms, with the remaining ring atoms being C. Preferably, a fused heterocyclyl is 6- to 14-membered, and more preferably 7- to 10-membered. According to the number of membered rings, a fused heterocyclyl is divided into bicyclic, tricyclic, tetracyclic, or polycyclic fused heterocyclyl, preferably refers to bicyclic or tricyclic fused heterocyclyl, and more preferably 5-membered/5-membered, or 5-membered/6-membered bicyclic fused heterocyclyl.

“Bridged heterocyclyl” refers to a 5- to 14-membered polycyclic heterocyclic alkyl group, wherein every two rings in the system share two disconnected atoms, the rings can have one or more double bonds, but none of the rings has a completely conjugated pi-electron system, and the rings have one or more heteroatoms selected from the group consisting of N, O, S, SO or SO₂ heteroatoms as ring atoms, with the remaining ring atoms being C. Preferably, a bridged heterocyclyl is 6- to 14-membered, and more preferably 7- to 10-membered. According to the number of membered rings, a bridged heterocyclyl is divided into bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include, but are not limited to, the following groups: azabicyclo[2.2.1]heptanyl, diazabicyclo[2.2.1]heptanyl, azabicyclo[3.1.1]heptanyl, diazabicyclo[3.1.1]heptanyl, azabicyclo[2.2.2]octanyl, diazabicyclo[2.2.2]octanyl, azabicyclo[3.2.1]octanyl or diazabicyclo[3.2.1]octanyl, preferably 2-azabicyclo[2.2.1]heptan-2-yl, 6-azabicyclo[3.1.1]heptan-3-yl, 2-azabicyclo[2.2.2]octan-5-yl, 3-azabicyclo[3.2.1]octan-8-yl.

The heterocyclyl ring may be fused to aryl, heteroaryl or cycloalkyl ring, wherein the ring structure is connected to the parent heterocyclic group together. Heterocyclyl optionally may be substituted or unsubstituted.

In some embodiments, the groups such as alkyl, cycloalkyl, heterocyclyl, aryl or heteroaryl is optionally deuterated. The term “deuterated” is used herein to modify a chemical structure or an organic group or radical, wherein one or more carbon-bound hydrogen(s) are replaced by one or more deuterium(s), e.g., “deuterated-alkyl”, “deuterated-cycloalkyl”, “deuterated-heterocyclyl”, “deuterated-aryl”, “deuterated-heteroaryl”, and the like. For example, the term “deuterated-alkyl” defined above refers to an alkyl group as defined herein, wherein at least one hydrogen atom bound to carbon is replaced by a deuterium. In a deuterated alkyl group, at least one carbon atom is bound to a deuterium; and it is possible for a carbon atom to be bound to more than one deuterium; it is also possible that more than one carbon atom in the alkyl group is bound to a deuterium.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, a reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such a ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H, et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

“Prodrug” refers to a derivative of an active agent that requires a transformation within the body to release the active agent. In some embodiments, the transformation is an enzymatic transformation. Prodrugs are frequently, although not necessarily, pharmacologically inactive until converted to the active agent.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as a compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, the severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cn-m” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### General Reaction Scheme for Compound Preparation

The subject compounds and pharmaceutically acceptable salts thereof, can be prepared from (a) commercially available starting materials (b) known starting materials which may be prepared as described in literature procedures (c) new intermediates described in the schemes and experimental procedures herein. In making the compounds of the invention, the order of synthetic steps may be varied to increase the yield of the desired product. Some of the compounds in this invention may be generated by the methods as shown in the following reaction schemes and the description thereof.

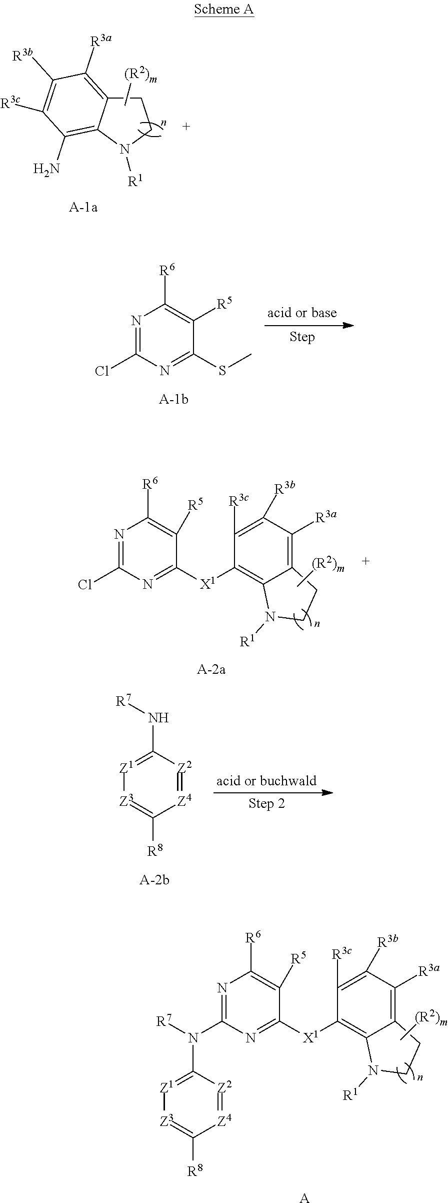

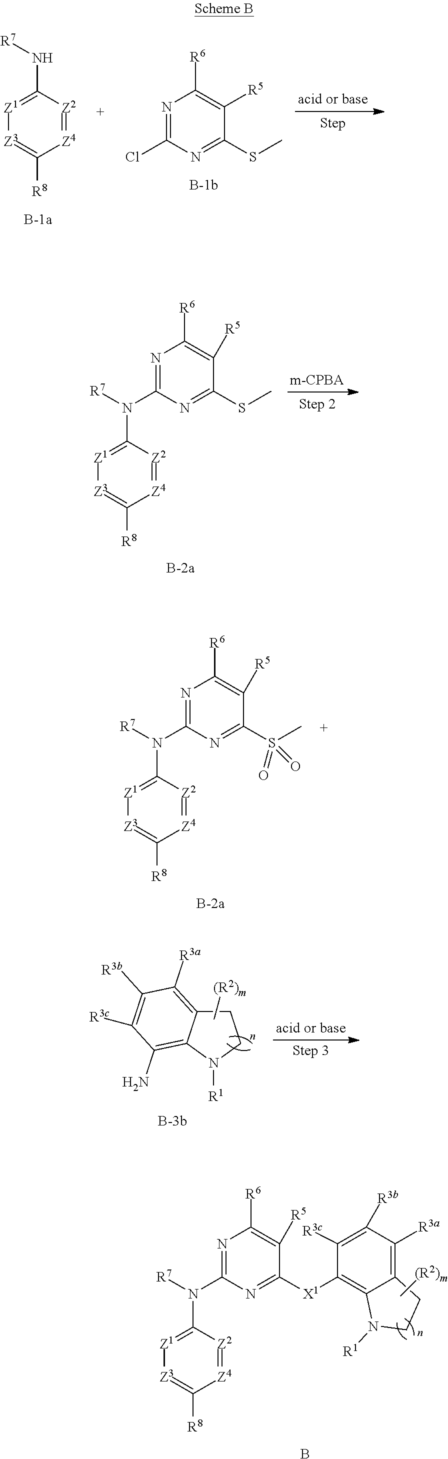

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, the temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on an Agilent instrument operating at 400 MHz. ¹HNMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 nm), Mass detector: 6120 SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS, LCMS-3: LC-MS spectrometer (Agilent 1260 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1290 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.2 mL/min Time (min) A (%) B (%)

| Time (min) | A(%) | B(%) |
|---|---|---|
| 0.00 | 90 | 10 |
| 1.5 | 5 | 95 |
| 2.0 | 5 | 95 |
| 2.1 | 90 | 10 |
| 3.0 | 90 | 10 |

Preparative HPLC was conducted on a column (150×21.2 mm ID, 5 pm, Gemini NXC 18) at a flow rate of 20 ml/min, injection volume 2 ml, at room temperature and UV Detection at 214 nm and 254 nm.

In the following examples, the abbreviations below are used

| | |
|---|---|
| (BPin)₂ | 4,4,4′,4′,5,5,5′,5′-Octamethyl-2,2′-bi-1,3,2-dioxaborolane |
| Ac₂O | acetic anhydride |
| AcCl | Acetyl chloride |
| ACN | Acetonitrile |
| AcOH or HOAc | Acetic acid |
| AcONa or NaOAc | Sodium acetate |
| Aq | Aqueous |
| BINAP | (±)-2,2′-Bis(diphenylphosphino)-1,1′-BINAPhthyl |
| Bn | benzyl |
| BnBr | Benzyl Bromide |
| Boc | t-Butyloxy carbonyl |
| C: 40691-33-6 | dichlorobis(tri-o-tolylphosphine)palladium(II) |
| Cbz | Benzyloxycarbonyl |
| DCM | Dichloromethane |
| Con. | Concentrated |
| DavePhos | 2′-(Dicyclohexylphosphino)-N,N-dimethyl-2-biphenylamine |
| DBU | 1,8-diazabicyclo[5.4.0]undec-7-ene |
| DCE | dichloroethane |
| DHP | 3,4-Dihydro-2H-pyran |
| DIBAL-H | Diisobutylaluminium hydride |
| DIEA or DIPEA | N,N-diisopropylethylamine |
| DMAP | 4-N,N-dimethylaminopyridine |
| DMF | N,N-Dimethylformamide |
| DMSO | Dimethyl sulfoxide |
| Dppf | 1,1″-bis(diphenylphosphino)ferrocene |
| EA or EtOAc | Ethyl acetate |
| EtOH | ethanol |
| FA | Formic acid |
| h or hr | Hour |
| HATU | 2-(7-Azabenzotriazol-1-yl)-N,N,N′,N′-tetramethyluronium |
| | hexafluorophosphate |
| HBTU | O-(7-Benzotriazole-1-yl)-N,N,N′,N′-tetramethyluronium |
| | hexafluorophosphate |
| Hex | Hexane |
| HPLC | High Performance Liquid Chromatography |
| hrs | hours |
| IBX | 2-Iodoxybenzoic acid |
| IPA | 2-propanol |
| i-PrOH | Isopropyl alcohol |
| KHMDS | Potassium bis(trimethylsilyl)amide |
| KOAc | Potassium Acetate |
| MeCN or ACN | Acetonitrile |
| MeOH | Methanol |
| Min | Minutes |
| ms or MS | Mass spectrum |
| MsCl | Methanesulfonyl chloride |
| MsOH | Methanesulfonic acid |
| MTBE | Methyl tert-butyl ether |
| o/n | overnight |
| Pd(dppf)Cl₂ | [1,1′-Bis(diphenylphosphino)ferrocene]dichloropalladium(II) |
| G3 BrettPhos Pd | Methanesulfonato(2-dicyclohexylphosphino-3,6-dimethoxy-2′,4′,6′- |
| | tri-i-propyl-1,1′-biphenyl)(2′-amino-1,1′-biphenyl-2-yl)palladium(II) |
| Pd₂(dba)₂ | |
| PE | Petroleum ether |
| PhMe | Toluene |
| PPA | Polyphosphoric acid |
| R.T. or r.t. | Room temperature |
| Rt | Retention time |
| SEMCl | 2-(Trimethylsilyl)ethoxymethyl chloride |
| STAB | Sodium Triacetoxyborohydride; Sodium triacetoborohydride |
| TBAF | Tetra-butyl ammonium fluoride |
| TBDPS | tert-Butyldiphenylsilyl |
| TBS | tert-Butyldimethylsilyl |
| TBSCl | tert-Butyldimethylsilyl chloride |
| t-Bu | tert-butyl |
| t-BuOH | tert-Butanol |
| t-BuONa | Sodium tert-butoxide |
| TEA | Triethylamine |
| Tf₂O | Triflic anhydride |
| TFA | Trifluoroacetic acid |
| THF | Tetrahydrofuran |
| TLC | Thin layer chromatography |
| TMSOK | Potassium trimethylsilanolate |
| Ts | para-Toluenesulfonyl |
| TsCl | 4-Toluenesulfonyl chloride |
| TsOH | p-toluenesulfonic acid |
| TsOH, Py | Pyridinium toluene-4-sulphonate |
| Xphos | 2-Dicyclohexylphosphino-2′,4′,6′-triisopropylbiphenyl |
| XantPhos | 4,5-Bis(diphenylphosphino)-9,9-dimethylxanthene |

### Example 30: 5-chloro-N²-(2-methoxy-4-(4-(4-methylpiperazin-1-yl)piperidin-1-yl)phenyl)-N⁴-(1-(methylsulfonyl)indolin-7-yl)pyrimidine-2,4-diamine

### Step 1: 1-(methylsulfonyl)-7-nitroindoline

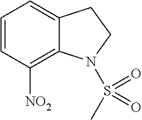 [SMILES: CS(=O)(=O)N1CCc2cccc([N+](=O)[O-])c21]

To a stirred solution of 7-nitroindoline (300 mg, 1.8 mmol) and NaH (146 mg, 3.6 mmol, 60% dispersion in mineral oil) in DMF (5 mL) was added methanesulfonyl chloride dropwise (315 mg, 2.7 mmol). The resulting mixture was stirred at room temperature for 1 hour. The reaction was quenched with saturated NH₄Cl (aq.) solution and extracted with EtOAc (2×50.0 mL). The combined organic layers were washed with brine (2×50.0 mL), dried over Na₂SO₄ and concentrated under vacuum to afford the crude residue, which was purified with silica gel column chromatography (PE: EA=100:0˜ 2:1 gradient elution) to give the title product (390 mg, 88%). [M+H]⁺=243.1.

### Step 2: 1-(methylsulfonyl)indolin-7-amine

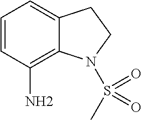

Under N₂, to a solution of 1-(methylsulfonyl)-7-nitroindoline (390 mg, 1.6 mmol) in MeOH (20 mL) was added 10% Pd/C (50 mg) at room temperature. And then the mixture was exchanged with H₂ two times and stirred under H₂ atmosphere at room temperature for 2 h. Reaction was monitored by LC-MS. The mixture was filtered through a pad of Celite and washed with MeOH (20 mL). The filtrate was concentrated under vacuum to obtain the title product (340 mg, 99%). [M+H]⁺=213.1.

### Step 3: N-(2,5-dichloropyrimidin-4-yl)-1-(methylsulfonyl)indolin-7-amine

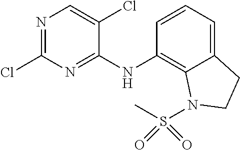 [SMILES: CS(=O)(=O)N1CCc2cccc(Nc3nc(Cl)ncc3Cl)c21]

A mixture of 1-(methylsulfonyl)indolin-7-amine (340 mg, 1.6 mmol), 2,4,5-trichloropyrimidine (584 mg, 3.2 mmol) and DIEA (412 mg, 3.2 mmol) in i-PrOH (20 mL) was stirred in a round bottom flask at 100° C. for 16 h. The mixture was evaporated in vacuum to afford the crude product, which was purified with silica gel column chromatography (PE: EA=100: 0˜ 2: 1 gradient elution) to give the title product (540 mg, 94%). [M+H]⁺=359.2.

### Step 4: 5-chloro-N²-(2-methoxy-4-(4-(4-methylpiperazin-1-yl)piperidin-1-yl)phenyl)-N⁴-(1-(methylsulfonyl)indolin-7-yl)pyrimidine-2,4-diamine

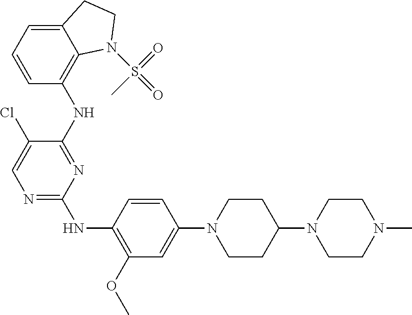 [SMILES: COc1cc(N2CCC(N3CCN(C)CC3)CC2)ccc1Nc1ncc(Cl)c(Nc2cccc3c2N(S(C)(=O)=O)CC3)n1]

A mixture of N-(2,5-dichloropyrimidin-4-yl)-1-(methylsulfonyl)indolin-7-amine (50 mg, 0.14 mmol), 2-methoxy-4-(4-(4-methylpiperazin-1-yl)piperidin-1-yl)aniline (50 mg, 0.17 mmol), Pd₂(dba)₃ (13 mg, 0.014 mmol), BINAP (18 mg, 0.028 mmol) and K₃PO₄ (88 mg, 0.42 mmol) in toluene (8 mL) was stirred in a round bottom flask at 100° C. overnight under N₂. The mixture was evaporated in vacuum to afford the crude residue, which was purified with silica gel column chromatography (DCM: MeOH=100: 0˜ 10: 1 gradient elution) to give the title product (32 mg, 37%). ¹H NMR (400 MHz, DMSO) δ_{H}8 0.9 3 (s, 1H), 8.08 (s, 1H), 7.89 (s, 2H), 7.44 (d, J=8.3 Hz, 1H), 7.15 (s, 2H), 6.60 (s, 1H), 6.40 (d, J=8.7 Hz, 1H), 4.05 (s, 2H), 3.76 (s, 3H), 3.69 (d, J=11.7 Hz, 2H), 3.10 (s, 2H), 3.04 (s, 3H), 2.64 (t, J=11.9 Hz, 2H), 2.51 (s, 4H), 2.45-2.25 (m, 5H), 2.16 (s, 3H), 1.84 (d, J=10.6 Hz, 2H), 1.51 (d, J=10.9 Hz, 2H); [M+H]⁺=627.3.

### Example 1: 5-chloro-N⁴-(4-fluoro-1-(methylsulfonyl)indolin-7-yl)-N²-(2-methoxy-4-(4-(4-methylpiperazin-1-yl)piperidin-1-yl)phenyl)pyrimidine-2,4-diamine

### Step 1: N-(2,5-dichloropyrimidin-4-yl)-4-fluoroindolin-7-amine

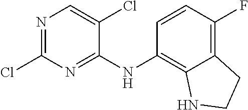 [SMILES: Fc1ccc(Nc2nc(Cl)ncc2Cl)c2c1CCN2]

A mixture of 4-fluoroindolin-7-amine (200 mg, 1.3 mmol), 2,4,5-trichloropyrimidine (360 mg, 2.0 mmol) and DIEA (340 mg, 2.6 mmol) in i-PrOH (20 mL) was stirred in a round bottom flask at 100° C. for 16 hours. The mixture was evaporated in vacuum to afford the crude product, which was purified with silica gel column chromatography (PE: EA=100: 0˜ 2: 1 gradient elution) to give the title product (170 mg, 43%). [M+H]⁺=299.1.

### Step 2: N-(2,5-dichloropyrimidin-4-yl)-4-fluoro-1-(methylsulfonyl)indolin-7-amine

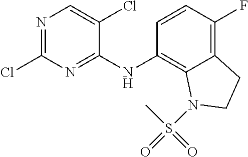 [SMILES: CS(=O)(=O)N1CCc2c(F)ccc(Nc3nc(Cl)ncc3Cl)c21]

To a stirred solution of N-(2,5-dichloropyrimidin-4-yl)-4-fluoroindolin-7-amine (70 mg, 0.23 mmol), DMAP (34 mg, 0.28 mmol) and TEA (47 mg, 0.47 mmol) in DCM (5 mL) was added methanesulfonyl chloride dropwise (40 mg, 0.35 mmol) at 0° C. The resulting mixture was stirred at room temperature for 1 hour. The reaction was extracted with EtOAc, the organic layer was washed with brine (2×50.0 mL), dried over Na₂SO₄ and concentrated under vacuum to afford the crude residue, which was purified with silica gel column chromatography (PE: EA=100: 0˜ 2: 1 gradient elution) to give the title product (50 mg, 58%). [M+H]⁺=377.1.

### Step 3: 5-chloro-N⁴-(4-fluoro-1-(methylsulfonyl)indolin-7-yl)-N²-(2-methoxy-4-(4-(4-methylpiperazin-1-yl)piperidin-1-yl)phenyl)pyrimidine-2,4-diamine

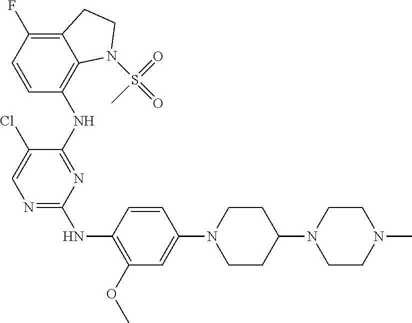 [SMILES: COc1cc(N2CCC(N3CCN(C)CC3)CC2)ccc1Nc1ncc(Cl)c(Nc2ccc(F)c3c2N(S(C)(=O)=O)CC3)n1]

The titled compound was prepared in a manner similar to that in Example 30. ¹H NMR (400 MHz, DMSO) δ_{H} 8.84 (s, 1H), 8.07 (s, 1H), 7.89 (s, 1H), 7.83 (s, 1H), 7.39 (d, J=7.4 Hz, 1H), 7.04 (s, 1H), 6.59 (s, 1H), 6.40 (d, J=8.2 Hz, 1H), 4.10 (t, J=6.5 Hz, 2H), 3.76 (s, 3H), 3.69 (d, J=11.5 Hz, 2H), 3.36 (s, 3H), 3.11 (s, 5H), 2.64 (t, J=11.4 Hz, 2H), 2.52 (s, 2H), 2.38-2.23 (m, 4H), 2.14 (s, 3H), 1.84 (d, J=10.4 Hz, 2H), 1.50 (dd, J=21.4, 10.5 Hz, 2H); [M+H]⁺=645.3.

### Example 2: N⁴-(4-bromo-1-(methylsulfonyl)indolin-7-yl)-5-chloro-N²-(2-methoxy-4-(4-(4-methylpiperazin-1-yl)piperidin-1-yl)phenyl)pyrimidine-2,4-diamine

### Step 1: 4-bromo-N-(2,5-dichloropyrimidin-4-yl)indolin-7-amine

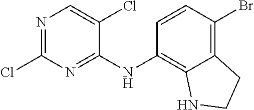 [SMILES: Clc1ncc(Cl)c(Nc2ccc(Br)c3c2NCC3)n1]

The titled compound (76 mg, 56%) was prepared in a manner similar to that in Example 1 step 1 from 4-bromoindolin-7-amine and 2,4,5-trichloropyrimidine. [M+H]⁺=359.1/361.1.

### Step 2: 4-bromo-N-(2,5-dichloropyrimidin-4-yl)-1-(methylsulfonyl)indolin-7-amine

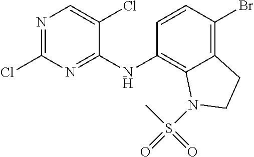 [SMILES: CS(=O)(=O)N1CCc2c(Br)ccc(Nc3nc(Cl)ncc3Cl)c21]

The titled compound (85 mg, 90%) was prepared in a manner similar to that in Example 1 step 2 from 4-bromo-N-(2,5-dichloropyrimidin-4-yl)indolin-7-amine. [M+H]±=436.1/438.1.

### Step 3: N⁴-(4-bromo-1-(methylsulfonyl)indolin-7-yl)-5-chloro-N²-(2-methoxy-4-(4-(4-methylpiperazin-1-yl)piperidin-1-yl)phenyl)pyrimidine-2,4-diamine

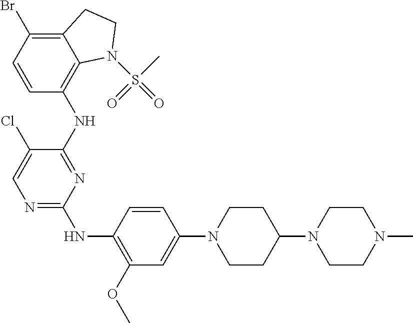 [SMILES: COc1cc(N2CCC(N3CCN(C)CC3)CC2)ccc1Nc1ncc(Cl)c(Nc2ccc(Br)c3c2N(S(C)(=O)=O)CC3)n1]

A mixture of 4-bromo-N-(2,5-dichloropyrimidin-4-yl)-1-(methylsulfonyl)indolin-7-amine (50 mg, 0.11 mmol), 2-methoxy-4-(4-(4-methylpiperazin-1-yl)piperidin-1-yl)aniline (42 mg, 0.14 mmol) and MsOH (6 mg, 0.06 mmol) in t-BuOH (5 mL) was stirred in a round bottom flask at 100° C. for 16 hours. The mixture was evaporated in vacuum to afford the crude product, which was purified with pre-HPLC (0.1% FA in water: acetonitrile=90:1˜50:50 gradient elution) to give the title product (9 mg, 11%). ¹H NMR (400 MHz, CD₃OD) δ_{H} 8.51 (s, 1H), 7.99 (s, 1H), 7.72 (d, J=8.6 Hz, 1H), 7.57 (d, J=8.7 Hz, 1H), 7.34 (d, J=9.0 Hz, 1H), 6.64 (s, 1H), 6.41 (d, J=8.5 Hz, 1H), 4.13 (t, J=7.5 Hz, 2H), 3.83 (s, 3H), 3.71 (d, J=12.4 Hz, 2H), 3.16 (t, J=7.7 Hz, 2H), 2.99 (s, 3H), 2.90-2.48 (m, 10H), 2.43 (t, J=9.7 Hz, 1H), 2.36 (s, 3H), 2.04 (d, J=11.7 Hz, 2H), 1.68 (q, J=13.9 Hz, 2H); [M+H]⁺=705.2/707.2.

### Example 4: 5-chloro-N²-(2-methoxy-4-(4-(4-methylpiperazin-1-yl)piperidin-1-yl)phenyl)-N⁴-(4-methyl-1-(methylsulfonyl)indolin-7-yl)pyrimidine-2,4-diamine Step 1: N-(2,5-dichloropyrimidin-4-yl)-4-methylindolin-7-amine

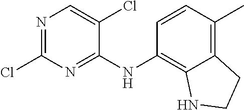 [SMILES: Cc1ccc(Nc2nc(Cl)ncc2Cl)c2c1CCN2]

The titled compound (170 mg, 52%) was prepared in a manner similar to that in Example 1 step 1 from 4-methylindolin-7-amine and 2,4,5-trichloropyrimidine. [M+H]⁺=295.2.

### Step 2: N-(2,5-dichloropyrimidin-4-yl)-4-methyl-1-(methylsulfonyl)indolin-7-amine

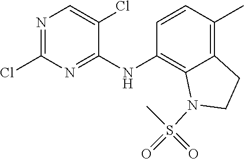 [SMILES: Cc1ccc(Nc2nc(Cl)ncc2Cl)c2c1CCN2S(C)(=O)=O]

The titled compound (50 mg, 76%) was prepared in a manner similar to that in Example 1 step 2 from N-(2,5-dichloropyrimidin-4-yl)-4-methylindolin-7-amine. [M+H]⁺=373.2.

### Step 3: 5-chloro-N²-(2-methoxy-4-(4-(4-methylpiperazin-1-yl)piperidin-1-yl)phenyl)-N⁴-(4-methyl-1-(methylsulfonyl)indolin-7-yl)pyrimidine-2,4-diamine

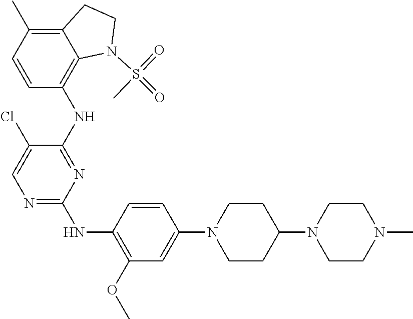 [SMILES: COc1cc(N2CCC(N3CCN(C)CC3)CC2)ccc1Nc1ncc(Cl)c(Nc2ccc(C)c3c2N(S(C)(=O)=O)CC3)n1]

The titled compound was prepared in a manner similar to that in Example 30. ¹H NMR (400 MHz, DMSO) δ_{H} 8.83 (s, 1H), 8.05 (s, 1H), 7.83 (s, 1H), 7.72 (d, J=7.9 Hz, 1H), 7.42 (d, J=8.3 Hz, 1H), 6.99 (d, J=7.7 Hz, 1H), 6.59 (s, 1H), 6.39 (d, J=8.6 Hz, 1H), 4.04 (t, J=6.9 Hz, 2H), 3.76 (s, 3H), 3.69 (d, J=11.4 Hz, 2H), 3.02 (s, 5H), 2.65 (t, J=11.5 Hz, 2H), 2.51 (s, 5H), 2.34 (dt, J=18.7, 7.5 Hz, 4H), 2.24 (s, 3H), 2.17 (s, 3H), 1.85 (d, J=11.5 Hz, 2H), 1.58-1.44 (m, 2H); [M+H]⁺=641.3.

### Example 5: 5-chloro-N²-(2-methoxy-4-(4-(4-methylpiperazin-1-yl)piperidin-1-yl)phenyl)-N⁴-(5-methyl-1-(methylsulfonyl)indolin-7-yl)pyrimidine-2,4-diamine

### Step 1: N-(2,5-dichloropyrimidin-4-yl)-5-methylindolin-7-amine

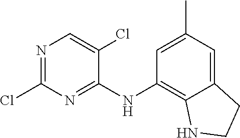 [SMILES: Cc1cc2c(c(Nc3nc(Cl)ncc3Cl)c1)NCC2]

The titled compound (170 mg, 52%) was prepared in a manner similar to that in Example 1 step 1 from 5-methylindolin-7-amine and 2,4,5-trichloropyrimidine. [M+H]⁺=295.2.

### Step 2: N-(2,5-dichloropyrimidin-4-yl)-5-methyl-1-(methylsulfonyl)indolin-7-amine

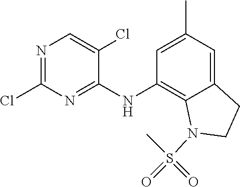 [SMILES: Cc1cc2c(c(Nc3nc(Cl)ncc3Cl)c1)N(S(C)(=O)=O)CC2]

The titled compound (35 mg, 56%) was prepared in a manner similar to that in Example 1 step 2 from N-(2,5-dichloropyrimidin-4-yl)-5-methylindolin-7-amine. [M+H]⁺=373.2.

### Step 3: 5-chloro-N²-(2-methoxy-4-(4-(4-methylpiperazin-1-yl)piperidin-1-yl)phenyl)-N⁴-(5-methyl-1-(methylsulfonyl)indolin-7-yl)pyrimidine-2,4-diamine

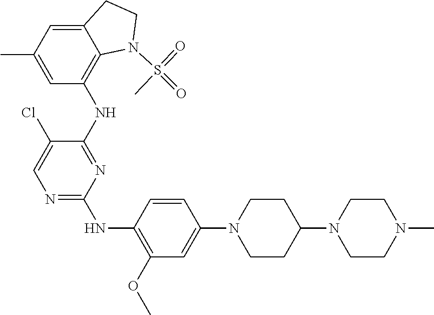

The titled compound was prepared in a manner similar to that in Example 30. ¹H NMR (400 MHz, DMSO) δ_{H} 8.92 (s, 1H), 8.21 (s, 1H), 7.78-7.65 (m, 2H), 7.32 (d, J=32.7 Hz, 1H), 7.11 (s, 1H), 6.60 (s, 1H), 6.47 (d, J=9.0 Hz, 1H), 4.25 (t, J=8.0 Hz, 2H), 3.80 (s, 3H), 3.67 (d, J=11.6 Hz, 2H), 3.07 (s, 2H), 2.94 (s, 3H), 2.62 (t, J=11.4 Hz, 2H), 2.57-2.51 (m, 5H), 2.38-2.22 (m, 7H), 2.16 (s, 3H), 1.83 (d, J=11.1 Hz, 2H), 1.50 (dd, J=22.1, 10.4 Hz, 2H); [M+H]⁺=641.3.

### Example 6: 5-chloro-N²-(2-methoxy-4-(4-(4-methylpiperazin-1-yl)piperidin-1-yl)phenyl)-N⁴-(1-(propylsulfonyl)indolin-7-yl)pyrimidine-2,4-diamine

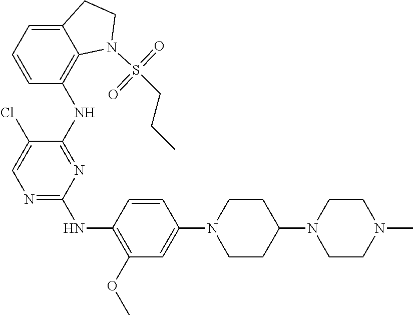 [SMILES: CCCS(=O)(=O)N1CCc2cccc(Nc3nc(Nc4ccc(N5CCC(N6CCN(C)CC6)CC5)cc4OC)ncc3Cl)c21]

The titled compound (8.25 mg, 22%) was prepared in a manner similar to that in Example 7 step 7 from 5-chloro-N⁴-(indolin-7-yl)-N²-(2-methoxy-4-(4-(4-methylpiperazin-1-yl)piperidin-1-yl)phenyl)pyrimidine-2,4-diamine and propane-1-sulfonyl chloride. ¹H NMR (400 MHz, DMSO) δ 8.90 (s, 1H), 8.08 (s, 1H), 7.86 (s, 1H), 7.82 (s, 1H), 7.44 (d, J=8.7 Hz, 1H), 7.14 (s, 2H), 6.59 (s, 1H), 6.38 (d, J=8.6 Hz, 1H), 4.02 (s, 2H), 3.76 (s, 3H), 3.69 (d, J=11.6 Hz, 2H), 3.25-3.16 (m, 2H), 3.09 (s, 2H), 2.62 (d, J=14.0 Hz, 7H), 2.36 (s, 4H), 2.24 (s, 3H), 1.85 (d, J=11.6 Hz, 2H), 1.67 (dd, J=14.7, 7.6 Hz, 2H), 1.51 (d, J=10.9 Hz, 2H), 0.94 (t, J=7.3 Hz, 3H); [M+H]⁺=655.29.

### Example 7: 5-chloro-N⁴-(1-(cyclopropylsulfonyl)indolin-7-yl)-N²-(2-methoxy-4-(4-(4-methylpiperazin-1-yl)piperidin-1-yl)phenyl)pyrimidine-2,4-diamine

### Step 1: 1H-indol-7-amine

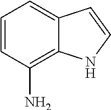

Under N₂, to a solution of 7-nitro-1H-indole (10 g, 61.7 mmol) in MeOH (20 mL) was added 10% Pd/C (2 g) at room temperature. And then the mixture was exchanged with H₂ two times and stirred under H₂ atmosphere at room temperature for 4 h. Reaction was monitored by LC-MS. The mixture was filtered through a pad of Celite and washed with MeOH (20 mL). The filtrate was concentrated under vacuum to obtain the title product (7.5 g, 92%). [M+H]⁺=133.1.

### Step 2: N-(2,5-dichloropyrimidin-4-yl)-1H-indol-7-amine

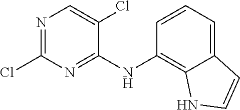 [SMILES: Clc1ncc(Cl)c(Nc2cccc3cc[nH]c23)n1]

A mixture of 1H-indol-7-amine (2 g, 15.2 mmol), 2,4,5-trichloropyrimidine (4.1 g, 22.7 mmol) and DIEA (3.9 g, 30.4 mmol) in i-PrOH (100 mL) was stirred in a round bottom flask at room temperature for 16 hours. The mixture was evaporated in vacuum to afford the crude product, which was purified with silica gel column chromatography (PE: EA=100: 0-2: 1 gradient elution) to give the title product (4 g, 94%). [M+H]⁺=279.1.

### Step 3: N-(2,5-dichloropyrimidin-4-yl)indolin-7-amine

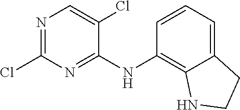 [SMILES: Clc1ncc(Cl)c(Nc2cccc3c2NCC3)n1]

To a solution of N-(2,5-dichloropyrimidin-4-yl)-1H-indol-7-amine (2 g, 7.2 mmol) in AcOH (60 mL) was added NaBH₃CN (540 mg, 8.6 mmol) at 0° C. And then the mixture was heated to room temperature for 4 h. Reaction was monitored by LC-MS. The reaction was quenched with sat. NH₄Cl and extracted with EtOAc. The combined organic layer was washed with brine (2×150 mL) and saturated NaHCO₃(aq.) solution, (2×150 mL), dried over Na₂SO₄ and concentrated under vacuum to afford the crude residue, which was purified with silica gel column chromatography (PE: EA=100: 0-2: 1 gradient elution) to give the title product (1.8 g, 91%). [M+H]⁺=281.1.

### Step 4: tert-butyl 7-((2,5-dichloropyrimidin-4-yl)amino)indoline-1-carboxylate

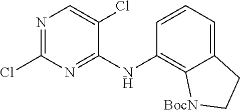 [SMILES: CC(C)(C)OC(=O)N1CCc2cccc(Nc3nc(Cl)ncc3Cl)c21]

A mixture of N-(2,5-dichloropyrimidin-4-yl)indolin-7-amine (600 mg, 2.1 mmol), di-tert-butyl dicarbonate (560 mg, 2.6 mmol) and TEA (636.3 mg, 6.3 mmol) in THF (50 mL) was stirred in a round bottom flask at room temperature for 16 hours. The mixture was evaporated in vacuum to afford the crude product, which was purified with silica gel column chromatography (PE: EA=100: 0˜2: 1 gradient elution) to give the title product (500 mg, 62%). [M+H]⁺=381.2.

### Step 5: tert-butyl 7-((5-chloro-2-((2-methoxy-4-(4-(4-methylpiperazin-1-yl)piperidin-1-yl)phenyl)amino)pyrimidin-4-yl)amino)indoline-1-carboxylate

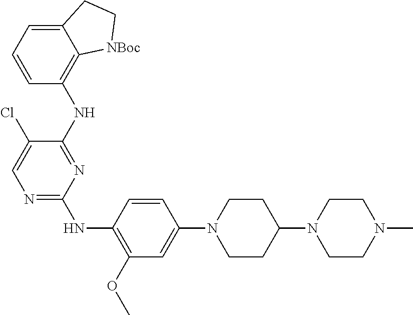 [SMILES: COc1cc(N2CCC(N3CCN(C)CC3)CC2)ccc1Nc1ncc(Cl)c(Nc2cccc3c2N(C(=O)OC(C)(C)C)CC3)n1]

The titled compound (500 mg, 49%) was prepared in a manner similar to that in Example 30 step 4 from tert-butyl 7-((2,5-dichloropyrimidin-4-yl)amino)indoline-1-carboxylate and 2-methoxy-4-(4-(4-methylpiperazin-1-yl)piperidin-1-yl)aniline. [M+H]⁺=649.3.

### Step 6: 5-chloro-N⁴-(indolin-7-yl)-N²-(2-methoxy-4-(4-(4-methylpiperazin-1-yl)piperidin-1-yl)phenyl)pyrimidine-2,4-diamine

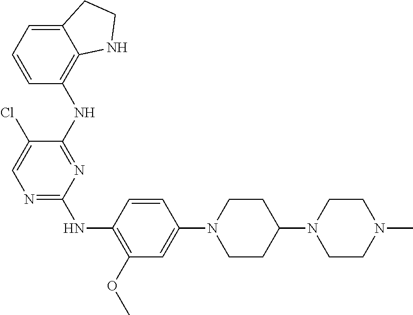 [SMILES: COc1cc(N2CCC(N3CCN(C)CC3)CC2)ccc1Nc1ncc(Cl)c(Nc2cccc3c2NCC3)n1]

A mixture of tert-butyl 7-((5-chloro-2-((2-methoxy-4-(4-(4-methylpiperazin-1-yl)piperidin-1-yl)phenyl)amino)pyrimidin-4-yl)amino)indoline-1-carboxylate (500 mg, 0.77 mmol) and trifluoroacetic acid (10 ml) in DCM (10 mL) was stirred in a round bottom flask at room temperature for 2 hours. The mixture was extracted with DCM (2×50 mL). The combined organic lays were washed with saturated NaHCO₃(aq.) solution (50 mL), dried over Na₂SO₄ and concentrated under vacuum to afford the title product (350 mg, 83%). [M+H]⁺=549.2.

### Step 7: 5-chloro-N⁴-(1-(cyclopropylsulfonyl)indolin-7-yl)-N²-(2-methoxy-4-(4-(4-methylpiperazin-1-yl)piperidin-1-yl)phenyl)pyrimidine-2,4-diamine

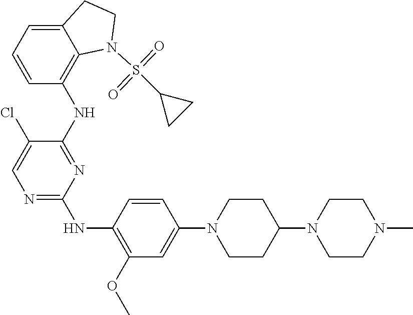 [SMILES: COc1cc(N2CCC(N3CCN(C)CC3)CC2)ccc1Nc1ncc(Cl)c(Nc2cccc3c2N(S(=O)(=O)C2CC2)CC3)n1]

To a stirred solution of 5-chloro-N⁴-(indolin-7-yl)-N²-(2-methoxy-4-(4-(4-methylpiperazin-1-yl)piperidin-1-yl)phenyl)pyrimidine-2,4-diamine (30 mg, 0.055 mmol), DMAP(8 mg, 0.066 mmol) and TEA (17 mg, 0.16 mmol) in DCM (5 mL) was added cyclopropanesulfonyl chloride (10 mg, 0.066 mmol) at 0° C. The resulting mixture was stirred at room temperature for 1 hour. The reaction was extracted with DCM, washed with brine (2×50.0 mL), dried over Na₂SO₄ and concentrated under vacuum to afford the crude residue, which was purified with pre-HPLC (0.1% FA in water: acetonitrile=90:1˜50:50 gradient elution) to give the title product (16 mg, 45%). H¹ NMR (400 MHz, DMSO) δ_{H} 8.88 (s, 1H), 8.08 (s, 1H,), 7.89 (s, 2H), 7.44 (d, J=8.4, 1H), 7.15 (s, 2H), 6.60 (s, 1H), 6.40 (d, J=8.4, 1H), 4.07 (m, 2H), 3.76 (s, 3H), 3.69 (d, J=11.2, 3H), 3.43-3.36 (m, 2H), 3.18-3.11 (m, 2H), 2.76 (s, 1H), 2.64 (t, J=11.2, 2H), 2.5-2.52 (m, 2H), 2.41-2.26 (m, 4H), 2.16 (s, 3H), 1.84 (d, J=11.6, 2H), 1.51 (q, J=12.0, 2H), 1.01-0.82 (m, 4H); [M+H]⁺=653.3.

### Example 8: 5-chloro-N²-(2-methoxy-4-(4-(4-methylpiperazin-1-yl)piperidin-1-yl)phenyl)-N⁴-(1-(phenethylsulfonyl)indolin-7-yl)pyrimidine-2,4-diamine

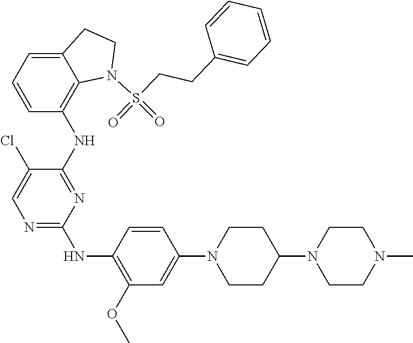 [SMILES: COc1cc(N2CCC(N3CCN(C)CC3)CC2)ccc1Nc1ncc(Cl)c(Nc2cccc3c2N(S(=O)(=O)CCc2ccccc2)CC3)n1]

The titled compound was prepared in a manner similar to that in Example 7. ¹H NMR (400 MHz, DMSO) δ_{H} 8.89 (s, 1H), 8.08 (s, 1H), 7.86 (s, 1H), 7.81 (s, 1H), 7.44 (d, J=8.0 Hz, 1H), 7.33-7.09 (m, 7H), 6.59 (s, 1H), 6.37 (d, J=8.4 Hz, 1H), 4.06 (s, 2H), 3.76 (s, 3H), 3.68 (d, J=11.5 Hz, 2H), 3.55 (s, 2H), 3.09 (s, 2H), 2.95 (s, 2H), 2.63 (t, J=11.7 Hz, 2H), 2.51 (s, 5H), 2.40-2.25 (m, 4H), 2.14 (s, 3H), 1.84 (d, J=11.5 Hz, 2H), 1.50 (q, J=11.4 Hz, 2H); [M+H]⁺=717.3.

### Example 9: N⁴-(1-(tert-butylsulfonyl)indolin-7-yl)-5-chloro-N²-(2-methoxy-4-(4-(4-methylpiperazin-1-yl)piperidin-1-yl)phenyl)pyrimidine-2,4-diamine

### Step 1: N⁴-(1-(tert-butylsulfinyl)indolin-7-yl)-5-chloro-N²-(2-methoxy-4-(4-(4-methylpiperazin-1-yl)piperidin-1-yl)phenyl)pyrimidine-2,4-diamine

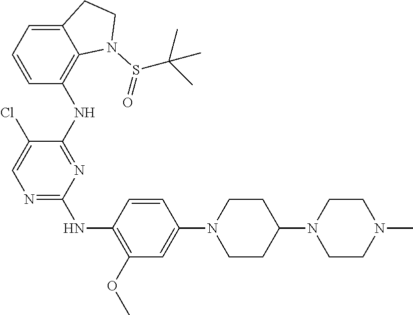 [SMILES: COc1cc(N2CCC(N3CCN(C)CC3)CC2)ccc1Nc1ncc(Cl)c(Nc2cccc3c2N(S(=O)C(C)(C)C)CC3)n1]

The titled compound (80 mg, 82%) was prepared in a manner similar to that in Example 7 step 7 from 5-chloro-N⁴-(indolin-7-yl)-N²-(2-methoxy-4-(4-(4-methylpiperazin-1-yl)piperidin-1-yl)phenyl)pyrimidine-2,4-diamine and 2-methylpropane-2-sulfinic chloride, [M+H]±=653.3.

### Step 2: N⁴-(1-(tert-butylsulfonyl)indolin-7-yl)-5-chloro-N²-(2-methoxy-4-(4-(4-methylpiperazin-1-yl)piperidin-1-yl)phenyl)pyrimidine-2,4-diamine

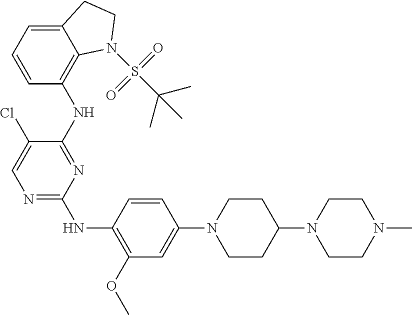 [SMILES: COc1cc(N2CCC(N3CCN(C)CC3)CC2)ccc1Nc1ncc(Cl)c(Nc2cccc3c2N(S(=O)(=O)C(C)(C)C)CC3)n1]

To a stirred solution of N⁴-(1-(tert-butylsulfinyl)indolin-7-yl)-5-chloro-N²-(2-methoxy-4-(4-(4-methylpiperazin-1-yl)piperidin-1-yl)phenyl)pyrimidine-2,4-diamine (80 mg, 0.12 mmol) in DCE (10 mL) was added m-CPBA (84 mg, 0.49 mmol) at 80° C. The resulting mixture was stirred at room temperature for 2 hours. The reaction was quenched with saturated Na₂S₂O₃ (aq.) solution and extracted with DCE (2×50 mL). The combined organic layer was washed with saturated NaHCO₃(aq.) solution (2×50 mL), dried over Na₂SO₄ and concentrated under vacuum to afford the crude residue, which was purified with pre-HPLC (0.1% FA in water:

acetonitrile=90:1˜50:50 gradient elution) to give the title product (42 mg, 49%). ¹H NMR (400 MHz, DMSO) δ_{H} 8.49 (s, 1H), 8.08 (s, 1H), 7.73 (s, 1H), 7.54 (s, 1H), 7.45 (d, J=8.4 Hz, 1H), 7.17 (s, 2H), 6.56 (s, 1H), 6.30 (d, J=8.1 Hz, 1H), 4.00 (s, 2H), 3.76 (s, 3H), 3.64 (d, J=10.6 Hz, 2H), 3.06 (s, 2H), 2.59 (d, J=11.6 Hz, 2H), 2.50-2.45 (m, 5H), 2.38-2.22 (m, 4H), 2.14 (s, 3H), 1.82 (d, J=11.4 Hz, 2H), 1.56-1.46 (m, 2H), 1.44 (s, 9H); [M+H]⁺=669.3.

### Example 10: 5-chloro-N²-(2-methoxy-4-(4-(4-methylpiperazin-1-yl)piperidin-1-yl)phenyl)-N⁴-(1-(methylsulfonyl)-1,2,3,4-tetrahydroquinolin-8-yl)pyrimidine-2,4-diamine

### Step 1: N-(2,5-dichloropyrimidin-4-yl)-1,2,3,4-tetrahydroquinolin-8-amine

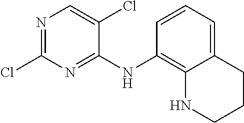 [SMILES: Clc1ncc(Cl)c(Nc2cccc3c2NCCC3)n1]

The titled compound (280 mg, 91%) was prepared in a manner similar to that in Example 1 step 1 from 1,2,3,4-tetrahydroquinolin-8-amine and 2,4,5-trichloropyrimidine. [M+H]⁺=295.2.

### Step 2: N-(2,5-dichloropyrimidin-4-yl)-1-(methylsulfonyl)-1,2,3,4-tetrahydroquinolin-8-amine

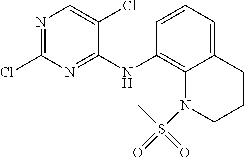 [SMILES: CS(=O)(=O)N1CCCc2cccc(Nc3nc(Cl)ncc3Cl)c21]

The titled compound (280 mg, 91%) was prepared in a manner similar to that in Example 1 step 2 from N-(2,5-dichloropyrimidin-4-yl)-1,2,3,4-tetrahydroquinolin-8-amine and methanesulfonyl chloride. [M+H]⁺=373.1.

### Step 3: 5-chloro-N²-(2-methoxy-4-(4-(4-methylpiperazin-1-yl)piperidin-1-yl)phenyl)-N₄-(1-(methylsulfonyl)-1,2,3,4-tetrahydroquinolin-8-yl)pyrimidine-2,4-diamine

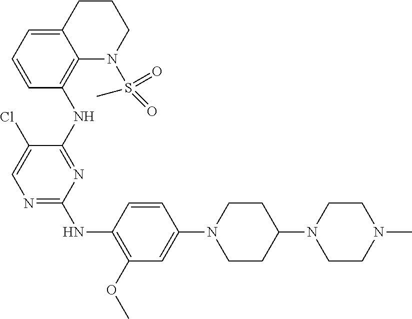 [SMILES: COc1cc(N2CCC(N3CCN(C)CC3)CC2)ccc1Nc1ncc(Cl)c(Nc2cccc3c2N(S(C)(=O)=O)CCC3)n1]

The titled compound was prepared in a manner similar to that in Example 30. ¹H NMR (400 MHz, DMSO) δ_{H} 8.57 (s, 1H), 8.06 (s, 1H), 7.81 (s, 1H), 7.78 (d, J=7.6 Hz, 1H), 7.40 (d, J=8.9 Hz, 1H), 7.20 (s, 1H), 7.05 (d, J=6.6 Hz, 1H), 6.58 (s, 1H), 6.35 (d, J=7.7 Hz, 1H), 3.75 (s, 3H), 3.68 (d, J=11.1 Hz, 2H), 3.54-3.40 (m, 5H), 3.09 (s, 3H), 2.76 (s, 2H), 2.69-2.55 (m, 5H), 2.48-2.43 (m, 2H), 2.43-2.31 (m, 2H), 2.25 (s, 3H), 2.00 (dd, J=11.7, 4.5 Hz, 1H), 1.85 (d, J=11.1 Hz, 2H), 1.51 (dd, J=22.8, 11.4 Hz, 2H); [M+H]⁺=641.3.

### Example 11: 5-chloro-N⁴-(1-(((3,3-difluorocyclobutyl)methyl)sulfonyl)indolin-7-yl)-N²-(2-methoxy-4-(4-(4-methylpiperazin-1-yl)piperidin-1-yl)phenyl)pyrimidine-2,4-diamine

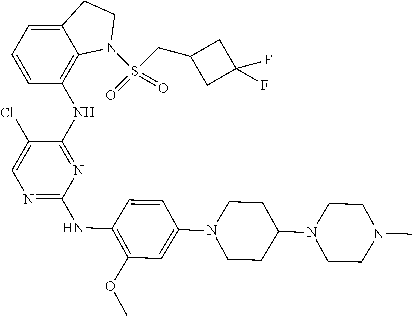 [SMILES: COc1cc(N2CCC(N3CCN(C)CC3)CC2)ccc1Nc1ncc(Cl)c(Nc2cccc3c2N(S(=O)(=O)CC2CC(F)(F)C2)CC3)n1]

The titled compound was prepared in a manner similar to that in Example 7. ¹H NMR (400 MHz, CD₃OD) δ_{H} 8.50 (s, 1H), 7.99 (s, 1H), 7.79-7.65 (m, 2H), 7.23 (s, 2H), 6.63 (s, 1H), 6.35 (dd, J=7.9, 1.2 Hz, 1H), 4.08 (t, J=6.2 Hz, 2H), 3.84 (s, 3H), 3.65 (d, J=10.9 Hz, 2H), 3.36 (d, J=6.4 Hz, 2H), 3.15 (t, J=6.5 Hz, 2H), 3.00-2.63 (m, 12H), 2.58-2.35 (m, 7H), 2.02 (d, J=11.4 Hz, 2H), 1.76-1.59 (m, 2H); [M+H]⁺=717.3.

### Example 12: 5-chloro-N⁴-(1-((fluoromethyl)sulfonyl)indolin-7-yl)-N²-(2-methoxy-4-(4-(4-methylpiperazin-1-yl)piperidin-1-yl)phenyl)pyrimidine-2,4-diamine

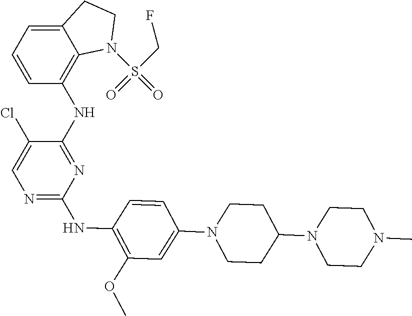 [SMILES: COc1cc(N2CCC(N3CCN(C)CC3)CC2)ccc1Nc1ncc(Cl)c(Nc2cccc3c2N(S(=O)(=O)CF)CC3)n1]

The titled compound was prepared in a manner similar to that in Example 7. ¹H NMR (400 MHz, DMSO) δ 8.72 (s, 1H), 8.09 (s, 1H), 7.83 (d, J=7.8 Hz, 2H), 7.43 (d, J=8.7 Hz, 1H), 7.15 (d, J=6.2 Hz, 2H), 6.59 (d, J=2.2 Hz, 1H), 6.37 (d, J=8.7 Hz, 1H), 5.81 (s, 1H), 5.70 (s, 1H), 4.08 (t, J=7.0 Hz, 2H), 3.76 (s, 3H), 3.68 (d, J=11.4 Hz, 2H), 3.09 (t, J=6.9 Hz, 2H), 2.64 (t, J=11.6 Hz, 2H), 2.50 (s, 5H), 2.34 (s, 4H), 2.17 (s, 3H), 1.84 (d, J=11.6 Hz, 2H), 1.51 (d, J=9.5 Hz, 2H); [M+H]⁺=645.29.

### Example 13: 7-((5-chloro-2-((2-methoxy-4-(4-(4-methylpiperazin-1-yl)piperidin-1-yl)phenyl)amino)pyrimidin-4-yl)amino)-N,N-dimethylindoline-1-sulfonamide

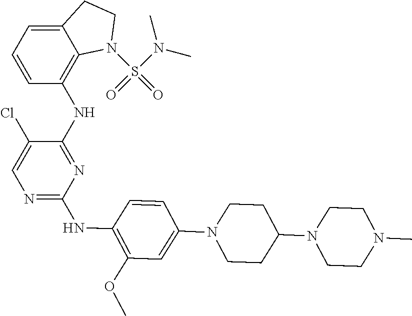 [SMILES: COc1cc(N2CCC(N3CCN(C)CC3)CC2)ccc1Nc1ncc(Cl)c(Nc2cccc3c2N(S(=O)(=O)N(C)C)CC3)n1]

The titled compound was prepared in a manner similar to that in Example 7. ¹H NMR (400 MHz, CD₃OD) δ_{H} 7.98 (s, 1H), 7.81-7.71 (m, 2H), 7.27-7.15 (m, 2H), 6.63 (s, 1H), 6.38 (d, J=8.5 Hz, 1H), 4.60 (s, 1H), 3.91 (t, J=5.8 Hz, 2H), 3.84 (s, 3H), 3.65 (d, J=11.4 Hz, 2H), 3.14 (t, J=6.9 Hz, 2H), 2.90 (s, 6H), 2.83-2.49 (m, 9H), 2.46-2.33 (m, 4H), 2.01 (d, J=11.7 Hz, 2H), 1.72-1.56 (m, 2H); [M+H]⁺=656.3.

### Example 14: 7-((5-chloro-2-((2-methoxy-4-(4-(4-methylpiperazin-1-yl)piperidin-1-yl)phenyl)amino)pyrimidin-4-yl)amino)-N-methylindoline-1-sulfonamide

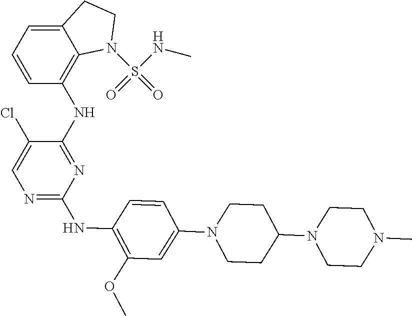 [SMILES: CNS(=O)(=O)N1CCc2cccc(Nc3nc(Nc4ccc(N5CCC(N6CCN(C)CC6)CC5)cc4OC)ncc3Cl)c21]

The titled compound was prepared in a manner similar to that in Example 7. ¹H NMR (400 MHz, CD₃OD) δ_{H} 8.51 (s, 1H), 7.98 (s, 1H), 7.79 (t, J=8.8 Hz, 2H), 7.29-7.10 (m, 2H), 6.64 (s, 1H), 6.40 (d, J=8.6 Hz, 1H), 3.97 (t, J=6.6 Hz, 2H), 3.85 (s, 3H), 3.66 (d, J=12.2 Hz, 2H), 3.15 (t, J=6.7 Hz, 2H), 3.07-2.57 (m, 14H), 2.49 (m, 4H), 2.02 (d, J=11.3 Hz, 2H), 1.80-1.59 (m, 2H); [M+H]⁺=642.3.

### Example 15: 7-((5-chloro-2-((2-methoxy-4-(4-(4-methylpiperazin-1-yl)piperidin-1-yl)phenyl)amino)pyrimidin-4-yl)amino)-N,N-dimethylindoline-1-carboxamide

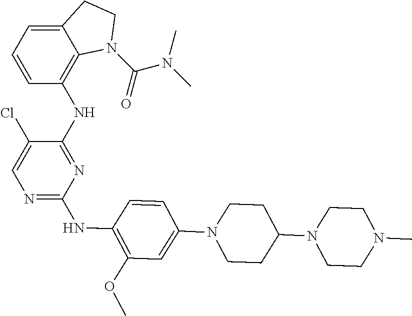 [SMILES: COc1cc(N2CCC(N3CCN(C)CC3)CC2)ccc1Nc1ncc(Cl)c(Nc2cccc3c2N(C(=O)N(C)C)CC3)n1]

The titled compound was prepared in a manner similar to that in Example 7. ¹H NMR (400 MHz, CD₃OD) δ_{H} 8.53 (s, 1H), 7.94 (s, 1H), 7.83 (d, J=8.8 Hz, 1H), 7.55 (d, J=7.5 Hz, 1H), 7.19-7.03 (m, 2H), 6.62 (s, 1H), 6.34 (d, J=8.2 Hz, 1H), 4.00 (t, J=7.8 Hz, 2H), 3.85 (s, 3H), 3.66 (t, J=19.7 Hz, 2H), 3.16 (t, J=7.7 Hz, 2H), 2.97 (s, 6H), 2.88-2.53 (m, 9H), 2.50-2.43 (m, 1H), 2.41 (s, 3H), 2.01 (d, J=10.8 Hz, 2H), 1.66 (q, J=11.4 Hz, 2H), 1.36 (d, J=6.5 Hz, 1H); [M+H]⁺=620.3.

### Example 16: 7-((5-chloro-2-((2-methoxy-4-(4-(4-methylpiperazin-1-yl)piperidin-1-yl)phenyl)amino)pyrimidin-4-yl)amino)-N-methylindoline-1-carboxamide

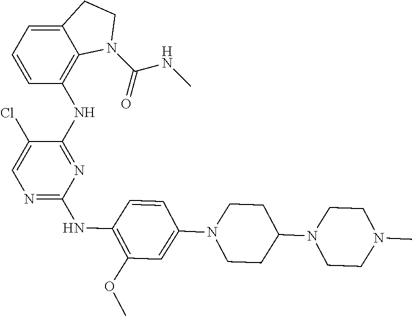 [SMILES: CNC(=O)N1CCc2cccc(Nc3nc(Nc4ccc(N5CCC(N6CCN(C)CC6)CC5)cc4OC)ncc3Cl)c21]

The titled compound was prepared in a manner similar to that in Example 7. ¹H NMR (400 MHz, DMSO) δ_{H} 9.92 (s, 1H), 8.01 (s, 1H), 7.60 (s, 3H), 7.17 (s, 1H), 7.00 (s, 2H), 6.58 (s, 1H), 6.37 (s, 1H), 3.96 (t, J=8.8 Hz, 2H), 3.77 (s, 3H), 3.69-3.62 (m, 2H), 3.28-3.20 (m, 4H), 3.07 (t, J=5.6 Hz, 2H), 2.71 (s, 3H), 2.68-2.58 (m, 3H), 2.39-2.27 (m, 4H), 2.17 (s, 3H), 1.83 (s, 2H), 1.50 (dd, J=21.9, 11.2 Hz, 2H); [M+H]⁺=606.3.

### Example 17: 1-(7-((5-chloro-2-((2-methoxy-4-(4-(4-methylpiperazin-1-yl)piperidin-1-yl)phenyl)amino)pyrimidin-4-yl)amino)indolin-1-yl)ethan-1-one

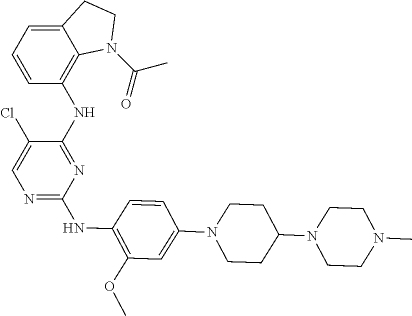 [SMILES: COc1cc(N2CCC(N3CCN(C)CC3)CC2)ccc1Nc1ncc(Cl)c(Nc2cccc3c2N(C(C)=O)CC3)n1]

The titled compound was prepared in a manner similar to that in Example 7. ¹H NMR (400 MHz, DMSO) δ_{H} 9.74 (s, 1H), 8.02 (s, 1H), 7.68 (d, J=8.0 Hz, 2H), 7.51 (d, J=8.7 Hz, 1H), 7.16-7.07 (m, 1H), 7.04 (d, J=7.2 Hz, 1H), 6.56 (d, J=2.5 Hz, 1H), 6.34 (dd, J=8.8, 2.5 Hz, 1H), 4.10 (t, J=7.7 Hz, 2H), 3.75 (s, 3H), 3.64 (d, J=12.2 Hz, 2H), 3.05 (t, J=7.7 Hz, 2H), 2.61 (dd, J=21.5, 10.8 Hz, 3H), 2.49 (s, 2H), 2.43 (d, J=1.9 Hz, 2H), 2.36-2.22 (m, 7H), 2.13 (s, 3H), 1.81 (d, J=11.6 Hz, 2H), 1.48 (q, J=11.8 Hz, 2H); [M+H]⁺=591.3.

### Example 18: 5-chloro-N²-(2-methoxy-4-(4-(4-methylpiperazin-1-yl)piperidin-1-yl)phenyl)-N⁴-(2-methyl-1-(methylsulfonyl)indolin-7-yl)pyrimidine-2,4-diamine

### Step 1: N-(2,5-dichloropyrimidin-4-yl)-2-methylindolin-7-amine

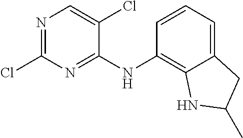 [SMILES: CC1Cc2cccc(Nc3nc(Cl)ncc3Cl)c2N1]

The titled compound (200 mg, 84%) was prepared in a manner similar to that in Example 1 step 1 from 2-methylindolin-7-amine and 2,4,5-trichloropyrimidine. [M+H]⁺=295.2.

### Step 2: N-(2,5-dichloropyrimidin-4-yl)-2-methyl-1-(methylsulfonyl)indolin-7-amine

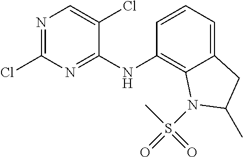 [SMILES: CC1Cc2cccc(Nc3nc(Cl)ncc3Cl)c2N1S(C)(=O)=O]

The titled compound (100 mg, 79%) was prepared in a manner similar to that in Example 1 step 2 from N-(2,5-dichloropyrimidin-4-yl)-2-methylindolin-7-amine and methanesulfonyl chloride. [M+H]⁺=373.2.

### Step 3: 5-chloro-N²-(2-methoxy-4-(4-(4-methylpiperazin-1-yl)piperidin-1-yl)phenyl)-N⁴-(2-methyl-1-(methylsulfonyl)indolin-7-yl)pyrimidine-2,4-diamine

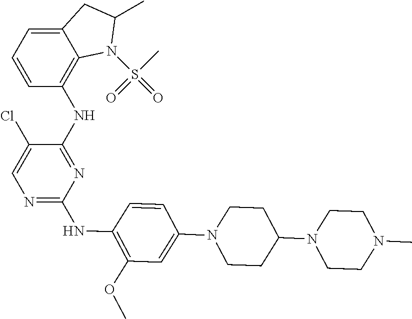 [SMILES: COc1cc(N2CCC(N3CCN(C)CC3)CC2)ccc1Nc1ncc(Cl)c(Nc2cccc3c2N(S(C)(=O)=O)C(C)C3)n1]

The titled compound was prepared in a manner similar to that in Example 30.

¹H NMR (400 MHz, DMSO) δ_{H} 8.96 (s, 1H), 8.06 (s, 1H), 7.92 (d, J=9.4 Hz, 2H), 7.40 (d, J=8.6 Hz, 1H), 7.12 (dd, J=8.6, 5.3 Hz, 2H), 6.58 (d, J=2.5 Hz, 1H), 6.40 (dd, J=8.8, 2.5 Hz, 1H), 4.58 (p, J=7.0 Hz, 1H), 3.73 (s, 3H), 3.68 (d, J=10.6 Hz, 2H), 3.55 (dd, J=16.3, 7.8 Hz, 3H), 3.00 (s, 3H), 2.62 (t, J=11.1 Hz, 2H), 2.53-2.49 (m, 3H), 2.41-2.24 (m, 5H), 2.16 (s, 3H), 1.83 (d, J=11.7 Hz, 2H), 1.49 (dd, J=20.0, 11.9 Hz, 2H), 1.10 (d, J=6.7 Hz, 3H); [M+H]⁺=641.3.

### Example 23: 5-chloro-N²-(2-methoxy-4-(4-(4-methylpiperazin-1-yl)piperidin-1-yl)phenyl)-N⁴-(3-methyl-1-(methylsulfonyl)indolin-7-yl)pyrimidine-2,4-diamine

### Step 1: N-(2,5-dichloropyrimidin-4-yl)-3-methylindolin-7-amine

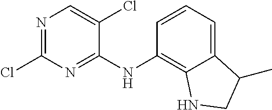 [SMILES: CC1CNc2c(Nc3nc(Cl)ncc3Cl)cccc21]

The titled compound (205 mg, 85%) was prepared in a manner similar to that in Example 1 step 1 from 3-methylindolin-7-amine and 2,4,5-trichloropyrimidine. [M+H]⁺=295.2.

### Step 2: N-(2,5-dichloropyrimidin-4-yl)-3-methyl-1-(methylsulfonyl)indolin-7-amine

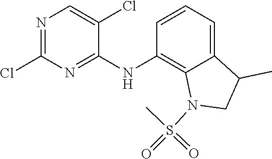 [SMILES: CC1CN(S(C)(=O)=O)c2c(Nc3nc(Cl)ncc3Cl)cccc21]

The titled compound (98 mg, 78%) was prepared in a manner similar to that in Example 1 step 2 from N-(2,5-dichloropyrimidin-4-yl)-3-methylindolin-7-amine and methanesulfonyl chloride. [M+H]⁺=373.2.

### Step 3: 5-chloro-N²-(2-methoxy-4-(4-(4-methylpiperazin-1-yl)piperidin-1-yl)phenyl)-N⁴-(3-methyl-1-(methylsulfonyl)indolin-7-yl)pyrimidine-2,4-diamine

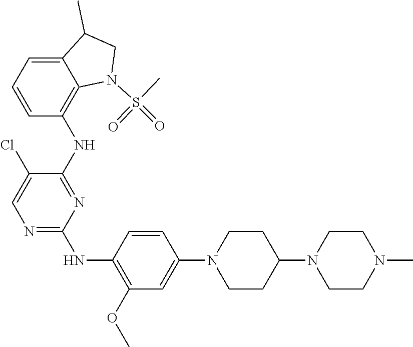 [SMILES: COc1cc(N2CCC(N3CCN(C)CC3)CC2)ccc1Nc1ncc(Cl)c(Nc2cccc3c2N(S(C)(=O)=O)CC3C)n1]

The titled compound was prepared in a manner similar to that in Example 30. ¹H NMR (400 MHz, CD₃OD) δ_{H} 7.98 (s, 1H), 7.83 (d, J=8.2 Hz, 1H), 7.72 (d, J=8.8 Hz, 1H), 7.27 (t, J=7.7 Hz, 1H), 7.16 (d, J=7.3 Hz, 1H), 6.64 (s, 1H), 6.40 (d, J=8.6 Hz, 1H), 4.60 (s, 1H), 4.43-4.31 (m, 1H), 3.84 (s, 3H), 3.66 (d, J=12.0 Hz, 3H), 3.49 (t, J=11.5 Hz, 1H), 2.99 (s, 3H), 2.87-2.45 (m, 9H), 2.38 (t, J=9.4 Hz, 1H), 2.31 (s, 3H), 2.02 (d, J=11.5 Hz, 2H), 1.73-1.57 (m, 2H), 1.33 (d, J=6.5 Hz, 3H); [M+H]⁺=641.3.

### Example 28: 5-bromo-N²-(2-methoxy-4-(4-(4-methylpiperazin-1-yl)piperidin-1-yl)phenyl)-N⁴-(1′-(methylsulfonyl)spiro[cyclopropane-1,3′-indolin]-7′-yl)pyrimidine-2,4-diamine

### Step 1: 1′-(methylsulfonyl)-7′-nitrospiro[cyclopropane-1,3′-indoline]

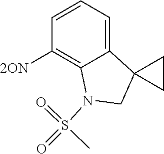

The titled compound (370 mg, 72%) was prepared in a manner similar to that in Example 30 step 1 from 7′-nitrospiro[cyclopropane-1,3′-indoline] and methanesulfonyl chloride. [M+H]⁺=269.1.

### Step 2: 1′-(methylsulfonyl)spiro[cyclopropane-1,3′-indolin]-7′-amine

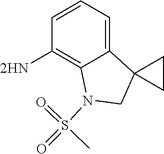

The titled compound (320 mg, 91%) was prepared in a manner similar to that in Example 30 step 1 from 1′-(methylsulfonyl)-7′-nitrospiro[cyclopropane-1,3′-indoline] and Pd. [M+H]⁺=239.1.

### Step 3: N-(5-bromo-2-chloropyrimidin-4-yl)-1′-(methylsulfonyl)spiro[cyclopropane-1,3′-indolin]-7-amine

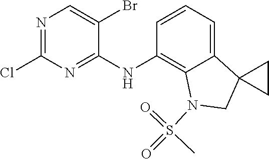 [SMILES: CS(=O)(=O)N1CC2(CC2)c2cccc(Nc3nc(Cl)ncc3Br)c21]

The titled compound (380 mg, 84%) was prepared in a manner similar to that in Example 30 step 3 from 1′-(methylsulfonyl)spiro[cyclopropane-1,3′-indolin]-7′-amine and 5-bromo-2,4-dichloropyrimidine. [M+H]⁺=429.1, 431.1.

### Step 4: 5-bromo-N²-(2-methoxy-4-(4-(4-methylpiperazin-1-yl)piperidin-1-yl)phenyl)-N⁴-(1′-(methylsulfonyl)spiro[cyclopropane-1.3′-indolin]-7′-yl)pyrimidine-2,4-diamine

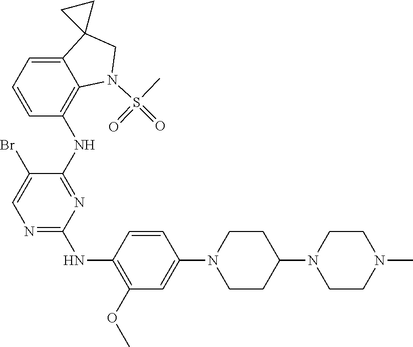 [SMILES: COc1cc(N2CCC(N3CCN(C)CC3)CC2)ccc1Nc1ncc(Br)c(Nc2cccc3c2N(S(C)(=O)=O)CC32CC2)n1]

The titled compound was prepared in a manner similar to that in Example 2. ¹H NMR (400 MHz, DMSO) δ_{H} 8.80 (s, 1H), 8.14 (s, 1H), 7.86 (s, 1H), 7.79 (d, J=7.6, 1H), 7.42 (d, J=8.6, 1H), 7.13 (t, J=7.7, 1H), 6.70 (d, J=6.8, 1H), 6.59 (d, J=2.3, 1H), 6.38 (dd, J=8.7, 2.5, 1H), 4.02 (s, 2H), 3.76 (s, 3H), 3.68 (d, J=11.9, 2H), 3.29-3.24 (m, 5H), 3.02 (s, 3H), 2.70-2.60 (m, 2H), 2.30 (dd, J=15.9, 10.5, 4H), 2.16 (s, 3H), 1.84 (d, J=11.6, 2H), 1.50 (dd, J=22.7, 11.2, 2H), 1.24 (s, 2H), 1.06 (s, 2H); [M+H]⁺=697.2, 699.2.

### Example 31: N²-(2-methoxy-4-(4-(4-methylpiperazin-1-yl)piperidin-1-yl)phenyl)-N⁴-(1-(methylsulfonyl)indolin-7-yl)-5-(trifluoromethyl)pyrimidine-2,4-diamine

### Step 1: 2-chloro-N-(3-(methylsulfonyl)-2,3-dihydro-1H-inden-4-yl)-5-(trifluoromethyl)pyrimidin-4-amine

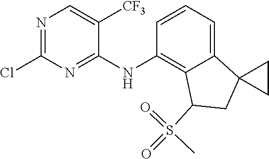 [SMILES: CS(=O)(=O)C1CC2(CC2)c2cccc(Nc3nc(Cl)ncc3C(F)(F)F)c21]

The titled compound (170 mg, 82%) was prepared in a manner similar to that in Example 30 step 3 from 1-(methylsulfonyl)indolin-7-amine and 2,4-dichloro-5-(trifluoromethyl)pyrimidine. [M+H]⁺=392.1.

### Step 2: N²-(2-methoxy-4-(4-(4-methylpiperazin-1-yl)piperidin-1-yl)phenyl)-N⁴-(1-(methylsulfonyl)indolin-7-yl)-5-(trifluoromethyl)pyrimidine-2,4-diamine

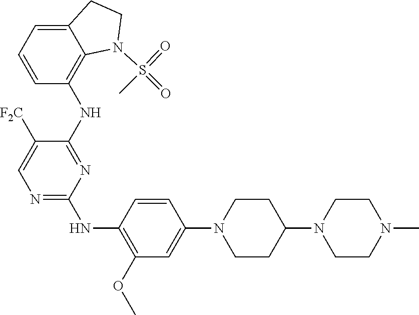

The titled compound was prepared in a manner similar to that in Example 2. ¹H NMR (400 MHz, DMSO) δ_{H} 8.83 (s, 1H), 8.48 (s, 1H), 8.36 (s, 1H), 7.76 (s, 1H), 7.41-7.29 (m, 1H), 7.27-7.09 (m, 2H), 6.65 (s, 1H), 6.50-6.37 (m, 1H), 4.44-4.20 (m, 2H), 4.13-3.98 (m, 3H), 3.79 (m, 5H), 3.15 (s, 2H), 3.09 (s, 3H), 2.72 (t, J=11.4 Hz, 2H), 2.61 (s, 2H), 2.44 (dd, J=12.6, 8.8 Hz, 4H), 2.27 (s, 3H), 1.91 (d, J=10.9 Hz, 2H), 1.57 (dd, J=20.6, 10.1 Hz, 2H); [M+H]⁺=661.3.

### Example 32: 5-fluoro-N²-(2-methoxy-4-(4-(4-methylpiperazin-1-yl)piperidin-1-yl)phenyl)-N⁴-(1-(methylsulfonyl)indolin-7-yl)pyrimidine-2,4-diamine

### Step 1: N-(2-chloro-5-fluoropyrimidin-4-yl)-1-(methylsulfonyl)indolin-7-amine

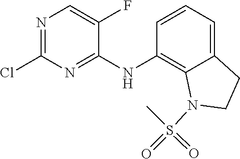 [SMILES: CS(=O)(=O)N1CCc2cccc(Nc3nc(Cl)ncc3F)c21]

The titled compound (620 mg, 74%) was prepared in a manner similar to that in Example 30 step 3 from 1-(methylsulfonyl)indolin-7-amine and 2,4-dichloro-5-fluoropyrimidine. [M+H]⁺=343.1.

### Step 2: 5-fluoro-N²-(2-methoxy-4-(4-(4-methylpiperazin-1-yl)piperidin-1-yl)phenyl)-N⁴-(1-(methylsulfonyl)indolin-7-yl)pyrimidine-2,4-diamine

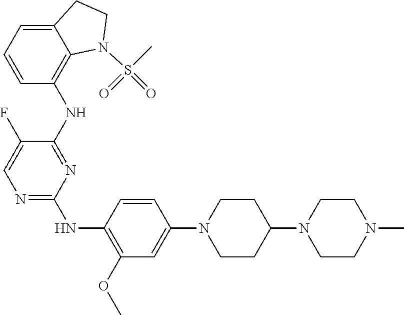 [SMILES: COc1cc(N2CCC(N3CCN(C)CC3)CC2)ccc1Nc1ncc(F)c(Nc2cccc3c2N(S(C)(=O)=O)CC3)n1]

The titled compound was prepared in a manner similar to that in Example 30. ¹H NMR (400 MHz, DMSO) δ_{H} 8.91 (s, 1H), 8.06 (s, 1H), 7.97 (d, J=7.8 Hz, 1H), 7.68 (s, 1H), 7.54 (d, J=8.6 Hz, 1H), 7.24-7.07 (m, 2H), 6.60 (s, 1H), 6.41 (d, J=8.2 Hz, 1H), 4.06 (t, J=7.7 Hz, 2H), 3.77 (s, 3H), 3.67 (d, J=10.8 Hz, 2H), 3.31-3.22 (m, 4H), 3.15-2.99 (m, 6H), 2.63 (t, J=12.2 Hz, 2H), 2.40-2.24 (m, 4H), 2.15 (s, 3H), 1.84 (d, J=11.6 Hz, 2H), 1.61-1.40 (m, 2H); [M+H]⁺=611.3.

### Example 33: 2-((2-methoxy-4-(4-(4-methylpiperazin-1-yl)piperidin-1-yl)phenyl)amino)-4-((1-(methylsulfonyl)indolin-7-yl)amino)pyrimidine-5-carbonitrile

### Step 1: 2-chloro-4-((1-(methylsulfonyl)indolin-7-yl)amino)pyrimidine-5-carbonitrile

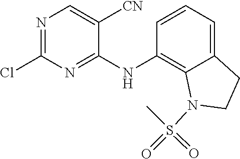 [SMILES: CS(=O)(=O)N1CCc2cccc(Nc3nc(Cl)ncc3C#N)c21]

The titled compound (80 mg, 64%) was prepared in a manner similar to that in Example 30 step 3 from 2,4-dichloropyrimidine-5-carbonitrile and 1-(methylsulfonyl)indolin-7-amine.[M+H]⁺=350.1.

### Step 2: 2-((2-methoxy-4-(4-(4-methylpiperazin-1-yl)piperidin-1-yl)phenyl)amino)-4-((1-(methylsulfonyl)indolin-7-yl)amino)pyrimidine-5-carbonitrile

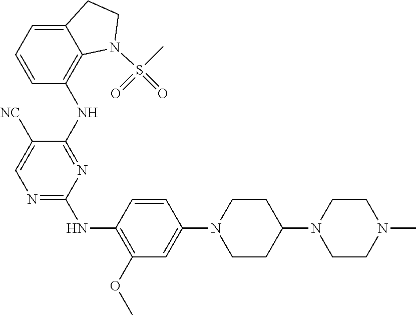 [SMILES: COc1cc(N2CCC(N3CCN(C)CC3)CC2)ccc1Nc1ncc(C#N)c(Nc2cccc3c2N(S(C)(=O)=O)CC3)n1]

The titled compound was prepared in a manner similar to that in Example 2. ¹H NMR (400 MHz, DMSO) δ_{H} 8.96 (s, 1H), 8.80 (s, 1H), 8.43 (s, 1H), 7.69 (s, 1H), 7.24 (d, J=8.1 Hz, 1H), 7.14 (s, 1H), 7.08 (s, 1H), 6.59 (s, 1H), 6.41 (d, J=6.3 Hz, 1H), 4.03 (s, 2H), 3.74 (s, 3H), 3.74-3.65 (m, 2H), 3.34-3.22 (m, 4H), 3.16-3.04 (m, 3H), 3.03 (s, 3H), 2.67 (t, J=11.4 Hz, 2H), 2.40-2.24 (m, 4H), 2.15 (s, 3H), 1.84 (d, J=11.9 Hz, 2H), 1.50 (q, J=9.8 Hz, 2H); [M+H]⁺=618.3.

### Example 34: 5-bromo-N₂-(2-methoxy-4-(4-(4-methylpiperazin-1-yl)piperidin-1-yl)phenyl)-N₄-(1-(methylsulfonyl)indolin-7-yl)pyrimidine-2,4-diamine

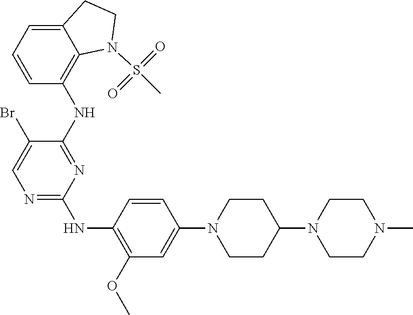 [SMILES: COc1cc(N2CCC(N3CCN(C)CC3)CC2)ccc1Nc1ncc(Br)c(Nc2cccc3c2N(S(C)(=O)=O)CC3)n1]

The titled compound (6.35 mg, 18%) was prepared in a manner similar to that in Example 21 step 3 from N-(5-bromo-2-chloropyrimidin-4-yl)-1-(methylsulfonyl)indolin-7-amine and 2-methoxy-4-(4-(4-methylpiperazin-1-yl)piperidin-1-yl)aniline. ¹H NMR (400 MHz, DMSO) δ 8.78 (s, 1H), 8.14 (s, 1H), 7.88 (s, 1H), 7.83 (s, 1H), 7.42 (d, J=8.7 Hz, 1H), 7.15 (s, 2H), 6.59 (s, 1H), 6.39 (d, J=8.9 Hz, 1H), 4.04 (s, 2H), 3.76 (s, 3H), 3.69 (d, J=11.0 Hz, 2H), 3.10 (s, 2H), 3.04 (s, 3H), 2.64 (t, J=11.8 Hz, 2H), 2.51 (s, 4H), 2.34 (s, 5H), 2.17 (s, 3H), 1.84 (d, J=12.5 Hz, 2H), 1.51 (d, J=11.2 Hz, 2H); [M+H]/[M+3]⁺=671.20/673.2.

### Example 35: N²-(2-methoxy-4-(4-(4-methylpiperazin-1-yl)piperidin-1-yl)phenyl)-5-methyl-N⁴-(1-(methylsulfonyl)indolin-7-yl)pyrimidine-2,4-diamine

### Step 1: N-(2-chloro-5-methylpyrimidin-4-yl)-1-(methylsulfonyl)indolin-7-amine

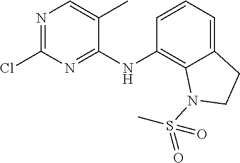 [SMILES: Cc1cnc(Cl)nc1Nc1cccc2c1N(S(C)(=O)=O)CC2]

The titled compound (60 mg, 42%) was prepared in a manner similar to that in Example 30 step 3 from 1-(methylsulfonyl)indolin-7-amine and 2,4-dichloro-5-methylpyrimidine. [M+H]⁺=339.1.

### Step 2: N²-(2-methoxy-4-(4-(4-methylpiperazin-1-yl)piperidin-1-yl)phenyl)-5-methyl-N⁴-(1-(methylsulfonyl)indolin-7-yl)pyrimidine-2,4-diamine

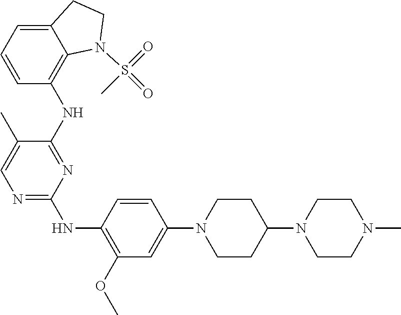 [SMILES: COc1cc(N2CCC(N3CCN(C)CC3)CC2)ccc1Nc1ncc(C)c(Nc2cccc3c2N(S(C)(=O)=O)CC3)n1]

The titled compound was prepared in a manner similar to that in Example 30. ¹H NMR (400 MHz, DMSO) δ_{H} 8.55 (s, 1H), 8.11 (d, J=7.7 Hz, 1H), 7.98 (s, 1H), 7.82 (d, J=8.6 Hz, 1H), 7.49 (s, 1H), 7.31 (t, J=7.6 Hz, 1H), 7.23 (d, J=7.1 Hz, 1H), 6.72 (s, 1H), 6.50 (d, J=8.9 Hz, 1H), 4.18 (s, 2H), 3.90 (s, 3H), 3.82-3.72 (m, 5H), 3.22 (s, 2H), 3.18 (s, 3H), 2.73 (t, J=11.8 Hz, 3H), 2.55-2.37 (m, 5H), 2.28 (s, 3H), 2.14 (s, 3H), 1.96 (d, J=11.3 Hz, 2H), 1.63 (dd, J=22.4, 11.0 Hz, 2H); [M+H]⁺=607.3.

### Example 36: 5-chloro-N²-(4-(4-(dimethylamino)piperidin-1-yl)-2-methoxyphenyl)-N⁴-(1-(methylsulfonyl)indolin-7-yl)pyrimidine-2,4-diamine

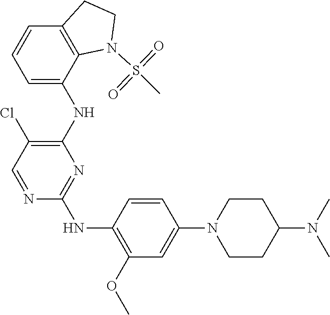 [SMILES: COc1cc(N2CCC(N(C)C)CC2)ccc1Nc1ncc(Cl)c(Nc2cccc3c2N(S(C)(=O)=O)CC3)n1]

The titled compound (8.45 mg, 30%) was prepared in a manner similar to that in Example 30 step 4 from N-(2,5-dichloropyrimidin-4-yl)-1-(methylsulfonyl)indolin-7-amine and 1-(4-amino-3-methoxyphenyl)-N,N-dimethylpiperidin-4-amine. ¹H NMR (400 MHz, DMSO) δ 8.93 (s, 1H), 8.08 (s, 1H), 7.89 (s, 2H), 7.45 (d, J=8.6 Hz, 1H), 7.15 (s, 2H), 6.61 (s, 1H), 6.41 (d, J=8.5 Hz, 1H), 4.05 (s, 2H), 3.76 (s, 3H), 3.69 (d, J=11.5 Hz, 2H), 3.10 (s, 3H), 3.04 (s, 3H), 2.64 (t, J=11.8 Hz, 2H), 2.26 (s, 6H), 1.86 (d, J=11.5 Hz, 2H), 1.65-1.41 (m, 2H); [M+H]⁺=572.2.

### Example 39: 5-chloro-N²-(2-methoxy-5-methyl-4-(4-(4-methylpiperazin-1-yl)piperidin-1-yl)phenyl)-N⁴-(1-(methylsulfonyl)indolin-7-yl)pyrimidine-2,4-diamine

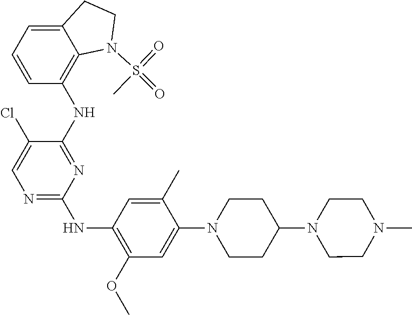 [SMILES: COc1cc(N2CCC(N3CCN(C)CC3)CC2)c(C)cc1Nc1ncc(Cl)c(Nc2cccc3c2N(S(C)(=O)=O)CC3)n1]

The titled compound was prepared in a manner similar to that in Example 30. H¹ NMR (400 MHz, DMSO) δ_{H} 8.95 (1H, s), 8.11 (1H, s), 7.88 (2H, s), 7.46 (1H, s), 7.16 (2H, s), 6.68 (1H, s), 4.06 (2H, s), 3.76 (3H, s), 3.33-3.28 (2H, m), 3.13-3.05 (4H, m), 3.04 (3 H, s), 2.71-2.52 (5 H, m), 2.40-2.25 (4H, m), 2.16 (3H, s), 2.08 (3H, s), 1.90-1.79 (2H, m), 1.56 (2 H, q, J=12 Hz); [M+H]⁺=641.3.

### Example 40: 5-bromo-N²-(2-methoxy-5-methyl-4-(4-(4-methylpiperazin-1-yl)piperidin-1-yl)phenyl)-N⁴-(1-(methylsulfonyl)indolin-7-yl)pyrimidine-2,4-diamine

### Step 1: N-(5-bromo-2-chloropyrimidin-4-yl)-1-(methylsulfonyl)indolin-7-amine

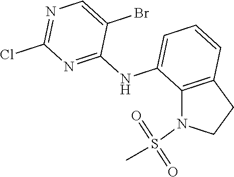 [SMILES: CS(=O)(=O)N1CCc2cccc(Nc3nc(Cl)ncc3Br)c21]

The titled compound (520 mg, 88%) was prepared in a manner similar to that in Example 30 step 3 from 1-(methylsulfonyl)indolin-7-amine and 5-bromo-2,4-dichloropyrimidine. [M+H]⁺=402.8/404.9.

### Step 2: 5-bromo-N²-(2-methoxy-5-methyl-4-(4-(4-methylpiperazin-1-yl)piperidin-1-yl)phenyl)-N⁴-(1-(methylsulfonyl)indolin-7-yl)pyrimidine-2,4-diamine

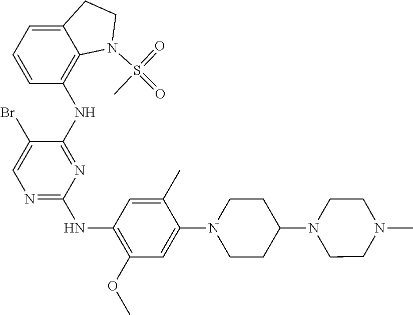 [SMILES: COc1cc(N2CCC(N3CCN(C)CC3)CC2)c(C)cc1Nc1ncc(Br)c(Nc2cccc3c2N(S(C)(=O)=O)CC3)n1]

The titled compound was prepared in a manner similar to that in Example 2. ¹H NMR (400 MHz, CD₃OD) δ_{H} 8.05 (s, 1H), 7.75 (d, J=4.0 Hz, 1H), 7.63 (s, 1H), 7.18 (s, 2H), 6.62 (s, 1H), 4.10 (t, J=7.1 Hz, 2H), 3.82 (s, 3H), 3.35-3.25 (m, 2H), 3.16-2.94 (m, 13H), 2.70-2.58 (m, 6H), 2.00 (m, 5H), 1.71 (m, 2H); [M+H]⁺=685.2/687.2.

### Example 41: 5-chloro-N²-(5-ethyl-2-methoxy-4-(4-(4-methylpiperazin-1-yl)piperidin-1-yl)phenyl)-N⁴-(1-(methylsulfonyl)indolin-7-yl)pyrimidine-2,4-diamine

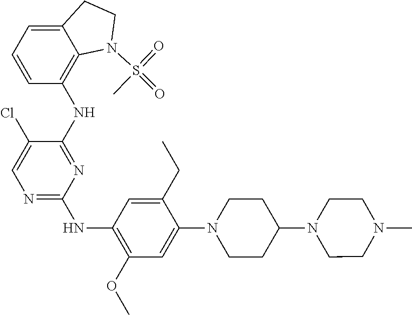 [SMILES: CCc1cc(Nc2ncc(Cl)c(Nc3cccc4c3N(S(C)(=O)=O)CC4)n2)c(OC)cc1N1CCC(N2CCN(C)CC2)CC1]

The titled compound was prepared in a manner similar to that in Example 30. ¹H NMR (400 MHz, DMSO) δ 8.93 (s, 1H), 8.13 (d, J=7.7 Hz, 1H), 7.94 (s, 1H), 7.82 (s, 1H), 7.48 (s, 1H), 7.13 (d, J=12.2 Hz, 2H), 6.74 (s, 1H), 4.05 (t, J=7.7 Hz, 2H), 3.76 (s, 3H), 3.34 (s, 4H), 3.10 (t, J=6.4 Hz, 2H), 3.05 (s, 3H), 3.00 (d, J=10.5 Hz, 3H), 2.90-2.80 (m, 2H), 2.68 (t, J=11.4 Hz, 3H), 2.49-2.30 (m, 6H), 1.88 (s, 2H), 1.62 (s, 2H), 1.01 (t, J=7.1 Hz, 3H); [M+H]⁺=655.3.

### Example 42: 5-bromo-N²-(2-methoxy-5-methyl-4-((3S,5R)-3,4,5-trimethylpiperazin-1-yl)phenyl)-N⁴-(1-(methylsulfonyl)indolin-7-yl)pyrimidine-2,4-diamine

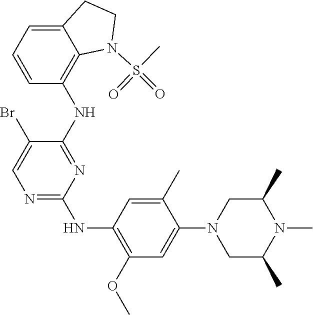 [SMILES: COc1cc(N2C[C@@H](C)N(C)[C@@H](C)C2)c(C)cc1Nc1ncc(Br)c(Nc2cccc3c2N(S(C)(=O)=O)CC3)n1]

The titled compound was prepared in a manner similar to that in Example 2. ¹H NMR (400 MHz, DMSO) δ_{H} 8.80 (s, 1H), 8.18 (s, 1H), 7.88 (s, 1H), 7.80 (s, 1H), 7.45 (s, 1H), 7.16 (s, 2H), 6.65 (s, 1H), 4.05 (t, J=7.1, 2H), 3.77 (s, 3H), 3.11 (t, J=7.1, 2H), 3.05 (s, 3H), 2.86 (d, J=10.7, 2H), 2.45 (d, J=11.0, 2H), 2.39-2.27 (m, 2H), 2.23 (s, 3H), 2.08 (s, 3H), 1.05 (d, J=6.0, 6H); [M+H]⁺=630.2/632.2.

### Example 43: (S)-5-bromo-N²-(4-(3,4-dimethylpiperazin-1-yl)-2-methoxy-5-methylphenyl)-N⁴-(1-(methylsulfonyl)indolin-7-yl)pyrimidine-2,4-diamine

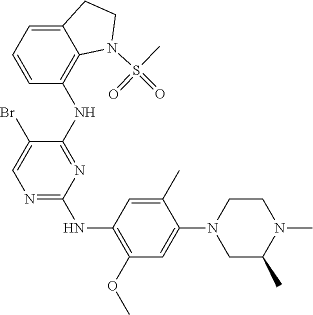 [SMILES: COc1cc(N2CCN(C)[C@@H](C)C2)c(C)cc1Nc1ncc(Br)c(Nc2cccc3c2N(S(C)(=O)=O)CC3)n1]

The titled compound was prepared in a manner similar to that in Example 2. ¹H NMR (400 MHz, DMSO) δ_{H} 8.85-8.74 (m, 1H), 8.18 (s, 1H), 7.88 (s, 1H), 7.81 (d, J=6.7, 1H), 7.44 (s, 1H), 7.15 (s, 2H), 6.67 (s, 1H), 4.05 (t, J=6.8, 2H), 3.77 (s, 3H), 3.10 (t, J=7.5, 2H), 3.05 (s, 3H), 2.91-2.74 (m, 4H), 2.44 (t, J=10.2, 1H), 2.31 (t, J=10.4, 1H), 2.23 (s, 4H), 2.08 (s, 3H), 1.03 (d, J=6.1, 3H); [M+H]⁺=616.2/618.2.

### Example 44: 5-bromo-N²-(5-chloro-2-methoxy-4-(9-methyl-3,9-diazaspiro[5.5]undecan-3-yl)phenyl)-N⁴-(1-(methylsulfonyl)indolin-7-yl)pyrimidine-2,4-diamine

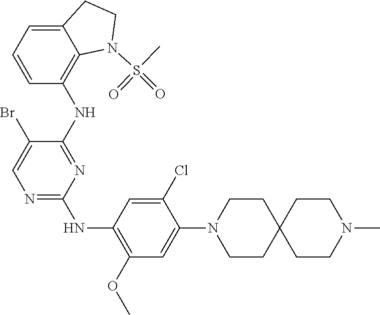 [SMILES: COc1cc(N2CCC3(CCN(C)CC3)CC2)c(Cl)cc1Nc1ncc(Br)c(Nc2cccc3c2N(S(C)(=O)=O)CC3)n1]

The titled compound was prepared in a manner similar to that in Example 2. ¹H NMR (400 MHz, DMSO) δ_{H} 8.85 (s, 1H), 8.22 (d, J=1.9, 1H), 7.95 (s, 1H), 7.80 (d, J=7.7, 1H), 7.73 (s, 1H), 7.28-7.11 (m, 2H), 6.83 (s, 1H), 4.05 (t, J=7.3, 2H), 3.81 (s, 3H), 3.11 (t, J=7.1, 2H), 3.05 (s, 3H), 2.91 (s, 4H), 2.40 (s, 4H), 2.23 (s, 3H), 1.55 (d, J=14.6, 8H); [M+H]⁺=690.2/692.2.

### Example 45: 5-bromo-N²-(2-methoxy-5-methyl-4-(2-methyl-2,7-diazaspiro[3.5]nonan-7-yl)phenyl)-N⁴-(1-(methylsulfonyl)indolin-7-yl)pyrimidine-2,4-diamine

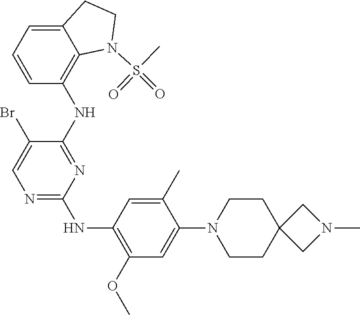 [SMILES: COc1cc(N2CCC3(CC2)CN(C)C3)c(C)cc1Nc1ncc(Br)c(Nc2cccc3c2N(S(C)(=O)=O)CC3)n1]

The titled compound was prepared in a manner similar to that in Example 2. ¹H NMR (400 MHz, DMSO) δ_{H} 8.80 (s, 1H), 8.17 (s, 1H), 7.85 (s, 1H), 7.81 (d, J=5.8, 1H), 7.44 (s, 1H), 7.19-7.08 (m, 2H), 6.65 (s, 1H), 4.05 (t, J=7.3, 2H), 3.76 (s, 3H), 3.10 (t, J=7.0, 2H), 3.05 (d, J=2.5, 7H), 2.71 (s, 4H), 2.30 (s, 3H), 2.06 (s, 3H), 1.80 (s, 4H); [M+H]⁺=642.2/644.2.

### Example 46: 5-bromo-N²-(4-(4-(dimethylamino)piperidin-1-yl)-2-methoxy-5-methylphenyl)-N⁴-(1-(methylsulfonyl)indolin-7-yl)pyrimidine-2,4-diamine

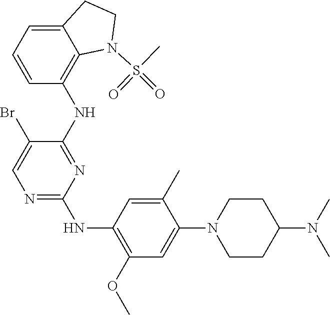 [SMILES: COc1cc(N2CCC(N(C)C)CC2)c(C)cc1Nc1ncc(Br)c(Nc2cccc3c2N(S(C)(=O)=O)CC3)n1]

The titled compound was prepared in a manner similar to that in Example 2. ¹H NMR (400 MHz, DMSO) δ_{H} 8.80 (s, 1H), 8.17 (s, 1H), 7.88 (s, 1H), 7.81 (d, J=6.3, 1H), 7.44 (s, 1H), 7.20-7.09 (m, 2H), 6.68 (s, 1H), 4.05 (t, J=7.2, 2H), 3.76 (s, 3H), 3.14-3.05 (m, 4H), 3.05 (s, 3H), 2.61 (t, J=11.1, 2H), 2.33 (s, 1H), 2.29 (s, 6H), 2.08 (s, 3H), 1.88 (d, J=11.1, 2H), 1.57 (q, J=10.9, 2H); [M+H]⁺=630.2/632.2.

### Example 47: 5-chloro-N²-(4-(4-(dimethylamino)piperidin-1-yl)-2,3-dihydrobenzofuran-7-yl)-N⁴-(1-(methylsulfonyl)indolin-7-yl)pyrimidine-2,4-diamine

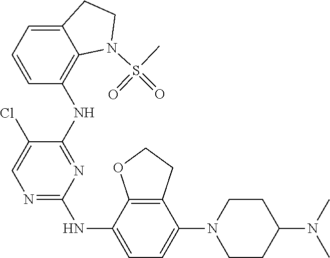 [SMILES: CN(C)C1CCN(c2ccc(Nc3ncc(Cl)c(Nc4cccc5c4N(S(C)(=O)=O)CC5)n3)c3c2CCO3)CC1]

The titled compound was prepared in a manner similar to that in Example 30. ¹H NMR (400 MHz, CD₃OD) δ_{H} 8.53 (s, 1H), 7.97 (s, 1H), 7.87 (s, 1H), 7.42 (d, J=8.7 Hz, 1H), 7.16 (s, 2H), 6.38 (d, J=8.7 Hz, 1H), 4.62-4.50 (m, 2H), 4.12 (s, 2H), 3.45 (d, J=12.4 Hz, 2H), 3.18 (dd, J=15.1, 7.4 Hz, 4H), 2.95 (s, 3H), 2.79-2.70 (m, 3H), 2.69 (s, 6H), 2.11 (d, J=11.7 Hz, 2H), 1.78 (dd, J=21.5, 10.8 Hz, 2H); [M+H]⁺=584.2

### Example 48: 5-bromo-N²-(4-(4-(dimethylamino)piperidin-1-yl)-2,3-dihydrobenzofuran-7-yl)-N⁴-(1-(methylsulfonyl)indolin-7-yl)pyrimidine-2,4-diamine

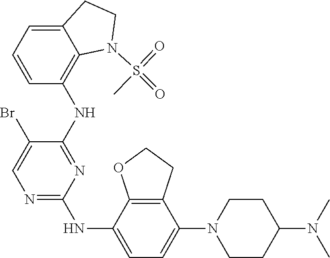 [SMILES: CN(C)C1CCN(c2ccc(Nc3ncc(Br)c(Nc4cccc5c4N(S(C)(=O)=O)CC5)n3)c3c2CCO3)CC1]

The titled compound was prepared in a manner similar to that in Example 2. ¹H NMR (400 MHz, DMSO) δ_{H} 8.75 (s, 1H), 8.29 (s, 1H), 8.13 (s, 1H), 7.86 (s, 1H), 7.15-7.00 (m, 3H), 6.35 (d, J=8.6 Hz, 1H), 4.44 (t, J=8.4 Hz, 2H), 4.04 (t, J=6.0 Hz, 2H), 3.15-3.03 (m, 8H), 2.60 (t, J=11.4 Hz, 2H), 2.24 (s, 8H), 1.87 (d, J=11.3 Hz, 2H), 1.52 (q, J=12.1 Hz, 2H); [M+H]⁺=628.2/630.2.

### Example 49: 5-chloro-N²-(4-(4-(4-methylpiperazin-1-yl)piperidin-1-yl)-2,3-dihydrobenzofuran-7-yl)-N⁴-(1-(methylsulfonyl)indolin-7-yl)pyrimidine-2,4-diamine

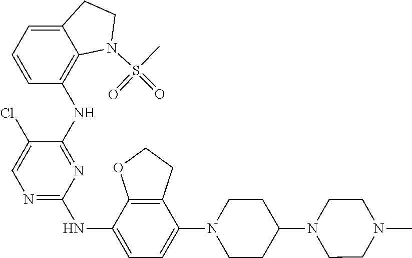 [SMILES: CN1CCN(C2CCN(c3ccc(Nc4ncc(Cl)c(Nc5cccc6c5N(S(C)(=O)=O)CC6)n4)c4c3CCO4)CC2)CC1]

The titled compound was prepared in a manner similar to that in Example 30. ¹H NMR (400 MHz, CD₃OD) δ_{H} 8.49 (s, 1H), 7.96 (s, 1H), 7.86 (s, 1H), 7.40 (d, J=8.6 Hz, 1H), 7.16 (s, 2H), 6.36 (d, J=8.7 Hz, 1H), 4.52 (t, J=8.5 Hz, 2H), 4.11 (t, J=7.4 Hz, 2H), 3.43 (t, J=17.1 Hz, 2H), 3.16 (dd, J=19.1, 8.0 Hz, 4H), 2.95 (s, 3H), 2.94-2.63 (m, 10H), 2.54 (s, 4H), 2.02 (d, J=11.1 Hz, 2H), 1.77-1.60 (m, 2H); [M+H]⁺=639.2

### Example 50: 5-bromo-N²-(4-(4-(4-methylpiperazin-1-yl)piperidin-1-yl)-2,3-dihydrobenzofuran-7-yl)-N⁴-(1-(methylsulfonyl)indolin-7-yl)pyrimidine-2,4-diamine

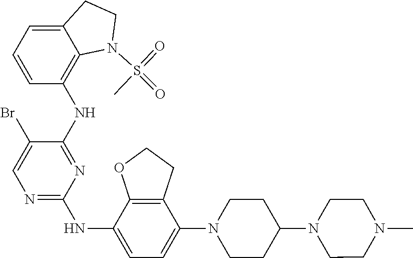 [SMILES: CN1CCN(C2CCN(c3ccc(Nc4ncc(Br)c(Nc5cccc6c5N(S(C)(=O)=O)CC6)n4)c4c3CCO4)CC2)CC1]

The titled compound was prepared in a manner similar to that in Example 2. ¹H NMR (400 MHz, DMSO) δ_{H} 8.74 (s, 1H), 8.28 (s, 1H), 8.13 (s, 1H), 7.87 (s, 1H), 7.15-7.00 (m, 3H), 6.35 (d, J=8.3 Hz, 1H), 4.44 (s, 2H), 4.04 (s, 2H), 3.30-3.23 (m, 3H), 3.15-3.02 (m, 9H), 2.61 (d, J=11.6 Hz, 3H), 2.41-2.27 (m, 5H), 2.17 (s, 3H), 1.86 (d, J=10.2 Hz, 2H), 1.53 (dd, J=21.0, 10.2 Hz, 2H); [M+H]⁺=683.2/685.2.

### Example 51: 5-chloro-N₂-(7-(4-(dimethylamino)piperidin-1-yl)benzo[d][1,3]dioxol-4-yl)-N₄-(1-(methylsulfonyl)indolin-7-yl)pyrimidine-2,4-diamine

### Step 1: 7-bromobenzo[d][1,3]dioxol-4-amine

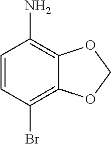 [SMILES: Nc1ccc(Br)c2c1OCO2]

To a stirred solution of benzo[d][1,3]dioxol-4-amine (500 mg, 3.6 mmol) in CH₃CN (15 mL) was added NBS (633 mg, 3.6 mmol). The resulting mixture was stirred at room temperature for 1 hour. The reaction was quenched with H₂O and extracted with EtOAc (2×50 mL). The combined organic layer was washed with brine (2×50 mL), dried over Na₂SO₄ and concentrated under vacuum to afford the crude residue, which was purified with silica gel column chromatography (PE: EA=100: 0˜ 2: 1 gradient elution) to give the title product (400 mg, 52%). [M+H]⁺=215.9/217.8.

### Step 2: 4-bromo-7-nitrobenzo[d][1,3]dioxole

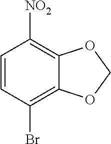 [SMILES: O=[N+]([O-])c1ccc(Br)c2c1OCO2]

To a stirred solution of m-CPBA (1.3 g, 7.4 mmol) in DCE (25 mL) was added 7-bromobenzo[d][1,3]dioxol-4-amine (400 mg, 1.8 mmol) at 80° C. The resulting mixture was stirred at 80° C. for 2 hours. The reaction was quenched with H₂O and extracted with EtOAc (2×50 mL). The combined organic layer was washed with brine (2×50 mL), dried over Na₂SO₄ and concentrated under vacuum to afford the crude residue, which was purified with silica gel column chromatography (PE: EA=100: 0˜ 2: 1 gradient elution) to give the title product (320 mg, 73%). [M+H]⁺=245.9/247.7.

### Step 3: N,N-dimethyl-1-(7-nitrobenzo[d][1,3]dioxol-4-yl)piperidin-4-amine

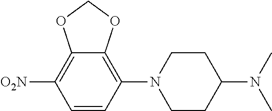 [SMILES: CN(C)C1CCN(c2ccc([N+](=O)[O-])c3c2OCO3)CC1]

A mixture of 4-bromo-7-nitrobenzo[d][1,3]dioxole (320 mg, 1.3 mmol), N,N-dimethylpiperidin-4-amine(250 mg, 2.0 mmol), Pd₂(dba)₃ (118 mg, 0.13 mmol), BINAP (162 mg, 0.26 mmol) and K₃PO₄ (551 mg, 2.6 mmol) in toluene (20 mL) was stirred in a round bottom flask at 120° C. overnight under N₂. The mixture was evaporated in vacuum to afford the crude residue, which was purified with silica gel column chromatography (DCM: MeOH=100: 0˜10: 1 gradient elution) to give title product(280 mg, 73%). [M+H]⁺=294.2.

### Step 4: 1-(7-aminobenzo[d][1,3]dioxol-4-yl)-N,N-dimethylpiperidin-4-amine

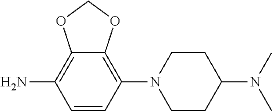 [SMILES: CN(C)C1CCN(c2ccc(N)c3c2OCO3)CC1]

The titled compound (180 mg, 72%) was prepared in a manner similar to that in Example 30 step 3 from N,N-dimethyl-1-(7-nitrobenzo[d][1,3]dioxol-4-yl)piperidin-4-amine, [M+H]⁺=264.2.

### Step 5: 5-chloro-N²-(7-(4-(dimethylamino)piperidin-1-yl)benzo[d] [1,3]dioxol-4-yl)-N⁴-(1-(methylsulfonyl)indolin-7-yl)pyrimidine-2,4-diamine

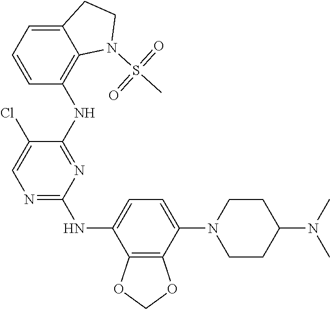 [SMILES: CN(C)C1CCN(c2ccc(Nc3ncc(Cl)c(Nc4cccc5c4N(S(C)(=O)=O)CC5)n3)c3c2OCO3)CC1]

The titled compound (9.36 mg, 25%) was prepared in a manner similar to that in Example 30 step 4 from N-(2,5-dichloropyrimidin-4-yl)-1-(methylsulfonyl)indolin-7-amine and 1-(7-aminobenzo[d][1,3]dioxol-4-yl)-N,N-dimethylpiperidin-4-amine. ¹H NMR (400 MHz, CD₃OD) δ 8.52 (s, 1H), 7.96 (s, 1H), 7.90 (s, 1H), 7.12 (s, 2H), 6.98 (d, J=8.8 Hz, 1H), 6.42 (d, J=8.8 Hz, 1H), 5.85 (s, 2H), 4.59 (s, 2H), 4.11 (s, 2H), 3.75 (d, J=12.0 Hz, 2H), 3.14 (s, 2H), 2.94 (s, 3H), 2.70 (s, 7H), 2.10 (d, J=11.7 Hz, 2H), 1.79 (d, J=10.2 Hz, 2H); [M+H]⁺=586.2.

### Example 52: 5-bromo-N²-(7-(4-(dimethylamino)piperidin-1-yl)benzo[d][1,3]dioxol-4-yl)-N⁴-(1-(methylsulfonyl)indolin-7-yl)pyrimidine-2,4-diamine

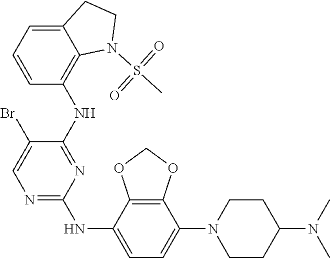 [SMILES: CN(C)C1CCN(c2ccc(Nc3ncc(Br)c(Nc4cccc5c4N(S(C)(=O)=O)CC5)n3)c3c2OCO3)CC1]

The titled compound (12.35 mg, 35%) was prepared in a manner similar to that in Example 21 step 3 from N-(5-bromo-2-chloropyrimidin-4-yl)-1-(methylsulfonyl) indolin-7-amine and 1-(7-aminobenzo[d][1,3]dioxol-4-yl)-N,N-dimethylpiperidin-4-amine. ¹H NMR (400 MHz, CD₃OD) δ 8.05 (s, 1H), 7.85 (s, 1H), 7.12 (s, 2H), 6.97 (d, J=8.9 Hz, 1H), 6.41 (d, J=8.9 Hz, 1H), 5.84 (s, 2H), 4.61 (s, 1H), 4.11 (t, J=7.2 Hz, 2H), 3.77 (d, J=10.4 Hz, 2H), 3.15 (d, J=7.8 Hz, 3H), 2.95 (s, 3H), 2.82-2.78 (m, 9H), 2.14 (d, J=11.7 Hz, 2H), 1.84 (d, J=11.6 Hz, 2H). [M+H]⁺=630.5/632.6.

### Example 53: 5-bromo-N²-(7-(4-(dimethylamino)piperidin-1-yl)-2,3-dihydro-1H-inden-4-yl)-N⁴-(1-(methylsulfonyl)indolin-7-yl)pyrimidine-2,4-diamine

### Step 1: 7-bromo-2,3-dihydro-TH-inden-4-amine

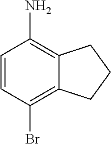 [SMILES: Nc1ccc(Br)c2c1CCC2]

The titled compound (300 mg, 70%) was prepared in a manner similar to that in Example 51 step 1 from 2,3-dihydro-TH-inden-4-amine. [M+H]⁺=211.9/213.1.

### Step 2: 4-bromo-7-nitro-2,3-dihydro-TH-indene

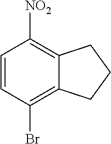 [SMILES: O=[N+]([O-])c1ccc(Br)c2c1CCC2]

The titled compound (233 mg, 68%) was prepared in a manner similar to that in Example 51 step 2 from 7-bromo-2,3-dihydro-1H-inden-4-amine. [M+H]⁺=241.9.

### Step 3: N,N-dimethyl-1-(7-nitro-2,3-dihydro-1H-inden-4-yl)piperidin-4-amine

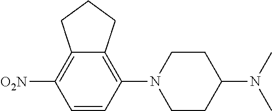 [SMILES: CN(C)C1CCN(c2ccc([N+](=O)[O-])c3c2CCC3)CC1]

The titled compound (182 mg, 65%) was prepared in a manner similar to that in Example 51 step 3 from 4-bromo-7-nitro-2,3-dihydro-1H-indene and N,N-dimethylpiperidin-4-amine. [M+H]⁺=290.2.

### Step 4: 1-(7-amino-2,3-dihydro-1H-inden-4-yl)-N,N-dimethylpiperidin-4-amine

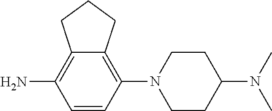 [SMILES: CN(C)C1CCN(c2ccc(N)c3c2CCC3)CC1]

The titled compound (150 mg, 90%) was prepared in a manner similar to that in Example 30 step 3 from N,N-dimethyl-1-(7-nitro-2,3-dihydro-1H-inden-4-yl)piperidin-4-amine. [M+H]⁺=260.2.

### Step 5: 5-bromo-N²-(7-(4-(dimethylamino)piperidin-1-yl)-2,3-dihydro-1H-inden-4-yl)-N⁴-(1-(methylsulfonyl)indolin-7-yl)pyrimidine-2,4-diamine

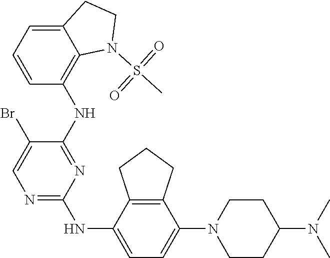 [SMILES: CN(C)C1CCN(c2ccc(Nc3ncc(Br)c(Nc4cccc5c4N(S(C)(=O)=O)CC5)n3)c3c2CCC3)CC1]

The titled compound (8.35 mg, 25%) was prepared in a manner similar to that in Example 21 step 3 from N-(5-bromo-2-chloropyrimidin-4-yl)-1-(methylsulfonyl)indolin-7-amine and 1-(7-amino-2,3-dihydro-1H-inden-4-yl)-N,N-dimethylpiperidin-4-amine. ¹H NMR (400 MHz, DMSO) δ 8.72 (s, 1H), 8.60 (s, 1H), 8.13 (s, 1H), 7.80 (s, 1H), 7.13 (s, 2H), 7.02 (s, 1H), 6.68 (d, J=8.6 Hz, 1H), 4.03 (s, 2H), 3.06 (m, 6H), 2.75 (m, 2H), 2.67 (m, 2H), 2.58 (m, 4H), 2.29 (s, 6H), 1.87 (s, 4H), 1.55 (d, J=11.5 Hz, 2H); [M+H]⁺=626.18.

### Example 54: 5-bromo-N²-(8-(4-(dimethylamino)piperidin-1-yl)-2,3-dihydrobenzo[b][1,4]dioxin-5-yl)-N⁴-(1-(methylsulfonyl)indolin-7-yl)pyrimidine-2,4-diamine

### Step 1: 5-bromo-8-nitro-2,3-dihydrobenzo[b][1,4]dioxine

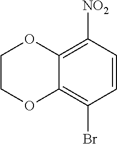 [SMILES: O=[N+]([O-])c1ccc(Br)c2c1OCCO2]

The titled compound (1.1 g, 86%) was prepared in a manner similar to that in Example 51 step 2 from 8-bromo-2,3-dihydrobenzo[b][1,4]dioxin-5-amine and m-CPBA. [M+H]⁺=260.0/262.0.

### Step 2: N,N-dimethyl-1-(8-nitro-2,3-dihydrobenzo[b][1,4]dioxin-5-yl)piperidin-4-amine

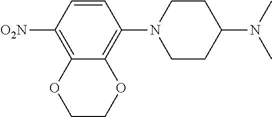 [SMILES: CN(C)C1CCN(c2ccc([N+](=O)[O-])c3c2OCCO3)CC1]

The titled compound (230 mg, 62%) was prepared in a manner similar to that in Example 51 step 3 from 5-bromo-8-nitro-2,3-dihydrobenzo[b][1,4]dioxine and N,N-dimethylpiperidin-4-amine. [M+H]⁺=308.2.

### Step 3: 1-(8-amino-2,3-dihydrobenzo[b][1,4]dioxin-5-yl)-N,N-dimethylpiperidin-4-amine

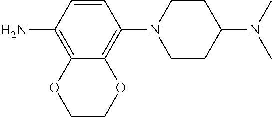 [SMILES: CN(C)C1CCN(c2ccc(N)c3c2OCCO3)CC1]

The titled compound (190 mg, 92%) was prepared in a manner similar to that in Example 51 step 4 from N,N-dimethyl-1-(8-nitro-2,3-dihydrobenzo[b][1,4]dioxin-5-yl)piperidin-4-amine. [M+H]⁺=278.2.

### Step 4: 5-bromo-N²-(8-(4-(dimethylamino)piperidin-1-yl)-2,3-dihydrobenzo[b][1,4]dioxin-5-yl)-N⁴-(1-(methylsulfonyl)indolin-7-yl)pyrimidine-2,4-diamine

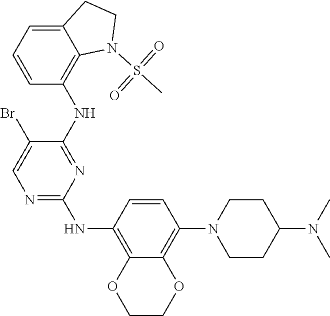 [SMILES: CN(C)C1CCN(c2ccc(Nc3ncc(Br)c(Nc4cccc5c4N(S(C)(=O)=O)CC5)n3)c3c2OCCO3)CC1]

The titled compound was prepared in a manner similar to that in Example 2. ¹H NMR (400 MHz, DMSO) δ_{H} 8.80 (s, 1H), 8.17 (s, 1H), 7.89 (s, 1H), 7.84 (d, J=7.1, 1H), 7.21-7.04 (m, 3H), 6.38 (d, J=8.7, 1H), 4.21 (s, 4H), 4.05 (t, J=7.1, 2H), 3.28-3.24 (m, 3H), 3.11 (t, J=6.7, 2H), 3.05 (s, 3H), 2.54 (s, 1H), 2.26 (s, 7H), 1.83 (d, J=11.5, 2H), 1.63-1.43 (m, 2H); [M+H]⁺=644.2/646.2.

### Example 55: 5-bromo-N²-(4-(4-(dimethylamino)piperidin-1-yl)-2,2-dimethyl-2,3-dihydrobenzofuran-7-yl)-N⁴-(1-(methylsulfonyl)indolin-7-yl)pyrimidine-2,4-diamine

### Step 1: 1-(2,2-dimethyl-7-nitro-2,3-dihydrobenzofuran-4-yl)-N,N-dimethylpiperidin-4-amine

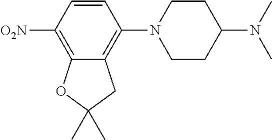 [SMILES: CN(C)C1CCN(c2ccc([N+](=O)[O-])c3c2CC(C)(C)O3)CC1]

The titled compound (180 mg, 45%) was prepared in a manner similar to that in Example 51 step 3 from 4-bromo-2,2-dimethyl-7-nitro-2,3-dihydrobenzofuran (the intermediate was prepared according to the method described in WO 2016169504

A1) and N,N-dimethylpiperidin-4-amine. [M+H]⁺=320.2.

### Step 2: 1-(7-amino-2,2-dimethyl-2,3-dihydrobenzofuran-4-yl)-N,N-dimethylpiperidin-4-amine

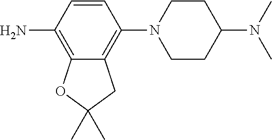 [SMILES: CN(C)C1CCN(c2ccc(N)c3c2CC(C)(C)O3)CC1]

The titled compound (160 mg, 87%) was prepared in a manner similar to that in Example 51 step 4 from 1-(2,2-dimethyl-7-nitro-2,3-dihydrobenzofuran-4-yl)-N,N-dimethylpiperidin-4-amine and Pd/C. [M+H]⁺=290.2.

### Step 3: 5-bromo-N²-(4-(4-(dimethylamino)piperidin-1-yl)-2,2-dimethyl-2,3-dihydrobenzofuran-7-yl)-N⁴-(1-(methylsulfonyl)indolin-7-yl)pyrimidine-2,4-diamine

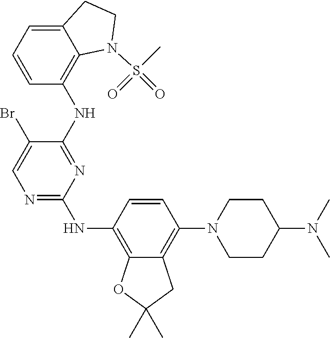 [SMILES: CN(C)C1CCN(c2ccc(Nc3ncc(Br)c(Nc4cccc5c4N(S(C)(=O)=O)CC5)n3)c3c2CC(C)(C)O3)CC1]

The titled compound was prepared in a manner similar to that in Example 2. ¹H NMR (400 MHz, DMSO) δ_{H} 8.74 (s, 1H), 8.12 (s, 1H), 8.04 (s, 1H), 7.86 (d, J=7.6, 1H), 7.21-7.09 (m, 2H), 7.04 (t, J=5.6, 1H), 6.33 (d, J=8.6, 1H), 4.03 (t, J=7.1, 2H), 3.30-3.27 (m, 2H), 3.09 (t, J=6.9, 2H), 3.04 (s, 3H), 2.93 (s, 2H), 2.58 (t, J=11.6, 2H), 2.22 (s, 7H), 1.85 (d, J=11.3, 2H), 1.50 (q, J=11.6, 2H), 1.33 (s, 6H); [M+H]⁺=656.2, 658.2.

### Example 56: 5-bromo-N²-(2,2-dimethyl-4-(4-methylpiperazin-1-yl)-2,3-dihydrobenzofuran-7-yl)-N⁴-(1-(methylsulfonyl)indolin-7-yl)pyrimidine-2,4-diamine

### Step 1: 1-(2,2-dimethyl-7-nitro-2,3-dihydrobenzofuran-4-yl)-4-methylpiperazine

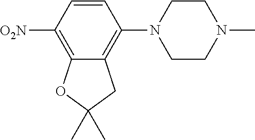 [SMILES: CN1CCN(c2ccc([N+](=O)[O-])c3c2CC(C)(C)O3)CC1]

The titled compound (170 mg, 62%) was prepared in a manner similar to that in Example 51 step 3 from 4-bromo-2,2-dimethyl-7-nitro-2,3-dihydrobenzofuran and 1-methylpiperazine. [M+H]⁺=292.2.

### Step 2: 2,2-dimethyl-4-(4-methylpiperazin-1-yl)-2,3-dihydrobenzofuran-7-amine

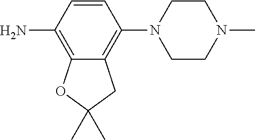 [SMILES: CN1CCN(c2ccc(N)c3c2CC(C)(C)O3)CC1]

The titled compound (110 mg, 84%) was prepared in a manner similar to that in Example 51 step 4 from 1-(2,2-dimethyl-7-nitro-2,3-dihydrobenzofuran-4-yl)-4-methylpiperazine. [M+H]⁺=262.2.

### Step 3: 5-bromo-N²-(2,2-dimethyl-4-(4-methylpiperazin-1-yl)-2,3-dihydrobenzofuran-7-yl)-N⁴-(1-(methylsulfonyl)indolin-7-yl)pyrimidine-2,4-diamine

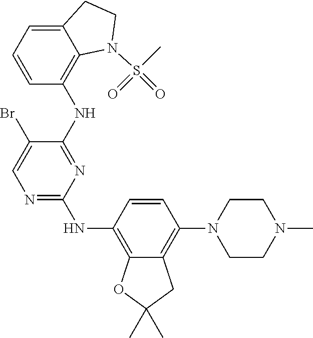 [SMILES: CN1CCN(c2ccc(Nc3ncc(Br)c(Nc4cccc5c4N(S(C)(=O)=O)CC5)n3)c3c2CC(C)(C)O3)CC1]

The titled compound was prepared in a manner similar to that in Example 2. ¹H NMR (400 MHz, DMSO) δ_{H} 8.74 (s, 1H), 8.13 (s, 1H), 8.05 (s, 1H), 7.86 (d, J=7.4, 1H), 7.20-7.10 (m, 2H), 7.04 (t, J=7.2, 1H), 6.32 (d, J=8.5, 1H), 4.03 (t, J=7.0, 2H), 3.09 (t, J=7.1, 2H), 3.03 (s, 3H), 2.93 (s, 6H), 2.46 (s, 4H), 2.23 (s, 3H), 1.33 (s, 6H); [M+H]⁺=628.2.

### Example 57: 5-bromo-N²-(2,2-dimethyl-4-(4-(4-methylpiperazin-1-yl) piperidin-1-yl)-2,3-dihydrobenzofuran-7-yl)-N⁴-(1-(methylsulfonyl)indolin-7-yl)pyrimidine-2,4-diamine

### Step 1: 1-(1-(2,2-dimethyl-7-nitro-2,3-dihydrobenzofuran-4-yl)piperidin-4-yl)-4-methylpiperazine

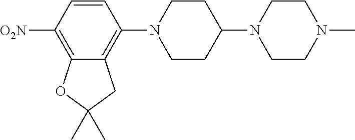 [SMILES: CN1CCN(C2CCN(c3ccc([N+](=O)[O-])c4c3CC(C)(C)O4)CC2)CC1]

The titled compound (190 mg, 51%) was prepared in a manner similar to that in Example 51 step 3 from 4-bromo-2,2-dimethyl-7-nitro-2,3-dihydrobenzofuran and 1-methyl-4-(piperidin-4-yl)piperazine. [M+H]⁺=375.3.

### Step 2: 2,2-dimethyl-4-(4-(4-methylpiperazin-1-yl)piperidin-1-yl)-2,3-dihydrobenzofuran-7-amine

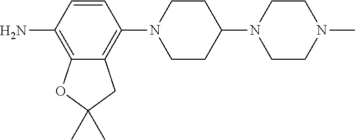 [SMILES: CN1CCN(C2CCN(c3ccc(N)c4c3CC(C)(C)O4)CC2)CC1]

The titled compound (190 mg, 51%) was prepared in a manner similar to that in Example 51 step 3 from 1-(1-(2,2-dimethyl-7-nitro-2,3-dihydrobenzofuran-4-yl)piperidin-4-yl)-4-methylpiperazine. [M+H]⁺=345.3.

### Step 3: 5-bromo-N²-(2,2-dimethyl-4-(4-(4-methylpiperazin-1-yl)piperidin-1-yl)-2,3-dihydrobenzofuran-7-yl)-N⁴-(1-(methylsulfonyl)indolin-7-yl)pyrimidine-2,4-diamine

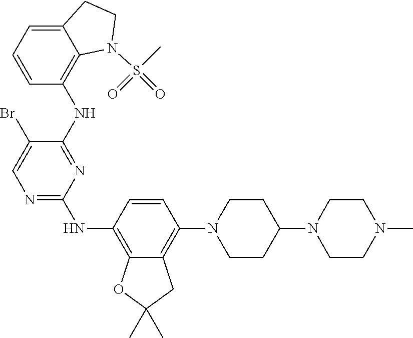 [SMILES: CN1CCN(C2CCN(c3ccc(Nc4ncc(Br)c(Nc5cccc6c5N(S(C)(=O)=O)CC6)n4)c4c3CC(C)(C)O4)CC2)CC1]

The titled compound was prepared in a manner similar to that in Example 2. ¹H NMR (400 MHz, DMSO) δ_{H} 8.74 (s, 1H), 8.12 (s, 1H), 8.02 (s, 1H), 7.85 (d, J=7.9, 1H), 7.18-7.09 (m, 2H), 7.04 (t, J=7.1, 1H), 6.32 (d, J=8.5, 1H), 4.03 (t, J=7.1, 2H), 3.32-3.28 (m, 5H), 3.09 (t, J=6.9, 2H), 3.03 (s, 3H), 2.92 (s, 2H), 2.66-2.52 (m, 4H), 2.39-2.25 (m, 4H), 2.17 (s, 3H), 1.84 (d, J=11.5, 2H), 1.52 (dd, J=21.6, 10.8, 2H), 1.33 (s, 6H); [M+H]⁺=711.2/713.2.

### Example 58: 5-bromo-N²-(5-fluoro-4-(4-methylpiperazin-1-yl)-2,3-dihydrobenzofuran-7-yl)-N⁴-(1-(methylsulfonyl)indolin-7-yl)pyrimidine-2,4-diamine

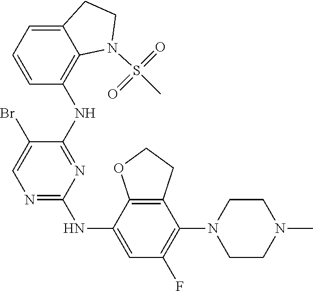 [SMILES: CN1CCN(c2c(F)cc(Nc3ncc(Br)c(Nc4cccc5c4N(S(C)(=O)=O)CC5)n3)c3c2CCO3)CC1]

The titled compound was synthesized in the procedures similar to Example 59. ¹H NMR (400 MHz, DMSO) δ 8.83 (s, 1H), 8.19 (s, 2H), 7.85 (d, J=7.5 Hz, 1H), 7.22-7.04 (m, 3H), 4.47 (t, J=8.6 Hz, 2H), 4.05 (t, J=7.2 Hz, 2H), 3.26 (d, J=8.8 Hz, 2H), 3.08-3.03 (m, 9H), 2.45 (s, 4H), 2.24 (s, 3H); [M+H]⁺=618.1/620.2.

### Example 59: 5-bromo-N²-(4-(4-(dimethylamino)piperidin-1-yl)-5-fluoro-2,3-dihydrobenzofuran-7-yl)-N⁴-(1-(methylsulfonyl)indolin-7-yl)pyrimidine-2,4-diamine

### Step 1: 4-bromo-5-fluoro-2,3-dihydrobenzofuran-7-amine

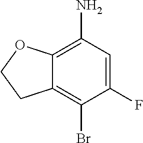 [SMILES: Nc1cc(F)c(Br)c2c1OCC2]

The titled compound (1.3 g, 91%) was prepared in a manner similar to that in Example 51 step 1 from 5-fluoro-2,3-dihydrobenzofuran-7-amine and NBS. [M+H]⁺=232.0/234.0.

### Step 2: 4-bromo-5-fluoro-7-nitro-2,3-dihydrobenzofuran

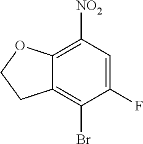 [SMILES: O=[N+]([O-])c1cc(F)c(Br)c2c1OCC2]

The titled compound (1.1 g, 87%) was prepared in a manner similar to that in Example 51 step 2 from 4-bromo-5-fluoro-2,3-dihydrobenzofuran-7-amine. [M+H]±=262.0/264.0.

### Step 3: 1-(5-fluoro-7-nitro-2,3-dihydrobenzofuran-4-yl)-N,N-dimethylpiperidin-4-amine

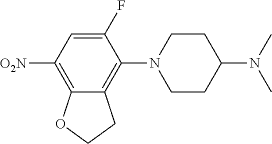 [SMILES: CN(C)C1CCN(c2c(F)cc([N+](=O)[O-])c3c2CCO3)CC1]

The titled compound (160 mg, 62%) was prepared in a manner similar to that in Example 51 step 3 from 4-bromo-5-fluoro-7-nitro-2,3-dihydrobenzofuran and N,N-dimethylpiperidin-4-amine. [M+H]⁺=310.2.

### Step 4: 1-(7-amino-5-fluoro-2,3-dihydrobenzofuran-4-yl)-N,N-dimethylpiperidin-4-amine

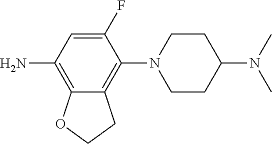 [SMILES: CN(C)C1CCN(c2c(F)cc(N)c3c2CCO3)CC1]

The titled compound (120 mg, 89%) was prepared in a manner similar to that in Example 51 step 4 from 1-(5-fluoro-7-nitro-2,3-dihydrobenzofuran-4-yl)-N,N-dimethylpiperidin-4-amine. [M+H]⁺=280.2.

### Step 5: 5-bromo-N²-(4-(4-(dimethylamino)piperidin-1-yl)-5-fluoro-2,3-dihydrobenzofuran-7-yl)-N⁴-(1-(methylsulfonyl)indolin-7-yl)pyrimidine-2,4-diamine

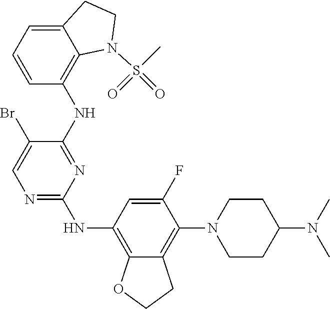 [SMILES: CN(C)C1CCN(c2c(F)cc(Nc3ncc(Br)c(Nc4cccc5c4N(S(C)(=O)=O)CC5)n3)c3c2CCO3)CC1]

The titled compound was prepared in a manner similar to that in Example 2. ¹H NMR (400 MHz, DMSO) δ_{H} 8.83 (s, 1H), 8.20 (t, J=6.1, 2H), 7.85 (d, J=7.7, 1H), 7.14 (dt, J=15.3, 10.6, 3H), 4.47 (t, J=8.5, 2H), 4.05 (t, J=7.0, 2H), 3.25-3.17 (m, 4H), 3.10 (t, J=6.8, 2H), 3.05 (s, 3H), 2.94 (t, J=11.4, 2H), 2.25 (s, 7H), 1.82 (d, J=11.6, 2H), 1.48 (dd, J=21.0, 10.7, 2H); [M+H]⁺=646.2/648.2.

### Example 60: 5-bromo-N²-(5-fluoro-4-(4-(4-methylpiperazin-1-yl)piperidin-1-yl)-2,3-dihydrobenzofuran-7-yl)-N⁴-(1-(methylsulfonyl)indolin-7-yl)pyrimidine-2,4-diamine

### Step 1: 1-(1-(5-fluoro-7-nitro-2,3-dihydrobenzofuran-4-yl)piperidin-4-yl)-4-methylpiperazine

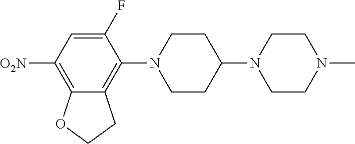 [SMILES: CN1CCN(C2CCN(c3c(F)cc([N+](=O)[O-])c4c3CCO4)CC2)CC1]

The titled compound (180 mg, 47%) was prepared in a manner similar to that in Example 51 step 3 from 4-bromo-5-fluoro-7-nitro-2,3-dihydrobenzofuran and 1-methyl-4-(piperidin-4-yl)piperazine. [M+H]⁺=365.2.

### Step 2: 5-fluoro-4-(4-(4-methylpiperazin-1-yl)piperidin-1-yl)-2,3-dihydrobenzofuran-7-amine

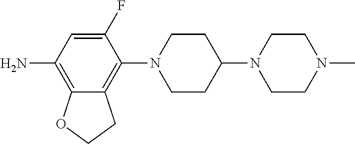 [SMILES: CN1CCN(C2CCN(c3c(F)cc(N)c4c3CCO4)CC2)CC1]

The titled compound (150 mg, 91%) was prepared in a manner similar to that in Example 51 step 4 from 1-(1-(5-fluoro-7-nitro-2,3-dihydrobenzofuran-4-yl)piperidin-4-yl)-4-methylpiperazine and Pd. [M+H]⁺=335.2.

### Step 3: 5-bromo-N²-(5-fluoro-4-(4-(4-methylpiperazin-1-yl)piperidin-1-yl)-2,3-dihydrobenzofuran-7-yl)-N⁴-(1-(methylsulfonyl)indolin-7-yl)pyrimidine-2,4-diamine

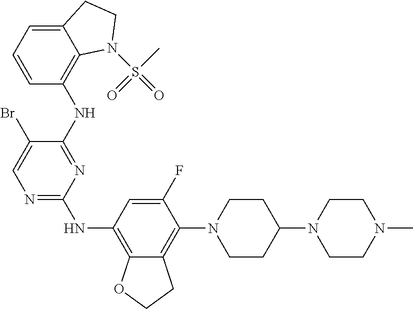 [SMILES: CN1CCN(C2CCN(c3c(F)cc(Nc4ncc(Br)c(Nc5cccc6c5N(S(C)(=O)=O)CC6)n4)c4c3CCO4)CC2)CC1]

The titled compound was prepared in a manner similar to that in Example 2. ¹H NMR (400 MHz, DMSO) δ_{H} 8.83 (s, 1H), 8.25-8.12 (m, 2H), 7.84 (d, J=7.3, 1H), 7.25-7.07 (m, 3H), 4.47 (t, J=8.5, 2H), 4.05 (t, J=7.2, 2H), 3.29-3.14 (m, 8H), 3.10 (t, J=6.9, 2H), 3.04 (s, 3H), 2.94 (t, J=11.3, 2H), 2.41-2.24 (m, 5H), 2.15 (s, 3H), 1.80 (d, J=11.3, 2H), 1.49 (dd, J=21.8, 10.4, 2H); [M+H]⁺=701.2/703.2.

### Example 61: 5-bromo-N²-(2-methyl-4-(4-(4-methylpiperazin-1-yl)piperidin-1-yl)-2,3-dihydrobenzofuran-7-yl)-N⁴-(1-(methylsulfonyl)indolin-7-yl)pyrimidine-2,4-diamine

### Step 1: 1-methyl-4-(1-(2-methyl-7-nitro-2,3-dihydrobenzofuran-4-yl)piperidin-4-vl)piperazine

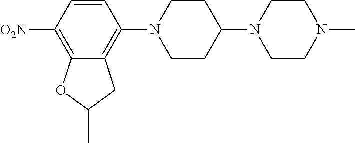 [SMILES: CC1Cc2c(N3CCC(N4CCN(C)CC4)CC3)ccc([N+](=O)[O-])c2O1]

The titled compound (160 mg, 42%) was prepared in a manner similar to that in Example 51 step 3 from 4-bromo-2-methyl-7-nitro-2,3-dihydrobenzofuran and 1-methyl-4-(piperidin-4-yl)piperazine. [M+H]⁺=361.2

### Step 2: 2-methyl-4-(4-(4-methylpiperazin-1-yl)piperidin-1-yl)-2,3-dihydrobenzofuran-7-amine

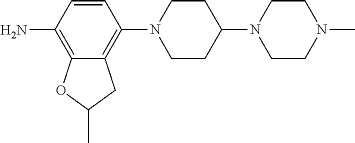 [SMILES: CC1Cc2c(N3CCC(N4CCN(C)CC4)CC3)ccc(N)c2O1]

The titled compound (120 mg, 87%) was prepared in a manner similar to that in Example 51 step 4 from 1-methyl-4-(1-(2-methyl-7-nitro-2,3-dihydrobenzofuran-4-yl)piperidin-4-yl)piperazine. [M+H]⁺=331.2

### Step 3: 5-bromo-N²-(2-methyl-4-(4-(4-methylpiperazin-1-yl)piperidin-1-yl)-2,3-dihydrobenzofuran-7-yl)-N⁴-(1-(methylsulfonyl)indolin-7-yl)pyrimidine-2,4-diamine

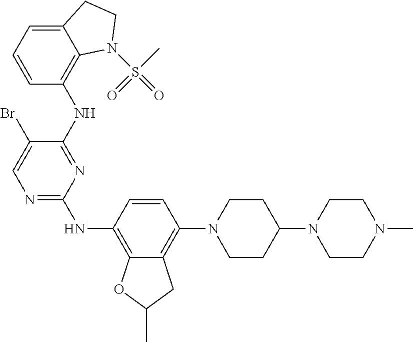 [SMILES: CC1Cc2c(N3CCC(N4CCN(C)CC4)CC3)ccc(Nc3ncc(Br)c(Nc4cccc5c4N(S(C)(=O)=O)CC5)n3)c2O1]

The titled compound was prepared in a manner similar to that in Example 2. ¹H NMR (400 MHz, DMSO) δ_{H} 8.74 (s, 1H), 8.16 (s, 1H), 8.12 (s, 1H), 7.87 (d, J=7.8, 1H), 7.11 (d, J=5.4, 2H), 7.05 (s, 1H), 6.33 (d, J=8.6, 1H), 4.82 (dd, J=13.3, 7.0, 1H), 4.03 (t, J=6.7, 2H), 3.29-3.19 (m, 5H), 3.09 (t, J=6.2, 2H), 3.04 (s, 3H), 2.72 (dd, J=15.0, 7.6, 1H), 2.65-2.52 (m, 5H), 2.40-2.24 (m, 4H), 2.17 (s, 3H), 1.85 (d, J=10.6, 2H), 1.52 (dd, J=22.3, 10.1, 2H), 1.32 (d, J=5.9, 3H); [M+H]⁺=697.2/699.2.

### Example 62: 5-bromo-N²-(2-methyl-4-(4-methylpiperazin-1-yl)-2,3-dihydrobenzofuran-7-yl)-N⁴-(1-(methylsulfonyl)indolin-7-yl)pyrimidine-2,4-diamine

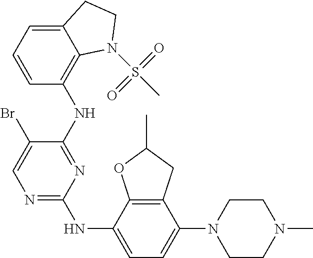 [SMILES: CC1Cc2c(N3CCN(C)CC3)ccc(Nc3ncc(Br)c(Nc4cccc5c4N(S(C)(=O)=O)CC5)n3)c2O1]

The titled compound was synthesized in the procedures similar to Example 2. ¹H NMR (400 MHz, CD₃OD) δ 8.05 (s, 1H), 7.80 (d, J=7.4 Hz, 1H), 7.41 (d, J=8.5 Hz, 1H), 7.17 (s, 2H), 6.34 (d, J=8.3 Hz, 1H), 4.90 (d, J=6.0 Hz, 1H), 4.11 (s, 3H), 3.14 (d, J=7.5 Hz, 2H), 3.07 (s, 4H), 2.96 (s, 3H), 2.77 (s, 6H), 2.47 (s, 3H), 1.39 (d, J=6.2 Hz, 3H); [M+H]⁺=614.1/616.2.

### Example 65: 5-bromo-N²-(4-(4-(dimethylamino)piperidin-1-yl)-2-methyl-2,3-dihydrobenzofuran-7-yl)-N⁴-(1-(methylsulfonyl)indolin-7-yl)pyrimidine-2,4-diamine

### Step 1: N,N-dimethyl-1-(2-methyl-7-nitro-2,3-dihydrobenzofuran-4-yl)piperidin-4-amine

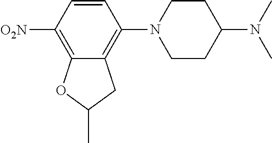 [SMILES: CC1Cc2c(N3CCC(N(C)C)CC3)ccc([N+](=O)[O-])c2O1]

The titled compound (180 mg, 61%) was prepared in a manner similar to that in Example 51 step 3 from 4-bromo-2-methyl-7-nitro-2,3-dihydrobenzofuran and N,N-dimethylpiperidin-4-amine. [M+H]⁺=306.1.

### Step 2: 1-(7-amino-2-methyl-2,3-dihydrobenzofuran-4-yl)-N,N-dimethylpiperidin-4-amine

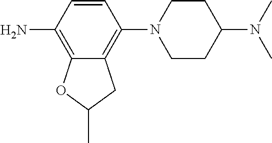 [SMILES: CC1Cc2c(N3CCC(N(C)C)CC3)ccc(N)c2O1]

The titled compound (160 mg, 91%) was prepared in a manner similar to that in Example 51 step 4 from N,N-dimethyl-1-(2-methyl-7-nitro-2,3-dihydrobenzofuran-4-yl)piperidin-4-amine. [M+H]J=276.3.

### Step 3: 5-bromo-N²-(4-(4-(dimethylamino)piperidin-1-yl)-2-methyl-2,3-dihydrobenzofuran-7-yl)-N⁴-(1-(methylsulfonyl)indolin-7-yl)pyrimidine-2,4-diamine

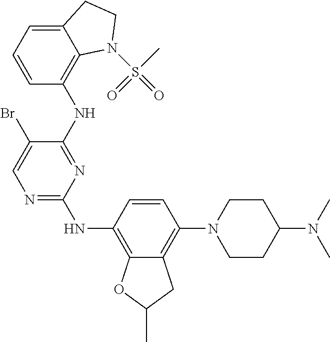 [SMILES: CC1Cc2c(N3CCC(N(C)C)CC3)ccc(Nc3ncc(Br)c(Nc4cccc5c4N(S(C)(=O)=O)CC5)n3)c2O1]

The titled compound was prepared in a manner similar to that in Example 2. ¹H NMR (400 MHz, DMSO) δ_{H} 8.80-8.68 (m, 1H), 8.20-8.14 (m, 1H), 8.13 (s, 1H), 7.93-7.80 (m, 1H), 7.17-7.08 (m, 2H), 7.05 (dd, J=11.0, 3.8 Hz, 1H), 6.34 (d, J=8.6 Hz, 1H), 4.82 (dd, J=13.2, 6.8 Hz, 1H), 4.03 (t, J=6.1 Hz, 2H), 3.30-3.22 (m, 3H), 3.09 (t, J=6.8 Hz, 2H), 3.04 (s, 3H), 2.73 (dd, J=15.3, 8.0 Hz, 1H), 2.59 (dd, J=23.9, 12.1 Hz, 2H), 2.25 (s, 7H), 1.86 (d, J=11.5 Hz, 2H), 1.51 (dd, J=21.4, 10.0 Hz, 2H), 1.32 (d, J=6.1 Hz, 3H); [M+H]⁺=642.2/644.2.

### Example 68: N²-(2-methoxy-4-(4-(4-methylpiperazin-1-yl)piperidin-1-yl)phenyl)-9-methyl-N⁶-(1-(methylsulfonyl)indolin-7-yl)-9H-purine-2,6-diamine

### Step 1: 2-chloro-9-methyl-N-(1-(methylsulfonyl)indolin-7-yl)-9H-purin-6-amine

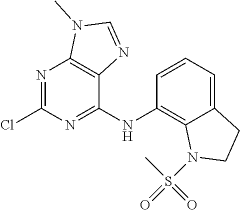 [SMILES: Cn1cnc2c(Nc3cccc4c3N(S(C)(=O)=O)CC4)nc(Cl)nc21]

A mixture of 1-(methylsulfonyl)indolin-7-amine (50 mg, 0.23 mmol), 2,6-dichloro-9-methyl-9H-purine (70 mg, 0.35 mmol) and DIEA (60 mg, 0.46 mmol) in i-PrOH (8 mL) was stirred in a round bottom flask at 100° C. for 16 hours. The mixture was evaporated in vacuum to afford the crude product, which was purified with silica gel column chromatography (PE: EA=100: 0˜ 1: 1 gradient elution) to give the title product (45 mg, 51%). [M+H]⁺=379.2.

### Step 2: N²-(2-methoxy-4-(4-(4-methylpiperazin-1-yl)piperidin-1-yl)phenyl)-9-methyl-N⁶-(1-(methylsulfonyl)indolin-7-yl)-9H-purine-2,6-diamine

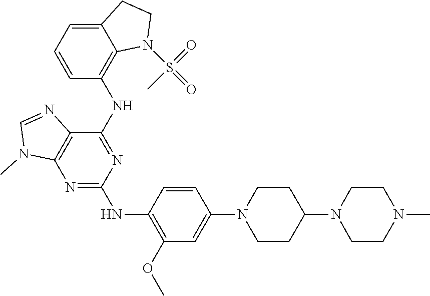

A mixture of 2-chloro-9-methyl-N-(1-(methylsulfonyl)indolin-7-yl)-9H-purin-6-amine (45 mg, 0.12 mmol), 2-methoxy-4-(4-(4-methylpiperazin-1-yl)piperidin-1-yl)aniline (43 mg, 0.14 mmol), Pd₂(dba)₃ (11 mg, 0.012 mmol), BINAP (15 mg, 0.024 mmol) and K₃PO₄ (76 mg, 0.36 mmol) in toluene (8 mL) was stirred in a round bottom flask at 100° C. overnight under N₂. The mixture was evaporated in vacuum to afford the crude residue, which was purified with silica gel column chromatography (DCM: MeOH=100: 0˜ 5: 1 gradient elution) to give the crude product, which was further purified with pre-HPLC to give the title product (7 mg, 9.1%). ¹H NMR (400 MHz, DMSO) δ_{H}9 0.15 (s, 1H), 8.17 (d, J=7.7 Hz, 1H), 7.90 (s, 1H), 7.85 (d, J=7.9 Hz, 1H), 7.53 (s, 1H), 7.17 (d, J=7.7 Hz, 1H), 7.12 (s, 1H), 6.63 (s, 1H), 6.46 (d, J=8.8 Hz, 1H), 4.07 (s, 2H), 3.81 (s, 3H), 3.67 (d, J=13.3 Hz, 5H), 3.11 (s, 2H), 3.05 (s, 3H), 2.63 (t, J=11.7 Hz, 2H), 2.51 (s, 4H), 2.33 (s, 5H), 2.16 (s, 3H), 1.85 (d, J=11.7 Hz, 2H), 1.53 (d, J=11.8 Hz, 2H); [M+H]⁺=647.5.

### Example 69: N⁵-(2-methoxy-4-(4-(4-methylpiperazin-1-yl)piperidin-1-yl)phenyl)-2-methyl-N⁷-(1-(methylsulfonyl)indolin-7-yl)-2H-pyrazolo[4,3-d]pyrimidine-5,7-diamine

### Step 1: 5-chloro-2-methyl-N-(1-(methylsulfonyl)indolin-7-yl)-2H-pyrazolo[4,3-d]pyrimidin-7-amine

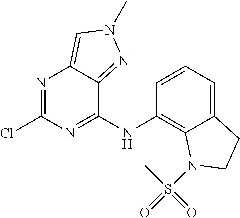 [SMILES: Cn1cc2nc(Cl)nc(Nc3cccc4c3N(S(C)(=O)=O)CC4)c2n1]

A mixture of 1-(methylsulfonyl)indolin-7-amine (80 mg, 0.38 mmol), 5,7-dichloro-2-methyl-2H-pyrazolo[4,3-d]pyrimidine (114 mg, 0.57 mmol) and DIEA (98 mg, 0.76 mmol) in i-PrOH (10 mL) was stirred in a round bottom flask at 100° C. for 16 hours. The mixture was evaporated in vacuum to afford the crude product, which was purified with silica gel column chromatography (PE: EA=100: 0˜ 1: 2 gradient elution) to give the title product (90 mg, 63%). [M+H]⁺=379.2.

### Step 2: N⁵-(2-methoxy-4-(4-(4-methylpiperazin-1-yl)piperidin-1-yl)phenyl)-2-methyl-N⁷-(1-(methylsulfonyl)indolin-7-yl)-2H-pyrazolo[4,3-d]pyrimidine-5,7-diamine

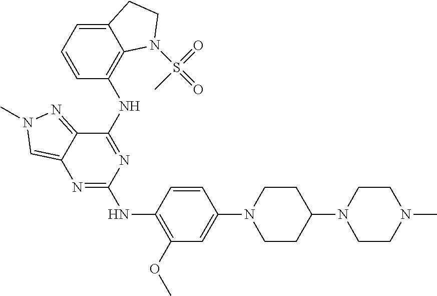

A mixture of 5-chloro-2-methyl-N-(1-(methylsulfonyl)indolin-7-yl)-2H-pyrazolo[4,3-d]pyrimidin-7-amine (40 mg, 0.11 mmol), 2-methoxy-4-(4-(4-methylpiperazin-1-yl)piperidin-1-yl)aniline (40 mg, 0.13 mmol), Pd₂(dba)₃ (11 mg, 0.011 mmol), BINAP (15 mg, 0.022 mmol) and K₃PO₄ (70 mg, 0.33 mmol) in toluene (6 mL) was stirred in a round bottom flask at 100° C. overnight under N₂. The mixture was evaporated in vacuum to afford the crude residue, which was purified with silica gel column chromatography (DCM: MeOH=100: 0˜ 8: 1 gradient elution) to give the title product (4 mg, 6%). ¹H NMR (400 MHz, DMSO) δ_{H} 9.32 (s, 1H), 8.27 (s, 1H), 8.07-8.02 (m, 1H), 8.00 (s, 1H), 7.24 (s, 2H), 7.19 (s, 1H), 6.62 (s, 1H), 6.47 (s, 1H), 4.09 (s, 4H), 3.82 (s, 3H), 3.66 (d, J=11.0 Hz, 2H), 3.14 (s, 2H), 3.05 (s, 3H), 2.62 (s, 2H), 2.52 (s, 2H), 2.45-2.25 (m, 8H), 2.16 (s, 3H), 1.83 (s, 2H), 1.52 (d, J=9.0 Hz, 2H); [M+H]⁺=647.5.

### Example 70: N²-(2-methoxy-4-(4-(4-methylpiperazin-1-yl)piperidin-1-yl)phenyl)-7-methyl-N⁶-(1-(methylsulfonyl)indolin-7-yl)-7H-purine-2,6-diamine

### Step 1: 2-chloro-7-methyl-N-(1-(methylsulfonyl)indolin-7-yl)-7H-purin-6-amine

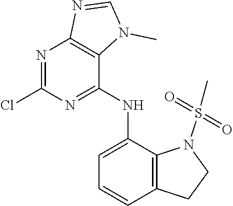 [SMILES: Cn1cnc2nc(Cl)nc(Nc3cccc4c3N(S(C)(=O)=O)CC4)c21]

A mixture of 1-(methylsulfonyl)indolin-7-amine (60 mg, 0.28 mmol), 2,6-dichloro-7-methyl-7H-purine (86 mg, 0.42 mmol) and DIEA (72 mg, 0.56 mmol) in i-PrOH (8 mL) was stirred in a round bottom flask at 100° C. for 16 hours. The mixture was evaporated in vacuum to afford the crude product, which was purified with silica gel column chromatography (PE: EA=100: 0˜ 1: 1 gradient elution) to give the title product (55 mg, 51%). [M+H]⁺=379.2.

### Step 2: N²-(2-methoxy-4-(4-(4-methylpiperazin-1-yl)piperidin-1-yl)phenyl)-7-methyl-N⁶-(1-(methylsulfonyl)indolin-7-yl)-7H-purine-2,6-diamine

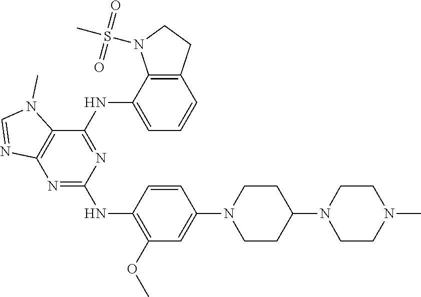 [SMILES: COc1cc(N2CCC(N3CCN(C)CC3)CC2)ccc1Nc1nc(Nc2cccc3c2N(S(C)(=O)=O)CC3)c2c(ncn2C)n1]

Under N₂ atmosphere, A mixture of 2-chloro-7-methyl-N-(1-(methylsulfonyl)indolin-7-yl)-7H-purin-6-amine (55 mg, 0.15 mmol), 2-methoxy-4-(4-(4-methylpiperazin-1-yl)piperidin-1-yl)aniline (52 mg, 0.17 mmol), G3 BrettPhos Pd (13 mg, 0.015 mmol) and Cs₂CO₃ (98 mg, 0.30 mmol) in 1,4-dioxane (8 mL) was stirred in a round bottom flask at 100° C. for 16h. The mixture was evaporated in vacuum to afford the crude product, which was purified with silica gel column chromatography (DCM: MeOH=100: 0˜ 5: 1 gradient elution) to give the crude product, which was purified by pre-HPLC to give the title product (8 mg, 8.5%). ¹H NMR (400 MHz, DMSO) δ_{H}8 0.60 (s, 1H), 8.07 (s, 1H), 7.90 (s, 2H), 7.24 (d, J=7.8 Hz, 1H), 7.19 (s, 2H), 6.60 (s, 1H), 6.41 (d, J=9.1 Hz, 1H), 4.04 (d, J=19.9 Hz, 5H), 3.80 (s, 3H), 3.65 (d, J=11.1 Hz, 2H), 3.11 (d, J=17.2 Hz, 5H), 2.70-2.52 (m, 9H), 2.40 (s, 2H), 2.32 (s, 3H), 1.87 (d, J=10.6 Hz, 2H), 1.54 (d, J=10.2 Hz, 2H); [M+H]⁺=647.5.

### Example 71: N²-(2-methoxy-4-(4-(4-methylpiperazin-1-yl)piperidin-1-yl)phenyl)-N⁴-(1-(methylsulfonyl)indolin-7-yl)-5H-pyrrolo[3,2-d]pyrimidine-2,4-diamine

### Step 1: 2-chloro-N-(1-(methylsulfonyl)indolin-7-yl)-5H-pyrrolo[3,2-d]pyrimidin-4-amine

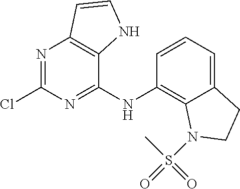 [SMILES: CS(=O)(=O)N1CCc2cccc(Nc3nc(Cl)nc4cc[nH]c34)c21]

To a solution of 1-(methylsulfonyl)indolin-7-amine (60 mg, 0.28 mmol) and 2,4-dichloro-5H-pyrrolo[3,2-d]pyrimidine (79 mg, 0.42 mmol) in i-PrOH (8 mL) was added conc. HCl (0.2 mL). The resulting mixture was heated at 80° C. overnight. The organic solvent was removed under reduced pressure, the residue was basified with saturated NaHCO₃(aq.) solution and extracted with DCM (2×30 mL). The combined organic layer was dried over Na₂SO₄ and concentrated under vacuum to afford the crude residue, which was purified with silica gel column chromatography (PE: EA=100: 0˜ 1: 1 gradient elution) to give the title product (100 mg, 97%). [M+H]⁺=364.2.

### Step 2: N²-(2-methoxy-4-(4-(4-methylpiperazin-1-yl)piperidin-1-yl)phenyl)-N⁴-(1-(methylsulfonyl)indolin-7-yl)-5H-pyrrolo[3,2-d]pyrimidine-2,4-diamine

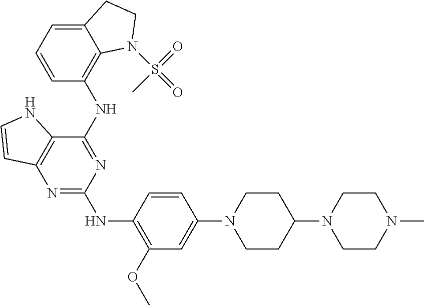 [SMILES: COc1cc(N2CCC(N3CCN(C)CC3)CC2)ccc1Nc1nc(Nc2cccc3c2N(S(C)(=O)=O)CC3)c2[nH]ccc2n1]

Under N₂ atmosphere, A mixture of 2-chloro-N-(1-(methylsulfonyl)indolin-7-yl)-5H-pyrrolo[3,2-d]pyrimidin-4-amine (40 mg, 0.11 mmol), 2-methoxy-4-(4-(4-methylpiperazin-1-yl)piperidin-1-yl)aniline (37 mg, 0.12 mmol), G3 BrettPhos Pd (10 mg, 0.011 mmol) and Cs₂CO₃ (72 mg, 0.22 mmol) in 1,4-dioxane (6 mL) was stirred in a round bottom flask at 100° C. overnight. The mixture was evaporated in vacuum to afford the crude product, which was purified with silica gel column chromatography (DCM: MeOH=100: 0˜ 5: 1 gradient elution) to give crude product, which was further purified with pre-HPLC to give the title product (12 mg, 17%). ¹H NMR (400 MHz, DMSO) δ_{H} 11.17 (s, 1H), 8.47 (s, 1H), 8.14 (s, 1H), 8.04 (d, J=8.1 Hz, 1H), 7.43 (s, 1H), 7.22 (s, 1H), 7.13 (s, 1H), 7.03 (s, 1H), 6.61 (s, 1H), 6.43 (d, J=8.9 Hz, 1H), 6.19 (s, 1H), 4.07 (s, 2H), 3.83 (s, 3H), 3.62 (d, J=12.0 Hz, 2H), 3.13 (s, 2H), 2.96 (s, 3H), 2.61 (d, J=12.6 Hz, 6H), 2.33 (d, J=32.7 Hz, 5H), 2.18 (s, 3H), 1.85 (d, J=12.2 Hz, 2H), 1.53 (d, J=11.3 Hz, 2H); [M+H]⁺=632.5.

### Example 72: N²-(2-methoxy-4-(4-(4-methylpiperazin-1-yl)piperidin-1-yl)phenyl)-N⁶-(1-(methylsulfonyl)indolin-7-yl)-9H-purine-2,6-diamine

### Step 1: 2-chloro-N-(1-(methylsulfonyl)indolin-7-yl)-9H-purin-6-amine

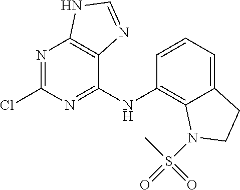 [SMILES: CS(=O)(=O)N1CCc2cccc(Nc3nc(Cl)nc4[nH]cnc34)c21]

To a solution of 1-(methylsulfonyl)indolin-7-amine (100 mg, 0.47 mmol) and 2,6-dichloro-9H-purine (133 mg, 0.71 mmol) in i-PrOH (10 mL) was added conc. HCl (0.4 mL). The resulting mixture was heated at 100° C. overnight. The organic solvent was removed under reduced pressure to afford the crude residue which was basified with saturated NaHCO₃(aq.) solution and extracted with DCM (2×50 mL). The combined organic layer was dried over Na₂SO₄ and concentrated under vacuum to afford the crude residue, which was purified with silica gel column chromatography (DCM: MeOH=100: 0˜ 10: 1 gradient elution) to give the title product (120 mg, 70%). [M+H]⁺=365.2.

### Step 2: N²-(2-methoxy-4-(4-(4-methylpiperazin-1-yl)piperidin-1-yl)phenyl)-N⁶-(1-(methylsulfonyl)indolin-7-yl)-9H-purine-2,6-diamine

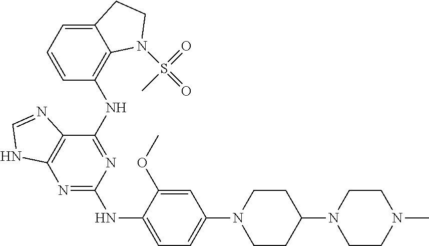 [SMILES: COc1cc(N2CCC(N3CCN(C)CC3)CC2)ccc1Nc1nc(Nc2cccc3c2N(S(C)(=O)=O)CC3)c2nc[nH]c2n1]

A mixture of 2-chloro-N-(1-(methylsulfonyl)indolin-7-yl)-9H-purin-6-amine (50 mg, 0.14 mmol), 2-methoxy-4-(4-(4-methylpiperazin-1-yl)piperidin-1-yl)aniline (49 mg, 0.16 mmol), G3 BrettPhos Pd (13 mg, 0.014 mmol) and Cs₂CO₃ (91 mg, 0.28 mmol) in dry DMF (5 mL) was stirred in a round bottom flask at 100° C. overnight under N₂. The mixture was evaporated in vacuum to afford the crude product, which was purified with silica gel column chromatography (DCM: MeOH=100: 0˜ 5: 1 gradient elution) to give crude product, which was further purified with pre-HPLC to give the title product (9 mg, 10%). ¹H NMR (400 MHz, DMSO) δ_{H} 12.57 (s, 1H), 9.13 (s, 1H), 8.23 (d, J=8.4 Hz, 1H), 7.87 (s, 1H), 7.80 (d, J=8.7 Hz, 1H), 7.43 (s, 1H), 7.21 (t, J=7.8 Hz, 1H), 7.11 (d, J=7.4 Hz, 1H), 6.62 (d, J=2.3 Hz, 1H), 6.45 (dd, J=8.8, 2.2 Hz, 1H), 4.07 (t, J=7.4 Hz, 2H), 3.80 (s, 3H), 3.67 (d, J=12.3 Hz, 2H), 3.12 (t, J=7.3 Hz, 2H), 3.05 (s, 3H), 2.63 (t, J=11.7 Hz, 2H), 2.51 (s, 4H), 2.43-2.25 (m, 5H), 2.17 (s, 3H), 1.85 (d, J=11.4 Hz, 2H), 1.59-1.46 (m, 2H); [M+H]⁺=633.5.

### Example 73: 5-chloro-N²-(2-methoxy-4-(4-(4-methylpiperazin-1-yl)piperidin-1-yl)phenyl)-N⁴-(1-(methylsulfonyl)indolin-7-yl)-7H-pyrrolo[2,3-d]pyrimidine-2,4-diamine

### Step 1: 2,4,5-trichloro-7-((2-(trimethylsilyl)ethoxy)methyl)-7H-pyrrolo[2,3-d]pyrimidine

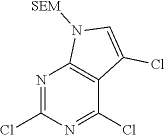 [SMILES: C[Si](C)(C)CCOCn1cc(Cl)c2c(Cl)nc(Cl)nc21]

To a stirred solution of 2,4,5-trichloro-7H-pyrrolo[2,3-d]pyrimidine (500 mg, 2.3 mmol) and NaH (136 mg, 3.4 mmol, 60% dispersion in mineral oil) in DMF(10 mL) was added SEM-C₁ (564 mg, 3.4 mmol) dropwise at 0° C. The resulting mixture was stirred at room temperature for 1 hour. The reaction was quenched with saturated NH₄Cl (aq.) solution and extracted with EtOAc (2×75 mL). The combined organic layer was washed with brine (2×50 mL), dried over Na₂SO₄ and concentrated under vacuum to afford the crude residue, which was purified with silica gel column chromatography (PE: EA=100: 0˜ 5: 1 gradient elution) to give the title product (780 mg, 98%). [M+H]⁺=352.2.

### Step 2: 2,5-dichloro-N-(1-(methylsulfonyl)indolin-7-yl)-7-((2-(trimethylsilyl)ethoxy)methyl)-7H-pyrrolo[2,3-d]pyrimidin-4-amine

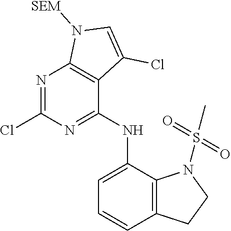 [SMILES: C[Si](C)(C)CCOCn1cc(Cl)c2c(Nc3cccc4c3N(S(C)(=O)=O)CC4)nc(Cl)nc21]

To a stirred solution of 1-(methylsulfonyl)indolin-7-amine (150 mg, 0.7 mmol) and 2,4,5-trichloro-7-((2-(trimethylsilyl)ethoxy)methyl)-7H-pyrrolo[2,3-d]pyrimidine (370 mg, 1.1 mmol) in dry DMF (8 mL) was added NaH (44 mg, 1.1 mmol, 60% dispersion in mineral oil) portionwise at 0° C. The resulting mixture was stirred at room temperature overnight. The reaction was quenched with saturated NH₄Cl (aq.) solution and extracted with EtOAc (2×50 mL). The combined organic layer was washed with brine (2×50 mL), dried over Na₂SO₄ and concentrated under vacuum to afford the crude residue, which was purified with silica gel column chromatography (PE: EA=100: 0˜ 2: 1 gradient elution) to give the title product (290 mg, 78%). [M+H]⁺=528.2.

### Step 3: 5-chloro-N²-(2-methoxy-4-(4-(4-methylpiperazin-1-yl)piperidin-1-yl)phenyl)-N⁴-(1-(methylsulfonyl)indolin-7-yl)-7-((2-(trimethylsilyl)ethoxy)methyl)-7H-pyrrolo[2,3-d]pyrimidine-2,4-diamine

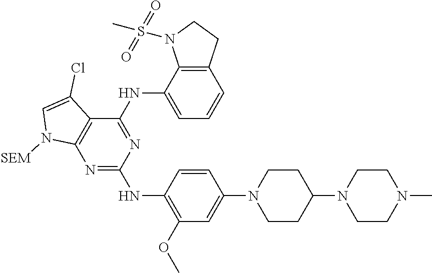 [SMILES: COc1cc(N2CCC(N3CCN(C)CC3)CC2)ccc1Nc1nc(Nc2cccc3c2N(S(C)(=O)=O)CC3)c2c(Cl)cn(COCC[Si](C)(C)C)c2n1]

A mixture of 2,5-dichloro-N-(1-(methylsulfonyl)indolin-7-yl)-7-((2-(trimethylsilyl)ethoxy)methyl)-7H-pyrrolo[2,3-d]pyrimidin-4-amine (80 mg, 0.15 mmol), 2-methoxy-4-(4-(4-methylpiperazin-1-yl)piperidin-1-yl)aniline (52 mg, 0.17 mmol), G3 BrettPhos Pd (13 mg, 0.015 mmol) and Cs₂CO₃ (98 mg, 0.30 mmol) in 1,4-dioxane (10 mL) was stirred in a round bottom flask at 100° C. overnight under N₂. The mixture was evaporated in vacuum to afford the crude product, which was purified with silica gel column chromatography (DCM: MeOH=100: 0˜ 10: 1 gradient elution) to give the title product (90 mg, 75%). [M+H]⁺=796.5.

### Step 4: 5-chloro-N²-(2-methoxy-4-(4-(4-methylpiperazin-1-yl)piperidin-1-yl)phenyl)—N⁴-(1-(methylsulfonyl)indolin-7-yl)-7H-pyrrolo[2,3-d]pyrimidine-2,4-diamine

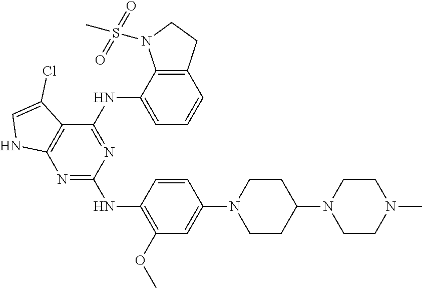 [SMILES: COc1cc(N2CCC(N3CCN(C)CC3)CC2)ccc1Nc1nc(Nc2cccc3c2N(S(C)(=O)=O)CC3)c2c(Cl)c[nH]c2n1]

To a stirred solution of 5-chloro-N²-(2-methoxy-4-(4-(4-methylpiperazin-1-yl)piperidin-1-yl)phenyl)—N⁴-(1-(methylsulfonyl)indolin-7-yl)-7-((2-(trimethylsilyl)ethoxy)methyl)-7H-pyrrolo[2,3-d]pyrimidine-2,4-diamine (90 mg, 0.11 mmol) in DCM (6 mL) was added TFA (3 mL) at room temperature. The resulting mixture was stirred at room temperature overnight. The reaction was concentrated under vacuum to afford the crude intermediate, which was dissolved in MeOH (5 mL). A solution of NH₃·H₂O (0.5 mL) was added and the resulting mixture was stirred at room temperature for 1 h. The solvent was removed under reduced pressure to afford the crude residue, which was purified with pre-HPLC to give the title product (48 mg, 64%). ¹H NMR (400 MHz, DMSO) δ_{H} 11.46 (s, 1H), 8.66 (s, 1H), 8.12 (s, 1H), 7.76 (s, 1H), 7.35 (s, 1H), 7.23-6.99 (m, 3H), 6.61 (s, 1H), 6.43 (s, 1H), 4.06 (s, 2H), 3.74 (d, J=37.4 Hz, 5H), 3.15-2.85 (m, 11H), 2.64 (s, 6H), 2.37-2.27 (m, 2H), 1.88 (s, 2H), 1.56 (s, 2H); [M+H]⁺=666.5.

### Example 74: N²-(2-methoxy-4-(4-(4-methylpiperazin-1-yl)piperidin-1-yl)phenyl)—N⁴-(1-(methylsulfonyl)indolin-7-yl)-6,7-dihydro-5H-cyclopenta[d]pyrimidine-2,4-diamine

### Step 1: 2-chloro-N-(1-(methylsulfonyl)indolin-7-yl)-6,7-dihydro-5H-cyclopenta[d]pyrimidin-4-amine

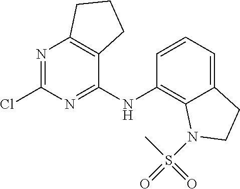 [SMILES: CS(=O)(=O)N1CCc2cccc(Nc3nc(Cl)nc4c3CCC4)c21]

To a solution of 1-(methylsulfonyl)indolin-7-amine (100 mg, 0.47 mmol) and 2,4-dichloro-6,7-dihydro-5H-cyclopenta[d]pyrimidine (132 mg, 0.70 mmol) in i-PrOH (8 mL) was added conc. HCl (0.4 mL). The resulting mixture was heated at 80° C. overnight. The organic solvent was removed under reduced pressure, the residue was basified with saturated NaHCO₃(aq.) solution and extracted with DCM (2×30 mL). The combined organic layer was dried over Na₂SO₄ and concentrated under vacuum to afford the crude residue, which was purified with silica gel column chromatography (PE: EA=100: 0˜ 1: 1 gradient elution) to give the title product (50 mg, 29%). [M+H]⁺=365.2.

### Step 2: N²-(2-methoxy-4-(4-(4-methylpiperazin-1-yl)piperidin-1-yl)phenyl)—N⁴-(1-(methylsulfonyl)indolin-7-yl)-6,7-dihydro-5H-cyclopenta[d]pyrimidine-2,4-diamine

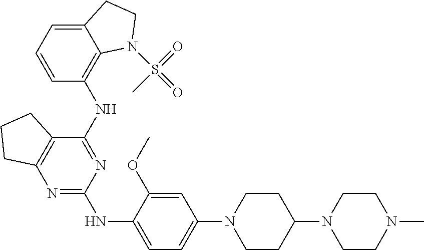 [SMILES: COc1cc(N2CCC(N3CCN(C)CC3)CC2)ccc1Nc1nc2c(c(Nc3cccc4c3N(S(C)(=O)=O)CC4)n1)CCC2]

A mixture of 2-chloro-N-(1-(methylsulfonyl)indolin-7-yl)-6,7-dihydro-5H-cyclopenta[d]pyrimidin-4-amine (50 mg, 0.14 mmol), 2-methoxy-4-(4-(4-methylpiperazin-1-yl)piperidin-1-yl)aniline (50 mg, 0.17 mmol), Pd₂(dba)₃ (13 mg, 0.014 mmol), BINAP (18 mg, 0.028 mmol) and K₃PO₄ (88 mg, 0.42 mmol) in toluene (8 mL) was stirred in a round bottom flask at 100° C. overnight under N₂. The mixture was evaporated in vacuum to afford the crude residue, which was purified with silica gel column chromatography (DCM: MeOH=100: 0-8:1 gradient elution) to give the product (17 mg, 20%). ¹H NMR (400 MHz, DMSO) δ_{H} 8.41 (s, 1H), 8.06 (d, J=7.5 Hz, 1H), 7.74 (d, J=8.4 Hz, 1H), 7.44 (s, 1H), 7.15 (d, J=7.8 Hz, 1H), 7.09 (s, 1H), 6.61 (s, 1H), 6.41 (d, J=8.6 Hz, 1H), 4.06 (s, 2H), 3.79 (s, 3H), 3.66 (d, J=10.8 Hz, 2H), 3.10 (s, 2H), 3.05 (s, 3H), 2.74-2.53 (m, 10H), 2.37 (d, J=36.5 Hz, 5H), 2.21 (s, 3H), 2.03 (s, 2H), 1.85 (d, J=11.0 Hz, 2H), 1.52 (d, J=11.5 Hz, 2H); [M+H]⁺=633.5.

### Example 75: N²-(2-methoxy-4-(4-(4-methylpiperazin-1-yl)piperidin-1-yl)phenyl)—N⁴-(1-(methylsulfonyl)indolin-7-yl)thieno[3,2-d]pyrimidine-2,4-diamine

### Step 1: 2-chloro-N-(1-(methylsulfonyl)indolin-7-yl)thieno[3,2-d]pyrimidin-4-amine

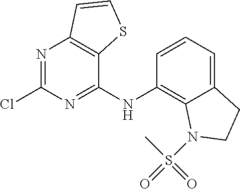 [SMILES: CS(=O)(=O)N1CCc2cccc(Nc3nc(Cl)nc4ccsc34)c21]

A mixture of 1-(methylsulfonyl)indolin-7-amine (50 mg, 0.23 mmol), 2,4-dichlorothieno[3,2-d]pyrimidine (70 mg, 0.35 mmol) and DIEA (60 mg, 0.46 mmol) in i-PrOH (8 mL) was stirred in a round bottom flask at 100° C. for 16 hours. The mixture was evaporated in vacuum to afford the crude product, which was purified with silica gel column chromatography (PE: EA=100: 0˜ 2: 1 gradient elution) to give the title product (55 mg, 61%). [M+H]⁺=381.1.

### Step 2: N²-(2-methoxy-4-(4-(4-methylpiperazin-1-yl)piperidin-1-yl)phenyl)—N⁴-(1-(methylsulfonyl)indolin-7-yl)thieno[32-d]pyrimidine-2,4-diamine

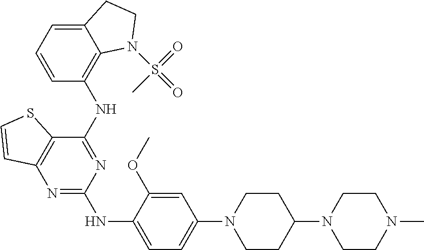 [SMILES: COc1cc(N2CCC(N3CCN(C)CC3)CC2)ccc1Nc1nc(Nc2cccc3c2N(S(C)(=O)=O)CC3)c2sccc2n1]

A mixture of 2-chloro-N-(1-(methylsulfonyl)indolin-7-yl)thieno[3,2-d]pyrimidin-4-amine (55 mg, 0.14 mmol), 2-methoxy-4-(4-(4-methylpiperazin-1-yl)piperidin-1-yl)aniline (50 mg, 0.17 mmol), Pd₂(dba)₃ (13 mg, 0.014 mmol), BINAP (18 mg, 0.028 mmol) and K₃PO₄ (88 mg, 0.42 mmol) in toluene (8 mL) was stirred in a round bottom flask at 100° C. overnight under N₂. The mixture was evaporated in vacuum to afford the crude residue, which was purified with silica gel column chromatography (DCM: MeOH=100: 0˜ 8: 1 gradient elution) to give an impure product, which was further purified with pre-HPLC to give the title product (30 mg, 32%). ¹H NMR (400 MHz, DMSO) δ_{H} 8.98 (s, 1H), 8.06 (s, 1H), 8.00 (d, J=7.7 Hz, 1H), 7.79 (d, J=8.5 Hz, 1H), 7.55 (s, 1H), 7.20 (d, J=12.1 Hz, 3H), 6.62 (s, 1H), 6.44 (d, J=8.3 Hz, 1H), 4.08 (s, 2H), 3.80 (s, 3H), 3.68 (d, J=10.5 Hz, 2H), 3.13 (s, 2H), 3.07 (s, 3H), 2.63 (t, J=11.8 Hz, 2H), 2.51 (s, 4H), 2.34 (s, 5H), 2.17 (s, 3H), 1.85 (d, J=10.6 Hz, 2H), 1.53 (d, J=11.2 Hz, 2H); [M+H]⁺=649.4.

### Example 76: N⁶-(4-(4-(4-ethylpiperazin-1-yl)piperidin-1-yl)-3-fluorophenyl)—N⁴-(1-(methylsulfonyl)indolin-7-yl)-1H-pyrazolo[3,4-d]pyrimidine-4,6-diamine

### Step 1: 4,6-dichloro-1-((2-(trimethylsilyl)ethoxy)methyl)-1H-pyrazolo[3,4-d]pyrimidine

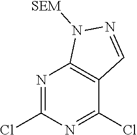 [SMILES: C[Si](C)(C)CCOCn1ncc2c(Cl)nc(Cl)nc21]

To a stirred solution of 4,6-dichloro-1H-pyrazolo[3,4-d]pyrimidine (800 mg, 4.2 mmol) and DIEA (1.1 g, 8.4 mmol) in DCM (20 mL) was added SEM-C₁ (1.1 g, 6.6 mmol) dropwise at 0° C.

The resulting mixture was stirred at room temperature for 1 hour. The reaction was quenched with water and extracted with DCM (2×75 mL). The combined organic layer was washed with brine (1×50 mL), dried over Na₂SO₄ and concentrated under vacuum to afford the crude residue, which was purified with silica gel column chromatography (PE: EA=100: 0˜ 10: 1 gradient elution) to give the title product (1.1 g, 81%). [M+H]⁺=319.2.

### Step 2: 6-chloro-N-(1-(methylsulfonyl)indolin-7-yl)-1-((2-(trimethylsilyl)ethoxy)methyl)-1H-pyrazolo[3,4-d]pyrimidin-4-amine

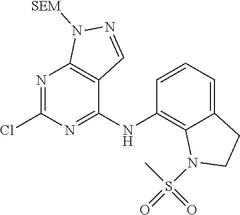 [SMILES: C[Si](C)(C)CCOCn1ncc2c(Nc3cccc4c3N(S(C)(=O)=O)CC4)nc(Cl)nc21]

A mixture of 1-(methylsulfonyl)indolin-7-amine (80 mg, 0.38 mmol), 4,6-dichloro-1-((2-(trimethylsilyl)ethoxy)methyl)-1H-pyrazolo[3,4-d]pyrimidine (179 mg, 0.56 mmol) and DIEA (97 mg, 0.76 mmol) in i-PrOH (10 mL) was stirred in a round bottom flask at 100° C. overnight. The mixture was evaporated in vacuum to afford the crude product, which was purified with silica gel column chromatography (PE: EA=100: 0˜ 2: 1 gradient elution) to give the title product (150 mg, 81%). [M+H]⁺=495.2.

### Step 3: N⁶-(4-(4-(4-ethylpiperazin-1-yl)piperidin-1-yl)-3-fluorophenyl)—N⁴-(1-(methylsulfonyl)indolin-7-yl)-1-((2-(trimethylsilyl)ethoxy)methyl)-1H-pyrazolo[3,4-d]pyrimidine-4,6-diamine

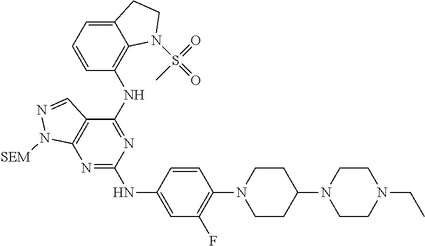 [SMILES: CCN1CCN(C2CCN(c3ccc(Nc4nc(Nc5cccc6c5N(S(C)(=O)=O)CC6)c5cnn(COCC[Si](C)(C)C)c5n4)cc3F)CC2)CC1]

A mixture of 6-chloro-N-(1-(methylsulfonyl)indolin-7-yl)-1-((2-(trimethylsilyl)ethoxy)methyl)-1H-pyrazolo[3,4-d]pyrimidin-4-amine (60 mg, 0.12 mmol), 4-(4-(4-ethylpiperazin-1-yl)piperidin-1-yl)-3-fluoroaniline (38 mg, 0.12 mmol) (The preparation of this intermediate was described in WO 2020060268 A1), G3 BrettPhos Pd (11 mg, 0.012 mmol) and Cs₂CO₃ (79 mg, 0.24 mmol) in 1,4-dioxane (8 mL) was stirred in a round bottom flask at 100° C. overnight under N₂. The mixture was evaporated in vacuum to afford the crude product, which was purified with silica gel column chromatography (DCM: MeOH=100: 0˜ 10: 1 gradient elution) to give the title product (70 mg, 75%). [M+H]⁺=765.5.

### Step 4: N⁶-(4-(4-(4-ethylpiperazin-1-yl)piperidin-1-yl)-3-fluorophenyl)—N⁴-(1-(methylsulfonyl)indolin-7-yl)-1H-pyrazolo[3,4-d]pyrimidine-4,6-diamine

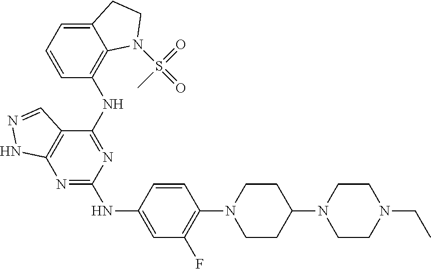 [SMILES: CCN1CCN(C2CCN(c3ccc(Nc4nc(Nc5cccc6c5N(S(C)(=O)=O)CC6)c5cn[nH]c5n4)cc3F)CC2)CC1]

To a stirred solution of N⁶-(4-(4-(4-ethylpiperazin-1-yl)piperidin-1-yl)-3-fluorophenyl)—N⁴-(1-(methylsulfonyl)indolin-7-yl)-1-((2-(trimethylsilyl)ethoxy)methyl)-1H-pyrazolo[3,4-d]pyrimidine-4,6-diamine (70 mg, 0.1 mmol) in DCM (5 mL) was added TFA (2 mL) at room temperature. The resulting mixture was stirred at room temperature for 2 h. The reaction was concentrated under vacuum to afford the intermediate, which was dissolved in MeOH (5 mL). Then a solution of NH₃·H₂O (0.5 mL) was added and the resulting mixture was stirred at room temperature for 1h. The solvent was removed under reduced pressure to afford the crude residue, which was purified with pre-HPLC to give the title product (35 mg, 60%). ¹H NMR (400 MHz, DMSO) δ_{H} 13.04 (s, 1H), 9.31 (s, 1H), 9.24 (s, 1H), 8.03 (s, 1H), 7.83 (d, J=14.3 Hz, 1H), 7.69 (s, 1H), 7.27 (dd, J=21.6, 13.8 Hz, 3H), 6.94 (d, J=9.3 Hz, 1H), 4.09 (s, 2H), 3.30 (d, J=9.5 Hz, 2H), 3.14 (s, 2H), 3.05 (s, 3H), 2.68-2.53 (m, 11H), 2.39 (s, 2H), 1.86 (d, J=11.3 Hz, 2H), 1.58 (d, J=11.2 Hz, 2H), 1.05 (s, 3H); [M+H]⁺=635.5.

### Example 77: N²-(4-(4-(4-ethylpiperazin-1-yl)piperidin-1-yl)-3-fluorophenyl)—N⁴-(1-(methylsulfonyl)indolin-7-yl)-7H-pyrrolo[2,3-d]pyrimidine-2,4-diamine

### Step 1: tert-butyl 4-(1-(2-fluoro-4-nitrophenyl)piperidin-4-yl)piperazine-1-carboxylate

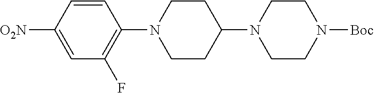 [SMILES: CC(C)(C)OC(=O)N1CCN(C2CCN(c3ccc([N+](=O)[O-])cc3F)CC2)CC1]

A mixture of 1,2-difluoro-4-nitrobenzene (500 mg, 3.1 mmol), tert-butyl 4-(piperidin-4-yl)piperazine-1-carboxylate (915 mg, 3.4 mmol) and K₂CO₃ (855 mg, 6.2 mmol) in DMF (15 mL) was stirred in a round bottom flask at 80° C. overnight. The reaction was cooled to room temperature, the mixture was poured into water (50 mL) and stirred for 10 mins. The solid was filtered and washed with water (30 mL×2), dried to give the product (750 mg, 58%). [M+H]⁺=409.4.

### Step 2: 1-(1-(2-fluoro-4-nitrophenyl)piperidin-4-yl)piperazine

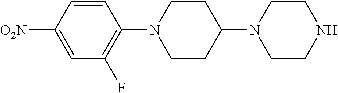 [SMILES: O=[N+]([O-])c1ccc(N2CCC(N3CCNCC3)CC2)c(F)c1]

A solution of tert-butyl 4-(1-(2-fluoro-4-nitrophenyl)piperidin-4-yl)piperazine-1-carboxylate (750 mg, 1.84 mmol) in HCl/1,4-dioxane (4M, 15 mL) was stirred in a round bottom flask at room temperature for 2 h. The mixture was evaporated in vacuum to afford the crude product (560 mg, 99%), which was used for next step without further purification. [M+H]⁺=309.4.

### Step 3: 1-ethyl-4-(1-(2-fluoro-4-nitrophenyl)piperidin-4-yl)piperazine

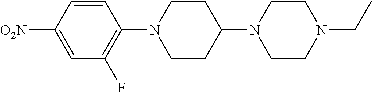 [SMILES: CCN1CCN(C2CCN(c3ccc([N+](=O)[O-])cc3F)CC2)CC1]

A mixture of 1-(1-(2-fluoro-4-nitrophenyl)piperidin-4-yl)piperazine (260 mg, 0.84 mmol), acetaldehyde (111 mg, 2.52 mmol) and NaOAc (207 mg, 2.52 mmol) in DCM (20 mL) and MeOH (4 mL) was stirred in a round bottom flask at room temperature for 1 hour. Then NaBH₃CN (156 mg, 2.52 mmol) was added and the mixture was stirred in a round bottom flask at room temperature for 2 h. The reaction was quenched with water (50 mL) and extracted with DCM (50 mL×2). The combined organic layers were dried over anhydrous Na₂SO₄, and evaporated in vacuum to afford the crude product, which was purified with silica gel column chromatography (DCM: MeOH=100: 0˜ 10: 1 gradient elution) to give the title product (220 mg, 78%). [M+H]⁺=337.4.

### Step 4: 4-(4-(4-ethylpiperazin-1-yl)piperidin-1-yl)-3-fluoroaniline

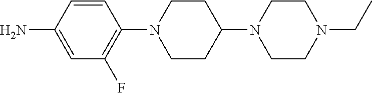 [SMILES: CCN1CCN(C2CCN(c3ccc(N)cc3F)CC2)CC1]

Under N₂, to a solution of 1-ethyl-4-(1-(2-fluoro-4-nitrophenyl)piperidin-4-yl)piperazine (220 mg, 0.65 mmol) in MeOH (20 mL) was added 10% Pd/C (50 mg) at 25° C. And then the mixture was exchanged with H₂ two times and stirred under H₂ atmosphere at 25° C. for 2 h. Reaction was monitored by LC-MS. The mixture was filtered through a pad of Celite and washed with MeOH (20 mL). The filtrate was concentrated under vacuum to obtain the product (190 mg, 95%). [M+H]⁺=307.4.

### Step 5: N²-(4-(4-(4-ethylpiperazin-1-yl)piperidin-1-yl)-3-fluorophenyl)—N⁴-(1-(methylsulfonyl)indolin-7-yl)-7H-pyrrolo[2,3-d]pyrimidine-2,4-diamine

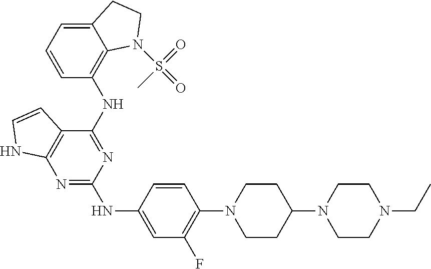 [SMILES: CCN1CCN(C2CCN(c3ccc(Nc4nc(Nc5cccc6c5N(S(C)(=O)=O)CC6)c5cc[nH]c5n4)cc3F)CC2)CC1]

A mixture of 2-chloro-N-(1-(methylsulfonyl)indolin-7-yl)-7H-pyrrolo[2,3-d]pyrimidin-4-amine (40 mg, 0.11 mmol), 4-(4-(4-ethylpiperazin-1-yl)piperidin-1-yl)-3-fluoroaniline (37 mg, 0.12 mmol), G3 BrettPhos Pd (10 mg, 0.011 mmol) and Cs₂CO₃ (72 mg, 0.22 mmol) in 1,4-dioxane (6 mL) was stirred in a round bottom flask at 100° C. overnight under N₂. The mixture was evaporated in vacuum to afford the crude product, which was purified with silica gel column chromatography (DCM: MeOH=100: 0-5: 1 gradient elution) to give the title product (10 mg, 14%). ¹H NMR (400 MHz, DMSO) δ_{H} 11.33 (s, 1H), 9.03 (s, 1H), 8.93 (s, 1H), 8.30 (d, J=8.0 Hz, 1H), 7.87 (d, J=15.4 Hz, 1H), 7.31 (d, J=7.7 Hz, 1H), 7.25 (s, 1H), 7.13 (d, J=6.7 Hz, 1H), 6.96 (s, 1H), 6.91 (s, 1H), 6.21 (s, 1H), 4.11 (s, 2H), 3.28 (d, J=10.0 Hz, 2H), 3.12 (d, J=15.6 Hz, 5H), 2.61 (d, J=11.0 Hz, 6H), 2.46-2.22 (m, 7H), 1.84 (d, J=12.1 Hz, 2H), 1.56 (d, J=10.8 Hz, 2H), 1.00 (d, J=7.1 Hz, 3H); [M+H]⁺=634.5.

### Example 78: N²-(4-(4-(4-ethylpiperazin-1-yl)piperidin-1-yl)-3,5-difluorophenyl)—N⁴-(1-(methylsulfonyl)indolin-7-yl)-7H-pyrrolo[2,3-d]pyrimidine-2,4-diamine

### Step 1: tert-butyl 4-(1-(2,6-difluoro-4-nitrophenyl)piperidin-4-yl)piperazine-1-carboxylate

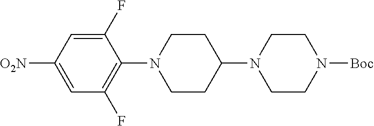 [SMILES: CC(C)(C)OC(=O)N1CCN(C2CCN(c3c(F)cc([N+](=O)[O-])cc3F)CC2)CC1]

The titled compound (1.2 g, 93%) was prepared in a manner similar to that in Example 77 step 1 from 1,2,3-trifluoro-5-nitrobenzene and tert-butyl 4-(piperidin-4-yl)piperazine-1-carboxylate. [M+H]⁺=427.2

### Step 2: 1-(1-(2,6-difluoro-4-nitrophenyl)piperidin-4-yl)piperazine

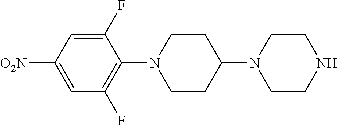 [SMILES: O=[N+]([O-])c1cc(F)c(N2CCC(N3CCNCC3)CC2)c(F)c1]

The titled compound (920 mg, 88%) was prepared in a manner similar to that in Example 77 step 2 from tert-butyl 4-(1-(2,6-difluoro-4-nitrophenyl)piperidin-4-yl)piperazine-1-carboxylate. [M+H]⁺=327.2

### Step 3: 1-(1-(2,6-difluoro-4-nitrophenyl)piperidin-4-yl)-4-ethylpiperazine

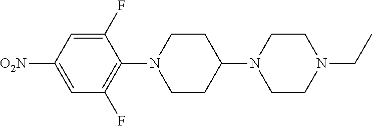 [SMILES: CCN1CCN(C2CCN(c3c(F)cc([N+](=O)[O-])cc3F)CC2)CC1]

The titled compound (210 mg, 72%) was prepared in a manner similar to that in Example 77 step 3 from 1-(1-(2,6-difluoro-4-nitrophenyl)piperidin-4-yl)piperazine. [M+H]⁺=355.2

### Step 4: 4-(4-(4-ethylpiperazin-1-yl)piperidin-1-yl)-3,5-difluoroaniline

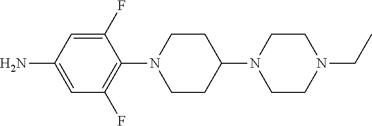 [SMILES: CCN1CCN(C2CCN(c3c(F)cc(N)cc3F)CC2)CC1]

The titled compound (170 mg, 81%) was prepared in a manner similar to that in Example 77 step 4 from 1-(1-(2,6-difluoro-4-nitrophenyl)piperidin-4-yl)-4-ethylpiperazine. [M+H]⁺=325.2

### Step 5: N²-(4-(4-(4-ethylpiperazin-1-yl)piperidin-1-yl)-3,5-difluorophenyl)—N⁴-(1-(methylsulfonyl)indolin-7-yl)-7H-pyrrolo[2,3-d]pyrimidine-2,4-diamine

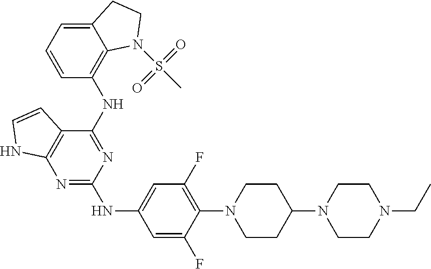 [SMILES: CCN1CCN(C2CCN(c3c(F)cc(Nc4nc(Nc5cccc6c5N(S(C)(=O)=O)CC6)c5cc[nH]c5n4)cc3F)CC2)CC1]

The titled compound was prepared in a manner similar to that in Example 77. ¹H NMR (400 MHz, DMSO) δ_{H} 11.41 (s, 1H), 9.17 (s, 1H), 9.08 (s, 1H), 8.20 (d, J=7.7, 1H), 7.52 (d, J=12.5, 2H), 7.25 (t, J=7.8, 1H), 7.14 (d, J=7.3, 1H), 7.01 (s, 1H), 6.23 (s, 1H), 4.11 (t, J=7.2, 2H), 3.18-3.12(m, 3H), 3.09 (s, 3H), 3.06 (s, 3H), 3.02-2.94 (m, 3H), 2.53 (s, 2H), 2.45-2.26 (m, 6H), 1.77 (d, J=11.3, 2H), 1.51 (dd, J=21.7, 10.8, 2H), 0.99 (t, J=7.1, 3H); [M+H]⁺=652.3.

### Example 79: N²-(3-fluoro-4-(4-(4-methylpiperazin-1-yl)piperidin-1-yl)phenyl)—N⁴-(1-(methylsulfonyl)indolin-7-yl)-7H-pyrrolo[2,3-d]pyrimidine-2,4-diamine

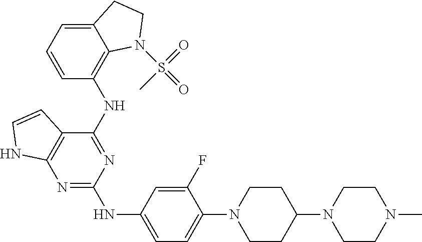 [SMILES: CN1CCN(C2CCN(c3ccc(Nc4nc(Nc5cccc6c5N(S(C)(=O)=O)CC6)c5cc[nH]c5n4)cc3F)CC2)CC1]

The titled compound was prepared in a manner similar to that in Example 77. ¹H NMR (400 MHz, DMSO) δ_{H} 11.33 (s, 1H), 9.03 (s, 1H), 8.93 (s, 1H), 8.30 (d, J=8.3 Hz, 1H), 7.87 (d, J=15.3 Hz, 1H), 7.31 (d, J=8.2 Hz, 1H), 7.25 (t, J=7.7 Hz, 1H), 7.12 (d, J=7.0 Hz, 1H), 6.97 (s, 1H), 6.91 (t, J=9.7 Hz, 1H), 6.21 (s, 1H), 4.11 (t, J=6.7 Hz, 2H), 3.29-3.26 (m, 4H), 3.16-3.07 (m, 7H), 2.60 (t, J=10.5 Hz, 6H), 2.34 (s, 1H), 2.27 (s, 3H), 1.85 (d, J=10.8 Hz, 2H), 1.57 (dd, J=21.7, 11.3 Hz, 2H); [M+H]⁺=620.3.

### Example 80: N²-(3-chloro-4-(4-(4-methylpiperazin-1-yl)piperidin-1-yl)phenyl)—N⁴-(1-(methylsulfonyl)indolin-7-yl)-7H-pyrrolo[2,3-d]pyrimidine-2,4-diamine

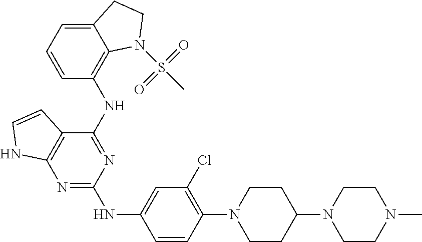 [SMILES: CN1CCN(C2CCN(c3ccc(Nc4nc(Nc5cccc6c5N(S(C)(=O)=O)CC6)c5cc[nH]c5n4)cc3Cl)CC2)CC1]

The titled compound was synthesized in the procedures similar to Example 77. ¹H NMR (400 MHz, CD₃OD) δ 8.31 (d, J=8.1 Hz, 1H), 7.94 (d, J=2.4 Hz, 1H), 7.37 (d, J=2.4 Hz, 1H), 7.25 (s, 1H), 7.09 (d, J=6.9 Hz, 1H), 6.98 (d, J=8.7 Hz, 1H), 6.90 (d, J=3.5 Hz, 1H), 6.35 (d, J=3.5 Hz, 1H), 4.16 (t, J=7.5 Hz, 2H), 3.15 (t, J=7.3 Hz, 2H), 3.08-2.70 (m, 12H), 2.67-2.51 (m, 7H), 1.97 (d, J=11.2 Hz, 2H), 1.75 (d, J=11.4 Hz, 2H); [M+H]⁺=636.26.

### Example 81: N²-(3-methoxy-4-(4-(4-methylpiperazin-1-yl)piperidin-1-yl)phenyl)—N⁴-(1-(methylsulfonyl)indolin-7-yl)-7H-pyrrolo[2,3-d]pyrimidine-2,4-diamine

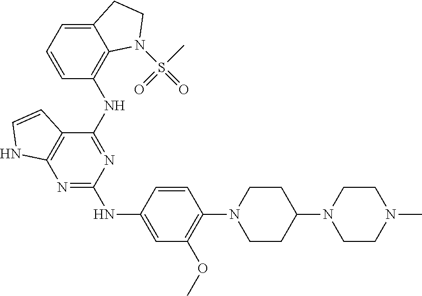 [SMILES: COc1cc(Nc2nc(Nc3cccc4c3N(S(C)(=O)=O)CC4)c3cc[nH]c3n2)ccc1N1CCC(N2CCN(C)CC2)CC1]

The titled compound was prepared in a manner similar to that in Example 77. ¹H NMR (400 MHz, DMSO) δ_{H} 11.25 (s, 1H), 8.97 (s, 1H), 8.68 (s, 1H), 8.38 (d, J=7.7, 1H), 7.45 (s, 1H), 7.33 (d, J=8.7, 1H), 7.22 (t, J=7.9, 1H), 7.10 (d, J=7.0, 1H), 6.94 (s, 1H), 6.76 (d, J=8.5, 1H), 6.19 (s, 1H), 4.10 (t, J=7.8, 2H), 3.73 (s, 3H), 3.26-3.22 (m, 5H), 3.13 (s, 2H), 3.09 (s, 3H), 2.60 (s, 4H), 2.45 (dd, J=6.6, 4.8, 3H), 2.33 (s, 1H), 2.26 (s, 3H), 1.81 (s, 2H), 1.55 (dd, J=21.2, 11.6, 2H); [M+H]⁺=632.4.

### Example 82: N²-(3-methyl-4-(4-(4-methylpiperazin-1-yl)piperidin-1-yl)phenyl)—N⁴-(1-(methylsulfonyl)indolin-7-yl)-7H-pyrrolo[2,3-d]pyrimidine-2,4-diamine

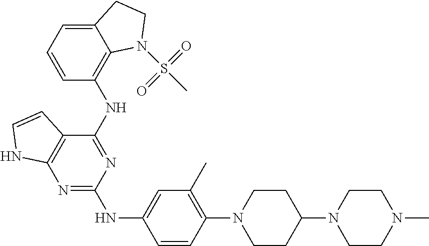 [SMILES: Cc1cc(Nc2nc(Nc3cccc4c3N(S(C)(=O)=O)CC4)c3cc[nH]c3n2)ccc1N1CCC(N2CCN(C)CC2)CC1]

The titled compound was prepared in a manner similar to that in Example 77. ¹H NMR (400 MHz, CD₃OD) δ 8.47 (s, 1H), 7.44 (s, 1H), 7.38 (d, J=8.5 Hz, 1H), 7.20 (t, J=7.8 Hz, 1H), 7.09 (d, J=7.2 Hz, 1H), 6.93 (d, J=8.6 Hz, 1H), 6.87 (d, J=3.5 Hz, 1H), 6.32 (d, J=3.5 Hz, 1H), 4.16 (t, J=7.4 Hz, 2H), 3.14-3.11 (m, 4H), 3.00-2.86 (m, 10H), 2.65 (t, J=11.1 Hz, 4H), 2.57 (s, 3H), 2.25 (s, 3H), 2.00 (d, J=11.6 Hz, 2H), 1.72 (q, J=11.4 Hz, 2H); [M+H]⁺=616.31.

### Example 83: N²-(4-(4-(4-ethylpiperazin-1-yl)piperidin-1-yl)-3-fluorophenyl)—N⁴-(4′-fluoro-1′-(methylsulfonyl)spiro[cyclobutane-1,3′-indolin]-7′-yl)-7H-pyrrolo[2,3-d]pyrimidine-2,4-diamine

### Step 1: 4′-fluoro-7′-nitrospiro[Cyclobutane-1,3′-indoline]

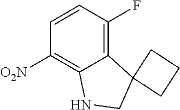 [SMILES: O=[N+]([O-])c1ccc(F)c2c1NCC21CCC1]

To a stirred solution of 4′-fluorospiro[cyclobutane-1,3′-indoline](400 mg, 2.26 mmol) in DCM (5 mL) was added conc. HNO₃ (0.5 ml) at 0° C. The resulting mixture was stirred at room temperature for 2 hours. The reaction was extracted with EtOAc. The organic lay was washed with saturated NaHCO₃(aq.) solution (2×50 mL) and brine (2×50 mL), dried over Na₂SO₄ and concentrated under vacuum to afford the crude residue, which was purified with silica gel column chromatography (PE: EA=100: 0˜ 2: 1 gradient elution) to give the title product (350 mg, 70%). [M+H]⁺=223.2.

### Step 2: 4‘-fluoro-1’-(methylsulfonyl)-7′-nitrospiro[cyclobutane-1,3′-indoline]

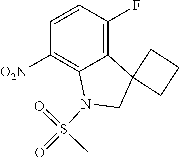 [SMILES: CS(=O)(=O)N1CC2(CCC2)c2c(F)ccc([N+](=O)[O-])c21]

The titled compound (310 mg, 78%) was prepared in a manner similar to that in Example 30 step 1 from 4′-fluoro-7′-nitrospiro[cyclobutane-1,3′-indoline] and methanesulfonyl chloride dropwise. [M+H]⁺=301.1.

### Step 3: 4‘-fluoro-1’-(methylsulfonyl)spiro[cyclobutane-1,3′-indolin]-7′-amine

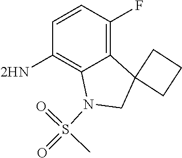

The titled compound (270 mg, 91%) was prepared in a manner similar to that in Example 30 step 2 from 4‘-fluoro-1’-(methylsulfonyl)-7′-nitrospiro[cyclobutane-1,3′-indoline] and Pd. [M+H]⁺=271.1.

### Step 4: N-(2-chloro-7H-pyrrolo[2,3-d]pyrimidin-4-yl)-4‘-fluoro-1’-(methylsulfonyl)spiro[Cyclobutane-1,3′-indolin]-7′-amine

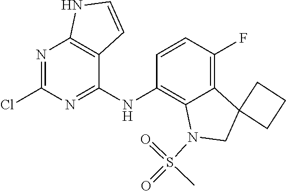 [SMILES: CS(=O)(=O)N1CC2(CCC2)c2c(F)ccc(Nc3nc(Cl)nc4[nH]ccc34)c21]

The titled compound (120 mg, 51%) was prepared in a manner similar to that in Example 71 step 1 from 4‘-fluoro-1’-(methylsulfonyl)spiro[cyclobutane-1,3′-indolin]-7′-amine and 2,4-dichloro-7H-pyrrolo[2,3-d]pyrimidine. [M+H]⁺=422.2.

### Step 5: N²-(4-(4-(4-ethylpiperazin-1-yl)piperidin-1-yl)-3-fluorophenyl)—N⁴-(4‘-fluoro-1’-(methylsulfonyl)spiro[cyclobutane-1,3′-indolin]-7′-yl)-7H-pyrrolo[2,3-d]pyrimidine-2,4-diamine

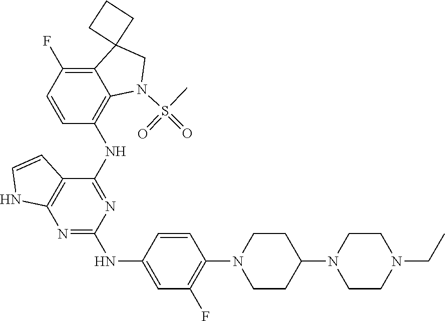 [SMILES: CCN1CCN(C2CCN(c3ccc(Nc4nc(Nc5ccc(F)c6c5N(S(C)(=O)=O)CC65CCC5)c5cc[nH]c5n4)cc3F)CC2)CC1]

The titled compound was prepared in a manner similar to that in Example 77. ¹H NMR (400 MHz, DMSO) δ_{H} 11.45 (s, 1H), 9.35 (s, 1H), 8.93 (s, 1H), 8.45 (d, J=8.5, 1H), 7.90 (d, J=16.1, 1H), 7.33 (d, J=7.8, 1H), 7.15 (t, J=8.2, 1H), 7.03 (s, 1H), 6.96 (t, J=9.4, 1H), 6.71 (s, 1H), 4.67 (s, 2H), 3.26-3.20 (m, 3H), 3.00 (s, 3H), 2.73-2.54 (m, 8H), 2.46-2.28 (m, 8H), 2.17-2.05 (m, 2H), 1.85 (d, J=11.3, 2H), 1.57 (dd, J=21.8, 12.1, 2H), 1.01 (t, J=7.1, 3H); [M+H]⁺*=692.3.

### Example 84: N₂-(3-fluoro-4-(4-methylpiperazin-1-yl)phenyl)—N₄-(1-(methylsulfonyl)indolin-7-yl)-7H-pyrrolo[2,3-d]pyrimidine-2,4-diamine

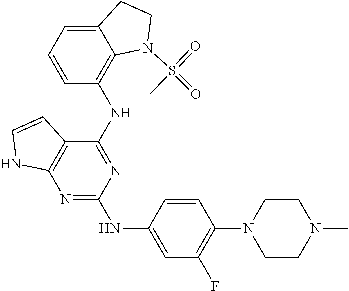 [SMILES: CN1CCN(c2ccc(Nc3nc(Nc4cccc5c4N(S(C)(=O)=O)CC5)c4cc[nH]c4n3)cc2F)CC1]

The titled compound (6.42 mg, 20%) was prepared in a manner similar to that in Example 77 step 3 from 2-chloro-N-(1-(methylsulfonyl)indolin-7-yl)-7H-pyrrolo[2,3-d]pyrimidin-4-amine and 3-fluoro-4-(4-methylpiperazin-1-yl)aniline. ¹H NMR (400 MHz, CD₃OD) 8.29 (d, J=8.1 Hz, 1H), =7.8, T), 715.4, 2.3 Hz, 1H), 7.3 (s, 1H), 6.95 (s, 1H), 6.90 (d, J=3.6 Hz, 1H), 630 (i, 3H), 4.17 (t, J=7.5 Hz, 2H), 3.25-3.07 (m, 10H), 2.98 (s, 3H), 2.70 (s, 3H); [M+H]=537.6.

### Example 85: N²-(2,3-difluoro-4-(4-methylpiperazin-1-yl)phenyl)—N⁴-(1-(methylsulfonyl)indolin-7-yl)-7H-pyrrolo[2,3-d]pyrimidine-2,4-diamine

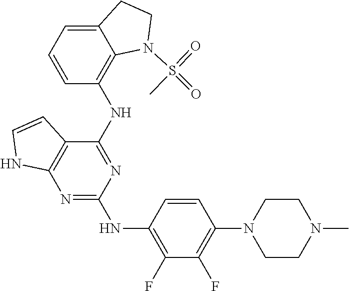 [SMILES: CN1CCN(c2ccc(Nc3nc(Nc4cccc5c4N(S(C)(=O)=O)CC5)c4cc[nH]c4n3)c(F)c2F)CC1]

The titled compound (4.28 mg, 22%) was prepared in a manner similar to that in Example 77 step 3 from 2-chloro-N-(1-(methylsulfonyl)indolin-7-yl)-7H-pyrrolo[2,3-d]pyrimidin-4-amine and 2,3-difluoro-4-(4-methylpiperazin-1-yl)aniline (This intermediate was prepared according to the way described in WO 2015027222 A2). ¹H NMR (400 MHz, CD₃OD) δ 8.14 (d, J=8.1 Hz, 1H), 7.81 (t, J=8.6 Hz, 1H), 7.18 (s, 1H), 7.10 (s, 1H), 6.90 (d, J=3.5 Hz, 1H), 6.77 (t, J=8.6 Hz, 1H), 6.33 (d, J=3.4 Hz, 1H), 4.16 (t, J=7.4 Hz, 2H), 3.22 (s, 4H), 3.15 (t, J=7.3 Hz, 2H), 3.03 (s, 4H), 2.98 (s, 3H), 2.65 (s, 3H); [M+H]⁺=555.6.

### Example 90: N²-(2-methoxy-4-(4-(4-methylpiperazin-1-yl)piperidin-1-yl)phenyl)—N⁴-(1-(methylsulfonyl)indolin-7-yl)-7H-pyrrolo[2,3-d]pyrimidine-2,4-diamine

### Step 1: 2-chloro-N-(1-(methylsulfonyl)indolin-7-yl)-7H-pyrrolo[2,3-d]pyrimidin-4-amine

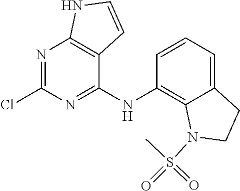 [SMILES: CS(=O)(=O)N1CCc2cccc(Nc3nc(Cl)nc4[nH]ccc34)c21]

To a solution of 1-(methylsulfonyl)indolin-7-amine (150 mg, 0.70 mmol) and 2,4-dichloro-7H-pyrrolo[2,3-d]pyrimidine (158 mg, 0.84 mmol) in i-PrOH (8 mL) was added conc. HCl (0.4 mL). The resulting mixture was heated at 100° C. overnight. The organic solvent was removed under reduced pressure, the residue was basified with saturated NaHCO₃(aq.) solution and extracted with DCM (2×30 mL). The combined organic layer was dried over Na₂SO₄ and concentrated under vacuum to afford the crude residue, which was purified with silica gel column chromatography (PE: EA=100: 0-1: 1 gradient elution) to give the title product (75 mg, 29%). [M+H]⁺=364.2.

### Step 2: N²-(2-methoxy-4-(4-(4-methylpiperazin-1-yl)piperidin-1-yl)phenyl)—N⁴-(1-(methylsulfonyl)indolin-7-yl)-7H-pyrrolo[2,3-d]pyrimidine-2,4-diamine

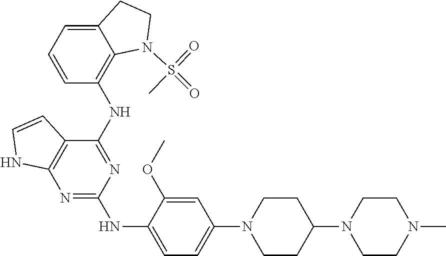 [SMILES: COc1cc(N2CCC(N3CCN(C)CC3)CC2)ccc1Nc1nc(Nc2cccc3c2N(S(C)(=O)=O)CC3)c2cc[nH]c2n1]

A mixture of 2-chloro-N-(1-(methylsulfonyl)indolin-7-yl)-7H-pyrrolo[2,3-d]pyrimidin-4-amine (40 mg, 0.11 mmol), 2-methoxy-4-(4-(4-methylpiperazin-1-yl)piperidin-1-yl)aniline (37 mg, 0.12 mmol), G3 BrettPhos Pd (10 mg, 0.011 mmol) and Cs₂CO₃ (72 mg, 0.22 mmol) in 1,4-dioxane (6 mL) was stirred in a round bottom flask at 100° C. overnight under N₂. The mixture was evaporated in vacuum to afford the crude product, which was purified with silica gel column chromatography (DCM: MeOH=100: 0˜ 5: 1 gradient elution) to give the title product (8 mg, 12%). ¹H NMR (400 MHz, DMSO) δ_{H} 11.25 (s, 1H), 8.99 (s, 1H), 8.22 (d, J=9.2 Hz, 1H), 7.97 (d, J=8.9 Hz, 1H), 7.22 (s, 2H), 7.10 (s, 1H), 6.92 (s, 1H), 6.62 (s, 1H), 6.45 (d, J=8.5 Hz, 1H), 6.19 (s, 1H), 4.09 (s, 2H), 3.82 (s, 3H), 3.66 (d, J=11.0 Hz, 2H), 3.11 (d, J=14.0 Hz, 5H), 2.66-2.52 (m, 6H), 2.34 (d, J=27.6 Hz, 5H), 2.18 (s, 3H), 1.85 (d, J=12.2 Hz, 2H), 1.53 (d, J=9.6 Hz, 2H); [M+H]⁺=632.4.

### Example 91: N²-(2-methoxy-5-methyl-4-(4-(4-methylpiperazin-1-yl)piperidin-1-yl)phenyl)—N⁴-(1-(methylsulfonyl)indolin-7-yl)-7H-pyrrolo[2,3-d]pyrimidine-2,4-diamine

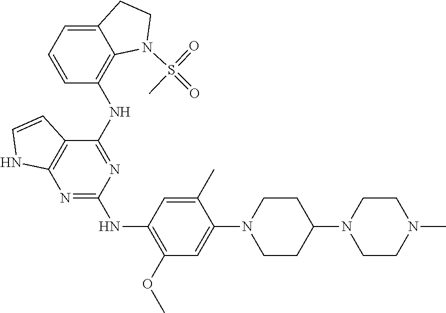 [SMILES: COc1cc(N2CCC(N3CCN(C)CC3)CC2)c(C)cc1Nc1nc(Nc2cccc3c2N(S(C)(=O)=O)CC3)c2cc[nH]c2n1]

The titled compound was prepared in a manner similar to that in Example 77. ¹H NMR (400 MHz, DMSO) δ_{H} 11.25 (s, 1H), 9.02 (s, 1H), 8.18 (s, 1H), 7.98 (s, 1H), 7.24 (s, 1H), 7.18 (d, J=7.6 Hz, 1H), 7.09 (d, J=3.3 Hz, 1H), 6.93 (s, 1H), 6.67 (d, J=4.1 Hz, 1H), 6.19 (s, 1H), 4.07 (s, 2H), 3.79 (d, J=4.0 Hz, 3H), 3.14-2.96 (m, 10H), 2.65-2.52 (m, 4H), 2.32 (s, 4H), 2.14 (dd, J=10.4, 4.0 Hz, 6H), 1.81 (s, 2H), 1.53 (d, J=21.9 Hz, 2H); [M+H]⁺=646.3.

### Example 92: N²-(2-isopropoxy-5-methyl-4-(4-(4-methylpiperazin-1-yl)piperidin-1-yl)phenyl)—N⁴-(1-(methylsulfonyl)indolin-7-yl)-7H-pyrrolo[2,3-d]pyrimidine-2,4-diamine

### Step 1: 1-fluoro-5-isopropoxy-2-methyl-4-nitrobenzene

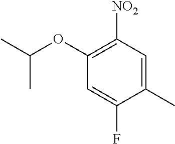

A mixture of 5-fluoro-4-methyl-2-nitrophenol (500 mg, 2.9 mmol), 2-iodopropane (1.0 g, 5.8 mmol) and K₂CO₃ (807 mg, 5.8 mmol) in DMF (10 mL) was stirred in a round bottom flask at room temperature overnight. The reaction was poured into water (50 mL) and extracted with EtOAc (2×75 mL). The combined organic layer was washed with brine, dried over Na₂SO₄ and concentrated under vacuum to afford the crude residue, which was purified with silica gel column chromatography (PE: EA=100: 0˜ 10: 1 gradient elution) to give the title product (410 mg, 66%). [M+H]⁺=214.2.

### Step 2: 1-(1-(5-isopropoxy-2-methyl-4-nitrophenyl)piperidin-4-yl)-4-methylpiperazine

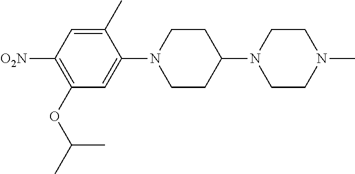 [SMILES: Cc1cc([N+](=O)[O-])c(OC(C)C)cc1N1CCC(N2CCN(C)CC2)CC1]

A mixture of 1-fluoro-5-isopropoxy-2-methyl-4-nitrobenzene (410 mg, 1.9 mmol), 1-methyl-4-(piperidin-4-yl)piperazine (420 mg, 2.3 mmol) and K₂CO₃ (528 mg, 3.8 mmol) in DMF (10 mL) was stirred in a round bottom flask at 100° C. overnight. The reaction was cooled to room temperature, the mixture was poured into water (50 mL) and stirred for 10 mins. The solid was filtered and washed with water (2×30 mL), dried to give the product (320 mg, 44%). [M+H]⁺=377.5.

### Step 3: 2-isopropoxy-5-methyl-4-(4-(4-methylpiperazin-1-yl)piperidin-1-yl)aniline

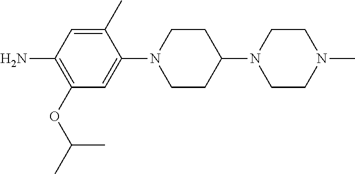 [SMILES: Cc1cc(N)c(OC(C)C)cc1N1CCC(N2CCN(C)CC2)CC1]

Under N₂, to a solution of 1-(1-(5-isopropoxy-2-methyl-4-nitrophenyl)piperidin-4-yl)-4-methylpiperazine (320 mg, 0.85 mmol) in MeOH (20 mL) was added 10% Pd/C (80 mg) at 25° C. And then the mixture was exchanged with H₂ two times and stirred under H₂ atmosphere at 25° C. for 2 h. The mixture was filtered through a pad of Celite and washed with MeOH (20 mL). The filtrate was concentrated under vacuum to obtain the product (260 mg, 88%). [M+H]⁺=347.5.

### Step 4: N²-(2-isopropoxy-5-methyl-4-(4-(4-methylpiperazin-1-yl)piperidin-1-yl)phenyl)-N⁴-(1-(methylsulfonyl)indolin-7-yl)-7H-pyrrolo[2,3-d]pyrimidine-2,4-diamine

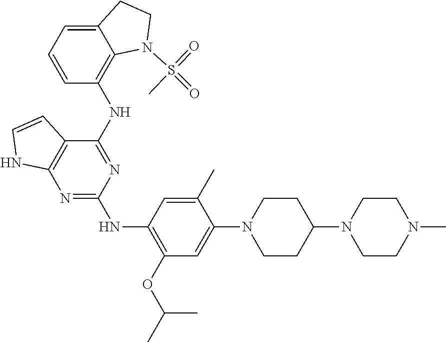 [SMILES: Cc1cc(Nc2nc(Nc3cccc4c3N(S(C)(=O)=O)CC4)c3cc[nH]c3n2)c(OC(C)C)cc1N1CCC(N2CCN(C)CC2)CC1]

The titled compound was synthesized in the method similar to Example 77. ¹H NMR (400 MHz, DMSO) δ_{H} 11.27 (s, 1H), 9.08 (s, 1H), 8.18 (d, J=8.2 Hz, 1H), 8.10 (s, 1H), 7.21 (s, 2H), 7.12 (d, J=7.3 Hz, 1H), 6.96 (s, 1H), 6.69 (s, 1H), 6.22 (s, 1H), 4.54 (s, 1H), 4.11 (s, 2H), 3.11 (d, J=17.6 Hz, 5H), 3.02 (d, J=10.4 Hz, 2H), 2.59 (d, J=11.2 Hz, 5H), 2.33 (s, 6H), 2.15 (d, J=6.4 Hz, 6H), 1.83 (d, J=11.7 Hz, 2H), 1.55 (d, J=9.7 Hz, 2H), 1.29 (d, J=5.8 Hz, 6H); [M+H]⁺=674.5.

### Example 93: N²-(2-ethoxy-5-methyl-4-(4-(4-methylpiperazin-1-yl)piperidin-1-yl)phenyl)-N⁴-(1-(methylsulfonyl)indolin-7-yl)-7H-pyrrolo[2,3-d]pyrimidine-2,4-diamine

### Step 1: 5-fluoro-4-methyl-2-nitrophenol

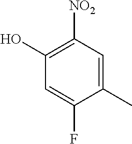

To a solution of 3-fluoro-4-methylphenol (1 g, 7.9 mmol) in DCM (25 mL) was added 65% HNO₃ (1 mL). The resulting mixture was stirred at room temperature for 1 h. The reaction was quenched with water (30 mL) and extracted with DCM (2×50 mL). The combined organic layer was dried over Na₂SO₄ and concentrated under vacuum to afford the crude residue, which was purified with silica gel column chromatography (PE: EA=100: 0˜ 10: 1 gradient elution) to give the title product (1.2 g, 88%). [M+H]⁺=172.2.

### Step 2: 1-ethoxy-5-fluoro-4-methyl-2-nitrobenzene

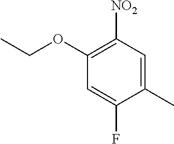

A mixture of 5-fluoro-4-methyl-2-nitrophenol (700 mg, 4.1 mmol), iodoethane (1.3 g, 8.2 mmol) and K₂CO₃ (1.2 g, 8.7 mmol) in DMF (15 mL) was stirred in a round bottom flask at room temperature overnight. The reaction was poured into water (50 mL) and extracted with EtOAc (2×75 mL). The combined organic layer was washed with brine, dried over Na₂SO₄ and concentrated under vacuum to afford the crude residue, which was purified with silica gel column chromatography (PE: EA=100: 0˜ 10: 1 gradient elution) to give the title product (620 mg, 76%). [M+H]⁺=200.2.

### Step 3: 1-(1-(5-ethoxy-2-methyl-4-nitrophenyl)piperidin-4-yl)-4-methylpiperazine

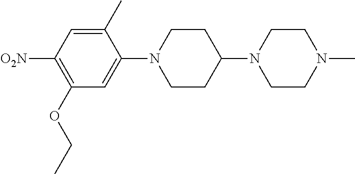 [SMILES: CCOc1cc(N2CCC(N3CCN(C)CC3)CC2)c(C)cc1[N+](=O)[O-]]

A mixture of 1-ethoxy-5-fluoro-4-methyl-2-nitrobenzene (300 mg, 1.5 mmol), 1-methyl-4-(piperidin-4-yl)piperazine (330 mg, 1.8 mmol) and K₂CO₃ (414 mg, 3.0 mmol) in DMF (10 mL) was stirred in a round bottom flask at 100° C. overnight. The reaction was cooled to room temperature, the mixture was poured into water (50 mL) and stirred for 10 mins. The solid was filtered and washed with water (2×30 mL), dried to give the product (260 mg, 48%). [M+H]⁺=363.5.

### Step 4: 2-ethoxy-5-methyl-4-(4-(4-methylpiperazin-1-yl)piperidin-1-yl)aniline

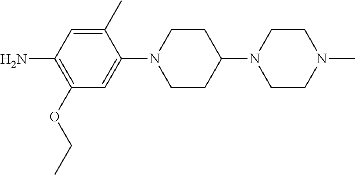 [SMILES: CCOc1cc(N2CCC(N3CCN(C)CC3)CC2)c(C)cc1N]

Under N₂, to a solution of 1-(1-(5-ethoxy-2-methyl-4-nitrophenyl)piperidin-4-yl)-4-methylpiperazine (260 mg, 0.72 mmol) in MeOH (20 mL) was added 10% Pd/C (80 mg) at 25° C. And then the mixture was exchanged with H₂ two times and stirred under H₂ atmosphere at 25° C. overnight. The mixture was filtered through a pad of Celite and washed with MeOH (20 mL). The filtrate was concentrated under vacuum to obtain the product (210 mg, 88%). [M+H]⁺=333.5.

### Step 5: N²-(2-ethoxy-5-methyl-4-(4-(4-methylpiperazin-1-yl)piperidin-1-yl)phenyl)—N⁴-(1-(methylsulfonyl)indolin-7-yl)-7H-pyrrolo[2,3-d]pyrimidine-2,4-diamine

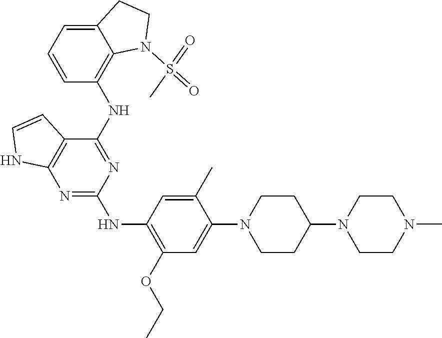 [SMILES: CCOc1cc(N2CCC(N3CCN(C)CC3)CC2)c(C)cc1Nc1nc(Nc2cccc3c2N(S(C)(=O)=O)CC3)c2cc[nH]c2n1]

The titled compound was synthesized in the method similar to Example 77. ¹H NMR (400 MHz, DMSO) δ_{H} 11.26 (s, 1H), 9.06 (s, 1H), 8.18 (d, J=7.0 Hz, 1H), 8.05 (s, 1H), 7.27-7.18 (m, 2H), 7.11 (d, J=7.3 Hz, 1H), 6.96 (s, 1H), 6.68 (s, 1H), 6.22 (s, 1H), 4.14-4.03 (m, 4H), 3.16-3.02 (m, 8H), 2.60 (d, J=10.7 Hz, 5H), 2.34 (s, 5H), 2.15 (d, J=10.4 Hz, 6H), 1.84 (d, J=11.6 Hz, 2H), 1.56 (d, J=11.1 Hz, 2H), 1.35 (t, J=6.9 Hz, 3H); [M+H]⁺=660.5.

### Example 94 N²-(5-ethyl-2-methoxy-4-(4-(4-methylpiperazin-1-yl)piperidin-1-yl)phenyl)-N⁴-(1-(methylsulfonyl)indolin-7-y)-7H-pyrrolo[2,3-d]pyrimidine-2,4-diamine

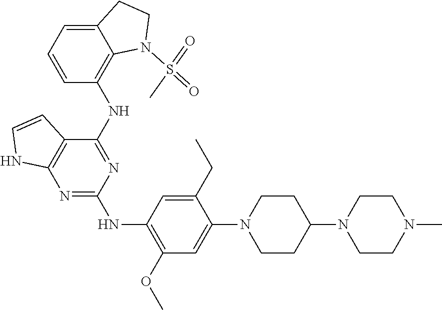 [SMILES: CCc1cc(Nc2nc(Nc3cccc4c3N(S(C)(=O)=O)CC4)c3cc[nH]c3n2)c(OC)cc1N1CCC(N2CCN(C)CC2)CC1]

The titled compound was prepared in a manner similar to that in Example 77. ¹H NMR (400 MHz, DMSO) δ_{H} 11.27 (s, 1H), 9.01 (s, 1H), 8.21 (s, 1H), 8.06 (s, 1H), 7.29 (s, 1H), 7.20 (t, J=7.7 Hz, 1H), 7.10 (d, J=7.1 Hz, 1H), 6.95 (s, 1H), 6.75 (s, 1H), 6.21 (s, 1H), 4.09 (t, J=7.0 Hz, 2H), 3.81 (s, 3H), 3.12 (dd, J=15.7, 9.6 Hz, 7H), 2.98 (d, J=11.3 Hz, 2H), 2.67 (t, J=11.2 Hz, 3H), 2.56 (d, J=7.6 Hz, 3H), 2.40-2.23 (m, 5H), 2.16 (s, 3H), 1.84 (d, J=12.1 Hz, 2H), 1.61-1.52 (m, 2H), 1.11 (t, J=7.4 Hz, 3H); [M+H]⁺=660.3.

### Example 95: N²-(5-chloro-2-methoxy-4-(4-(4-methylpiperazin-1-yl)piperidin-1-yl)phenyl)-N⁴-(1-(methylsulfonyl)indolin-7-yl)-7H-pyrrolo[2,3-d]pyrimidine-2,4-diamine

### Step 1: 1-(1-(2-chloro-5-methoxy-4-nitrophenyl)piperidin-4-yl)-4-methylpiperazine

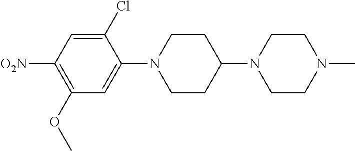 [SMILES: COc1cc(N2CCC(N3CCN(C)CC3)CC2)c(Cl)cc1[N+](=O)[O-]]

A mixture of 1-chloro-2-fluoro-4-methoxy-5-nitrobenzene (200 mg, 0.97 mmol), 1-methyl-4-(piperidin-4-yl)piperazine (196 mg, 1.07 mmol) and K₂CO₃ (267 mg, 1.94 mmol) in DMF (10 mL) was stirred in a round bottom flask at 100° C. overnight. The reaction was cooled to room temperature and poured into water (50 mL), extracted with EtOAc (2×50 mL). The combined organic layer was washed with brine, dried over Na₂SO₄ and concentrated under vacuum to afford the crude residue, which was purified with silica gel column chromatography (DCM: MeOH=100: 0˜10: 1 gradient elution) to give the title product (330 mg, 91%). [M+H]⁺=369.2.

### Step 2: 5-chloro-2-methoxy-4-(4-(4-methylpiperazin-1-yl)piperidin-1-yl)aniline

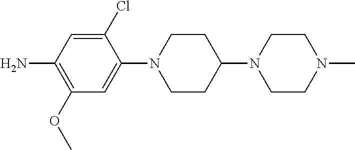 [SMILES: COc1cc(N2CCC(N3CCN(C)CC3)CC2)c(Cl)cc1N]

To a solution of 1-(1-(2-chloro-5-methoxy-4-nitrophenyl)piperidin-4-yl)-4-methylpiperazine (330 mg, 0.9 mmol) in EtOH (10 mL) and saturated NH₄Cl (aq.) solution (5 mL) was added Zn powder(580 mg, 9.0 mmol). The reaction was stirred at room temperature for 2 h. The mixture was filtered through a pad of Celite and washed with MeOH (20 mL). The filtrate was concentrated under vacuum to afford the crude residue, which was purified with silica gel column chromatography (DCM: MeOH=100: 0˜ 10: 1 gradient elution) to give the title product (280 mg, 92%). [M+H]⁺=339.2.

### Step 3: N²-(5-chloro-2-methoxy-4-(4-(4-methylpiperazin-1-yl)piperidin-1-yl)phenyl)—N⁴-(1-(methylsulfonyl)indolin-7-yl)-7H-pyrrolo[2,3-d]pyrimidine-2,4-diamine

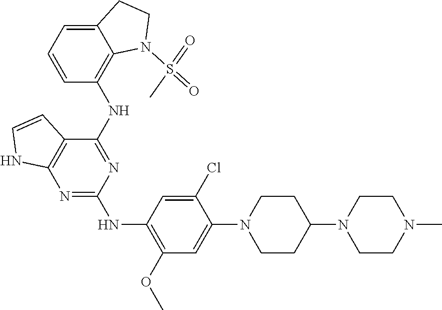 [SMILES: COc1cc(N2CCC(N3CCN(C)CC3)CC2)c(Cl)cc1Nc1nc(Nc2cccc3c2N(S(C)(=O)=O)CC3)c2cc[nH]c2n1]

The titled compound was synthesized in the method similar to Example 77. ¹H NMR (400 MHz, DMSO) δ_{H} 11.40 (s, 1H), 9.08 (s, 1H), 8.38 (s, 1H), 8.16 (d, J=8.0 Hz, 1H), 7.34 (s, 1H), 7.26 (t, J=7.8 Hz, 1H), 7.12 (d, J=7.3 Hz, 1H), 6.99 (s, 1H), 6.79 (s, 1H), 6.24 (s, 1H), 4.10 (s, 2H), 3.88 (s, 3H), 3.29-3.23 (m, 3H), 3.11 (d, J=16.0 Hz, 5H), 2.65 (d, J=10.5 Hz, 8H), 2.36 (d, J=35.2 Hz, 5H), 1.86 (s, 2H), 1.60 (d, J=9.8 Hz, 2H); [M+H]⁺=666.5.

### Example 96: N²-(5-chloro-2-methoxy-4-(9-methyl-3,9-diazaspiro[5.5]undecan-3-yl)phenyl)—N⁴-(1-(methylsulfonyl)indolin-7-yl)-7H-pyrrolo[2,3-d]pyrimidine-2,4-diamine

### Step 1: tert-butyl 9-(2-chloro-5-methoxy-4-nitrophenyl)-3,9-diazaspiro[5.5]undecane-3-carboxylate

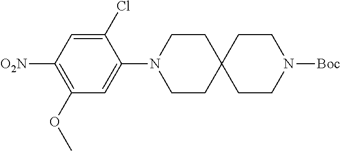 [SMILES: COc1cc(N2CCC3(CCN(C(=O)OC(C)(C)C)CC3)CC2)c(Cl)cc1[N+](=O)[O-]]

A mixture of 1-chloro-2-fluoro-4-methoxy-5-nitrobenzene (500 mg, 2.4 mmol), tert-butyl 3,9-diazaspiro[5.5]undecane-3-carboxylate (681 mg, 2.7 mmol) and K₂CO₃ (673 mg, 4.8 mmol) in DMF (15 mL) was stirred in a round bottom flask at 100° C. overnight. The reaction was cooled to room temperature, the mixture was poured into water (60 mL) and stirred for 10 mins. The solid was filtered and washed with water (30 mL×2), dried to give the product (950 mg, 89%). [M+H]⁺=440.2.

### Step 2: 3-(2-chloro-5-methoxy-4-nitrophenyl)-3,9-diazaspiro[5.5]undecane

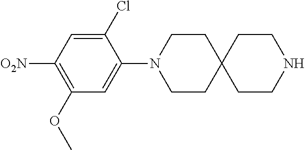 [SMILES: COc1cc(N2CCC3(CCNCC3)CC2)c(Cl)cc1[N+](=O)[O-]]

A solution of tert-butyl 9-(2-chloro-5-methoxy-4-nitrophenyl)-3,9-diazaspiro[5.5]undecane-3-carboxylate (950 mg, 2.2 mmol) in HCl/1,4-dioxane (4M, 15 mL) was stirred in a round bottom flask at room temperature for 2 h. The mixture was evaporated in vacuum to afford the crude product (730 mg, 99%), which was used for next step without further purification. [M+H]⁺=340.2.

### Step 3: 3-(2-chloro-5-methoxy-4-nitrophenyl)-9-methyl-3,9-diazaspiro[5.5]undecane

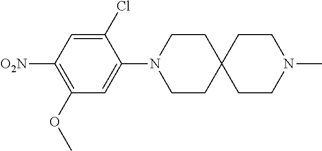 [SMILES: COc1cc(N2CCC3(CCN(C)CC3)CC2)c(Cl)cc1[N+](=O)[O-]]

A mixture of 3-(2-chloro-5-methoxy-4-nitrophenyl)-3,9-diazaspiro[5.5]undecane (712 mg, 2.1 mmol), (CH₂O)n (336 mg) and NaOAc (516 mg, 6.3 mmol) in DCM (25 mL) and MeOH (5 mL) was stirred in a round bottom flask at room temperature for 1 hour. Then NaBH₃CN (390 mg, 6.3 mmol) was added and the mixture was stirred in a round bottom flask at room temperature for 2 h. The reaction was quenched with water (50 mL) and extracted with DCM (75 mL×2). The combined organic layers were dried over anhydrous Na₂SO₄, and evaporated in vacuum to afford the crude product, which was purified with silica gel column chromatography (DCM: MeOH=100: 0˜ 10: 1 gradient elution) to give the title product (750 mg, 98%). [M+H]⁺=354.2.

### Step 4: 5-chloro-2-methoxy-4-(9-methyl-3,9-diazaspiro[5.5]undecan-3-yl)aniline

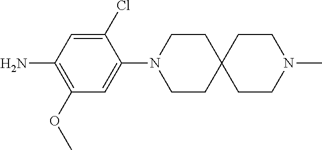 [SMILES: COc1cc(N2CCC3(CCN(C)CC3)CC2)c(Cl)cc1N]

To a solution of 3-(2-chloro-5-methoxy-4-nitrophenyl)-9-methyl-3,9-diazaspiro[5.5]undecane (680 mg, 1.9 mmol) in EtOH (20 mL) and saturated NH₄Cl (aq.) solution (8 mL) was added Zn powder (620 mg, 9.5 mmol). The reaction was stirred at room temperature for 2 h. The mixture was filtered through a pad of Celite and washed with MeOH (30 mL). The filtrate was concentrated under vacuum to afford the crude residue, which was purified with silica gel column chromatography (DCM: MeOH=100: 0˜10: 1 gradient elution) to give the title product (550 mg, 88%). [M+H]⁺=324.2.

### Step 5: N²-(5-chloro-2-methoxy-4-(9-methyl-3,9-diazaspiro[5.5]undecan-3-yl)phenyl)—N⁴-(1-(methylsulfonyl)indolin-7-yl)-7H-pyrrolo[2,3-d]pyrimidine-2,4-diamine

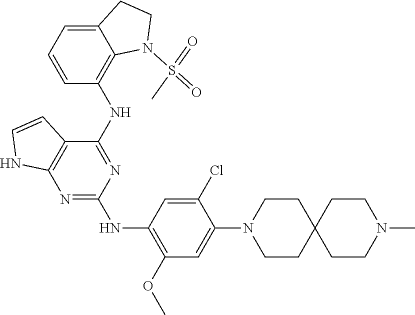 [SMILES: COc1cc(N2CCC3(CCN(C)CC3)CC2)c(Cl)cc1Nc1nc(Nc2cccc3c2N(S(C)(=O)=O)CC3)c2cc[nH]c2n1]

The titled compound was synthesized in the method similar to Example 77. ¹H NMR (400 MHz, DMSO) δ_{H} 11.39 (s, 1H), 9.08 (s, 1H), 8.36 (s, 1H), 8.16 (d, J=8.4 Hz, 1H), 7.33 (s, 1H), 7.26 (t, J=7.6 Hz, 1H), 7.12 (d, J=7.1 Hz, 1H), 6.98 (s, 1H), 6.85 (s, 1H), 6.23 (s, 1H), 4.10 (s, 2H), 3.88 (s, 3H), 3.11 (d, J=16.7 Hz, 5H), 2.91 (s, 4H), 2.37 (s, 4H), 2.22 (s, 3H), 1.55 (d, J=16.5 Hz, 8H); [M+H]⁺=651.5.

### Example 97: N²-(5-fluoro-2-methoxy-4-(6-methyl-2,6-diazaspiro[3.3]heptan-2-yl)phenyl)-N⁴-(1-(methylsulfonyl)indolin-7-yl)-7H-pyrrolo[2,3-d]pyrimidine-2,4-diamine

### Step 1: 2-(2-fluoro-5-methoxy-4-nitrophenyl)-6-methyl-2,6-diazaspiro[3.3]heptane

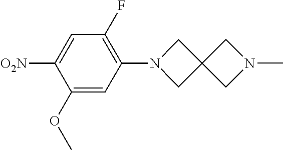 [SMILES: COc1cc(N2CC3(CN(C)C3)C2)c(F)cc1[N+](=O)[O-]]

The titled compound (190 mg, 63%) was prepared in a manner similar to that in Example 95 step 1 from 1,2-difluoro-4-methoxy-5-nitrobenzene and 2-methyl-2,6-diazaspiro[3.3]heptane. [M+H]⁺=282.1.

### Step 2: 5-fluoro-2-methoxy-4-(6-methyl-2,6-diazaspiro[3.3]heptan-2-yl)aniline

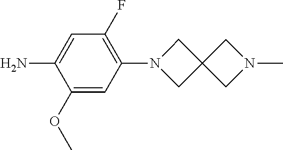 [SMILES: COc1cc(N2CC3(CN(C)C3)C2)c(F)cc1N]

The titled compound (165 mg, 85%) was prepared in a manner similar to that in Example 95 step 2 from 2-(2-fluoro-5-methoxy-4-nitrophenyl)-6-methyl-2,6-diazaspiro[3.3]heptane. [M+H]⁺=252.1.

### Step 3: N²-(5-fluoro-2-methoxy-4-(6-methyl-2,6-diazaspiro[3.3]heptan-2-yl)phenyl)—N⁴-(1-(methylsulfonyl)indolin-7-yl)-7H-pyrrolo[2,3-d]pyrimidine-2,4-diamine

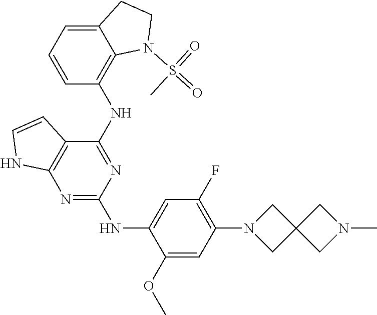 [SMILES: COc1cc(N2CC3(CN(C)C3)C2)c(F)cc1Nc1nc(Nc2cccc3c2N(S(C)(=O)=O)CC3)c2cc[nH]c2n1]

The titled compound was prepared in a manner similar to that in Example 77. ¹H NMR (400 MHz, DMSO) δ_{H} 11.31 (s, 1H), 9.05 (s, 1H), 8.14 (dd, J=16.6, 12.8 Hz, 2H), 7.22 (d, J=9.6 Hz, 2H), 7.13 (s, 1H), 6.96 (s, 1H), 6.22 (d, J=8.0 Hz, 2H), 4.18-4.07 (m, 5H), 4.03 (s, 4H), 3.84 (s, 3H), 3.15-3.07 (m, 6H), 2.73 (s, 3H); [M+H]⁺=579.2.

### Example 98: N²-(5-fluoro-2-methoxy-4-(4-(4-methylpiperazin-1-yl)piperidin-1-yl)phenyl)-N⁴-(1-(methylsulfonyl)indolin-7-yl)-7H-pyrrolo[2,3-d]pyrimidine-2,4-diamine

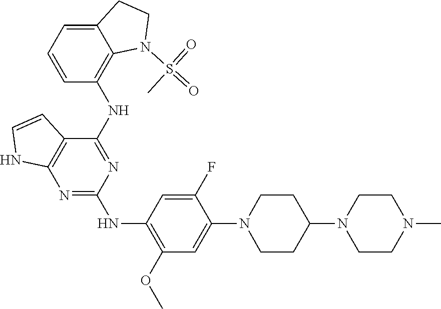 [SMILES: COc1cc(N2CCC(N3CCN(C)CC3)CC2)c(F)cc1Nc1nc(Nc2cccc3c2N(S(C)(=O)=O)CC3)c2cc[nH]c2n1]

The titled compound was prepared in a manner similar to that in Example 77. ¹H NMR (400 MHz, DMSO) δ_{H} 11.38 (s, 1H), 9.07 (s, 1H), 8.25 (s, 1H), 8.20 (d, J=10.0 Hz, 1H), 7.28 (s, 1H), 7.22 (t, J=7.8 Hz, 1H), 7.12 (d, J=7.3 Hz, 1H), 6.99-6.94 (m, 1H), 6.66 (d, J=8.3 Hz, 1H), 6.22-6.17 (m, 1H), 4.08 (t, J=7.4 Hz, 2H), 3.84 (s, 3H), 3.31-3.29 (m, 3H), 3.11 (t, J=7.4 Hz, 3H), 3.07 (s, 3H), 2.63 (t, J=11.1 Hz, 2H), 2.55-2.50 (m, 2H), 2.39-2.21 (m, 5H), 2.14 (s, 3H), 1.82 (d, J=11.2 Hz, 2H), 1.55 (dd, J=19.6, 11.3 Hz, 2H); [M+H]⁺=650.3.

### Example 99: N²-(2-methoxy-5-methyl-4-(1-methylpiperidin-4-yl)phenyl)—N⁴-(1-(methylsulfonyl)indolin-7-yl)-7H-pyrrolo[2,3-d]pyrimidine-2,4-diamine

### Step 1: 4-(5-methoxy-2-methyl-4-nitrophenyl)-1-methyl-1,2,3,6-tetrahydropyridine

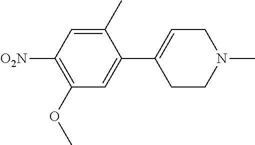 [SMILES: COc1cc(C2=CCN(C)CC2)c(C)cc1[N+](=O)[O-]]

Under N₂ atmosphere, a mixture of 1-bromo-5-methoxy-2-methyl-4-nitrobenzene (300 mg, 1.2 mmol), 1-methyl-4-(4,4,5,5-tetramethyl-1,3,2-dioxaborolan-2-yl)-1,2,3,6-tetrahydropyridine (326 mg, 1.5 mmol), Pd(PPh₃)₄ (140 mg, 0.12 mmol) and Cs₂CO₃ (596 mg, 1.8 mmol) in 1,4-dioxane (20 mL) and H₂O (4 mL) was stirred in a round bottom flask at 80° C. for 2 h. The mixture was evaporated in vacuum to afford the crude product, which was purified with silica gel column chromatography (DCM: MeOH=100: 0˜ 10: 1 gradient elution) to give the title product (315 mg, 98%). [M+H]⁺=263.2.

### Step 2: 2-methoxy-5-methyl-4-(1-methylpiperidin-4-yl)aniline

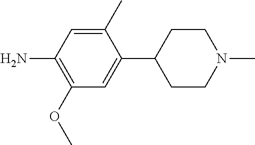 [SMILES: COc1cc(C2CCN(C)CC2)c(C)cc1N]

Under N₂, to a solution of 4-(5-methoxy-2-methyl-4-nitrophenyl)-1-methyl-1,2,3,6-tetrahydropyridine (315 mg, 1.2 mmol) in MeOH (20 mL) was added 10% Pd/C (80 mg) at 25° C. And then the mixture was exchanged with H₂ two times and stirred under H₂ atmosphere at 25° C. overnight. The mixture was filtered through a pad of Celite and washed with MeOH (20 mL). The filtrate was concentrated under vacuum to obtain the product (275 mg, 98%). [M+H]⁺=235.2.

### Step 3: N²-(2-methoxy-5-methyl-4-(1-methylpiperidin-4-yl)phenyl)—N⁴-(1-(methylsulfonyl)indolin-7-yl)-7H-pyrrolo[2,3-d]pyrimidine-2,4-diamine

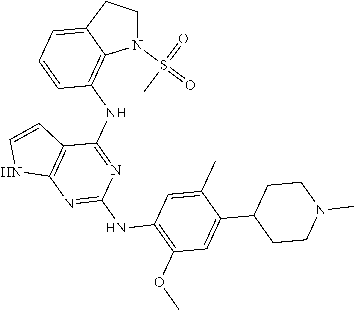 [SMILES: COc1cc(C2CCN(C)CC2)c(C)cc1Nc1nc(Nc2cccc3c2N(S(C)(=O)=O)CC3)c2cc[nH]c2n1]

The titled compound was synthesized in the method similar to Example 77. ¹H NMR (400 MHz, DMSO) δ_{H} 11.29 (s, 1H), 9.06 (s, 1H), 8.21 (s, 1H), 8.04 (s, 1H), 7.30 (s, 1H), 7.22 (s, 1H), 7.11 (s, 1H), 6.97 (s, 1H), 6.80 (s, 1H), 6.22 (s, 1H), 4.10 (s, 2H), 3.83 (s, 3H), 3.16-3.06 (m, 5H), 2.93 (s, 2H), 2.60 (s, 1H), 2.22 (d, J=21.2 Hz, 6H), 2.07 (s, 2H), 1.66 (s, 4H); [M+H]⁺ 30=562.5.

### Example 100 N²-(2-methoxy-5-methyl-4-(2-methyl-2,7-diazaspiro[3.5]nonan-7-yl)phenyl)-N⁴-(1-(methylsulfonyl)indolin-7-yl)-7H-pyrrolo[2,3-d]pyrimidine-2,4-diamine

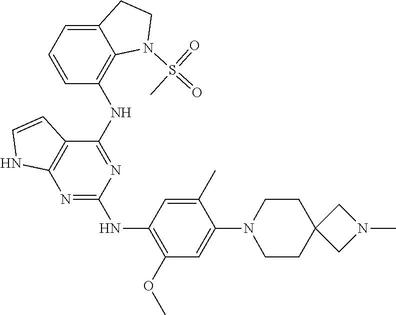 [SMILES: COc1cc(N2CCC3(CC2)CN(C)C3)c(C)cc1Nc1nc(Nc2cccc3c2N(S(C)(=O)=O)CC3)c2cc[nH]c2n1]

The titled compound was prepared in a manner similar to that in Example 77. ¹H NMR (400 MHz, DMSO) δ_{H} 11.24 (s, 1H), 9.01 (s, 1H), 8.17 (s, 1H), 7.98 (s, 1H), 7.19 (dd, J=15.3, 8.9 Hz, 2H), 7.07 (s, 1H), 6.92 (s, 1H), 6.64 (s, 1H), 6.18 (s, 1H), 4.06 (s, 2H), 3.78 (s, 3H), 3.12-3.03 (m, 8H), 2.68 (s, 5H), 2.42 (s, 3H), 2.11 (s, 3H), 1.80 (s, 4H); [M+H]⁺=603.3.

### Example 101: N²-(2-methoxy-5-methyl-4-(4-methyl-1,4-diazepan-1-yl)phenyl)—N⁴-(1-(methylsulfonyl)indolin-7-yl)-7H-pyrrolo[2,3-d]pyrimidine-2,4-diamine

### Step 1: 1-(5-methoxy-2-methyl-4-nitrophenyl)-4-methyl-1,4-diazepane

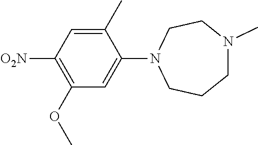 [SMILES: COc1cc(N2CCCN(C)CC2)c(C)cc1[N+](=O)[O-]]

The titled compound (230 mg, 60%) was prepared in a manner similar to that in Example 77 step 1 from 1-fluoro-5-methoxy-2-methyl-4-nitrobenzene and 1-methyl-1,4-diazepane. [M+H]⁺=280.2.

### Step 2: 2-methoxy-5-methyl-4-(4-methyl-1,4-diazepan-1-yl)aniline

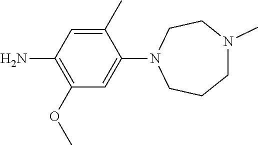 [SMILES: COc1cc(N2CCCN(C)CC2)c(C)cc1N]

The titled compound (180 mg, 88%) was prepared in a manner similar to that in Example 77 step 4 from 1-(5-methoxy-2-methyl-4-nitrophenyl)-4-methyl-1,4-diazepane. [M+H]±=250.2.

### Step 3: N²-(2-methoxy-5-methyl-4-(4-methyl-1,4-diazepan-1-yl)phenyl)—N₄-(1-(methylsulfonyl)indolin-7-yl)-7H-pyrrolo[2,3-d]pyrimidine-2,4-diamine

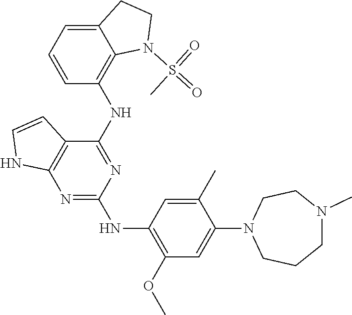 [SMILES: COc1cc(N2CCCN(C)CC2)c(C)cc1Nc1nc(Nc2cccc3c2N(S(C)(=O)=O)CC3)c2cc[nH]c2n1]

The titled compound (12.52 mg, 23%) was prepared in a manner similar to that in Example 77 step 5 from 2-chloro-N-(1-(methylsulfonyl)indolin-7-yl)-7H-pyrrolo[2,3-d]pyrimidin-4-amine and 2-methoxy-5-methyl-4-(4-methyl-1,4-diazepan-1-yl)aniline. ¹H NMR (400 MHz, CD₃OD) δ 8.48 (s, 1H), 8.19 (s, 1H), 7.24 (s, 1H), 7.11 (d, J=7.2 Hz, 1H), 6.89 (d, J=3.5 Hz, 1H), 6.77 (s, 1H), 6.33 (d, J=3.6 Hz, 1H), 4.18 (s, 2H), 3.89 (s, 3H), 3.60-3.50 (m, 2H), 3.43 (s, 2H), 3.37 (d, J=4.9 Hz, 3H), 3.20-3.11 (m, 4H), 2.99 (s, 3H), 2.97 (s, 3H), 2.23 (s, 3H), 2.21-2.13 (m, 2H); [M+H]⁺=577.7.

### Example 102: N²-(2-methoxy-5-methyl-4-((3aR,6aS)-5-methylhexahydropyrrolo[3,4-c]pyrrol-2(1H)-yl)phenyl)—N⁴-(1-(methylsulfonyl)indolin-7-yl)-7H-pyrrolo[2,3-d]pyrimidine-2,4-diamine

### Step 1: (3aR,6aS)-2-(5-methoxy-2-methyl-4-nitrophenyl)-5-methyloctahydropyrrolo[3,4-c]pyrrole

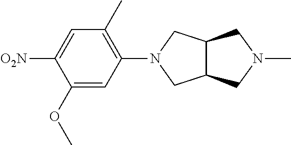 [SMILES: COc1cc(N2C[C@H]3CN(C)C[C@H]3C2)c(C)cc1[N+](=O)[O-]]

A mixture of 1-fluoro-5-methoxy-2-methyl-4-nitrobenzene (200 mg, 1.1 mmol), (3aR,6aS)-2-methyloctahydropyrrolo[3,4-c]pyrrole (150 mg, 1.2 mmol) and Cs₂CO₃ (704 mg, 2.2 mmol) in DMF (10 mL) was stirred in a round bottom flask at 100° C. overnight. The reaction was cooled to room temperature and poured into water (30 mL), extracted with EtOAc (2×30 mL). The combined organic layer was washed with brine, dried over Na₂SO₄ and concentrated under vacuum to afford the crude residue, which was purified with silica gel column chromatography (DCM: MeOH=100: 0˜ 10: 1 gradient elution) to give the title product (240 mg, 76%). [M+H]⁺=292.2.

### Step 2: 2-methoxy-5-methyl-4-((3aR,6aS)-5-methylhexahydropyrrolo[3,4-c]pyrrol-2(1H)-yl)aniline

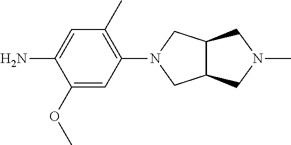 [SMILES: COc1cc(N2C[C@H]3CN(C)C[C@H]3C2)c(C)cc1N]

Under N₂, to a solution of (3aR,6aS)-2-(5-methoxy-2-methyl-4-nitrophenyl)-5-methyloctahydropyrrolo[3,4-c]pyrrole (240 mg, 0.82 mmol) in MeOH (20 mL) was added 10% Pd/C (50 mg) at 25° C. And then the mixture was exchanged with H₂ two times and stirred under H₂ atmosphere at 25° C. for 2 h. The mixture was filtered through a pad of Celite and washed with MeOH (20 mL). The filtrate was concentrated under vacuum to obtain the product (210 mg, 97%). [M+H]⁺=262.2.

### Step 3: N²-(2-methoxy-5-methyl-4-((3aR,6aS)-5-methylhexahydropyrrolo[3,4-c]pyrrol-2(1H)-yl)phenyl)—N⁴-(1-(methylsulfonyl)indolin-7-yl)-7H-pyrrolo[2,3-d]pyrimidine-2,4-diamine

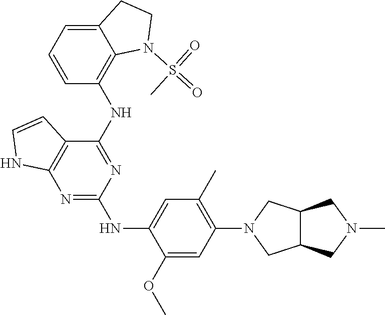 [SMILES: COc1cc(N2C[C@H]3CN(C)C[C@H]3C2)c(C)cc1Nc1nc(Nc2cccc3c2N(S(C)(=O)=O)CC3)c2cc[nH]c2n1]

The titled compound was synthesized in the method similar to Example 77. ¹H NMR (400 MHz, DMSO) δ_{H} 11.25 (s, 1H), 9.03 (s, 1H), 8.20 (s, 1H), 7.95 (s, 1H), 7.25 (s, 1H), 7.20 (d, J=8.0 Hz, 1H), 7.10 (d, J=7.3 Hz, 1H), 6.94 (s, 1H), 6.65 (s, 1H), 6.21 (s, 1H), 4.10 (t, J=7.4 Hz, 2H), 3.81 (s, 3H), 3.15-3.08 (m, 5H), 3.02 (s, 2H), 2.77 (dd, J=26.1, 8.7 Hz, 6H), 2.31 (d, J=5.5 Hz, 2H), 2.27 (s, 3H), 2.17 (s, 3H); [M+H]⁺=589.5.

### Example 103: N²-(4-(4-(dimethylamino)piperidin-1-yl)-2-methoxy-5-methylphenyl)—N⁴-(1-(methylsulfonyl)indolin-7-yl)-7H-pyrrolo[2,3-d]pyrimidine-2,4-diamine

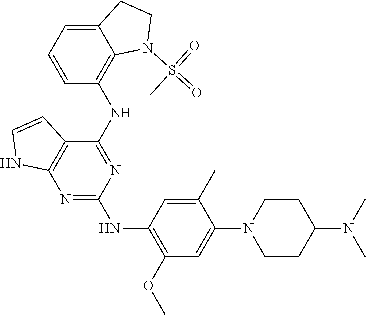 [SMILES: COc1cc(N2CCC(N(C)C)CC2)c(C)cc1Nc1nc(Nc2cccc3c2N(S(C)(=O)=O)CC3)c2cc[nH]c2n1]

The titled compound was prepared in a manner similar to that in Example 77. ¹H NMR (400 MHz, DMSO) δ_{H} 11.26 (s, 1H), 9.02 (s, 1H), 8.26-8.14 (m, 1H), 7.98 (s, 1H), 7.24 (s, 1H), 7.22-7.16 (m, 1H), 7.12-7.05 (m, 1H), 6.93 (s, 1H), 6.67 (s, 1H), 6.19 (s, 1H), 4.08 (s, 2H), 3.79 (s, 3H), 3.12-3.02 (m, 7H), 2.59 (s, 2H), 2.25 (s, 7H), 2.13 (s, 3H), 1.83 (s, 2H), 1.55 (t, J=17.9 Hz, 2H); [M+H]⁺=591.3.

### Example 104: N²-(4-(4-(dimethylamino)cyclohexyl)-2-methoxy-5-methylphenyl)—N₄-(1-(methylsulfonyl)indolin-7-yl)-7H-pyrrolo[2,3-d]pyrimidine-2,4-diamine

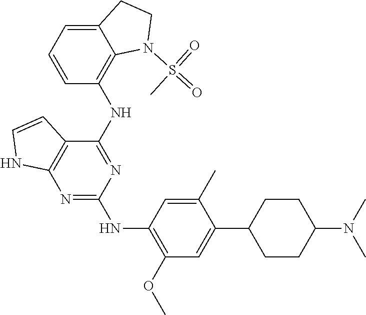 [SMILES: COc1cc(C2CCC(N(C)C)CC2)c(C)cc1Nc1nc(Nc2cccc3c2N(S(C)(=O)=O)CC3)c2cc[nH]c2n1]

The titled compound (10.32 mg, 18%) was prepared in a manner similar to that in Example 77 step 5 from 2-chloro-N-(1-(methylsulfonyl)indolin-7-yl)-7H-pyrrolo[2,3-d]pyrimidin-4-amine and 4-(4-(dimethylamino)cyclohexyl)-2-methoxy-5-methylaniline (This intermediate was prepared according to the described method in WO 2008073687 A2). ¹H NMR (400 MHz, CD₃OD) δ 8.19 (d, J=7.9 Hz, 1H), 8.15 (s, 1H), 7.23 (t, J=7.8 Hz, 1H), 7.12 (d, J=6.9 Hz, 1H), 6.89 (d, J=3.5 Hz, 1H), 6.78 (s, 1H), 6.33 (d, J=3.5 Hz, 1H), 4.18 (s, 2H), 3.88 (s, 3H), 3.17 (s, 2H), 2.99 (s, 3H), 2.80 (s, 7H), 2.22 (s, 3H), 2.17 (s, 3H), 1.99 (s, 2H), 1.66 (d, J=7.1 Hz, 4H); [M+H]⁺=590.7.

### Example 105: (S)—N²-(4-(3,4-dimethylpiperazin-1-yl)-2-methoxy-5-methylphenyl)—N⁴-(1-(methylsulfonyl)indolin-7-yl)-7H-pyrrolo[2,3-d]pyrimidine-2,4-diamine

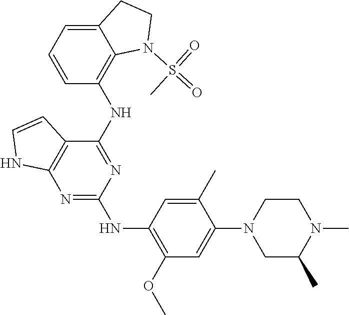 [SMILES: COc1cc(N2CCN(C)[C@@H](C)C2)c(C)cc1Nc1nc(Nc2cccc3c2N(S(C)(=O)=O)CC3)c2cc[nH]c2n1]

The titled compound (8.36 mg, 19%) was prepared in a manner similar to that in Example 106 step 4 from 2-chloro-N-(1-(methylsulfonyl)indolin-7-yl)-7H-pyrrolo[2,3-d]pyrimidin-4-amine and (S)-4-(3,4-dimethylpiperazin-1-yl)-2-methoxy-5-methylaniline. H NMR (400 MHz, CD₃OD) δ 8.49 (s, 1H), 8.19 (d, J=4.6 Hz, 1H), 7.26-7.19 (m, 1H), 7.12 (d, J=7.4 Hz, 1H), 6.89 (d, J=3.6 Hz, 1H), 6.73 (s, 1H), 6.33 (d, J=3.6 Hz, 1H), 4.18 (t, J=7.6 Hz, 2H), 3.90 (s, 3H), 3.13-3.09 (m, 6H), 2.99 (s, 3H), 2.71 (d, J=18.2 Hz, 4H), 2.20 (s, 3H), 2.03 (s, 2H), 1.32 (d, J=6.4 Hz, 3H); [M+H]⁺=577.3.

### Example 106: N²-(2-methoxy-5-methyl-4-((3S,5R)-3,4,5-trimethylpiperazin-1-yl)phenyl)-N⁴-(1-(methylsulfonyl)indolin-7-yl)-7H-pyrrolo[2,3-d]pyrimidine-2,4-diamine

### Step 1: (3S,5R)-1-(5-methoxy-2-methyl-4-nitrophenyl)-3,5-dimethylpiperazine

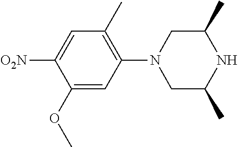 [SMILES: COc1cc(N2C[C@@H](C)N[C@@H](C)C2)c(C)cc1[N+](=O)[O-]]

A mixture of 1-fluoro-5-methoxy-2-methyl-4-nitrobenzene (500 mg, 2.7 mmol), (2S,6R)-2,6-dimethylpiperazine (370 mg, 3.2 mmol) and K₂CO₃ (746 mg, 5.4 mmol) in DMF (15 mL) was stirred in a round bottom flask at 60° C. overnight. The reaction was cooled to room temperature and poured into water (60 mL), extracted with EtOAc (2×75 mL). The combined organic layer was washed with brine, dried over Na₂SO₄ and concentrated under vacuum to afford the crude residue, which was purified with silica gel column chromatography (DCM: MeOH=100: 0˜ 10: 1 gradient elution) to give the title product (750 mg, 99%). [M+H]⁺=280.2.

### Step 2: (2S,6R)-4-(5-methoxy-2-methyl-4-nitrophenyl)-1,2,6-trimethylpiperazine

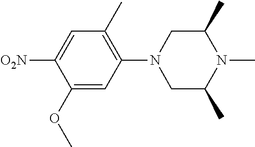 [SMILES: COc1cc(N2C[C@@H](C)N(C)[C@@H](C)C2)c(C)cc1[N+](=O)[O-]]

A mixture of (3S,5R)-1-(5-methoxy-2-methyl-4-nitrophenyl)-3,5-dimethylpiperazine (680 mg, 2.4 mmol), (CH₂O)n (450 mg) and AcOH(144 mg, 2.4 mmol) in DCM (20 mL) and MeOH (4 mL) was stirred in a round bottom flask at room temperature for 1 hour. Then NaBH₃CN (450 mg, 7.2 mmol) was added and the mixture was stirred in a round bottom flask at room temperature for 2 h. The reaction was quenched with water (75 mL) and extracted with DCM (100 mL×2). The combined organic layers were dried over anhydrous Na₂SO₄, and evaporated in vacuum to afford the crude product, which was purified with silica gel column chromatography (DCM: MeOH=100: 0˜ 10: 1 gradient elution) to give the title product (580 mg, 81%). [M+H]⁺=294.2.

### Step 3: 2-methoxy-5-methyl-4-((3S,5R)-3,4,5-trimethylpiperazin-1-yl)aniline

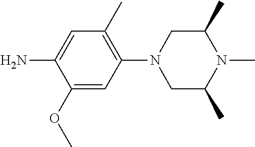 [SMILES: COc1cc(N2C[C@@H](C)N(C)[C@@H](C)C2)c(C)cc1N]

Under N₂, to a solution of (2S,6R)-4-(5-methoxy-2-methyl-4-nitrophenyl)-1,2,6-trimethylpiperazine (580 mg, 2.0 mmol) in MeOH (30 mL) was added 10% Pd/C (100 mg) at 25° C. And then the mixture was exchanged with H₂ two times and stirred under H₂ atmosphere at 25° C. overnight. The mixture was filtered through a pad of Celite and washed with MeOH (20 mL). The filtrate was concentrated under vacuum to obtain the product (515 mg, 99%). [M+H]⁺=264.4.

### Step 4: N²-(2-methoxy-5-methyl-4-((3S,5R)-3,4,5-trimethylpiperazin-1-yl)phenyl)—N⁴-(1-(methylsulfonyl)indolin-7-yl)-7H-pyrrolo[2,3-d]pyrimidine-2,4-diamine

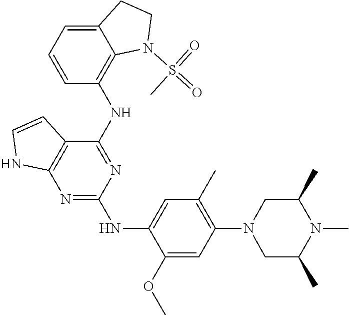 [SMILES: COc1cc(N2C[C@@H](C)N(C)[C@@H](C)C2)c(C)cc1Nc1nc(Nc2cccc3c2N(S(C)(=O)=O)CC3)c2cc[nH]c2n1]

The titled compound was synthesized in the method similar to Example 77 step 5. ¹H NMR (400 MHz, DMSO) δ_{H} 11.28 (s, 1H), 9.05 (s, 1H), 8.18 (s, 1H), 8.03 (s, 1H), 7.23 (dd, J=17.1, 9.4 Hz, 2H), 7.12 (s, 1H), 6.95 (s, 1H), 6.67 (s, 1H), 6.22 (s, 1H), 4.10 (s, 2H), 3.83 (s, 3H), 3.11 (d, J=15.1 Hz, 5H), 2.86 (d, J=10.5 Hz, 2H), 2.45 (d, J=11.1 Hz, 2H), 2.33 (s, 2H), 2.23 (s, 3H), 2.16 (s, 3H), 1.05 (d, J=5.6 Hz, 6H); [M+H]⁺=591.5.

### Example 107: (R)—N₂-(4-(3,4-dimethylpiperazin-1-yl)-2-methoxy-5-methyl phenyl)—N₄-(1-(methylsulfonyl)indolin-7-yl)-7H-pyrrolo[2,3-d]pyrimidine-2,4-diamine

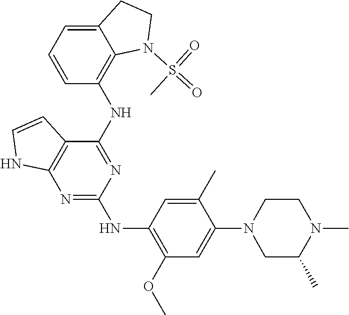 [SMILES: COc1cc(N2CCN(C)[C@H](C)C2)c(C)cc1Nc1nc(Nc2cccc3c2N(S(C)(=O)=O)CC3)c2cc[nH]c2n1]

The titled compound was synthesized in the procedures similar to Example 77. ¹H NMR (400 MHz, CD₃OD) δ 8.17 (s, 1H), 8.14 (s, 1H), 7.21 (t, J=7.8 Hz, 1H), 7.09 (d, J=6.9 Hz, 1H), 6.88 (d, J=3.3 Hz, 1H), 6.69 (s, 1H), 6.32 (d, J=3.2 Hz, 1H), 4.15 (t, J=7.3 Hz, 2H), 3.87 (s, 3H), 3.31 (s, 2H), 3.17-3.01 (m, 7H), 2.97 (s, 3H), 2.77 (d, J=12.6 Hz, 1H), 2.73 (s, 3H), 2.17 (s, 3H), 1.32 (d, J=5.6 Hz, 3H); [M+H]⁺=577.3.

### Example 108: 2-(4-(5-methoxy-2-methyl-4-((4-((1-(methylsulfonyl)indolin-7-yl)amino)-7H-pyrrolo[2,3-d]pyrimidin-2-yl)amino)phenyl)piperidin-1-yl)ethan-1-ol

### Step 1: tert-butyl 4-(5-methoxy-2-methyl-4-nitrophenyl)-3,6-dihydropyridine-1(2H)-carboxylate

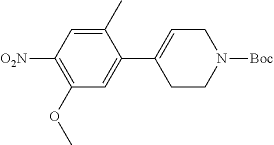 [SMILES: COc1cc(C2=CCN(C(=O)OC(C)(C)C)CC2)c(C)cc1[N+](=O)[O-]]

A mixture of 1-bromo-5-methoxy-2-methyl-4-nitrobenzene (500 mg, 2.0 mmol), tert-butyl 4-(4,4,5,5-tetramethyl-1,3,2-dioxaborolan-2-yl)-3,6-dihydropyridine-1(2H)-carboxylate (753 mg, 2.4 mmol), Pd(PPh₃)₄ (234 mg, 0.2 mmol) and Cs₂CO₃ (993 mg, 3.0 mmol) in 1,4-dioxane (30 mL) and H₂O (6 mL) was stirred in a round bottom flask at 80° C. for 2 h under N₂. The mixture was evaporated in vacuum to afford the crude product, which was purified with silica gel column chromatography (PE: EA=100: 0˜ 4: 1 gradient elution) to give the title product (700 mg, 98%). [M+H]⁺=349.2.

### Step 2: tert-butyl 4-(4-amino-5-methoxy-2-methylphenyl)piperidine-1-carboxylate

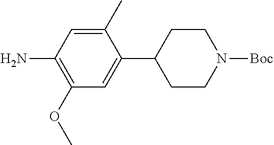 [SMILES: COc1cc(C2CCN(C(=O)OC(C)(C)C)CC2)c(C)cc1N]

Under N₂, to a solution of tert-butyl 4-(5-methoxy-2-methyl-4-nitrophenyl)-3,6-dihydropyridine-1(2H)-carboxylate (450 mg, 1.3 mmol) in MeOH (20 mL) was added 10% Pd/C (100 mg) at 25° C. And then the mixture was exchanged with H₂ two times and stirred under H₂ atmosphere at 25° C. overnight. The mixture was filtered through a pad of Celite and washed with MeOH (20 mL). The filtrate was concentrated under vacuum to obtain the product (320 mg, 77%). [M+H]⁺=321.4.

### Step 3: tert-butyl 4-(5-methoxy-2-methyl-4-((4-((1-(methylsulfonyl)indolin-7-yl)amino)-7H-pyrrolo[2,3-d]pyrimidin-2-yl)amino)phenyl)piperidine-1-carboxylate

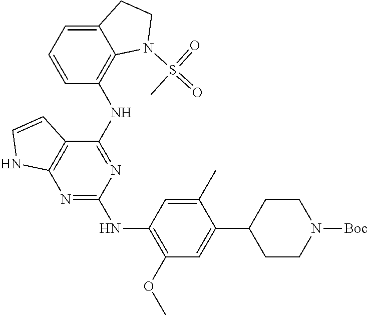 [SMILES: COc1cc(C2CCN(C(=O)OC(C)(C)C)CC2)c(C)cc1Nc1nc(Nc2cccc3c2N(S(C)(=O)=O)CC3)c2cc[nH]c2n1]

A mixture of 2-chloro-N-(1-(methylsulfonyl)indolin-7-yl)-7H-pyrrolo[2,3-d]pyrimidin-4-amine (100 mg, 0.27 mmol), tert-butyl 4-(4-amino-5-methoxy-2-methylphenyl)piperidine-1-carboxylate (97 mg, 0.30 mmol), G3 BrettPhos Pd (30 mg, 0.03 mmol) and Cs₂CO₃ (180 mg, 0.54 mmol) in 1,4-dioxane (10 mL) was stirred in a round bottom flask at 100° C. overnight under N₂. The mixture was evaporated in vacuum to afford the crude product, which was purified with silica gel column chromatography (PE: EA=100: 0˜ 1: 2 gradient elution) to give the title product (80 mg, 45%). [M+H]⁺=648.5.

### Step 4: N²-(2-methoxy-5-methyl-4-(piperidin-4-yl)phenyl)—N⁴-(1-(methylsulfonyl)indolin-7-yl)-7H-pyrrolo[2,3-d]pyrimidine-2,4-diamine

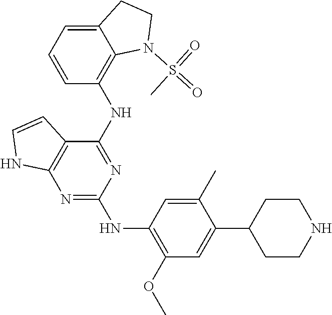 [SMILES: COc1cc(C2CCNCC2)c(C)cc1Nc1nc(Nc2cccc3c2N(S(C)(=O)=O)CC3)c2cc[nH]c2n1]

A solution of tert-butyl 4-(5-methoxy-2-methyl-4-((4-((1-(methylsulfonyl)indolin-7-yl)amino)-7H-pyrrolo[2,3-d]pyrimidin-2-yl)amino)phenyl)piperidine-1-carboxylate (80 mg, 0.12 mmol) in HCl/1,4-dioxane (4M, 10 mL) was stirred in a round bottom flask at room temperature for 2 h. The mixture was evaporated in vacuum to afford the crude product (65 mg, 96%), which was used for next step without further purification. [M+H]⁺=548.5.

### Step 5: 2-(4-(5-methoxy-2-methyl-4-((4-((1-(methylsulfonyl)indolin-7-yl)amino)-7H-pyrrolo[2,3-d]pyrimidin-2-yl)amino)phenyl)piperidin-1-yl)ethan-1-ol

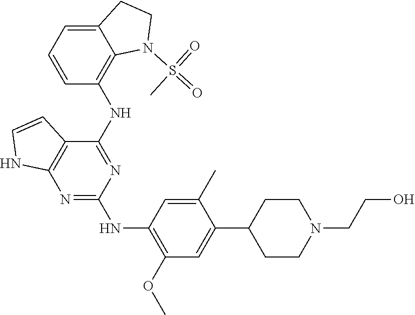 [SMILES: COc1cc(C2CCN(CCO)CC2)c(C)cc1Nc1nc(Nc2cccc3c2N(S(C)(=O)=O)CC3)c2cc[nH]c2n1]

A mixture of N²-(2-methoxy-5-methyl-4-(piperidin-4-yl)phenyl)—N⁴-(1-(methylsulfonyl)indolin-7-yl)-7H-pyrrolo[2,3-d]pyrimidine-2,4-diamine (30 mg, 0.05 mmol), 2-bromoethan-1-ol (25 mg, 0.2 mmol) and K₂CO₃ (14 mg, 0.1 mmol) in DMF (2 mL) was stirred in a round bottom flask at room temperature overnight. The inorganic salt was removed by filtration. The filtrate was purified with pre-HPLC to give the title product (7 mg, 22%). ¹H NMR (400 MHz, DMSO) δ_{H} 11.28 (s, 1H), 9.06 (s, 1H), 8.20 (d, J=8.7 Hz, 1H), 8.03 (s, 1H), 7.29 (s, 1H), 7.22 (t, J=7.7 Hz, 1H), 7.11 (d, J=7.1 Hz, 1H), 6.96 (s, 1H), 6.81 (s, 1H), 6.23 (s, 1H), 4.10 (s, 2H), 3.84 (s, 3H), 3.54 (s, 3H), 3.11 (d, J=16.5 Hz, 5H), 3.03 (d, J=10.7 Hz, 2H), 2.64 (s, 1H), 2.48-2.45 (m, 2H), 2.25-2.09 (m, 5H), 1.79-1.60 (m, 4H); [M+H]⁺=592.5.

### Example 109: N²-(4-(1-(2-(dimethylamino)ethyl)piperidin-4-yl)-2-methoxy-5-methylphenyl)—N⁴-(1-(methylsulfonyl)indolin-7-yl)-7H-pyrrolo[2,3-d]pyrimidine-2,4-diamine

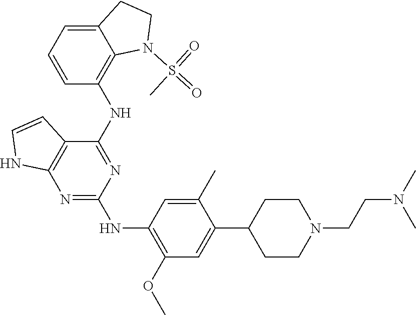 [SMILES: COc1cc(C2CCN(CCN(C)C)CC2)c(C)cc1Nc1nc(Nc2cccc3c2N(S(C)(=O)=O)CC3)c2cc[nH]c2n1]

To a solution of N²-(2-methoxy-5-methyl-4-(piperidin-4-yl)phenyl)—N⁴-(1-(methylsulfonyl)indolin-7-yl)-7H-pyrrolo[2,3-d]pyrimidine-2,4-diamine (40 mg, 0.07 mmol), 2-(dimethylamino)acetaldehyde hydrochloride (26 mg, 0.21 mmol) and AcOH (9 mg, 0.15 mmol) in DCM(2 mL)/MeOH (5 mL) was added DIEA (45 mg, 0.35 mmol). After stirring at room temperature for 2 minutes, NaBH₃CN (22 mg, 0.35 mmol) was added and the resulting mixture was stirred at room temperature for 2 hours. The reaction was quenched with water and extracted with DCM (2×30 mL). The combined organic layers were washed once with brine, dried over Na₂SO₄, filtered and concentrated in vacuum to afford the crude residue, which was further purified with pre-HPLC to give the product (5.5 mg, 12%). ¹H NMR (400 MHz, DMSO) δ_{H} 11.28 (s, 1H), 9.06 (s, 1H), 8.18 (s, 1H), 8.05 (s, 1H), 7.29 (s, 1H), 7.22 (s, 1H), 7.11 (d, J=7.1 Hz, 1H), 6.97 (s, 1H), 6.80 (s, 1H), 6.23 (s, 1H), 4.10 (s, 2H), 3.84 (s, 3H), 3.13 (s, 2H), 3.07 (d, J=13.6 Hz, 5H), 2.68 (s, 2H), 2.55 (s, 3H), 2.30 (s, 6H), 2.18 (d, J=18.7 Hz, 5H), 1.77-1.63 (m, 4H); [M+H]⁺=619.5.

### Example 110: N²-(2-methoxy-4-((1S,4S)-5-methyl-2,5-diazabicyclo[2.2.1]heptan-2-yl)phenyl)—N⁴-(1-(methylsulfonyl)indolin-7-yl)-7H-pyrrolo[2,3-d]pyrimidine-2,4-diamine

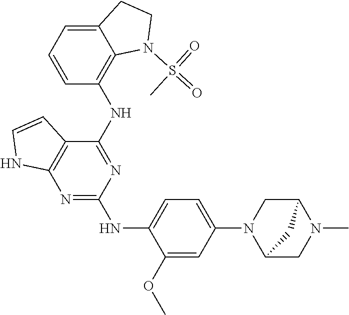 [SMILES: COc1cc(N2C[C@@H]3C[C@H]2CN3C)ccc1Nc1nc(Nc2cccc3c2N(S(C)(=O)=O)CC3)c2cc[nH]c2n1]

The titled compound (3.12 mg, 15%) was prepared in a manner similar to that in Example 77 step 3 from 2-chloro-N-(1-(methylsulfonyl)indolin-7-yl)-7H-pyrrolo[2,3-d]pyrimidin-4-amine and 2-methoxy-4-((1S,4S)-5-methyl-2,5-diazabicyclo[2.2.1]heptan-2-yl)aniline. ¹H NMR (400 MHz, CD₃OD) δ 8.17 (d, J=7.7 Hz, 1H), 8.08 (d, J=8.6 Hz, 1H), 7.23 (t, J=7.8 Hz, 1H), 7.12 (d, J=7.1 Hz, 1H), 6.86 (s, 1H), 6.35 (s, 1H), 6.29 (d, J=3.4 Hz, 1H), 6.20 (d, J=8.6 Hz, 1H), 4.63 (s, 2H), 4.33 (s, 1H), 4.17 (t, J=7.4 Hz, 2H), 3.90 (s, 3H), 3.73-3.70 (m, 2H), 3.40 (d, J=10.8 Hz, 1H), 3.16 (t, J=7.1 Hz, 2H), 2.98 (s, 3H), 2.93 (s, 3H), 2.34 (s, 2H); [M+H]⁺=561.23.

### Example 111: N²-(2-methoxy-4-((1R,5S)-3-methyl-3,8-diazabicyclo[3.2.1]octan-8-yl)phenyl)—N⁴-(1-(methylsulfonyl)indolin-7-yl)-7H-pyrrolo[2,3-d]pyrimidine-2,4-diamine

### Step 1: (1R,5S)-8-(3-methoxy-4-nitrophenyl)-3-methyl-3,8-diazabicyclo[3.2.1]octane

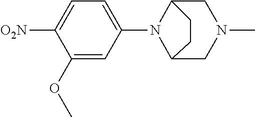 [SMILES: COc1cc(N2C3CCC2CN(C)C3)ccc1[N+](=O)[O-]]

The titled compound (300 mg, 86%) was prepared in a manner similar to that in Example 95 step 1 from 4-fluoro-2-methoxy-1-nitrobenzene and (1R,5S)-3-methyl-3,8-diazabicyclo[3.2.1]octane. [M+H]⁺=278.2.

### Step 2: 2-methoxy-4-((1R,5S)-3-methyl-3,8-diazabicyclo[3.2.1]octan-8-yl)aniline

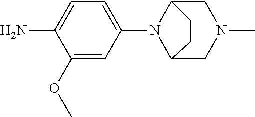 [SMILES: COc1cc(N2C3CCC2CN(C)C3)ccc1N]

The titled compound (240 mg, 84%) was prepared in a manner similar to that in Example 95 step 2 from (1R,5S)-8-(3-methoxy-4-nitrophenyl)-3-methyl-3,8-diazabicyclo[3.2.1]octane. [M+H]⁺=248.2.

### Step 3 N²-(2-methoxy-4-((1R,5S)-3-methyl-3,8-diazabicyclo[3.2.1]octan-8-yl)phenyl)—N⁴-(1-(methylsulfonyl)indolin-7-yl)-7H-pyrrolo[2,3-d]pyrimidine-2,4-diamine

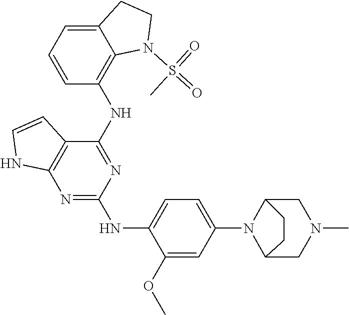 [SMILES: COc1cc(N2C3CCC2CN(C)C3)ccc1Nc1nc(Nc2cccc3c2N(S(C)(=O)=O)CC3)c2cc[nH]c2n1]

The titled compound was prepared in a manner similar to that in Example 77. ¹H NMR (400 MHz, DMSO) δ_{H} 11.21 (s, 1H), 8.97 (s, 1H), 8.25 (d, J=7.8, 1H), 7.85 (d, J=8.6, 1H), 7.24-7.16 (m, 2H), 7.08 (d, J=6.7, 1H), 6.91 (s, 1H), 6.48 (s, 1H), 6.34 (d, J=8.4, 1H), 6.18 (s, 1H), 4.21 (s, 2H), 4.09 (t, J=7.2, 2H), 3.79 (s, 3H), 3.13 (d, J=7.4, 2H), 3.09 (s, 3H), 2.47 (s, 2H), 2.34 (d, J=9.8, 2H), 2.09 (s, 3H), 1.85 (dd, J=19.9, 8.0, 4H); [M+H]⁺=575.3.

### Example 112: N²-(4-((2-(dimethylamino)ethyl)(methyl)amino)-2-methoxyphenyl)—N⁴-(1-(methylsulfonyl)indolin-7-yl)-7H-pyrrolo[2,3-d]pyrimidine-2,4-diamine

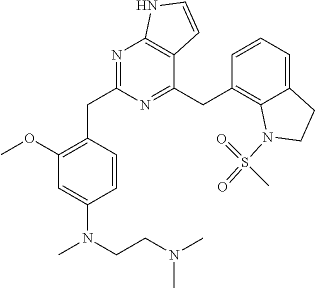 [SMILES: COc1cc(N(C)CCN(C)C)ccc1Cc1nc(Cc2cccc3c2N(S(C)(=O)=O)CC3)c2cc[nH]c2n1]

The titled compound was prepared in a manner similar to that in Example 77. ¹H NMR (400 MHz, DMSO) δ_{H} 11.18 (s, 1H), 8.95 (s, 1H), 8.25 (d, J=8.4, 1H), 7.79 (d, J=8.9, 1H), 7.24-7.13 (m, 2H), 7.07 (d, J=7.3, 1H), 6.90 (s, 1H), 6.38 (s, 1H), 6.24 (d, J=8.5, 1H), 6.18 (s, 1H), 4.09 (t, J=7.2, 2H), 3.80 (s, 3H), 3.32-3.29 (m, 2H), 3.13 (d, J=7.4, 2H), 3.08 (s, 3H), 2.90 (s, 3H), 2.43 (d, J=6.8, 2H), 2.22 (s, 6H); [M+H]⁺=551.3.

### Example 113: N²-(4-(1-(2-(dimethylamino)ethyl)-1H-pyrazol-4-yl)-2-methoxyphenyl)—N⁴-(1-(methylsulfonyl)indolin-7-yl)-7H-pyrrolo[2,3-d]pyrimidine-2,4-diamine

### Step 1: 2-(4-(3-methoxy-4-nitrophenyl)-1H-pyrazol-1-yl)—N,N-dimethylethan-1-amine

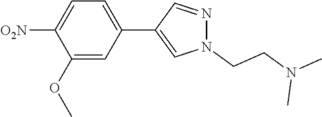 [SMILES: COc1cc(-c2cnn(CCN(C)C)c2)ccc1[N+](=O)[O-]]

A mixture of 4-chloro-2-methoxy-1-nitrobenzene (500 mg, 2.7 mmol), N,N-dimethyl-2-(4-(4,4,5,5-tetramethyl-1,3,2-dioxaborolan-2-yl)-1H-pyrazol-1-yl)ethan-1-amine (780 mg, 2.9 mmol), Pd(PPh₃)₄ (309 mg, 0.27 mmol) and Cs₂CO₃ (1.3 g, 4.0 mmol) in 1,4-dioxane (30 mL) and H₂O (6 mL) was stirred in a round bottom flask at 80° C. for 2 h under N₂. The mixture was evaporated in vacuum to afford the crude product, which was purified with silica gel column chromatography (DCM: MeOH=100: 0˜ 10: 1 gradient elution) to give the title product (760 mg, 97%). [M+H]⁺=291.2.

### Step 2: 4-(1-(2-(dimethylamino)ethyl)-1H-pyrazol-4-yl)-2-methoxyaniline

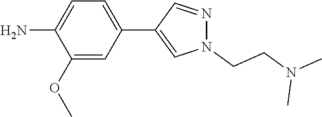 [SMILES: COc1cc(-c2cnn(CCN(C)C)c2)ccc1N]

Under N₂, to a solution of 2-(4-(3-methoxy-4-nitrophenyl)-1H-pyrazol-1-yl)—N,N-dimethylethan-1-amine (760 mg, 2.6 mmol) in MeOH (30 mL) was added 10% Pd/C (200 mg) at 25° C. And then the mixture was exchanged with H₂ two times and stirred under H₂ atmosphere at 25° C. overnight. The mixture was filtered through a pad of Celite and washed with MeOH (20 mL). The filtrate was concentrated under vacuum to obtain the product (680 mg, 99%). [M+H]⁺=261.2.

### Step 3: N²-(4-(1-(2-(dimethylamino)ethyl)-1H-pyrazol-4-yl)-2-methoxyphenyl)—N⁴-(1-(methylsulfonyl)indolin-7-yl)-7H-pyrrolo[2,3-d]pyrimidine-2,4-diamine

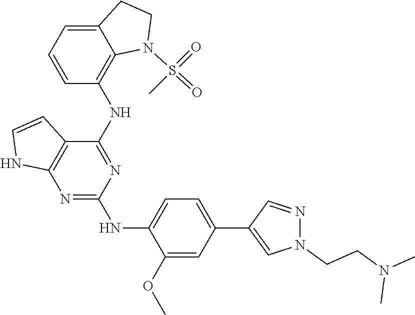 [SMILES: COc1cc(-c2cnn(CCN(C)C)c2)ccc1Nc1nc(Nc2cccc3c2N(S(C)(=O)=O)CC3)c2cc[nH]c2n1]

The titled compound was synthesized in the method similar to Example 77. ¹H NMR (400 MHz, DMSO) δ_{H} 11.36 (s, 1H), 9.07 (s, 1H), 8.36 (d, J=8.2 Hz, 1H), 8.18 (d, J=7.9 Hz, 1H), 8.14 (s, 1H), 7.85 (s, 1H), 7.39 (s, 1H), 7.28 (t, J=7.8 Hz, 1H), 7.18 (s, 1H), 7.14 (d, J=7.3 Hz, 1H), 7.08 (d, J=8.1 Hz, 1H), 6.99 (s, 1H), 6.23 (s, 1H), 4.20 (s, 2H), 4.10 (t, J=7.2 Hz, 2H), 3.93 (s, 3H), 3.13 (d, J=6.7 Hz, 2H), 3.09 (s, 3H), 2.68 (s, 2H), 2.19 (s, 6H); [M+H]⁺=588.5.

### Example 114: N²-(2-methoxy-4-(1-(pyridin-3-ylmethyl)-1H-pyrazol-4-yl)phenyl)—N⁴-(1-(methylsulfonyl)indolin-7-yl)-7H-pyrrolo[2,3-d]pyrimidine-2,4-diamine

### Step 1: 3-((4-(3-methoxy-4-nitrophenyl)-1H-pyrazol-1-yl)methyl)pyridine

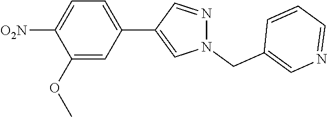 [SMILES: COc1cc(-c2cnn(Cc3cccnc3)c2)ccc1[N+](=O)[O-]]

A mixture of 4-chloro-2-methoxy-1-nitrobenzene (500 mg, 2.7 mmol), 3-((4-(4,4,5,5-tetramethyl-1,3,2-dioxaborolan-2-yl)-1H-pyrazol-1-yl)methyl)pyridine (838 mg, 2.9 mmol), Pd(PPh₃)₄ (309 mg, 0.27 mmol) and Cs₂CO₃ (1.3 g, 4.0 mmol) in 1,4-dioxane (30 mL) and H₂O (6 mL) was stirred in a round bottom flask at 80° C. for 2 h under N₂. The mixture was evaporated in vacuum to afford the crude product, which was purified with silica gel column chromatography (DCM: MeOH=100: 0˜ 20: 1 gradient elution) to give the title product (820 mg, 98%). [M+H]⁺=311.2.

### Step 2: 2-methoxy-4-(1-(pyridin-3-ylmethyl)-1H-pyrazol-4-yl)aniline

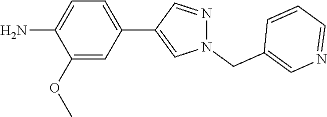 [SMILES: COc1cc(-c2cnn(Cc3cccnc3)c2)ccc1N]

Under N₂, to a solution of 3-((4-(3-methoxy-4-nitrophenyl)-1H-pyrazol-1-yl)methyl)pyridine (820 mg, 2.6 mmol) in MeOH (30 mL) was added 10% Pd/C (200 mg) at 25° C. And then the mixture was exchanged with H₂ two times and stirred under H₂ atmosphere at 25° C. overnight. The mixture was filtered through a pad of Celite and washed with MeOH (20 mL). The filtrate was concentrated under vacuum to obtain the product (735 mg, 99%). [M+H]⁺=281.2.

### Step 3: N²-(2-methoxy-4-(1-(pyridin-3-ylmethyl)-1H-pyrazol-4-yl)phenyl)—N⁴-(1-(methylsulfonyl)indolin-7-yl)-7H-pyrrolo[2,3-d]pyrimidine-2,4-diamine

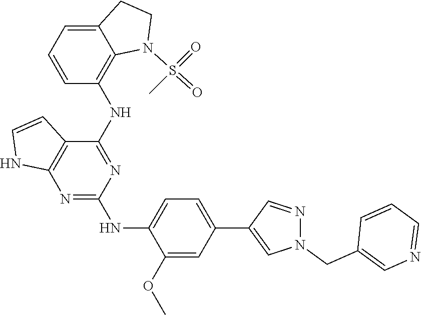 [SMILES: COc1cc(-c2cnn(Cc3cccnc3)c2)ccc1Nc1nc(Nc2cccc3c2N(S(C)(=O)=O)CC3)c2cc[nH]c2n1]

The titled compound was synthesized in the method similar to Example 77. ¹H NMR (400 MHz, DMSO) δ_{H} 11.37 (s, 1H), 9.07 (s, 1H), 8.56 (s, 1H), 8.52 (d, J=3.4 Hz, 1H), 8.37 (d, J=8.3 Hz, 1H), 8.29 (s, 1H), 8.17 (d, J=8.1 Hz, 1H), 7.93 (s, 1H), 7.68 (d, J=7.7 Hz, 1H), 7.39 (s, 2H), 7.28 (t, J=7.8 Hz, 1H), 7.20 (s, 1H), 7.12 (dd, J=15.4, 7.7 Hz, 2H), 6.99 (s, 1H), 6.23 (s, 1H), 5.40 (s, 2H), 4.10 (t, J=7.5 Hz, 2H), 3.93 (s, 3H), 3.14 (t, J=7.0 Hz, 2H), 3.09 (s, 3H); [M+H]⁺=608.5.

### Example 115: N²-(2-methoxy-4-(1-(2-(4-methylpiperazin-1-yl)ethyl)-1H-pyrazol-4-yl)phenyl)—N⁴-(1-(methylsulfonyl)indolin-7-yl)-7H-pyrrolo[2,3-d]pyrimidine-2,4-diamine

### Step 1: 1-(2-(4-(3-methoxy-4-nitrophenyl)-1H-pyrazol-1-yl)ethyl)-4-methylpiperazine

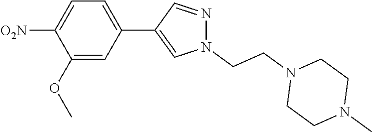 [SMILES: COc1cc(-c2cnn(CCN3CCN(C)CC3)c2)ccc1[N+](=O)[O-]]

A mixture of 4-chloro-2-methoxy-1-nitrobenzene (200 mg, 1.1 mmol), 1-methyl-4-(2-(4-(4,4,5,5-tetramethyl-1,3,2-dioxaborolan-2-yl)-1H-pyrazol-1-yl)ethyl)piperazine (376 mg, 1.2 mmol), Pd(PPh₃)₄ (123 mg, 0.11 mmol) and Cs₂CO₃ (523 mg, 1.6 mmol) in 1,4-dioxane (20 mL) and H₂O (4 mL) was stirred in a round bottom flask at 80° C. for 2 h under N₂. The mixture was evaporated in vacuum to afford the crude product, which was purified with silica gel column chromatography (DCM: MeOH=100: 0˜ 10: 1 gradient elution) to give the title product (255 mg, 69%). [M+H]⁺=346.2.

### Step 2: 2-methoxy-4-(1-(2-(4-methylpiperazin-1-yl)ethyl)-1H-pyrazol-4-yl)aniline

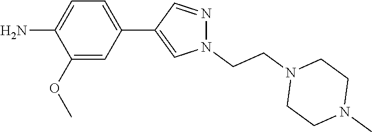 [SMILES: COc1cc(-c2cnn(CCN3CCN(C)CC3)c2)ccc1N]

Under N₂, to a solution of 1-(2-(4-(3-methoxy-4-nitrophenyl)-1H-pyrazol-1-yl)ethyl)-4-methylpiperazine (255 mg, 0.74 mmol) in MeOH (20 mL) was added 10% Pd/C (50 mg) at 25° C. And then the mixture was exchanged with H₂ two times and stirred under H₂ atmosphere at 25° C. overnight. The mixture was filtered through a pad of Celite and washed with MeOH (20 mL). The filtrate was concentrated under vacuum to obtain the product (210 mg, 90%). [M+H]⁺=316.2.

### Step 3: N²-(2-methoxy-4-(1-(2-(4-methylpiperazin-1-yl)ethyl)-1H-pyrazol-4-yl)phenyl)—N⁴-(1-(methylsulfonyl)indolin-7-yl)-7H-pyrrolo[2,3-d]pyrimidine-2,4-diamine

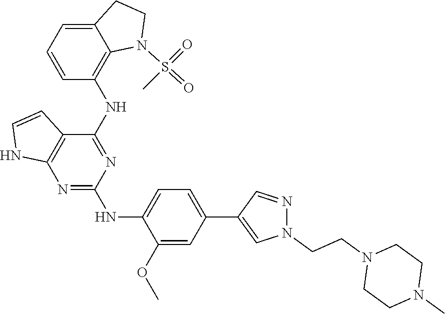 [SMILES: COc1cc(-c2cnn(CCN3CCN(C)CC3)c2)ccc1Nc1nc(Nc2cccc3c2N(S(C)(=O)=O)CC3)c2cc[nH]c2n1]

The titled compound was synthesized in the method similar to Example 77 step 5. ¹H NMR (400 MHz, DMSO) δ_{H} 11.36 (s, 1H), 9.07 (s, 1H), 8.35 (d, J=8.3 Hz, 1H), 8.19 (s, 1H), 8.13 (s, 1H), 7.85 (s, 1H), 7.39 (s, 1H), 7.27 (t, J=7.8 Hz, 1H), 7.18 (s, 1H), 7.14 (d, J=7.3 Hz, 1H), 7.07 (d, J=8.3 Hz, 1H), 6.99 (s, 1H), 6.23 (s, 1H), 4.21 (s, 2H), 4.10 (s, 2H), 3.93 (s, 3H), 3.14 (s, 2H), 3.09 (s, 3H), 2.74 (d, J=6.7 Hz, 2H), 2.44 (s, 4H), 2.32 (s, 4H), 2.15 (s, 3H); [M+H]⁺=643.5.

### Example 123: N²-(6-(4-methylpiperazin-1-yl)pyridin-3-yl)—N⁴-(1-(methylsulfonyl)indolin-7-yl)-7H-pyrrolo[2,3-d]pyrimidine-2,4-diamine

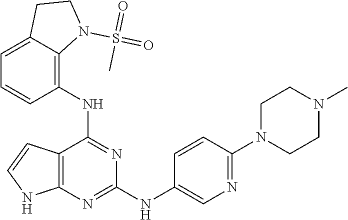 [SMILES: CN1CCN(c2ccc(Nc3nc(Nc4cccc5c4N(S(C)(=O)=O)CC5)c4cc[nH]c4n3)cn2)CC1]

The titled compound was synthesized in the procedures similar to Example 001. ¹H NMR (400 MHz, CD₃OD) δ 8.39 (s, 1H), 8.27 (s, 1H), 8.03 (s, 1H), 7.20 (s, 1H), 7.09 (s, 1H), 6.87 (s, 2H), 6.32 (s, 1H), 4.16 (s, 2H), 3.62 (s, 3H), 3.11-3.08 (m, 7H), 2.98 (s, 3H), 2.72 (s, 3H); [M+H]⁺=520.6.

### Example 124: N²-(4-(4-(dimethylamino)piperidin-1-yl)-2,3-dihydrobenzofuran-7-yl)—N⁴-(1-(methylsulfonyl)indolin-7-yl)-7H-pyrrolo[2,3-d]pyrimidine-2,4-diamine

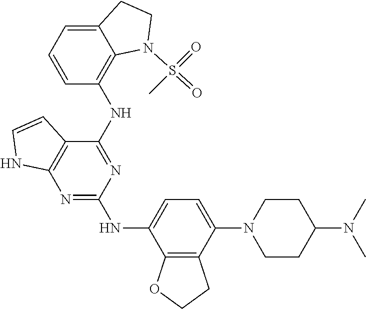 [SMILES: CN(C)C1CCN(c2ccc(Nc3nc(Nc4cccc5c4N(S(C)(=O)=O)CC5)c4cc[nH]c4n3)c3c2CCO3)CC1]

The titled compound was prepared in a manner similar to that in Example 77. ¹H NMR (400 MHz, DMSO) δ_{H} 11.20 (s, 1H), 8.95 (s, 1H), 8.26 (d, J=7.7 Hz, 1H), 7.57 (d, J=8.4 Hz, 1H), 7.38 (s, 1H), 7.12 (t, J=7.7 Hz, 1H), 7.03 (d, J=7.4 Hz, 1H), 6.88 (d, J=2.2 Hz, 1H), 6.38 (d, J=8.4 Hz, 1H), 6.16 (s, 1H), 4.49 (t, J=8.7 Hz, 2H), 4.05 (t, J=7.5 Hz, 2H), 3.15-3.03 (m, 9H), 2.71-2.54 (m, 9H), 1.99 (s, 2H), 1.66 (dd, J=24.2, 13.1 Hz, 2H); [M+H]⁺=589.3.

### Example 125: N²-(4-(4-(4-methylpiperazin-1-yl)piperidin-1-yl)-2,3-dihydrobenzofuran-7-yl)—N⁴-(1-(methylsulfonyl)indolin-7yl)-7H-pyrrolo[2,3-d]pyrimidine-2,4-diamine

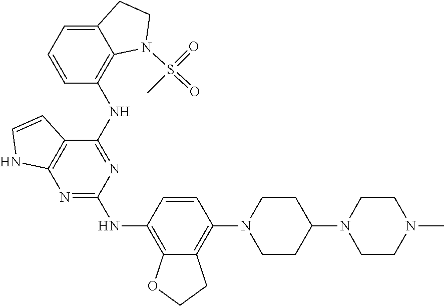 [SMILES: CN1CCN(C2CCN(c3ccc(Nc4nc(Nc5cccc6c5N(S(C)(=O)=O)CC6)c5cc[nH]c5n4)c4c3CCO4)CC2)CC1]

The titled compound was prepared in a manner similar to that in Example 77.

¹H NMR (400 MHz, CD₃OD) δ_{H} 8.48 (s, 1H), 8.19 (d, J=8.4 Hz, 1H), 7.81 (d, J=8.7 Hz, 1H), 7.22-7.16 (m, 1H), 7.10 (d, J=8.3 Hz, 1H), 6.87 (d, J=3.6 Hz, 1H), 6.44 (d, J=8.7 Hz, 1H), 6.31 (d, J=3.6 Hz, 1H), 4.78 (s, 1H), 4.64-4.57 (m, 7H), 4.16 (t, J=7.6 Hz, 2H), 3.41 (d, J=13.0 Hz, 2H), 3.21-3.14 (m, 4H), 2.98 (s, 4H), 2.68 (t, J=10.9 Hz, 3H), 2.56 (s, 4H), 2.01 (s, 2H), 1.71 (d, J=12.1 Hz, 2H); [M+H]⁺=644.3.

### Example 134: 5-bromo-N₄-(2,2-dimethyl-1-(methylsulfonyl)indolin-7-yl)—N₂-(2-methoxy-4-(4-(4-methylpiperazin-1-yl)piperidin-1-yl)phenyl)pyrimidine-2,4-diamine

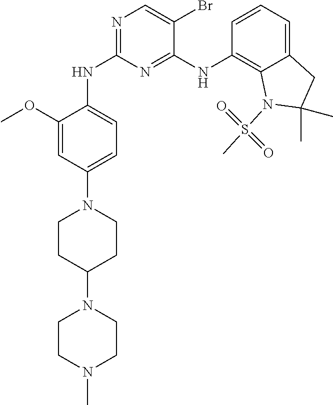

The title compound was prepared in a procedure similar to that in Example 77. ¹H NMR (500 MHz, DMSO) δ_{H} 8.82 (s, 1H), 8.13 (s, 1H), 7.86 (d, J=13.2 Hz, 2H), 7.41 (d, J=8.5 Hz, 1H), 7.15 (s, 2H), 6.59 (d, J=2.2 Hz, 1H), 6.37 (d, J=8.8 Hz, 1H), 3.75 (s, 3H), 3.68 (d, J=12.4 Hz, 2H), 3.02-2.98 (m, 5H), 2.64 (t, J=11.3 Hz, 2H), 2.55-2.52 (m, 4H), 2.44-2.25 (m, 5H), 2.16 (s, 3H), 1.85 (d, J=11.9 Hz, 2H), 1.56-1.47 (m, 2H), 1.41 (s, 6H). [M+H]⁺=699.5.

### Example 136: 5-bromo-N₂-(2-methoxy-4-(4-(4-methylpiperazin-1-yl)piperidin-1-yl)phenyl)—N₄-(1′-(methylsulfonyl)spiro[cyclopropane-1,2′-indolin]-7′-yl)pyrimidine-2,4-diamine

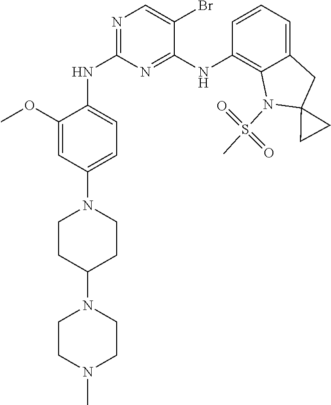 [SMILES: COc1cc(N2CCC(N3CCN(C)CC3)CC2)ccc1Nc1ncc(Br)c(Nc2cccc3c2N(S(C)(=O)=O)C2(CC2)C3)n1]

The title compound was prepared in a procedure similar to that in Example 28. ¹H NMR (500 MHz, DMSO) δ_{H} 8.74 (s, 1H), 8.14 (s, 1H), 7.96 (s, 1H), 7.92 (s, 1H), 7.39 (d, J=8.7 Hz, 1H), 7.24 (d, J=6.7 Hz, 2H), 6.60 (d, J=2.4 Hz, 1H), 6.41 (dd, J=8.8, 2.3 Hz, 1H), 4.94 (d, J=6.5 Hz, 1H), 4.09 (s, 1H), 3.75 (s, 3H), 3.70 (d, J=12.0 Hz, 2H), 2.83 (s, 3H), 2.65 (t, J=11.2 Hz, 2H), 2.49-2.22 (m, 11H), 2.15 (s, 3H), 1.98-1.89 (m, 1H), 1.84 (d, J=12.4 Hz, 2H), 1.62 (s, 1H), 1.51 (d, J=8.6 Hz, 2H). [M+H]⁺=697.5.

### Example 142: 5-chloro-N₂-(3,3-dimethyl-4-(4-(4-methylpiperazin-1-yl)piperidin-1-yl)-2,3-dihydrobenzofuran-7-yl)—N₄-(1-(methylsulfonyl)indolin-7-yl)pyrimidine-2,4-diamine

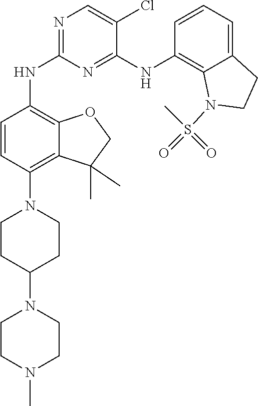 [SMILES: CN1CCN(C2CCN(c3ccc(Nc4ncc(Cl)c(Nc5cccc6c5N(S(C)(=O)=O)CC6)n4)c4c3C(C)(C)CO4)CC2)CC1]

The title compound was prepared in a procedure similar to that in Example 30. ¹H NMR (500 MHz, DMSO) δ 8.96 (s, 1H), 8.18 (s, 1H), 8.01 (s, 1H), 7.85 (s, 1H), 7.68 (s, 1H), 7.15 (d, J=6.9 Hz, 2H), 6.58 (s, 1H), 4.13 (s, 2H), 4.07 (t, J=7.2 Hz, 2H), 3.12 (t, J=7.2 Hz, 2H), 3.06 (s, 3H), 2.94 (d, J=10.8 Hz, 2H), 2.66-2.53 (m, 5H), 2.36-2.29 (m, 6H), 2.17 (s, 3H), 1.81 (d, J=11.9 Hz, 2H), 1.47 (d, J=11.4 Hz, 2H), 1.17 (s, 6H).

### Example 143: 5-chloro-N₂-(4-(4-(4-methylpiperazin-1-yl)piperidin-1-yl)benzofuran-7-yl)-N₄-(1-(methylsulfonyl)indolin-7-yl)pyrimidine-2,4-diamine

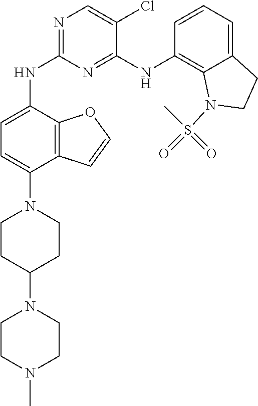 [SMILES: CN1CCN(C2CCN(c3ccc(Nc4ncc(Cl)c(Nc5cccc6c5N(S(C)(=O)=O)CC6)n4)c4occc34)CC2)CC1]

The title compound was prepared in a procedure similar to that in Example 30. ¹H NMR (500 MHz, DMSO) δ 8.98 (s, 1H), 8.89 (s, 1H), 8.11 (s, 1H), 7.90 (d, J=2.1 Hz, 1H), 7.51 (s, 1H), 7.13 (d, J=8.2 Hz, 1H), 7.03 (d, J=7.3 Hz, 1H), 6.96 (d, J=2.2 Hz, 1H), 6.82 (s, 1H), 6.60 (d, J=8.3 Hz, 1H), 4.01 (t, J=7.4 Hz, 2H), 3.58 (d, J=11.9 Hz, 2H), 3.12-2.93 (m, 6H), 2.70 (s, 2H), 2.54 (s, 3H), 2.43-2.24 (m, 5H), 2.17 (s, 3H), 1.89 (s, 2H), 1.73-1.62 (m, 2H).

### Example 147: N₂-(3-(difluoromethoxy)-4-(4-(4-methylpiperazin-1-yl)piperidin-1-yl)phenyl)—N₄-(1-(methylsulfonyl)indolin-7-yl)-7H-pyrrolo[2,3-d]pyrimidine-2,4-diamine

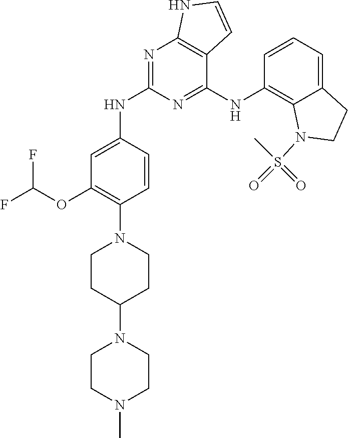 [SMILES: CN1CCN(C2CCN(c3ccc(Nc4nc(Nc5cccc6c5N(S(C)(=O)=O)CC6)c5cc[nH]c5n4)cc3OC(F)F)CC2)CC1]

The title compound was prepared in a procedure similar to that in Example 94. ¹H NMR (500 MHz, MeOD) δ 8.29 (d, J=8.2 Hz, 1H), 7.65 (s, 1H), 7.40 (d, J=8.7 Hz, 1H), 7.25 (t, J=7.8 Hz, 1H), 7.10 (d, J=7.3 Hz, 1H), 6.98 (d, J=8.6 Hz, 1H), 6.89 (d, J=3.5 Hz, 1H), 6.33 (d, J=3.4 Hz, 1H), 4.17 (t, J=7.5 Hz, 2H), 3.38 (d, J=11.3 Hz, 2H), 3.16 (t, J=7.5 Hz, 3H), 2.98 (s, 3H), 2.90-2.82 (s, 8H), 2.65 (t, J=11.6 Hz, 3H), 2.49 (s, 3H), 2.01 (d, J=12.4 Hz, 2H), 1.70 (d, J=11.1 Hz, 2H).

### Example 148: N₂-(3-fluoro-5-methoxy-4-(4-(4-methylpiperazin-1-yl)piperidin-1-yl)phenyl)—N₄-(1-(methylsulfonyl)indolin-7-yl)-7H-pyrrolo[2,3-d]pyrimidine-2,4-diamine

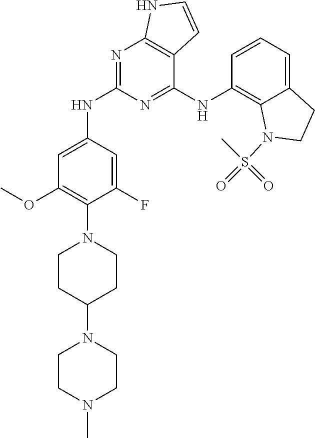 [SMILES: COc1cc(Nc2nc(Nc3cccc4c3N(S(C)(=O)=O)CC4)c3cc[nH]c3n2)cc(F)c1N1CCC(N2CCN(C)CC2)CC1]

The title compound was prepared in a procedure similar to that in Example 77. ¹H NMR (500 MHz, DMSO) δ_{H} 11.30 (s, 1H), 9.04 (s, 1H), 8.20 (d, J=8.1 Hz, 1H), 7.81 (d, J=9.0 Hz, 1H), 7.59 (s, 1H), 7.20 (t, J=7.8 Hz, 1H), 7.11 (d, J=7.3 Hz, 1H), 6.98-6.93 (m, 1H), 6.69 (t, J=9.0 Hz, 1H), 6.20 (d, J=1.9 Hz, 1H), 4.09 (t, J=7.4 Hz, 2H), 3.83 (s, 3H), 3.12 (t, J=7.3 Hz, 2H), 3.09 (s, 3H), 2.91-2.55 (m, 10H), 2.48-2.36 (m, 6H), 1.87 (s, 2H), 1.61 (s, 2H). [M+H]⁺=650.5.

### Example 149: N₂-(2-fluoro-5-methoxy-4-(4-(4-methylpiperazin-1-yl)piperidin-1-yl)phenyl)—N₄-(1-(methylsulfonyl)indolin-7-yl)-7H-pyrrolo[2,3-d]pyrimidine-2,4-diamine

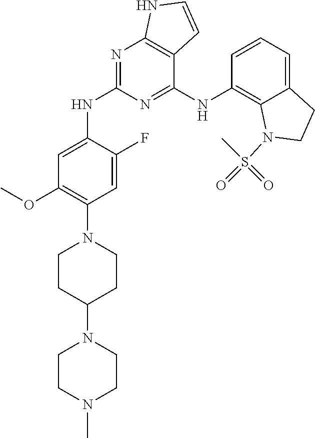 [SMILES: COc1cc(Nc2nc(Nc3cccc4c3N(S(C)(=O)=O)CC4)c3cc[nH]c3n2)c(F)cc1N1CCC(N2CCN(C)CC2)CC1]

The title compound was prepared in a procedure similar to that in Example 77. ¹H NMR (500 MHz, DMSO) δ_{H} 11.26 (s, 1H), 8.97 (s, 1H), 8.31 (d, J=7.5 Hz, 1H), 8.05 (s, 1H), 7.39 (d, J=7.6 Hz, 1H), 7.05 (s, 2H), 6.93 (d, J=2.6 Hz, 1H), 6.76 (d, J=12.5 Hz, 1H), 6.19 (s, 1H), 4.08 (s, 2H), 3.70 (s, 3H), 3.42-3.36 (m, 4H), 3.12-3.06 (m, 5H), 2.65-2.52 (m, 6H), 2.40-2.22 (m, 6H), 1.83 (s, 2H), 1.56 (d, J=10.2 Hz, 2H). [M+H]⁺=650.5.

### Example 154: N₂-(5-(4-(4-methylpiperazin-1-yl)piperidin-1-yl)pyridin-2-yl)—N₄-(1-(methylsulfonyl)indolin-7-yl)-7H-pyrrolo[2,3-d]pyrimidine-2,4-diamine

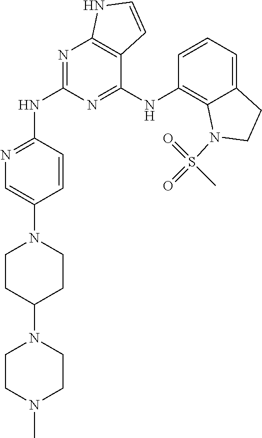 [SMILES: CN1CCN(C2CCN(c3ccc(Nc4nc(Nc5cccc6c5N(S(C)(=O)=O)CC6)c5cc[nH]c5n4)nc3)CC2)CC1]

The titled compound was prepared in a manner similar to that in Example 77. ¹H NMR (500 MHz, DMSO) δ 11.39 (s, 1H), 9.06 (s, 1H), 8.72 (t, J=8.1 Hz, 1H), 8.45 (d, J=8.2 Hz, 1H), 8.18 (d, J=8.7 Hz, 1H), 7.94 (s, 1H), 7.34 (d, J=9.2 Hz, 1H), 7.25 (t, J=7.8 Hz, 1H), 7.11 (d, J=7.2 Hz, 1H), 6.99 (s, 1H), 6.24 (s, 1H), 4.10 (t, J=7.0 Hz, 2H), 3.63 (d, J=11.6 Hz, 2H), 3.13 (t, J=7.3 Hz, 2H), 3.09 (s, 3H), 2.63 (t, J=11.7 Hz, 2H), 2.59-2.51 (m, 4H), 2.34 (dd, J=33.8, 22.9 Hz, 5H), 2.20 (s, 3H), 1.86 (d, J=11.7 Hz, 2H), 1.59-1.48 (m, 2H). [M+H]⁺=603.3.

### Example 181: 5-bromo-N₂-(2-methoxy-5-methyl-4-(4-(4-methylpiperazin-1-yl)piperidin-1-yl)phenyl)—N₄-(1′-(methylsulfonyl)spiro[cyclopropane-1,3′-indolin]-7′-yl)pyrimidine-2,4-diamine

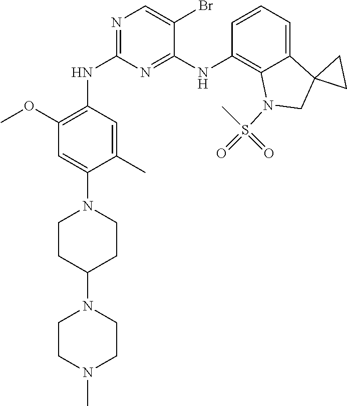 [SMILES: COc1cc(N2CCC(N3CCN(C)CC3)CC2)c(C)cc1Nc1ncc(Br)c(Nc2cccc3c2N(S(C)(=O)=O)CC32CC2)n1]

The titled compound was prepared in a manner similar to that in Example 28. ¹H NMR (500 MHz, DMSO) δ 8.82 (s, 1H), 8.18 (s, 1H), 7.87 (s, 1H), 7.76 (d, J=7.7 Hz, 1H), 7.42 (s, 1H), 7.12 (t, J=7.4 Hz, 1H), 6.72 (d, J=7.3 Hz, 1H), 6.67 (s, 1H), 4.03 (s, 2H), 3.75 (s, 3H), 3.29-3.23 (m, 3H), 3.04 (d, J=16.4 Hz, 5H), 2.65-2.53 (m, 4H), 2.41-2.25 (m, 4H), 2.17 (s, 3H), 2.06 (s, 3H), 1.84 (d, J=11.7 Hz, 2H), 1.55 (q, J=11.4 Hz, 2H), 1.25 (s, 2H), 1.06 (s, 2H). [M+H]⁺=711.2.

### Example 182: N₂-(7-(4-(4-methylpiperazin-1-yl)piperidin-1-yl)benzo[d][1,3]dioxol-4-yl)-N₄-(1-(methylsulfonyl)indolin-7-yl)-7H-pyrrolo[2,3-d]pyrimidine-2,4-diamine

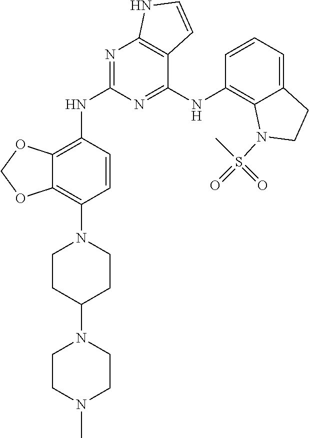 [SMILES: CN1CCN(C2CCN(c3ccc(Nc4nc(Nc5cccc6c5N(S(C)(=O)=O)CC6)c5cc[nH]c5n4)c4c3OCO4)CC2)CC1]

The titled compound was prepared in a manner similar to that in Example 77. ¹H NMR (500 MHz, DMSO) δ 11.22 (s, 1H), 8.95 (s, 1H), 8.35 (d, J=5.2 Hz, 2H), 7.94 (s, 1H), 7.09 (t, J=7.7 Hz, 1H), 7.02 (dd, J=13.4, 8.1 Hz, 2H), 6.90 (d, J=2.3 Hz, 1H), 6.40 (d, J=8.9 Hz, 1H), 6.18 (s, 1H), 5.90 (s, 2H), 4.08 (t, J=7.4 Hz, 2H), 3.58 (d, J=11.7 Hz, 2H), 3.35-3.30 (m, 3H), 3.13-3.05 (m, 5H), 2.61 (t, J=11.5 Hz, 2H), 2.54 (s, 2H), 2.40-2.25 (m, 4H), 2.15 (s, 3H), 1.84 (d, J=11.6 Hz, 2H), 1.52 (dd, J=20.5, 11.6 Hz, 2H). [M+H]⁺=646.3.

### Example 184: N₂-(6-(4-(4-methylpiperazin-1-yl)piperidin-1-yl)pyridin-3-yl)—N₄-(1-(methylsulfonyl)indolin-7-yl)-7H-pyrrolo[2,3-d]pyrimidine-2,4-diamine

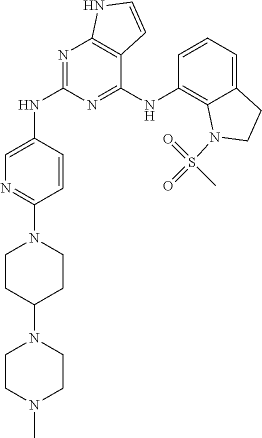 [SMILES: CN1CCN(C2CCN(c3ccc(Nc4nc(Nc5cccc6c5N(S(C)(=O)=O)CC6)c5cc[nH]c5n4)cn3)CC2)CC1]

The titled compound was prepared in a manner similar to that in Example 77. ¹H NMR (500 MHz, DMSO) δ 11.24 (s, 1H), 8.98 (s, 1H), 8.60 (s, 1H), 8.42 (s, 1H), 8.32 (d, J=8.2 Hz, 1H), 7.91 (dd, J=9.1, 2.5 Hz, 1H), 7.21 (t, J=7.8 Hz, 1H), 7.09 (d, J=7.3 Hz, 1H), 6.93-6.87 (m, 1H), 6.77 (d, J=9.1 Hz, 1H), 6.18 (d, J=1.9 Hz, 1H), 4.19 (d, J=12.9 Hz, 2H), 4.10 (t, J=7.5 Hz, 2H), 3.24-3.19 (m, 2H), 3.12 (t, J=7.4 Hz, 2H), 3.09 (s, 3H), 2.70 (t, J=11.6 Hz, 2H), 2.53 (d, J=15.7 Hz, 2H), 2.39-2.22 (m, 5H), 2.13 (s, 3H), 1.81 (d, J=12.2 Hz, 2H), 1.46-1.33 (m, 2H). [M+H]⁺=603.3.

### Example 185: 5-bromo-N₂-(4-(4-(4-ethylpiperazin-1-yl)piperidin-1-yl)-2,5-difluorophenyl)—N₄-(1-(methylsulfonyl)indolin-7-yl)pyrimidine-2,4-diamine

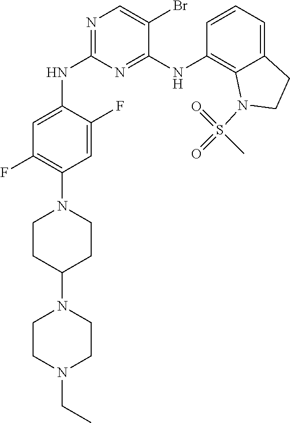 [SMILES: CCN1CCN(C2CCN(c3cc(F)c(Nc4ncc(Br)c(Nc5cccc6c5N(S(C)(=O)=O)CC6)n4)cc3F)CC2)CC1]

The titled compound was prepared in a manner similar to that in Example 77. ¹H NMR (500 MHz, DMSO) δ 8.86 (s, 2H), 8.21 (s, 1H), 7.81 (d, J=7.0 Hz, 1H), 7.39 (dd, J=13.8, 7.6 Hz, 1H), 7.18-7.07 (m, 2H), 6.91 (dd, J=12.3, 8.3 Hz, 1H), 4.04 (t, J=7.3 Hz, 2H), 3.25-3.17 (m, 3H), 3.09 (t, J=7.3 Hz, 2H), 3.05 (s, 3H), 2.62 (t, J=11.3 Hz, 2H), 2.51 (s, 4H), 2.37 (d, J=5.2 Hz, 2H), 2.29 (dd, J=14.3, 7.1 Hz, 4H), 1.85 (d, J=11.9 Hz, 2H), 1.54 (dd, J=20.9, 11.5 Hz, 2H), 0.98 (t, J=7.2 Hz, 3H). [M+H]⁺=691.3.

### Example 186: 4-(1-(3-methoxy-4-((4-((1-(methylsulfonyl)indolin-7-yl)amino)-7H-pyrrolo[2,3-d]pyrimidin-2-yl)amino)phenyl)piperidin-4-yl)-1-methylpiperazin-2-one

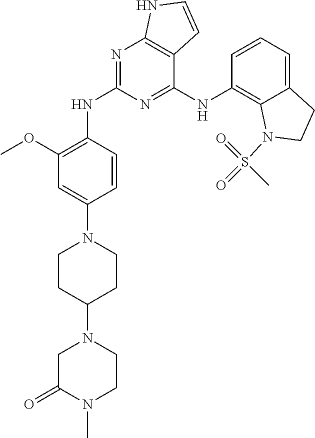 [SMILES: COc1cc(N2CCC(N3CCN(C)C(=O)C3)CC2)ccc1Nc1nc(Nc2cccc3c2N(S(C)(=O)=O)CC3)c2cc[nH]c2n1]

The titled compound was prepared in a manner similar to that in Example 77. ¹H NMR (500 MHz, DMSO) δ 11.26 (s, 1H), 9.00 (s, 1H), 8.22 (d, J=8.1 Hz, 1H), 7.97 (d, J=8.7 Hz, 1H), 7.29-7.17 (m, 2H), 7.10 (d, J=7.4 Hz, 1H), 6.94-6.88 (m, 1H), 6.63 (d, J=2.3 Hz, 1H), 6.45 (dd, J=8.8, 2.3 Hz, 1H), 6.19 (dd, J=3.3, 1.9 Hz, 1H), 4.09 (t, J=7.5 Hz, 2H), 3.82 (s, 3H), 3.65 (d, J=12.1 Hz, 2H), 3.26-3.23 (m, 2H), 3.14-3.08 (m, 7H), 2.82 (s, 3H), 2.75 (t, J=5.3 Hz, 2H), 2.63 (t, J=11.1 Hz, 2H), 2.37 (t, J=11.1 Hz, 1H), 1.88 (d, J=11.1 Hz, 2H), 1.53 (dd, J=20.3, 11.5 Hz, 2H). [M+H]⁺=646.3.

### Example 187: 5-chloro-N₂-(2-methoxy-4-(4-(4-methylpiperazin-1-yl)piperidin-1-yl)phenyl)—N₄-(1′-(methylsulfonyl)spiro[cyclopropane-1,3′-indolin]-7′-yl)pyrimidine-2,4-diamine

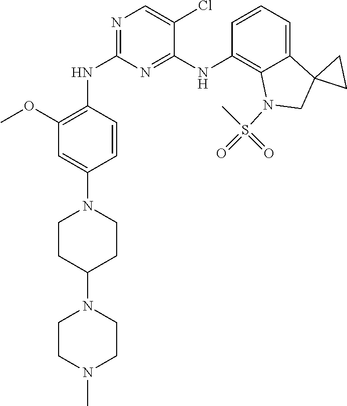 [SMILES: COc1cc(N2CCC(N3CCN(C)CC3)CC2)ccc1Nc1ncc(Cl)c(Nc2cccc3c2N(S(C)(=O)=O)CC32CC2)n1]

The titled compound was prepared in a manner similar to that in Example 28. ¹H NMR (500 MHz, DMSO) δ 8.95 (s, 1H), 8.09 (s, 1H), 7.91 (s, 1H), 7.83 (d, J=7.9 Hz, 1H), 7.42 (d, J=8.6 Hz, 1H), 7.14 (t, J=7.8 Hz, 1H), 6.70 (d, J=7.4 Hz, 1H), 6.59 (d, J=2.3 Hz, 1H), 6.39 (dd, J=8.8, 2.2 Hz, 1H), 4.03 (s, 2H), 3.76 (s, 3H), 3.69 (d, J=12.3 Hz, 2H), 3.32-3.25 (m, 3H), 3.03 (s, 3H), 2.64 (t, J=11.4 Hz, 2H), 2.29 (dd, J=14.9, 7.2 Hz, 6H), 2.14 (s, 3H), 1.84 (d, J=11.6 Hz, 2H), 1.50 (td, J=11.7, 8.7 Hz, 2H), 1.24 (t, J=5.5 Hz, 2H), 1.06 (q, J=4.5 Hz, 2H). [M+H]⁺=653.3.

### Example 188: 5-chloro-N₂-(2-methoxy-5-methyl-4-(4-(4-methylpiperazin-1-yl)piperidin-1-yl)phenyl)—N₄-(1′-(methylsulfonyl)spiro[cyclopropane-1,3′-indolin]-7′-yl)pyrimidine-2,4-diamine

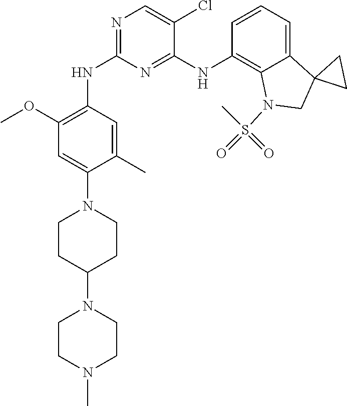 [SMILES: COc1cc(N2CCC(N3CCN(C)CC3)CC2)c(C)cc1Nc1ncc(Cl)c(Nc2cccc3c2N(S(C)(=O)=O)CC32CC2)n1]

The titled compound was prepared in a manner similar to that in Example 28. ¹H NMR (500 MHz, DMSO) δ 8.97 (s, 1H), 8.12 (s, 1H), 7.88 (s, 1H), 7.81 (d, J=8.1 Hz, 1H), 7.46 (s, 1H), 7.13 (t, J=7.8 Hz, 1H), 6.72 (d, J=7.4 Hz, 1H), 6.67 (s, 1H), 4.03 (s, 2H), 3.76 (s, 3H), 3.38-3.30 (m, 2H), 3.04 (d, J=15.6 Hz, 5H), 2.59 (dd, J=23.3, 12.2 Hz, 5H), 2.34 (dd, J=32.1, 20.8 Hz, 4H), 2.20 (s, 3H), 2.07 (s, 3H), 1.84 (d, J=11.4 Hz, 2H), 1.56 (td, J=11.5, 8.7 Hz, 2H), 1.25 (t, J=5.4 Hz, 2H), 1.06 (q, J=4.5 Hz, 2H). [M+H]⁺=667.3.

### Example 189: N₂-(2-methoxy-4-(4-(4-methyl-4,7-diazaspiro[2.5]octan-7-yl)piperidin-1-yl)phenyl)—N₄-(1-(methylsulfonyl)indolin-7-yl)-7H-pyrrolo[2,3-d]pyrimidine-2,4-diamine

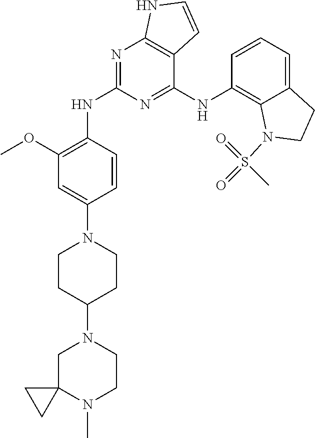 [SMILES: COc1cc(N2CCC(N3CCN(C)C4(CC4)C3)CC2)ccc1Nc1nc(Nc2cccc3c2N(S(C)(=O)=O)CC3)c2cc[nH]c2n1]

The titled compound was prepared in a manner similar to that in Example 77. ¹H NMR (500 MHz, DMSO) δ 11.24 (s, 1H), 8.99 (s, 1H), 8.22 (d, J=8.1 Hz, 1H), 7.96 (d, J=8.7 Hz, 1H), 7.29-7.17 (m, 2H), 7.09 (d, J=6.7 Hz, 1H), 6.92 (dd, J=3.4, 2.3 Hz, 1H), 6.61 (d, J=2.5 Hz, 1H), 6.44 (dd, J=8.8, 2.5 Hz, 1H), 6.19 (dd, J=3.4, 1.9 Hz, 1H), 4.09 (t, J=7.5 Hz, 2H), 3.82 (s, 3H), 3.64 (d, J=11.8 Hz, 2H), 3.12 (t, J=7.4 Hz, 2H), 3.09 (s, 3H), 2.76-2.72 (m, 2H), 2.63 (t, J=11.0 Hz, 2H), 2.56-2.52 (m, 2H), 2.30 (dt, J=18.0, 11.7 Hz, 3H), 2.18 (s, 3H), 1.86 (d, J=11.3 Hz, 2H), 1.58-1.46 (m, 2H), 0.57 (t, J=5.2 Hz, 2H), 0.32 (q, J=4.6 Hz, 2H). [M+H]⁺=658.3.

### Example 190: N₂-(4-methoxy-6-(4-(4-methylpiperazin-1-yl)piperidin-1-yl)pyridin-3-yl)-N₄-(1-(methylsulfonyl)indolin-7-yl)-7H-pyrrolo[2,3-d]pyrimidine-2,4-diamine

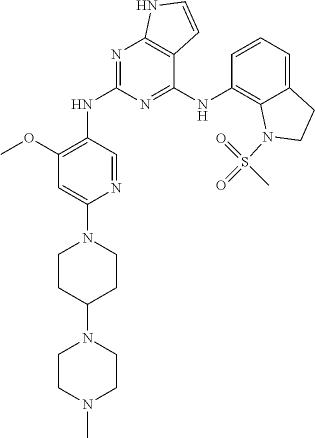 [SMILES: COc1cc(N2CCC(N3CCN(C)CC3)CC2)ncc1Nc1nc(Nc2cccc3c2N(S(C)(=O)=O)CC3)c2cc[nH]c2n1]

The titled compound was prepared in a manner similar to that in Example 77. ¹H NMR (500 MHz, DMSO) δ 11.18 (s, 1H), 8.93 (s, 1H), 8.26 (d, J=9.2 Hz, 2H), 7.39 (s, 1H), 7.11 (t, J=7.8 Hz, 1H), 7.03 (d, J=7.2 Hz, 1H), 6.91-6.77 (m, 1H), 6.44 (s, 1H), 6.23-6.08 (m, 1H), 4.31 (d, J=13.0 Hz, 2H), 4.09 (d, J=7.4 Hz, 2H), 3.80 (s, 3H), 3.17 (d, J=4.0 Hz, 1H), 3.12-3.07 (m, 5H), 2.76 (t, J=11.7 Hz, 2H), 2.37 (dd, J=25.4, 14.1 Hz, 8H), 2.15 (s, 3H), 1.82 (d, J=11.7 Hz, 2H), 1.39 (dd, J=20.5, 11.9 Hz, 2H). [M+H]⁺=633.3.

### Example 191: 1-(1-(3-methoxy-4-((4-((1-(methylsulfonyl)indolin-7-yl)amino)-7H-pyrrolo[2,3-d]pyrimidin-2-yl)amino)phenyl)piperidin-4-yl)-4-methylpiperazin-2-one

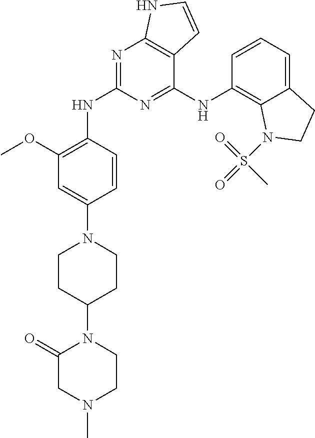 [SMILES: COc1cc(N2CCC(N3CCN(C)CC3=O)CC2)ccc1Nc1nc(Nc2cccc3c2N(S(C)(=O)=O)CC3)c2cc[nH]c2n1]

The titled compound was prepared in a manner similar to that in Example 77. ¹H NMR (500 MHz, DMSO) δ 11.26 (s, 1H), 9.00 (s, 1H), 8.22 (d, J=7.9 Hz, 1H), 7.99 (d, J=8.7 Hz, 1H), 7.28-7.18 (m, 2H), 7.10 (d, J=7.3 Hz, 1H), 6.92 (dd, J=3.4, 2.3 Hz, 1H), 6.64 (d, J=2.4 Hz, 1H), 6.47 (dd, J=8.8, 2.4 Hz, 1H), 6.19 (dd, J=3.4, 1.9 Hz, 1H), 4.37 (t, J=12.1 Hz, 1H), 4.09 (t, J=7.5 Hz, 2H), 3.83 (s, 3H), 3.71 (d, J=12.2 Hz, 2H), 3.24 (d, J=5.3 Hz, 2H), 3.12 (t, J=7.4 Hz, 2H), 3.09 (s, 3H), 2.95 (s, 2H), 2.70 (t, J=11.3 Hz, 2H), 2.59-2.55 (m, 2H), 2.20 (s, 3H), 1.85 (dt, J=12.1, 8.5 Hz, 2H), 1.59 (d, J=11.3 Hz, 2H). [M+H]⁺=646.3.

### Example 192: 5-bromo-N₂-(2-methoxy-4-(4-(4-methyl-4,7-diazaspiro[2.5]octan-7-yl)piperidin-1-yl)phenyl)—N₄-(1-(methylsulfonyl)indolin-7-yl)pyrimidine-2,4-diamine

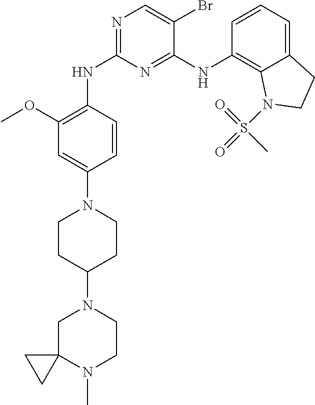 [SMILES: COc1cc(N2CCC(N3CCN(C)C4(CC4)C3)CC2)ccc1Nc1ncc(Br)c(Nc2cccc3c2N(S(C)(=O)=O)CC3)n1]

The titled compound was prepared in a manner similar to that in Example 30. ¹H NMR (500 MHz, DMSO) δ 8.77 (s, 1H), 8.14 (s, 1H), 7.89 (s, 1H), 7.83 (s, 1H), 7.41 (d, J=8.7 Hz, 1H), 7.17-7.12 (m, 2H), 6.58 (d, J=2.3 Hz, 1H), 6.39 (dd, J=8.7, 2.2 Hz, 1H), 4.04 (t, J=7.4 Hz, 2H), 3.75 (s, 3H), 3.68 (d, J=12.3 Hz, 2H), 3.09 (t, J=7.3 Hz, 2H), 3.05 (d, J=7.8 Hz, 3H), 2.79-2.72 (m, 2H), 2.66 (t, J=11.3 Hz, 2H), 2.57-2.53 (m, 2H), 2.41-2.27 (m, 3H), 2.19 (s, 3H), 1.86 (d, J=11.7 Hz, 2H), 1.56-1.44 (m, 2H), 0.58 (t, J=5.1 Hz, 2H), 0.32 (t, J=5.2 Hz, 2H). [M+H]⁺=697.2

### Example 193: 5-bromo-N₂-(8-(4-(4-methylpiperazin-1-yl)piperidin-1-yl)-2,3-dihydrobenzo[b][1,4]dioxin-5-yl)—N₄-(1-(methylsulfonyl)indolin-7-yl)pyrimidine-2,4-diamine

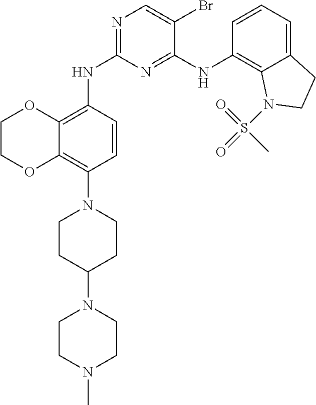 [SMILES: CN1CCN(C2CCN(c3ccc(Nc4ncc(Br)c(Nc5cccc6c5N(S(C)(=O)=O)CC6)n4)c4c3OCCO4)CC2)CC1]

The titled compound was prepared in a manner similar to that in Example 51. ¹H NMR (500 MHz, MeOD) δ 8.30 (s, 1H), 7.57 (d, J=7.9 Hz, 1H), 7.44 (d, J=7.3 Hz, 1H), 7.37-7.26 (m, 2H), 7.08 (d, J=9.0 Hz, 1H), 4.46 (s, 2H), 4.41 (s, 2H), 4.09 (t, J=7.5 Hz, 2H), 4.01-3.44 (m, 13H), 3.21 (t, J=7.5 Hz, 2H), 3.05 (s, 3H), 2.97 (s, 3H), 2.54 (d, J=9.5 Hz, 2H), 2.43 (d, J=11.4 Hz, 2H). [M+H]⁺=699.2.

### Example 194: N₂-(4-(4-(4-methylpiperazin-1-yl)piperidin-1-yl)phenyl)—N₄-(1-(methylsulfonyl)indolin-7-yl)-7H-pyrrolo[2,3-d]pyrimidine-2,4-diamine

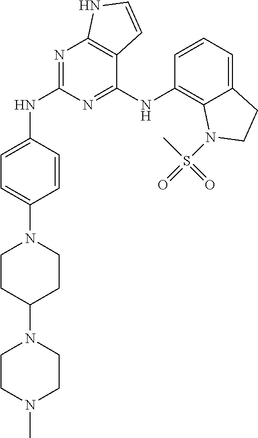 [SMILES: CN1CCN(C2CCN(c3ccc(Nc4nc(Nc5cccc6c5N(S(C)(=O)=O)CC6)c5cc[nH]c5n4)cc3)CC2)CC1]

The title compound was prepared in a procedure similar to that in Example 77. ¹H NMR (500 MHz, DMSO) δ_{H} 11.22 (s, 1H), 8.97 (s, 1H), 8.60 (s, 1H), 8.37 (d, J=8.1 Hz, 1H), 7.59 (d, J=9.0 Hz, 2H), 7.26-7.20 (m, 1H), 7.10 (d, J=7.3 Hz, 1H), 6.91 (dd, J=3.4, 2.3 Hz, 1H), 6.83 (d, J=9.1 Hz, 2H), 6.18 (dd, J=3.4, 1.9 Hz, 1H), 4.10 (t, J=7.4 Hz, 2H), 3.58 (d, J=12.3 Hz, 2H), 3.13 (t, J=7.4 Hz, 2H), 3.09 (s, 3H), 2.64-2.51 (m, 6H), 2.41-2.23 (m, 5H), 2.16 (s, 3H), 1.84 (d, J=11.6 Hz, 2H), 1.52 (dd, J=11.9, 3.4 Hz, 2H). [M+H]⁺=602.5.

### Example 195: N₂-(2-methoxy-4-(4-(4-methylpiperazin-1-yl)piperidin-1-yl)phenyl)—N₄-(1-(methylsulfonyl)indolin-7-yl)-5-(prop-1-yn-1-yl)pyrimidine-2,4-diamine

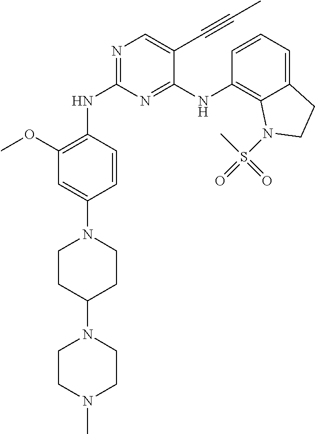 [SMILES: CC#Cc1cnc(Nc2ccc(N3CCC(N4CCN(C)CC4)CC3)cc2OC)nc1Nc1cccc2c1N(S(C)(=O)=O)CC2]

The title compound was prepared in a procedure similar to that in Example 28. ¹H NMR (500 MHz, DMSO) δ_{H} 9.02 (s, 1H), 8.05 (s, 1H), 7.98 (s, 1H), 7.91 (s, 1H), 7.49 (d, J=8.5 Hz, 1H), 7.12 (dd, J=14.2, 7.4 Hz, 2H), 6.60 (s, 1H), 6.42 (d, J=6.8 Hz, 1H), 4.05 (t, J=7.3 Hz, 2H), 3.76 (s, 3H), 3.69 (d, J=12.2 Hz, 2H), 3.09 (t, J=7.2 Hz, 2H), 3.05 (s, 3H), 2.64 (t, J=11.3 Hz, 2H), 2.49-2.42 (m, 4H), 2.38-2.24 (m, 5H), 2.14 (s, 3H), 2.08 (s, 3H), 1.84 (d, J=11.3 Hz, 2H), 1.51 (d, J=8.8 Hz, 2H). [M+H]⁺=631.5.

### Example 196: 5-ethynyl-N₂-(2-methoxy-4-(4-(4-methylpiperazin-1-yl)piperidin-1-yl)phenyl)—N₄-(1-(methylsulfonyl)indolin-7-yl)pyrimidine-2,4-diamine

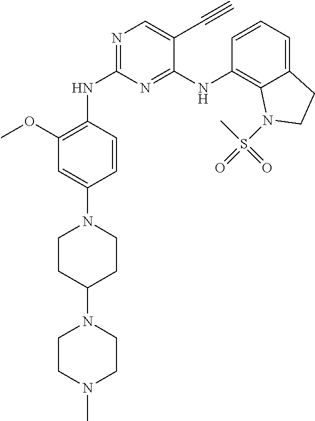 [SMILES: C#Cc1cnc(Nc2ccc(N3CCC(N4CCN(C)CC4)CC3)cc2OC)nc1Nc1cccc2c1N(S(C)(=O)=O)CC2]

The title compound was prepared in a procedure similar to that in Example 28. ¹H NMR (500 MHz, DMSO) δ_{H} 8.88 (s, 1H), 8.16 (s, 1H), 8.06 (s, 1H), 7.91 (s, 1H), 7.44 (d, J=8.2 Hz, 1H), 7.12 (s, 2H), 6.60 (s, 1H), 6.41 (d, J=8.6 Hz, 1H), 4.51 (s, 1H), 4.04 (s, 2H), 3.76 (s, 3H), 3.70 (d, J=11.2 Hz, 2H), 3.44 (d, J=6.0 Hz, 1H), 3.09 (s, 2H), 3.03 (s, 3H), 2.70-2.55 (m, 5H), 2.45-2.28 (m, 5H), 2.18 (s, 3H), 1.85 (d, J=10.7 Hz, 2H), 1.51 (d, J=10.8 Hz, 2H). [M+H]⁺=617.5.

### Example 197: 5-bromo-N₂-(2-methoxy-5-methyl-4-(4-(4-methylpiperazin-1-yl)piperidin-1-yl)phenyl)—N₄-(1-(methylsulfonyl)indolin-7-yl)pyrimidine-2,4,6-triamine

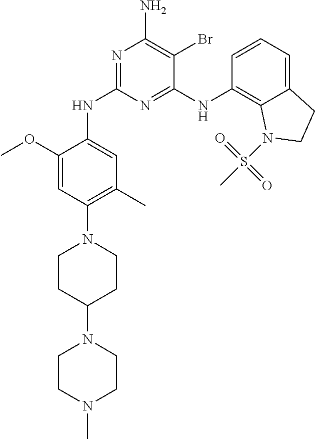 [SMILES: COc1cc(N2CCC(N3CCN(C)CC3)CC2)c(C)cc1Nc1nc(N)c(Br)c(Nc2cccc3c2N(S(C)(=O)=O)CC3)n1]

The title compound was prepared in a procedure similar to that in Example 28. ¹H NMR (500 MHz, DMSO) δ_{H} 8.54 (s, 1H), 8.18 (s, 1H), 7.75 (d, J=7.9 Hz, 1H), 7.71 (s, 1H), 7.16 (d, J=7.8 Hz, 1H), 7.11 (d, J=7.5 Hz, 2H), 6.65 (s, 1H), 6.47 (s, 2H), 4.05 (t, J=7.3 Hz, 2H), 3.78 (s, 3H), 3.11 (d, J=6.9 Hz, 2H), 3.06-3.00 (m, 6H), 2.62-2.55 (m, 3H), 2.40-2.26 (m, 7H), 2.16 (s, 3H), 2.06 (d, J=6.7 Hz, 3H), 1.83 (d, J=11.5 Hz, 2H), 1.54 (d, J=8.8 Hz, 2H). [M+H]⁺=700.5.

Biochemical EGFR Inhibition Assays

Compounds were tested for inhibition of kinase activity against EGFR (aa668-1210, Invitrogen), EGFR(L858R_T790M_C797S) (aa695-end, Invitrogen) and EGFR(Del19_T790M_C797S) (aa 669-1210, in house) in assays based on time-resolved fluorescence-resonance energy transfer (TR-FRET) methodology. Recombinant EGFR, EGFR(L858R_T790M_C797S) or EGFR(Del19_T790M_C797S) was pre-incubated with the compounds at room temperature for 15 minutes in an assay buffer containing 50 mM HEPES pH7.5, 10 mM MgCl₂, 2 mM DTT, 1 mM EGTA, 0.1% BSA, 0.01% Tween-20. The reactions were initiated by the addition of ATP (at the concentration of ATP Km) and substrate Biotin-Poly GT. After reaction at room temperature for 60 minutes, stop/detection solution was added. The stop/detection solution contained Eu³⁺ cryptate-conjugated mouse monoclonal antibody (PT66) anti-phosphotyrosine and XL665-conjugated streptavidin in buffer containing 50 mM HEPES pH7.0, 800 mM KF, 20 mM EDTA, and 0.1% BSA. Plates were sealed and incubated at room temperature for 1 hour, and the TR-FRET signals (ratio of fluorescence emission at 665 nm over emission at 620 nm with excitation at 337 nm wavelength) were recorded on a PHERAstar FS plate reader (BMG Labtech). The residual enzyme activity in presence of increasing concentrations of compounds was calculated based on the ratio of fluorescence at 665 nm to that at 620 nm. The IC50 for each compound was derived from fitting the data to the four-parameter logistic equation by Dotmatics or Graphpad Prism software.

These biochemical EGFR enzyme form compound dose-response assays quantify the kinase activity via phosphorylation of a tagged poly-GT substrate. The results of the assay are provided as IC50 values. The lower the reported IC50 values for a given compound, the more potent the compound inhibits the kinase activity of the EGFR enzyme on poly-GT substrate.

TABLE 1
Biochemical result for Example 1 to Example 197

| | | LR_TM_CS | Del_TM_CS |
|---|---|---|---|
| | WT (Km ATP) | Km ATP) | Km ATP) |
| Example | IC50 (nM) | IC50 (nM) | IC50 (nM) |
| 1 | 0.55 | 0.10 | 0.13 |
| 2 | 0.64 | 0.48 | 0.36 |
| 4 | 0.49 | 0.27 | 0.17 |
| 5 | 311 | 162 | 159 |
| 6 | 8.1 | 1.8 | 5.1 |
| 7 | 1.8 | 0.61 | 0.57 |
| 8 | 14 1 | 21 | 121 |
| 9 | 187 | 13 | 186 |
| 10 | 2.6 | 1.5 | 0.39 |
| 11 | 37 | 5.5 | 38 |
| 12 | 3.0 | 1.17 | 0.44 |
| 13 | 13 | 1.3 | 6.2 |
| 14 | 1.4 | 0.24 | 0.59 |
| 15 | 1365 | 369 | 2198 |
| 16 | 286 | 38 | 87 |
| 17 | 21 | 6.7 | 15 |
| 18 | 2.3 | 0.40 | 0.68 |
| 23 | 1.1 | 0.54 | 0.54 |
| 28 | 0.9 | 0.44 | 0.39 |
| 30 | 0.4 | 0.3 | 0.2 |
| 31 | 3.1 | 0.33 | 0.47 |
| 32 | 0.88 | 1.9 | 0.54 |
| 33 | 1.3 | 1.3 | 1.0 |
| 34 | 0.72 | 0.21 | 0.24 |
| 35 | 0.23 | 0.14 | 0.14 |
| 36 | 0.56 | 0.41 | 0.22 |
| 39 | 0.36 | 0.28 | 0.35 |
| 40 | 0.78 | 0.21 | 0.36 |
| 41 | 0.81 | 0.67 | 0.51 |
| 42 | 8.5 | 0.4 | 0.3 |
| 43 | 12.1 | 0.5 | 0.5 |
| 44 | 3.2 | 0.3 | 0.7 |
| 45 | 1.0 | 0.2 | 0.3 |
| 46 | 0.9 | 0.1 | 0.2 |
| 47 | 3.8 | 0.85 | 0.30 |
| 48 | 4.4 | 0.30 | 0.18 |
| 49 | 3.8 | 0.95 | 0.45 |
| 50 | 14 | 1.0 | 0.63 |
| 51 | 4.2 | 0.80 | 0.19 |
| 52 | 5.5 | 0.42 | 0.12 |
| 53 | 6.5 | 0.32 | 0.28 |
| 54 | 4.8 | 0.25 | 0.16 |
| 55 | 64.5 | 7.0 | 5.82 |
| 56 | 241.0 | 23.3 | 10.64 |
| 57 | 100.6 | 17.05 | 8.79 |
| 58 | 10.3 | 1.7 | 1.57 |
| 59 | 1.1 | 0.2 | 0.24 |
| 60 | 0.5 | 0.30 | 0.35 |
| 61 | 15.2 | 1.5 | 1.58 |
| 62 | 11.6 | 0.68 | 0.42 |
| 65 | 5.5 | 0.48 | 0.63 |
| 71 | 623 | 143 | 133 |
| 72 | 3.1 | 1.4 | 1.3 |
| 73 | 4.7 | 0.61 | 1.6 |
| 74 | 525 | 53 | 131 |
| 75 | 148 | 8.6 | 8.8 |
| 76 | 0.60 | 1.3 | 0.88 |
| 77 | 18 | 0.29 | 0.33 |
| 78 | 99.0 | 1.5 | 2.0 |
| 79 | 14 | 0.26 | 0.29 |
| 80 | 40.4 | 0.7 | 0.85 |
| 81 | 82.7 | 0.44 | 0.76 |
| 82 | 39.7 | 0.7 | 0.85 |
| 83 | 7292 | 42 | 37 |
| 84 | 150 | 1.5 | 1.6 |
| 85 | 1298 | 8.9 | 15 |
| 90 | 44 | 1.04 | 1.5 |
| 91 | 17 | 0.42 | 0.94 |
| 92 | 1526 | 17 | 14 |
| 93 | 110 | 1.5 | 2.6 |
| 94 | 29 | 1.1 | 1.5 |
| 95 | 28 | 0.79 | 1.4 |
| 96 | 109 | 1.4 | 1.7 |
| 97 | 206 | 2.4 | 4.3 |
| 98 | 22 | 0.56 | 0.96 |
| 99 | 427 | 3.0 | 8.6 |
| 100 | 49 | 0.92 | 1.0 |
| 101 | 170 | 2.0 | 3.1 |
| 102 | 66 | 1.1 | 2.0 |
| 103 | 28 | 0.47 | 0.78 |
| 104 | 332 | 3.4 | 5.3 |
| 105 | 455 | 5.3 | 10 |
| 106 | 575 | 5.3 | 4.9 |
| 107 | 417 | 4.8 | 7.1 |
| 108 | 104 | 1.9 | 2.2 |
| 109 | 433 | 3.1 | 10.01 |
| 110 | 571.6 | 5.1 | 10.74 |
| 111 | 145.8 | 2.8 | 11.1 |
| 112 | 840.1 | 2.9 | 14.59 |
| 113 | 1470 | 16 | 18.48 |
| 115 | 2494.6 | 20.7 | 42.81 |
| 123 | 573 | 4.8 | 6.3 |
| 124 | 107 | 1.0 | 1.0 |
| 125 | 106 | 1.2 | 1.6 |
| 126 | 611 | 20 | 59 |
| 134 | 11.5 | 0.3 | 0.8 |
| 136 | 1.4 | 0.3 | 0.9 |
| 143 | 1.6 | 0.4 | 0.2 |
| 147 | 471.6 | 1.0 | 1.6 |
| 148 | 1387.1 | 9.9 | 6.5 |
| 149 | 22.1 | 0.4 | 0.4 |
| 154 | 303.2 | 2.7 | 2.2 |
| 181 | 0.9 | 0.3 | 0.6 |
| 182 | 230.3 | 1.2 | 1.5 |
| 184 | 427.0 | 1.7 | 2.5 |
| 185 | 1.5 | 0.3 | 0.3 |
| 186 | 139.6 | 1.5 | 4.9 |
| 187 | 0.8 | 0.3 | 0.3 |
| 188 | 0.5 | 0.3 | 0.7 |
| 189 | 35.3 | 0.9 | 1.2 |
| 190 | 882.9 | 3.8 | 5.6 |
| 191 | 31.6 | 0.6 | 1.9 |
| 192 | 0.7 | 0.1 | 0.2 |
| 193 | 7.1 | 0.9 | 0.2 |
| 194 | 16.5 | 0.1 | 0.2 |
| 196 | 9.3 | 1.3 | 1.5 |
| 197 | 1.2 | 1.2 | 2.9 |

Cell Treatment

BaF3 cells are seeded at 5000 cells/well at a volume of 90 μl/well in cell culture medium (BaF3-WT cells need to be washed by PBS once to rinse IL-3) [RPMI1640(Gibco, Cat #2240089), 10% heat-inactive FBS (Gibco, #10099-141), 1% PS(Gibco, Cat #10378)] in Corning 96 well plate (Cat #3903). BaF3 cells are treated with compounds diluted in 0.2% DMSO, dilution is done according to the following protocol: (1) make 500×stock solution in DMSO from 5 mM by 5-fold dilution, total 8 doses were included; (2) make 10×solution in cell culture medium by transferring 2 μl 500×stock solution into 98 μl medium; (3) 10p of 10×solution is added to cells and incubate for 48h.

Cell Antiproliferation Assay

After 48h treatment, add 30 μl CellTiter-Glo reagent[CellTiter-Glo® 2.0(Promega, Cat #G9242)] to each well; seal the plate and incubate 2 min at room temperature on a plate shaker; Allow the plate to incubate at room temperature for 10 minutes to stabilize the luminescent signal. Record luminescence on BMG PheraStar with luminescence protocol.

The inhibition percentage of the compound was calculated by the following equation: Inhibition percentage of Compound=100-100× (Signal-low control)/(High control-low control), wherein signal=each test compound group

Low control=only medium group (without cells), indicating that cells proliferation are completely inhibited;

High control=Cell group with added DMSO and without compound, indicating cells proliferation with no inhibition;

Imax is the maximum percentage of inhibition.

The IC₅₀ value of a compound can be obtained by fitting the following equation

Y=Bottom+(TOP-Bottom)/(1+((IC₅₀/X)\hat{ }hillslope))

Wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the ability the inhibitory ability of the compound is; Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 2
Antiproliferation result for Example 1 to Example 126

| | BaF3-WT | BaF3-LR_TM_CS | BaF3-Del_TM_CS |
|---|---|---|---|
| Example | IC₅₀ (nM) | IC₅₀ (nM) | IC₅₀ (nM) |
| 1 | 634.6 | 151.7 | 48.16 |
| 2 | 510.8 | 66.14 | 14.6 |
| 4 | 465.7 | 57.23 | 12.04 |
| 5 | 2315.7 | 1287.5 | 970.6 |
| 6 | 1851.8 | 221.5 | 233.2 |
| 7 | 457.3 | 81.61 | 53.87 |
| 8 | 1861.3 | 730.1 | 630.1 |
| 9 | 1635.8 | 427.8 | 455.4 |
| 10 | 1865.9 | 143.2 | 45.99 |
| 11 | 2279.5 | 376.9 | 453.2 |
| 13 | 896.7 | 293.7 | 157.9 |
| 14 | 192.6 | 14.26 | 8.76 |
| 15 | 4120.8 | 2026.4 | 1795.0 |
| 16 | 1978.5 | 1121.0 | 802.0 |
| 17 | 1713.4 | 366.0 | 183.0 |
| 18 | 422.4 | 16.49 | 7.47 |
| 23 | 453.0 | 38.36 | 13.26 |
| 30 | 410.7 | 37.32 | 7.59 |
| 31 | 522.3 | 109.2 | 26.19 |
| 32 | 569.7 | 246.2 | 62.79 |
| 33 | 328.2 | 224.7 | 68.46 |
| 34 | 427.3 | 12.95 | 4.25 |
| 35 | 604.1 | 222.1 | 79.94 |
| 36 | 414.4 | 25.85 | 10.04 |
| 39 | 500.3 | 11.43 | 4.54 |
| 40 | 229.8 | 4.91 | 1.56 |
| 41 | 609.6 | 15.39 | 6.06 |
| 47 | 630.4 | 37.77 | 12.33 |
| 48 | 361.2 | 30.5 | 3.56 |
| 49 | 1511.8 | 79.32 | 19.31 |
| 50 | 2734.9 | 175.5 | 18.97 |
| 51 | 234.8 | 35.9 | 10.11 |
| 52 | 144.7 | 18.68 | 11.25 |
| 54 | 372.0 | 42.36 | 18.58 |
| 71 | 2209.7 | 1467.9 | 920.3 |
| 72 | 2599.0 | 2201.7 | 246.3 |
| 73 | 853.0 | 141.1 | 27.38 |
| 74 | 2347.0 | 616.5 | 436.0 |
| 75 | 1202.7 | 459.3 | 349.3 |
| 76 | 986.9 | 349.6 | 90.49 |
| 77 | 387.7 | 61.99 | 8.3 |
| 83 | 1859.3 | 1044.9 | 706.7 |
| 84 | 335.3 | 78.04 | 10.73 |
| 85 | 274.5 | 141.0 | 91.59 |
| 90 | 2558.8 | 191.3 | 87.53 |
| 91 | 949.3 | 40.46 | 12.83 |
| 92 | 1398.8 | 504.4 | 225.2 |
| 93 | 1518.9 | 217.9 | 54.15 |
| 94 | 640.2 | 84.84 | 10.93 |
| 95 | 1957.2 | 83.75 | 9.58 |
| 96 | 1482.9 | 66.46 | 29.26 |
| 97 | 1926.6 | 240.3 | 104.2 |
| 98 | 1463.5 | 76.55 | 24.71 |
| 99 | 1967.1 | 399.3 | 59.01 |
| 100 | 1589.8 | 116.3 | 39.92 |
| 101 | 1409.4 | 156.8 | 58.92 |
| 102 | 1709.2 | 314.2 | 30.4 |
| 103 | 1688.0 | 76.75 | 25.61 |
| 104 | 1491.5 | 195.2 | 128.0 |
| 105 | 2089.3 | 239.6 | 87.03 |
| 106 | 1789.0 | 93.82 | 57.25 |
| 107 | 1961.4 | 105.1 | 60.78 |
| 108 | 2140.9 | 464.5 | 180.6 |
| 123 | 375.9 | 169.2 | 64.33 |
| 124 | 460.2 | 106.2 | 39.13 |
| 125 | 393.6 | 61.35 | 38.3 |
| 126 | 1925.2 | 539.9 | 462.8 |

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

1. A compound of Formula (I): 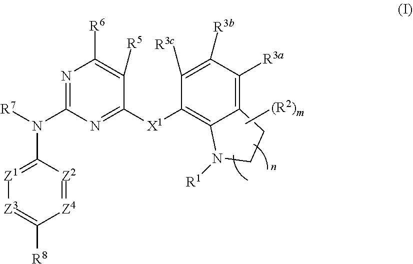
or an N-oxide thereof or a pharmaceutically acceptable salt thereof or a stereoisomer thereof or prodrug thereof, wherein:
X¹ is a single bond, NR⁴, O, S, S(O), S(O)₂ or CH₂;
Z¹ is N or CR⁹, Z² is N or CR¹⁰, Z³ is N or CR¹¹, Z⁴ is N or CR¹²;
R¹ is —S(O)R^{1a}, —S(O)₂R^{1a}, —C(O)R^{1a}, —P(O)R^{1a}R^{1b} or 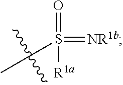
R^{1a} and R^{1b} are each independently H, —C₁₋₈alkyl, —C₂₋₈alkenyl, —C₂₋₈alkynyl, cycloalkyl, heterocyclyl, aryl, heteroaryl, —CN, —OR^{1d}, —CH₂CONR^{1d}R^{1e}, —CH₂CH₂CONR^{1d}R^{1e}, —CH₂CH₂CH₂CONR^{1d}R^{1e}, —NR^{1d}R^{1e}, —CH₂NR^{1d}R^{1e}, —CH₂CH₂NR^{1d}R^{1e}, —CH₂CH₂CH₂NR^{1d}R^{1e} or —NR^{1d}COR^{1e}, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{1f};
R^{1d} and R^{1e} are each independently hydrogen, —C₁₋₈alkyl, -haloC₁₋₈alkyl, —C₂₋₈alkenyl, —C₂₋₈alkynyl, cycloalkyl, heterocyclyl, aryl or heteroaryl; or
R^{1d} and R^{1e} together with the atom(s) to which they are attached, form a 3- to 12-membered ring, said ring comprising 0, 1 or 2 additional heteroatom(s) independently selected from the group consisting of nitrogen, oxygen and optionally oxidized sulfur as ring member(s), said ring is optionally substituted with at least one substituent R¹;
R^{1f}, at each of its occurrence, is independently hydrogen, halogen, —C₁₋₈alkyl, -haloC₁₋₈alkyl, —C₂₋₈alkenyl, —C₂₋₈alkynyl, cycloalkyl, halocycloalkyl, heterocyclyl, aryl, heteroaryl, oxo (═O), —CN, —OR^{1g}, —COR^{1g}, —CO₂R^{1g}, —CONR^{1g}R^{1h}, —NR^{1g}R^{1h}, —NR^{1g}COR^{1h} or —NR^{1g}CO₂R^{1h}; or
two R^{1f} together with the atom(s) to which they are attached, form a 3- to 12-membered ring, said ring comprising 0, 1 or 2 additional heteroatom(s) independently selected from the group consisting of nitrogen, oxygen and optionally oxidized sulfur as ring member(s), said ring is optionally substituted with at least one substituent hydrogen, halogen, hydroxyl, —C₁₋₈alkyl, -haloC₁₋₈alkyl, —C₂₋₈alkenyl, —C₂₋₈alkynyl, cycloalkyl, heterocyclyl, aryl, heteroaryl, oxo or —CN;
R^{1g} and R^{1h} are each independently hydrogen, halogen, hydroxyl, —C₁₋₈alkyl, -haloC₁₋₈alkyl, —C₂₋₈alkenyl, —C₂₋₈alkynyl, cycloalkyl, heterocyclyl, aryl or heteroaryl;
R², R^{3a}, R^{3b} and R^{3c} are each hydrogen, halogen, —C₁₋₈alkyl, cycloalkyl, heterocyclyl, aryl, heteroaryl, —CN, oxo (═O), —OR^{2a}, —COR^{2a}, —CO₂R^{2a}, —CONR^{2a}R^{2b}, —NR^{2a}R^{2b}, —NR^{2a}COR^{2b} or —NR^{2a}CO₂R^{2b}, wherein each of said —C₁₋₈alkyl, cycloalkyl, heterocyclyl, aryl or heteroaryl is optionally substituted with at least one substituent R^{2c}; or
R² and R^{3a} together with the atoms to which they are attached, form a 3- to 12-membered ring, said ring comprising 0, 1 or 2 additional heteroatom(s) independently selected from the group consisting of nitrogen, oxygen and optionally oxidized sulfur as ring member(s), said ring is optionally substituted with at least one substituent R^{2c}; or
when m≥2, two germinal R² together with the atom to which they are attached, form a 3-to 12-membered spiro ring, or two R² together with the atoms to which they are attached, form a 3- to 12-membered ring, said ring comprising 0, 1 or 2 additional heteroatom(s) independently selected from the group consisting of nitrogen, oxygen and optionally oxidized sulfur as ring member(s), said ring is optionally substituted with at least one substituent R^{2c}; or
R^{3a} and R^{3b} or R^{3b} and R^{3c} together with the atoms to which they are attached, form a 3-to 12-membered ring, said ring comprising 0, 1 or 2 additional heteroatom(s) independently selected from the group consisting of nitrogen, oxygen and optionally oxidized sulfur as ring member(s), said ring is optionally substituted with at least one substituent R^{2c};
R^{2a} and R^{1b} are each independently hydrogen, —C₁₋₈alkyl, —C₂₋₈alkenyl, —C₂₋₈alkynyl, C₁₋₈alkoxy-C₁₋₈alkyl-, cycloalkyl, heterocyclyl, aryl or heteroaryl, wherein each of said —C₁₋₈alkyl, —C₂₋₈alkenyl, —C₂₋₈alkynyl, C₁₋₈alkoxy-C₁₋₈alkyl-, cycloalkyl, heterocyclyl, aryl or heteroaryl is optionally substituted with at least one substituent R^{2e};
R^{2c}, at each of its occurrence, is independently halogen, amino, —C₁₋₈alkyl, —C₂₋₈alkenyl, —C₂₋₈alkynyl, cycloalkyl, heterocyclyl, aryl, heteroaryl, oxo (═O), —CN, —OR^{2d}, —COR^{2d}, —CO₂R^{2d}, —CONR^{2d}R^{2e}, —NR^{2d}R^{2e}, —NR^{2d}COR^{2e}, or —NR^{2d}CO₂R^{2e};
R^{2d} and R^{2e} are each independently hydrogen, —C₁₋₈alkyl, -haloC₁₋₈alkyl, —C₂₋₈alkenyl, —C₂₋₈alkynyl, cycloalkyl, heterocyclyl, aryl or heteroaryl;
R⁴ and R⁷ are each independently hydrogen, —C₁₋₈alkyl, —C₂₋₈alkenyl, —C₂₋₈alkynyl or cycloalkyl, wherein each of said —C₁₋₈alkyl, —C₂₋₈alkenyl, —C₂₋₈alkynyl or cycloalkyl is optionally substituted with at least one substituent R^{4a};
R^{4a} is independently hydrogen, halogen, —C₁₋₈alkyl, -haloC₁₋₈alkyl, —C₂₋₈alkenyl, —C₂₋₈alkynyl, cycloalkyl, —CN or —OR^{4b};
R^{4b} is hydrogen, —C₁₋₈alkyl, -haloC₁₋₈alkyl, C₁₋₈alkoxy-C₁₋₈alkyl- or —C₃₋₆cycloalkyl;
R⁵ and R⁶ are independently hydrogen, halogen, —C₁₋₈alkyl, —C₂₋₈alkenyl, —C₂₋₈alkynyl, cycloalkyl, heterocyclyl, aryl, heteroaryl, —CN, —OR^{5a}, —COR^{5a}, —CO₂R^{5a}, —CONR^{5a}R^{5b}, —NR^{5a}R^{5b}or —NR^{5a}COR^{5b}, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{5c};
R^{5a} are R^{5b} are each independently hydrogen, —C₁₋₈alkyl, —C₂₋₈alkenyl, —C₂₋₈alkynyl, C₁₋₈alkoxy-C₁₋₈alkyl-, cycloalkyl, heterocyclyl, aryl or heteroaryl, wherein each of said —C₁₋₈alkyl, —C₂₋₈alkenyl, —C₂₋₈alkynyl, C₁₋₈alkoxy-C₁₋₈alkyl-, cycloalkyl, heterocyclyl, aryl or heteroaryl is optionally substituted with at least one substituent R^{5c};
R^{5c}, at each occurrence, is independently halogen, —C₁₋₈alkyl, -haloC₁₋₈alkyl, —C₂₋₈alkenyl, —C₂₋₈alkynyl, cycloalkyl, heterocyclyl, aryl or heteroaryl;
or R⁵ and R⁶, together with the atoms to which they are attached, form a 3- to 12-membered ring, said ring comprising 0, 1 or 2 additional heteroatom(s) independently selected from the group consisting of nitrogen, oxygen, sulfur and optionally oxidized sulfur as ring member(s), said ring is optionally substituted with at least one substituent R⁵⁶;
R⁵⁶ is hydrogen, halogen, —C₁₋₈alkyl, -haloC₁₋₈alkyl, —C₂₋₈alkenyl, —C₂₋₈alkynyl, —C₃₋₆cycloalkyl, 3- to 8-membered heterocyclyl, —C₆₋₁₂aryl, 3- to 8-membered heteroaryl, C₁₋₈alkoxy-C₁₋₈alkyl-, oxo (═O), —CN, —OR^{56a}, —COR^{56a}, —CO₂R^{56a}, —CONR^{56a}R^{56b}, —NR^{56a}R^{56b} or —NR^{56a}COR^{56b};
R^{56a} and R^{56b} are each independently hydrogen, —C₁₋₈alkyl, -haloC₁₋₈alkyl, —C₂₋₈alkenyl, —C₂₋₈alkynyl, C₁₋₈alkoxy-C₁₋₈alkyl-, cycloalkyl, heterocyclyl, aryl or heteroaryl;
R⁸ is halogen, —C₁₋₈alkyl, —C₂₋₈alkenyl, —C₂₋₈alkynyl, cycloalkyl, heterocyclyl, aryl, heteroaryl, —OR^{8a} or —NR^{8a}R^{8b}, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{8c};
R^{8a} and R^{8b} are each independently hydrogen, —C₁₋₈alkyl, —C₂₋₈alkenyl, —C₂₋₈alkynyl, C₁₋₈alkoxy-C₁₋₈alkyl-, cycloalkyl, heterocyclyl, aryl or heteroaryl, wherein each of said —C₁₋₈alkyl, —C₂₋₈alkenyl, —C₂₋₈alkynyl, C₁₋₈alkoxy-C₁₋₈alkyl-, cycloalkyl, heterocyclyl, aryl or heteroaryl is optionally substituted with at least one substituent R^{8d}; or
R^{8a} and R^{8b}, together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen and optionally oxidized sulfur as ring member(s), said ring is optionally substituted with at least one substituent R^{8d};
R^{8c} is independently hydrogen, halogen, —C₁₋₈alkyl, —C₂₋₈alkenyl, —C₂₋₈alkynyl, cycloalkyl, heterocyclyl, aryl, heteroaryl, oxo (═O), —CN, —OR^{8f}, —COR^{8f}, —CO₂R^{8f}, —CONR^{8f}R^{8g}, —NR^{8f}R^{8g} or —NR^{8f}COR^{8g}, wherein each of said —C₁₋₈alkyl, —C₂₋₈alkenyl, —C₂₋₈alkynyl, cycloalkyl, heterocyclyl, aryl or heteroaryl is optionally substituted with at least one R^{8e}; or
two R^{8c} together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen and optionally oxidized sulfur as ring member(s), said ring is optionally substituted with at least one R^{8e};
R^{8d} and R^{8e} are each independently hydrogen, halogen, —C₁₋₈alkyl, —C₂₋₈alkenyl, —C₂₋₈alkynyl, cycloalkyl, heterocyclyl, aryl, heteroaryl, oxo (═O), —CN, —OR^{8h}, —COR^{8h}, —CO₂R^{8h}, —CONR^{8h}R⁸ⁱ, —NR^{8h}R⁸ⁱ or —NR^{8h}COR⁸ⁱ, wherein each of said —C₁₋₈alkyl, —C₂₋₈alkenyl, —C₂₋₈alkynyl, cycloalkyl, heterocyclyl, aryl or heteroaryl is optionally substituted with at least one halogen, —C₁₋₈alkyl, —C₂₋₈alkenyl, —C₂₋₈alkynyl, cycloalkyl, heterocyclyl, aryl, heteroaryl, hydroxyl, —C₁₋₈alkoxy or C₁₋₈alkoxy-C₁₋₈alkyl-; or
two R^{8e} together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen and optionally oxidized sulfur as ring member(s), said ring is optionally substituted with at least one halogen, —C₁₋₈alkyl, —C₂₋₈alkenyl, —C₂₋₈alkynyl, cycloalkyl, heterocyclyl, aryl, heteroaryl, hydroxyl, —C₁₋₈alkoxy or C₁₋₈alkoxy-C₁₋₈alkyl-;
R^{8f}, R^{8g}, R^{8h} and R⁸ⁱ are each independently hydrogen, —C₁₋₈alkyl, -haloC₁₋₈alkyl, C₁₋₈alkoxy-C₁₋₈alkyl-, —C₂₋₈alkenyl, —C₂₋₈alkynyl, cycloalkyl, heterocyclyl, aryl or heteroaryl;
R⁹, R¹⁰, R¹¹ and R¹² are each independently hydrogen, halogen, —C₁₋₈alkyl, —C₂₋₈alkenyl, —C₂₋₈alkynyl, cycloalkyl, heterocyclyl, —C₆₋₁₂aryl, heteroaryl, —CN, —OR^{9d}, —CH₂CONR^{9d}R^{9e}, —CH₂CH₂CONR^{9d}R^{9e}, —CH₂CH₂CH₂CONR^{9d}R^{9e}, —NR^{9d}R^{9e}, —CH₂NR^{9d}R^{9e}, —CH₂CH₂NR^{9d}R^{9e}, —CH₂CH₂CH₂NR^{9d}R^{9e} or —NR^{9d}COR^{9e}, wherein each of said —C₁₋₈alkyl, —C₂₋₈alkenyl, —C₂₋₈alkynyl, cycloalkyl, heterocyclyl, aryl or heteroaryl is optionally substituted with at least one substituent R^{9f}; or
R⁹ and R¹¹, or R¹⁰ and R¹², together with the atom(s) to which they are attached, form a 3- to 12-membered ring, said ring comprising 0, 1 or 2 additional heteroatom(s) independently selected from the group consisting of nitrogen, oxygen and optionally oxidized sulfur as ring member(s), said ring is optionally substituted with at least one substituent R^{9f};
R^{9d} and R^{9e} are each independently hydrogen, —C₁₋₈alkyl, —C₂₋₈alkenyl, —C₂₋₈alkynyl, cycloalkyl, heterocyclyl, aryl, or heteroaryl, wherein each of said —C₁₋₈alkyl, —C₂₋₈alkenyl, —C₂₋₈alkynyl, cycloalkyl, heterocyclyl, aryl or heteroaryl is optionally substituted with at least one halogen, —C₁₋₈alkyl, -haloC₁₋₈alkyl, —C₂₋₈alkenyl, —C₂₋₈alkynyl, cycloalkyl, heterocyclyl, aryl, heteroaryl, hydroxyl, —C₁₋₈alkoxy or C₁₋₈alkoxy-C₁₋₈alkyl-; or
R^{9d} and R^{9e} together with the atom(s) to which they are attached, form a 3- to 12-membered ring, said ring comprising 0, 1 or 2 additional heteroatom(s) independently selected from the group consisting of nitrogen, oxygen and optionally oxidized sulfur as ring member(s), said ring is optionally substituted with at least one substituent R^{9f},
R^{9f}, at each of its occurrence, is independently hydrogen, halogen, -haloC₁₋₈alkyl, —C₁₋₈alkyl, —C₂₋₈alkenyl, —C₂₋₈alkynyl, cycloalkyl, aryl, heteroaryl, oxo (═O), —CN, —OR^{9g}, —COR^{9g}, —CO₂R^{9g}, —CONR^{9g}R^{9h}, —NR^{9g}COR^{9h} or —NR^{9g}CO₂R^{9h}; or
two R^{9f} together with the atom(s) to which they are attached, form a 3- to 12-membered ring, said ring comprising 0, 1 or 2 additional heteroatom(s) independently selected from the group consisting of nitrogen, oxygen and optionally oxidized sulfur as ring member(s), said ring is optionally substituted with at least one halogen, —C₁₋₈alkyl, -haloC₁₋₈alkyl, —C₂₋₈alkenyl, —C₂₋₈alkynyl, cycloalkyl, heterocyclyl, aryl, heteroaryl, hydroxyl, —C₁₋₈alkoxy or C₁₋₈alkoxy-C₁₋₈alkyl-;
R^{9g} and R^{9h} are each independently hydrogen, halogen, hydroxyl, —C₁₋₈alkyl, -haloC₁₋₈alkyl, —C₂₋₈alkenyl, —C₂₋₈alkynyl, cycloalkyl, heterocyclyl, aryl or heteroaryl;
n is 0, 1, 2 or 3; and
m is 0, 1, 2, 3 or 4.

2. The compound of claim 1, wherein
R¹ is —S(O)₂R^{1a}, —C(O)R^{1a} or 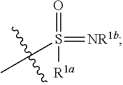
R^{1a} and R^{1b} are each independently H, methyl, ethyl, propyl, butyl, pentyl, hexyl, heptyl, octyl, cyclopropyl, cyclobutyl, cyclopentyl, cyclohexyl, cycloheptyl, cyclooctyl, azacyclopropanyl, azacyclobutanyl, tetrahydropyrrole, piperidinyl, piperazinyl, morphinyl, epoxyethyl, epoxybutanyl, oxacyclopentanyl, tetrahydropyran, phenyl, pyrrolyl, imidazolyl, pyrazolyl, thiazolyl, thienyl, oxazolyl, pyridinyl, indolyl, quinolinyl, isoquinolinyl, benzimidazolyl, benzothiazolyl, benzopyrazolyl, —CH₂CONR^{1d}R^{1e}, —CH₂CH₂CONR^{1d}R^{1e}, —CH₂CH₂CH₂CONR^{1d}R^{1e}, —NR^{1d}R^{1e}, —CH₂NR^{1d}R^{1e}, —CH₂CH₂NR^{1d}R^{1e} or —CH₂CH₂CH₂NR^{1d}R^{1e}, wherein each of said methyl, ethyl, propyl, butyl, pentyl, hexyl, heptyl, octyl, cyclopropyl, cyclobutyl, cyclopentyl, cyclohexyl, cycloheptyl, cyclooctyl, azacyclopropanyl, azacyclobutanyl, tetrahydropyrrolyl, piperidinyl, piperazinyl, morphinyl, epoxyethyl, epoxybutanyl, oxacyclopentanyl, tetrahydropyran, phenyl, pyrrolyl, imidazolyl, pyrazolyl, thiazolyl, thienyl, oxazolyl, pyridinyl, indolyl, quinolinyl, isoquinolinyl, benzimidazolyl, benzothiazolyl or benzopyrazolyl is optionally substituted with at least one substituent R^{1f};
R^{1d} and R^{1e} are each independently hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, azacyclopropanyl, azacyclobutanyl, tetrahydropyrrolyl, piperidinyl, piperazinyl, morphinyl, epoxyethyl, epoxybutanyl, oxacyclopentanyl, tetrahydropyran, phenyl, pyrrolyl, imidazolyl, pyrazolyl, thiazolyl, thienyl, oxazolyl, pyridinyl, indolyl, quinolinyl, isoquinolinyl, benzimidazolyl, benzothiazolyl or benzopyrazolyl; or
R^{1f}, at each of its occurrence, is independently hydrogen, —F, —Cl, —Br, —I, hydroxyl, methyl, ethyl, propyl, butyl, pentyl, hexyl, heptyl, octyl, cyclopropyl, cyclobutyl, cyclopentyl, cyclohexyl, cycloheptyl, cyclooctyl, azacyclopropanyl, azacyclobutanyl, tetrahydropyrrolyl, piperidinyl, piperazinyl, morphinyl, epoxyethyl, epoxybutanyl, oxacyclopentanyl, tetrahydropyran, phenyl, pyrrolyl, imidazolyl, pyrazolyl, thiazolyl, thienyl, oxazolyl, pyridinyl, indolyl, quinolinyl, isoquinolinyl, benzimidazolyl, benzothiazolyl, benzopyrazolyl or —CN, or
two R^{1f} together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen and optionally oxidized sulfur as ring member(s), said ring is optionally substituted with at least one substituent hydrogen, F, Cl, Br, I, hydroxyl, methyl, ethyl, propyl, butyl, pentyl, hexyl, heptyl, octyl, cyclopropyl, cyclobutyl, cyclopentyl, cyclohexyl, cycloheptyl, cyclooctyl, azacyclopropanyl, azacyclobutanyl, tetrahydropyrrolyl, piperidinyl, piperazinyl, morphinyl, epoxyethyl, epoxybutanyl, oxacyclopentanyl, tetrahydropyran, phenyl, pyrrolyl, imidazolyl, pyrazolyl, thiazolyl, thienyl, oxazolyl, pyridinyl, indolyl, quinolinyl, isoquinolinyl, benzimidazolyl, benzothiazolyl, benzopyrazolyl, oxo or —CN;
R^{1g} and R^{1h} are each independently hydrogen, F, Cl, Br, I, hydroxyl, methyl, ethyl, propyl, butyl, pentyl, hexyl, heptyl, octyl, cyclopropyl, cyclobutyl, cyclopentyl, cyclohexyl, cycloheptyl, cyclooctyl, azacyclopropanyl, azacyclobutanyl, tetrahydropyrrolyl, piperidinyl, piperazinyl, morphinyl, epoxyethyl, epoxy butane, oxacyclopentanyl, tetrahydropyran, phenyl, pyrrolyl, imidazolyl, pyrazolyl, thiazolyl, thienyl, oxazolyl, pyridinyl, indolyl, quinolinyl, isoquinolinyl, benzimidazolyl, benzothiazolyl or benzopyrazolyl.

3. The compound of claim 1, wherein
R¹ is S(O)₂R^{1a}, —C(O)R^{1a} or 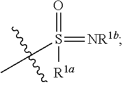
R^{1a} is —CH₃, —C₂H₅, —C₃H₇, —C₄H₉, —C₅H₁₁, -cyclopropyl, -tert-butyl, 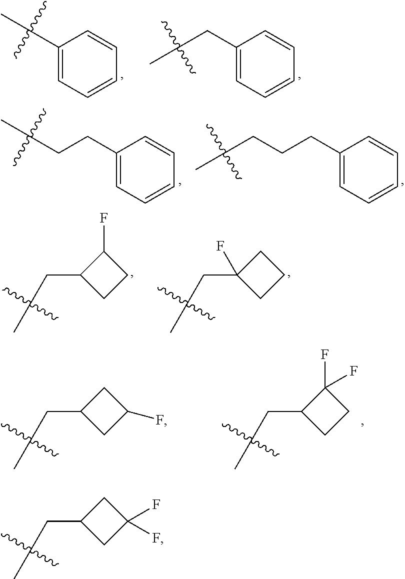
—CH₂F, —CHF₂, —CF₃, —N(CH₃)₂, —NHCH₃, —CH₂N(CH₃)₂, —CH₂CH₂N(CH₃)₂ or —CH₂CH₂CH₂N(CH₃)₂.

4. The compound of claim 1, wherein
R², R^{3a}, R^{3b} and R^{3c}, at each of their occurrences, are hydrogen, —F, —Cl, —Br, —I, —C₁₋₈alkyl, C₃₋₈cycloalkyl, —CN, oxo (═O), —OR^{2a} or —COR^{2a}, wherein each of said-C₁₋₈alkyl, C₃₋₈cycloalkyl, 3- to 8-membered heterocyclyl, C₆-C₁₂aryl or 5- to 12-membered heteroaryl is optionally substituted with at least one substituent R^{2c},
R² and R^{3a}, two R², R^{3a} and R^{3b}, or R^{3b} and R^{3c}, together with the atom(s) to which they are attached, form a 3- to 8-membered ring, said ring comprising 0, 1 or 2 additional heteroatom(s) independently selected from the group consisting of nitrogen, oxygen and optionally oxidized sulfur as ring member(s), said ring is optionally substituted with at least one substituent R^{2c};
R^{2a} is independently hydrogen, —C₁₋₈alkyl, —C₂₋₈alkenyl, —C₂₋₈alkynyl, C₁₋₈alkoxy-C₁₋₈alkyl-, C₃₋₈cycloalkyl, 3- to 8-membered heterocyclyl, C₆₋₁₂aryl or 5- to 12-membered heteroaryl, wherein each of said —C₁₋₈alkyl, —C₂₋₈alkenyl, —C₂₋₈alkynyl, C₁₋₈alkoxy-C₁₋₈alkyl- or C₃₋₈cycloalkyl is optionally substituted with at least one substituent R^{2c};
R^{2c}, at each of its occurrence, is independently —F, —Cl, —Br, —I, hydroxyl, —NH₂, —CH₃, —C₂H₅, —C₃H₇, —C₄H₉, —C₅H₁₁, —C₆H₁₃, —C₇H₁₅, —C₅H₁₇, phenyl, oxo (═O), —CN, —OCH₃, —OC₂H₅, —OC₃H₇, —OC₄H₉, —OC₅H₁₁, —OC₆H₁₃, —OC₇H₁₅, —OC₈H₁₇, —COCH₃, —COC₂H₅, —COC₃H₇, —COC₄H₉, —COC₅H₁₁, —COC₆H₁₃, —COC₇H₁₅, —COC₈H₁₇, —CO₂CH₃, —CO₂C₂H₅, —CO₂C₃H₇, —CO₂C₄H₉, —CO₂C₅H₁₁, —CO₂C₆H₁₃, —CO₂C₇H₁₅ or —CO₂C₈H₁₇.

5. The compound of claim 1, wherein
R² is hydrogen, —F, —Cl, —Br, —I, —CH₃, —C₂H₅, —C₃H₇, —C₄H₉, —C₅H₁₁, —C₆H₁₃, —C₇H₁₅, —C₈H₁₇, —CH₂OH, —CH₂CH₂OH, —CH₂CH₂CH₂OH, or oxo; or
when m≥2, two germinal R² together with the atom to which they are attached, form a 3-, 4-, 5- or 6-membered spiro ring, said ring comprising 0, 1 or 2 additional heteroatom(s) independently selected from the group consisting of nitrogen, oxygen and optionally oxidized sulfur as ring member(s).

6. The compound of claim 1, wherein
R² is hydrogen, F, Cl, Br, I, —CH₃, —C₂H₅, —C₃H₇, —C₄H₉, —C₅H₁₁, —CH₂OH, —CH₂CH₂OH, —CH₂CH₂CH₂OH or oxo; or
when m≥2, two germinal R² together with the atom to which they are attached, form a spiro cyclopropyl or spiro cyclobutyl.

7. The compound of claim 1, wherein
R^{3a}, R^{3b} and R^{3c} are each independently hydrogen, F, Cl, Br, I, -methyl, ethyl, propyl, butyl, pentyl, hexyl, heptyl, octyl, cyclopropyl, cyclobutyl, cyclopentyl, cyclohexyl, cycloheptyl, cyclooctyl, —CN, —OR^{2a}, —COR^{2a} or —CO₂R^{2a}, wherein each of said methyl, ethyl, propyl, butyl, pentyl, hexyl, heptyl, octyl, cyclopropyl, cyclobutyl, cyclopentyl, cyclohexyl, cycloheptyl or cyclooctyl is optionally substituted with at least one substituent R^{2c},
R^{2a} is each independently hydrogen, —C₁₋₈alkyl, —C₂₋₈alkenyl, —C₂₋₈alkynyl, C₁₋₈alkoxy-C₁₋₈alkyl- or C₃₋₈cycloalkyl, wherein each of said —C₁₋₈alkyl, —C₂₋₈alkenyl, —C₂₋₈alkynyl, C₁₋₈alkoxy-C₁₋₈alkyl- or C₃₋₈cycloalkyl is optionally substituted with at least one substituent R^{2c};
R^{2c}, at each of its occurrence, is independently hydroxyl, halogen, —C₁₋₈alkyl, —C₂₋₈alkenyl, —C₂₋₈alkynyl or —C₃₋₈cycloalkyl.

8. The compound of claim 1, wherein
R^{3a}, R^{3b} and R^{3c} are each independently —H, —F, —Cl, —Br, —I, hydroxyl, amino, —CH₃, —C₂H₅, —C₃H₇, —C₄H₉, —C₅H₁₁, —C₆H₁₃, —C₇H₁₅, —C₈H₁₇, phenyl, oxo (═O), —CN, —OCH₃, —OC₂H₅, —OC₃H₇, —OC₄H₉, —OC₅H₁₁, —OC₆H₁₃, —OC₇H₁₅, —OC₈H₁₇, —COCH₃, —COC₂H₅, —COC₃H₇, —COC₄H₉, —COC₅H₁₁, —COC₆H₁₃, —COC₇H₁₅, —COC₈H₁₇, —CO₂CH₃, —CO₂C₂H₅, —CO₂C₃H₇, —CO₂C₄H₉, —CO₂C₅H₁₁, —CO₂C₆H₁₃, —CO₂C₇H₁₅ or —CO₂C₈H₁₇.

9. The compound of claim 1, wherein

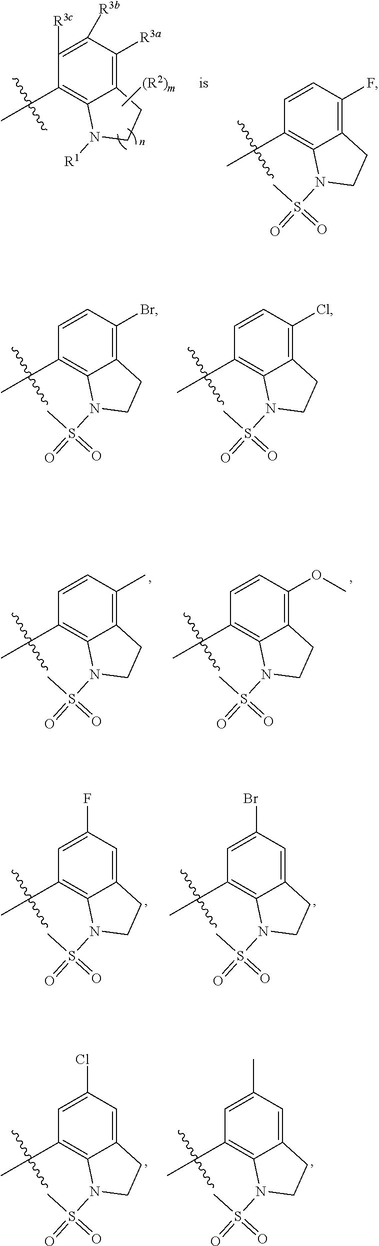

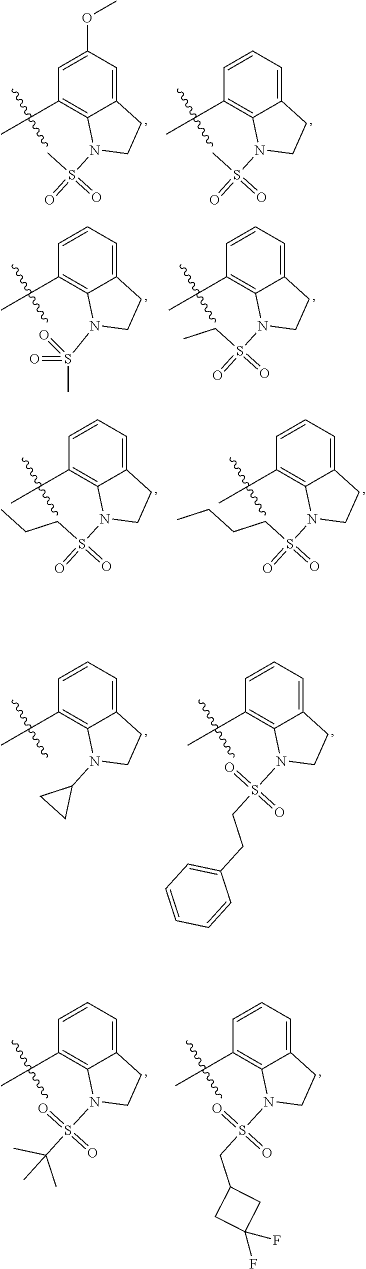

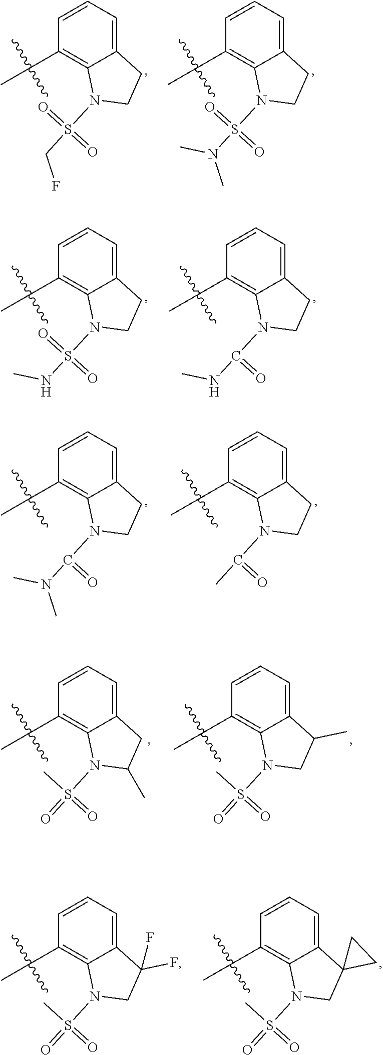

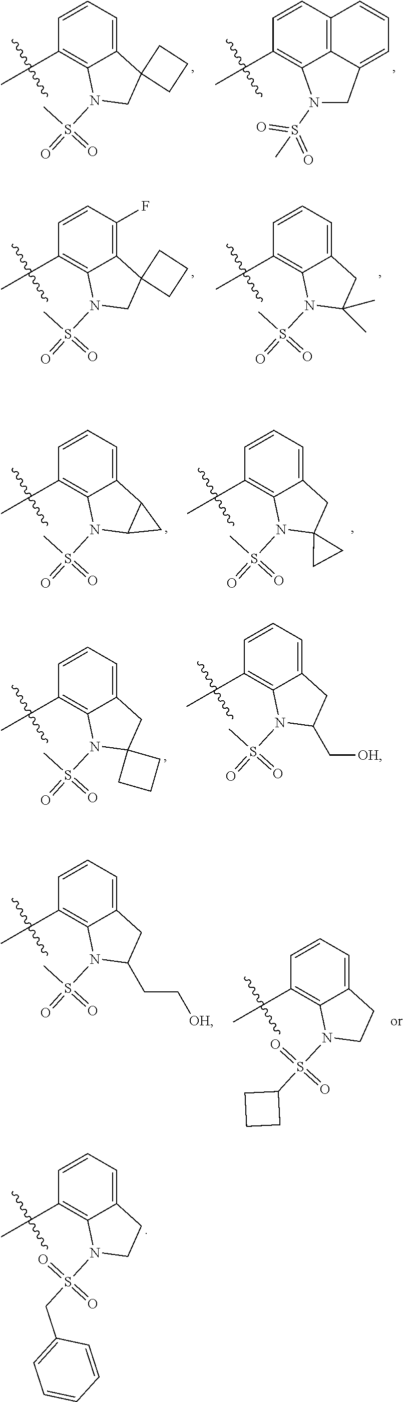

10. The compound of claim 1, wherein
R₄ and R₇ are each independently —H, —CH₃, —C₂H₅, —C₃H₇, —C₄H₉, —C₅H₁₁, —C₆H₁₃, —C₇H₁₅ or —C₈H₁₇.

11. The compound of claim 1, wherein
R⁵ and R⁶ are each independently hydrogen, —F, —Cl, —Br, —I, hydroxyl, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, —CN, —OR^{5a} or —NR^{5a}R^{5b}, wherein each of said methyl, ethyl, propyl, butyl, pentyl, hexyl, heptyl, octyl, cyclopropyl, cyclobutyl, cyclopentyl, cyclohexyl, cycloheptyl or cyclooctyl is optionally substituted with at least one substituent R^{5c};
R^{5a} are R^{5b} are each independently hydrogen, —C₁₋₈alkyl, —C₂₋₈alkenyl, —C₂₋₈alkynyl, C₁₋₈alkoxy-C₁₋₈alkyl- or —C₃₋₆cycloalkyl, wherein each of said —C₁₋₈alkyl, —C₂₋₈alkenyl, —C₂₋₈alkynyl, C₁₋₈alkoxy-C₁₋₈alkyl- or —C₃₋₆cycloalkyl is optionally substituted with at least one substituent R^{5c};
R^{5c}, at each of its occurrence, is independently —F, —Cl, —Br, —I, hydroxyl, methyl, ethyl, propyl, butyl, pentyl, hexyl, heptyl or octyl.

12. The compound of claim 1, wherein
R⁵ and R⁶ are each independently —H, —F, —Cl, —Br, —I, —CH₃, —C₂H₅, —C₃H₇, —C₄H₉, —C₅H₁₁, 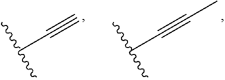
—OCH₃, —OC₂H₅, —OC₃H₇, —OC₄H₉, —OC₅H₁₁, —CH₂F, —CHF₂, —CF₃, —CN, —NH₂, —NHCH₃, —NHC₂H₅ or —N(CH₃)₂.

13. The compound of claim 1, wherein
R⁵ and R⁶, together with the atom(s) to which they are attached, form a 4-, 5-, 6- or 7-membered ring, said ring comprising 0, 1 or 2 additional heteroatom(s) independently selected from the group consisting of nitrogen, oxygen and optionally oxidized sulfur as ring member(s), said ring is optionally substituted with at least one substituent R⁵⁶;
R⁵⁶ is —H, —F, —Cl, —Br, —I, —CH₃, —C₂H₅, —C₃H₇, —C₄H₉, —C₅H₁₁, —C₆H₁₃, —C₇H₁₅, —C₈H₁₇, —C₂₋₈alkenyl, —C₂₋₈alkynyl, —CN, —OR^{56a}, —COR^{56a} or —CO₂R^{56a}, wherein each of said —CH₃, —C₂H₅, —C₃H₇, —C₄H₉, —C₅H₁₁, —C₆H₁₃, —C₇H₁₅, —C₈H₁₇, —C₂₋₈alkenyl or —C₂₋₈alkynyl is optionally substituted with at least one halogen.

14. The compound of claim 1, wherein

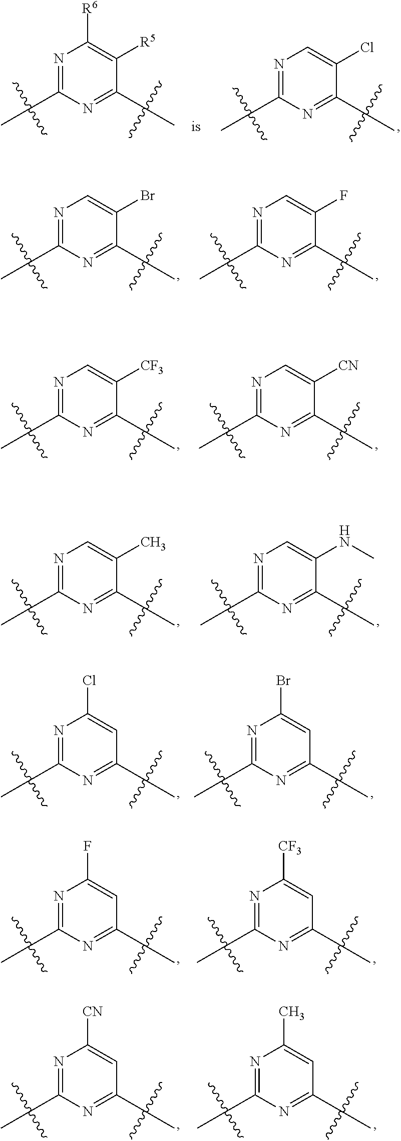

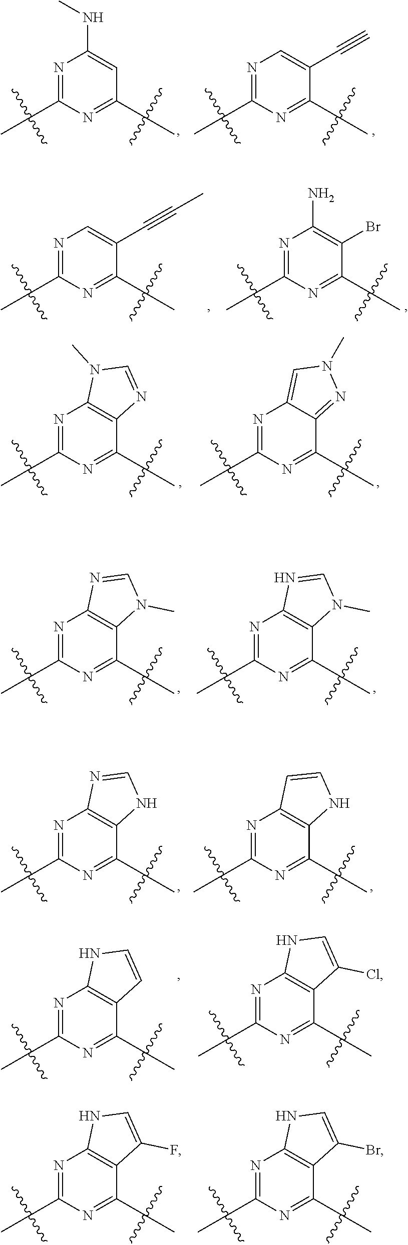

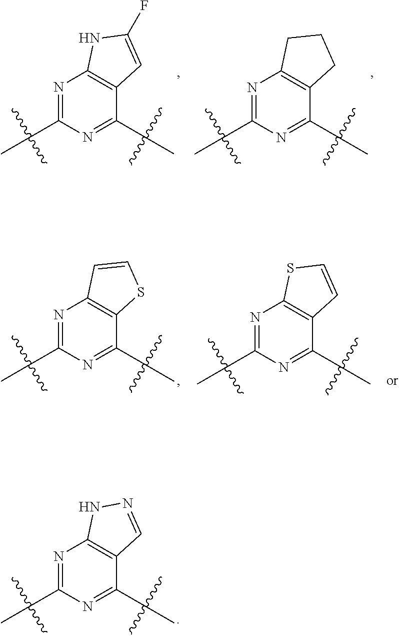 [SMILES: *c1nc(*)c2cc(F)[nH]c2n1 ; *c1nc(*)c2c(n1)CCC2 ; *c1nc(*)c2sccc2n1 ; *c1nc(*)c2ccsc2n1 ; *c1nc(*)c2cn[nH]c2n1]

15. The compound of claim 1, wherein 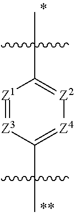
is 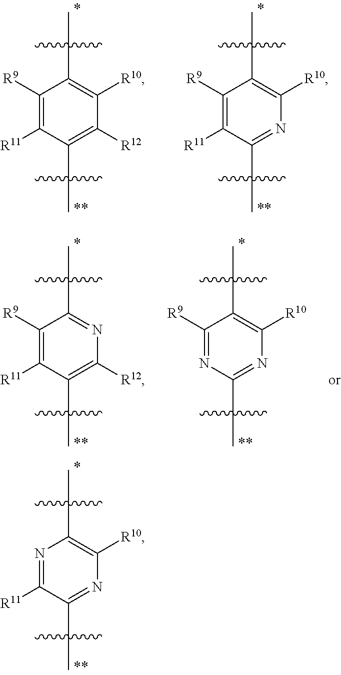
wherein * refers to the position linked to the —N(R⁷)— moiety, and ** refers to the position linked to R⁸.

16. The compound of claim 1, wherein
R⁹, R¹⁰, R¹¹ and R¹² are each independently hydrogen, halogen, —C₁₋₈alkyl, —C₂₋₈alkenyl, 3-to 8-membered heterocyclyl, —CN or —OR⁹, wherein each of said —C₁₋₈alkyl, —C₂₋₈alkenyl, —C₂₋₈alkynyl, —C₃₋₈cycloalkyl, 3- to 8-membered heterocyclyl, —C₆₋₁₂aryl or 5- to 8-membered heteroaryl is optionally substituted with at least one substituent R^{9f}; or
R⁹ and R¹¹, R¹⁰ and R¹², or R¹⁴ and R¹⁵, together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen and optionally oxidized sulfur as ring member(s), said ring is optionally substituted with at least one substituent R^{9f};
R^{9d} and R^{9e} are each independently —H, —CH₃, —C₂H₅, —C₃H₇, —C₄H₉, —C₅H₁₁, —C₆H₁₃, —C₇H₁₅ or —C₈H₁₇; or
R^{9d} and R^{9e} together with the atom(s) to which they are attached, form a 3- to 12-membered ring, said ring comprising 0, 1 or 2 additional heteroatom(s) independently selected from the group consisting of nitrogen, oxygen and optionally oxidized sulfur as ring member(s), said ring is optionally substituted with at least one substituent R^{9f};
R^{9f} at each of its occurrence, is independently hydrogen, halogen, hydroxyl, —C₁₋₈alkyl, —C₂₋₈alkenyl, —C₂₋₈alkynyl, —C₃₋₈cycloalkyl, 3- to 8-membered heterocyclyl, —C₆₋₁₂aryl, 5- to 8-membered heteroaryl, oxo (═O), —CN, —OR^{9g}, —COR^{9g}, —CO₂R^{9g}, —CONR^{9g}R^{9h}, —NR^{9g}R^{9h}, —NR^{9g}COR^{9h} or —NR^{9g}CO₂R^{9h};
R^{9g} and R^{9h} are each independently —H, —F, —Cl, —Br, —I, —CH₃, —C₂H₅, —C₃H₇, —C₄H₉, —C₅H₁₁, —C₆H₁₃, —C₇H₁₅, —C₈H₁₇ or —OH.

17. The compound of claim 1, wherein
R⁹, R¹⁰, R¹¹ and R¹² are each independently —H, —F, —Cl, —Br, —I, —CH₃, —C₂H₅, —C₃H₇, —C₄H₉, —C₅H₁₁, —C₆H₁₃, —C₇H₁₅, —C₈H₁₇, —CN, —OCH₃, —OC₂H₅, —OC₃H₇, —OC₄H₉, —OC₅H₁₁, —OC₆H₁₃, —OC₇H₁₅ or —OC₈H₁₇; or
R⁹ and R¹¹, or R¹⁰ and R¹², together with the atom(s) to which they are attached, form a 5-, 6- or 7-membered ring, said ring comprising 0, 1 or 2 additional heteroatom(s) independently selected from the group consisting of nitrogen and oxygen, said ring is optionally substituted with at least one substituent R^{9f};
R^{9f} at each of its occurrence, is independently —H, —F, —Cl, —Br, —I, —CH₃, —C₂H₅, —C₃H₇, —C₄H₉, —C₅H₁₁, —C₆H₁₃, —C₇H₁₅, —C₈H₁₇, —CN, —OCH₃, —OC₂H₅, —OC₃H₇, —OC₄H₉, —OC₅H₁₁, —OC₆H₁₃, —OC₇H₁₅ or —OC₈H₁₇; or
two R^{9f} together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen and optionally oxidized sulfur as ring member(s), said ring is optionally substituted with at least one —F, —Cl, —Br, —I, —CH₃, —C₂H₅, —C₃H₇, —C₄H₉, —C₅H₁₁, —C₆H₁₃, —C₇H₁₅, —C₈H₁₇, —CN, —OCH₃, —OC₂H₅, —OC₃H₇, —OC₄H₉, —OC₅H₁₁, —OC₆H₁₃, —OC₇H₁₅ or —OC₈H_{17.}

18. The compound of claim 1, wherein 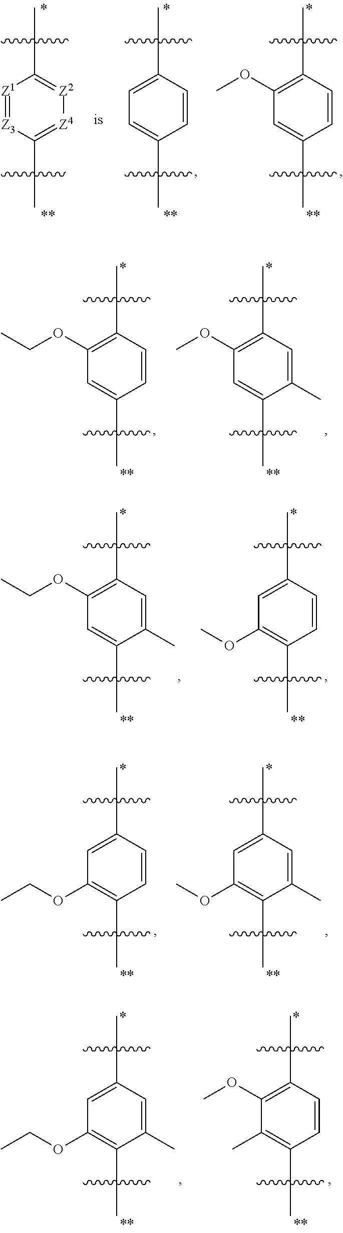 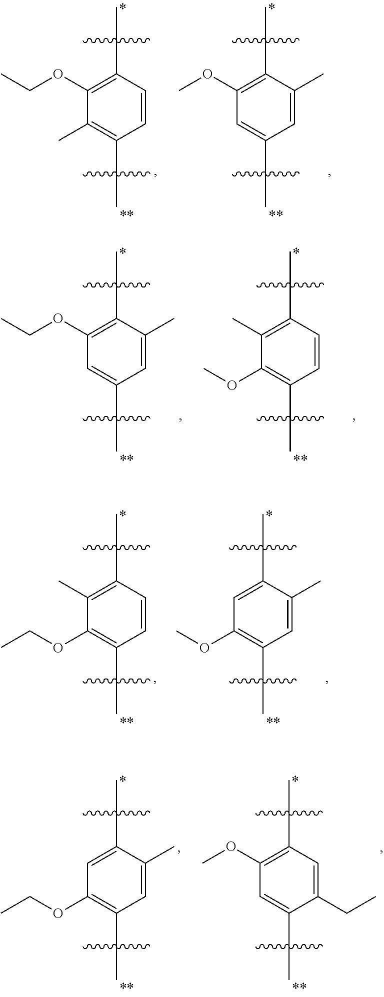 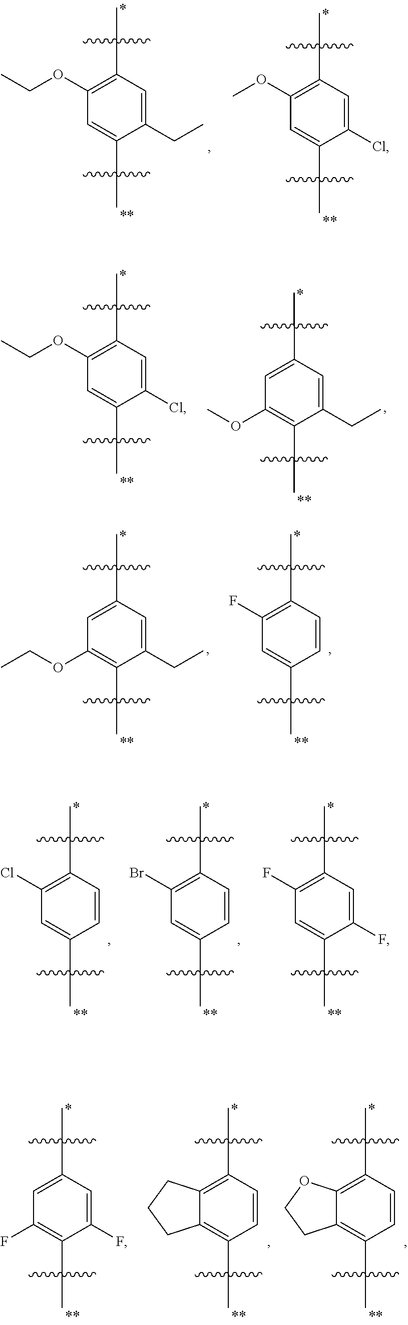 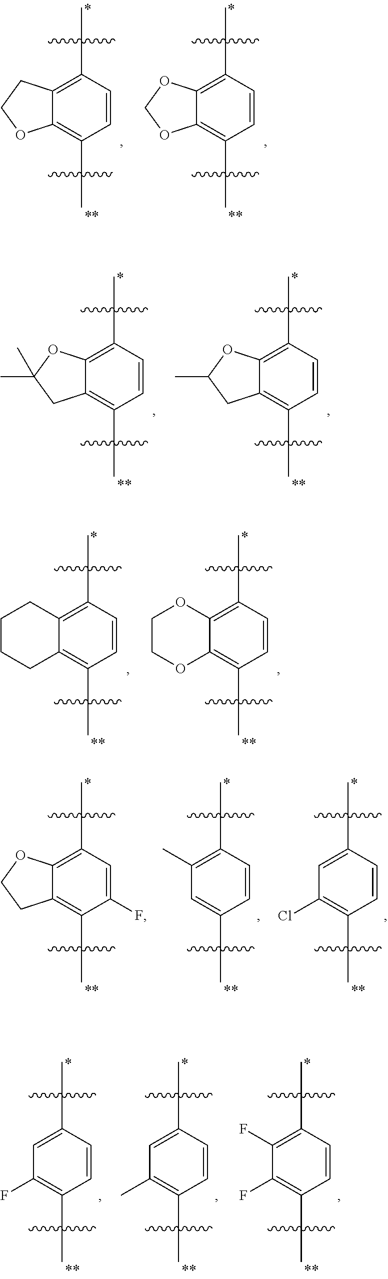 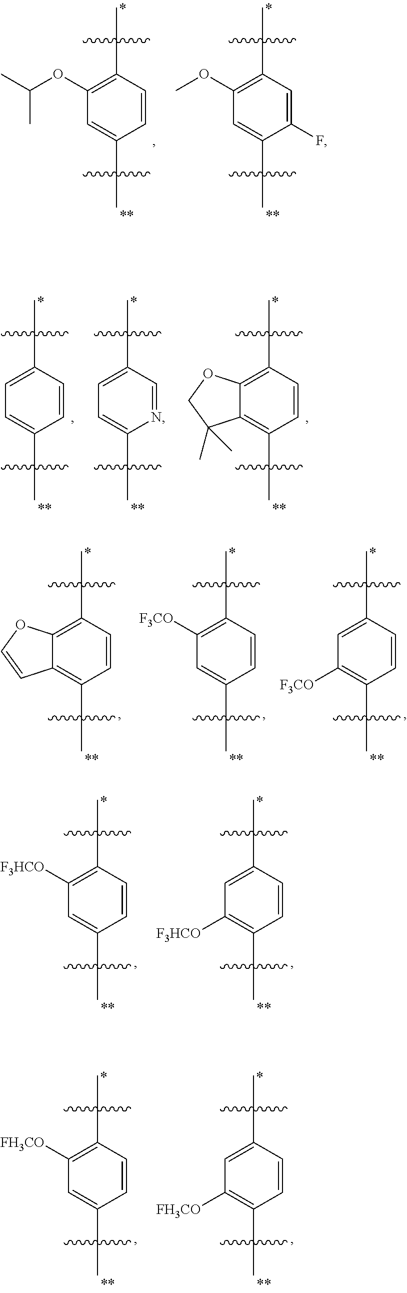 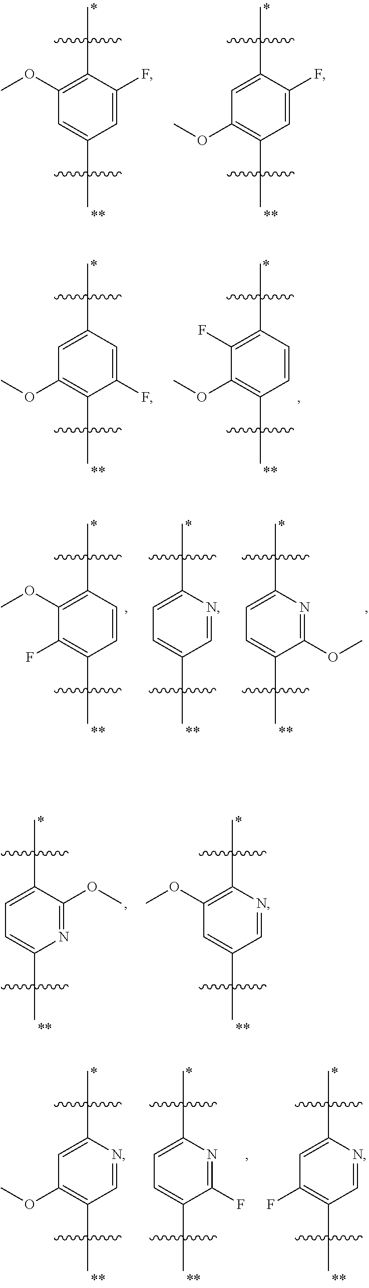 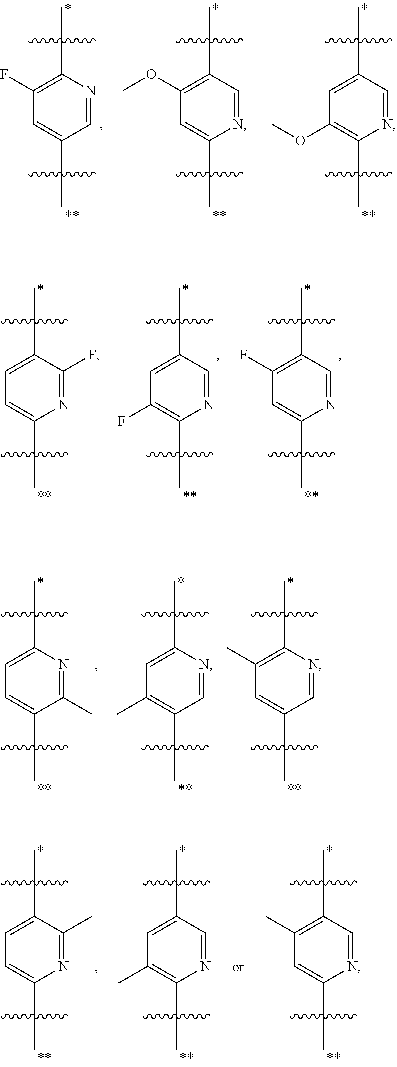
wherein * refers to the position linked to the —N(R⁷)— moiety, and * * refers to the position linked to R⁸.

19. The compound of claim 1, wherein
R⁸ is —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 4- to 8-membered monocyclic heterocyclyl comprising 1 or 2 nitrogen atoms as the ring member(s), spiro heterocyclyl selected from the group consisting of azaspiro[5.5]undecanyl, diazaspiro[5.5]undecanyl, azaspiro[4.5]decanyl, diazaspiro[4.5]decanyl, azaspiro[3.5]nonanyl, diazaspiro[3.5]nonanyl, azaspiro[4.4]nonanyl, diazaspiro[4.4]nonanyl, azaspiro[3.4]octanyl, diazaspiro[3.4]octanyl, azaspiro[3.3]heptanyl or diazaspiro[3.3]heptanyl (Preferably 3,9-diazaspiro[5.5]undecan-9-yl, 2,7-diazaspiro[3.5]nonan-7-yl, 2,8-diazaspiro[4.5]decan-8yl and 2,6-diazaspiro[3.3]heptan-6-yl), bridged heterocyclyl selected from the group consisting of azabicyclo[2.2.1]heptanyl, diazabicyclo[2.2.1]heptanyl, azabicyclo[3.1.1]heptanyl, diazabicyclo[3.1.1]heptanyl, azabicyclo[2.2.2]octanyl, diazabicyclo[2.2.2]octanyl, azabicyclo[3.2.1]octanyl and diazabicyclo[3.2.1]octanyl 5- to 8-membered heteroaryl, —OR^{a} or —N^{8a}R^{8b}, wherein each of said methyl, ethyl, propyl, butyl, pentyl, hexyl, heptyl, octyl, cyclobutyl, cyclopentyl, cyclohexyl, cycloheptyl, cyclooctyl, 4- to 8-membered monocyclic heterocyclyl, spiro heterocyclyl, or heteroaryl is optionally substituted with at least one substituent R^{8c};
R^{8a} and R^{8b} are each independently hydrogen, methyl, ethyl, propyl, butyl, pentyl, hexyl, heptyl, octyl, cyclobutyl, cyclopentyl, cyclohexyl, 4- to 8-membered heterocyclyl, 5- to 8-membered heteroaryl, C₁₋₈alkoxy-C₁₋₈alkyl-, phenyl or 5- to 8-membered heteroaryl, wherein each of said methyl, ethyl, propyl, butyl, pentyl, hexyl, heptyl, octyl, cyclobutyl, cyclopentyl, cyclohexyl, 4- to 8-membered heterocyclyl, 5- to 8-membered heteroaryl, C₁₋₈alkoxy-C₁₋₈alkyl-, phenyl or 5- to 8-membered heteroaryl is optionally substituted with at least one substituent R^{8d}; or
R^{8a} and R^{8b} together with the atom(s) to which they are attached, form a 3-, 4-, 5-, 6-, 7- or 8-membered ring, said ring comprising 0, 1 or 2 additional heteroatom(s) independently selected from the group consisting of nitrogen and oxygen as ring member(s), said ring is optionally substituted with at least one substituent R^{8d};
R^{8c} is independently hydrogen, —F, —Cl, —Br, —I, methyl, ethyl, propyl, butyl, pentyl, hexyl, heptyl, octyl, cyclobutyl, cyclopentyl, cyclohexyl, phenyl —C₂₋₈alkenyl, —C₂₋₈alkynyl, 3- to 12-membered heterocyclyl, 5- to 8-membered heteroaryl, oxo (═O), —CN, —OCH₃, —OC₂H₅, —OC₃H₇, —OC₄H₉, —OC₅H₁₁, —OC₆H₁₃, —OC₇H₁₅, —OC₈H₁₇, —COCH₃, —COC₂H₈, —COC₃H₇, —COC₄H₉, —COC₅H₁₁, —COC₆H₁₃, —COC₇H₁₅, —COC₈H₁₇, —CONR^{8f}R^{8g}, —NR^{8f}R^{8g} or —NR^{8f}COR^{8g}, wherein each of said methyl, ethyl, propyl, butyl, pentyl, hexyl, heptyl, octyl, cyclobutyl, cyclopentyl, cyclohexyl, phenyl —C₂₋₈alkenyl, —C₂₋₈alkynyl, 3- to 12-membered heterocyclyl, 5- to 8-membered heteroaryl is optionally substituted with at least one R^{8e}; or
two R^{8c} together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen and optionally oxidized sulfur as ring member(s), said ring is optionally substituted with at least one R^{8e};
R^{8d}, and R^{8e} are each independently hydrogen, —F, —Cl, —Br, —I, methyl, ethyl, propyl, butyl, pentyl, hexyl, heptyl, octyl, cyclobutyl, cyclopentyl, cyclohexyl, —C₂₋₈alkenyl, —C₂₋₈alkynyl, 3- to 8-membered heterocyclyl, phenyl, 5- to 8-membered heteroaryl, oxo (═O), —CN, —OH, —OCH₃, —OC₂H₅, —OC₃H₇, —OC₄H₉, —OC₅H₁₁, —OC₆H₁₃, —OC₇H₁₅, —OC₈H₁₇, —COCH₃, —COC₂H₅, —COC₃H₇, —COC₄H₉, —COC₅H₁₁, —COC₆H₁₃, —COC₇H₁₅, —COC₈H₁₇, —CO₂CH₃, —CO₂C₂H₅, —CO₂C₃H₇, —CO₂C₄H₉, —CO₂C₅H₁₁, —CO₂C₆H₁₃, —CO₂C₇H₁₅, —CO₂C₈H₁₇, —CONR^{8h}R¹, —NR^{8h}R¹ or —NR^{8h}COR⁸ⁱ, wherein each of said methyl, ethyl, propyl, butyl, pentyl, hexyl, heptyl, octyl, cyclobutyl, cyclopentyl, cyclohexyl, —C₂₋₈alkenyl, —C₂₋₈alkynyl, 3- to 8-membered heterocyclyl, phenyl, 5- to 8-membered heteroaryl is optionally substituted with at least one —F, —Cl, —Br, —I, methyl, ethyl, propyl, butyl, pentyl, hexyl, heptyl, octyl, cyclobutyl, cyclopentyl, cyclohexyl, —C₂₋₈alkenyl, —C₂₋₈alkynyl, 3- to 8-membered heterocyclyl, phenyl, 5- to 8-membered heteroaryl, phenyl, hydroxyl, —OCH₃, —OC₂H₅, —OC₃H₇, —OC₄H₉, —OC₅H₁₁, —OC₆H₁₃, —OC₇H₁₅, —OC₈H₁₇ or C₁₋₈alkoxy-C₁₋₈alkyl-;
R^{8g}, R^{8g}, R^{8h} and R⁸ⁱ are each independently hydrogen, methyl, ethyl, propyl, butyl, pentyl, hexyl, heptyl, octyl, cyclobutyl, cyclopentyl, cyclohexyl, C₁₋₈alkoxy-C₁₋₈alkyl-, —C₂₋₈alkenyl, —C₂₋₈alkynyl, 3- to 8-membered heterocyclyl, phenyl or 5- to 8-membered heteroaryl.

20. The compound of claim 1, wherein
R⁸ is F, Cl, Br, methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl, pyrazolyl, —OR^{8a} or —NR^{8a}R^{8b}, wherein each of said methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl, pyrazolyl is optionally substituted with at least one substituent R^{8c};
R^{8a} and R^{8b} are each independently methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl or pyrazolyl, wherein each of said methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl or pyrazolyl is optionally substituted with at least one substituent R^{8d}; or
R^{8a} and R^{8b} together with the atom(s) to which they are attached, form a 4-, 5-, 6-, 7- or 8-membered ring, said ring comprising 0, 1 or 2 additional heteroatom(s) independently selected from the group consisting of nitrogen and oxygen as ring member(s), said ring is optionally substituted with at least one substituent R^{8d};
R^{8c} is independently hydrogen, F, Cl, Br, methyl, ethyl, propyl, butyl, pentyl, oxo (═O), azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl, pyrazolyl, —CN, —OR^{8f} or —NR^{8f}R^{8g}, wherein each of said methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl, pyrazolyl is optionally substituted with at least one R^{8e}; or
two R^{8c} together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen and optionally oxidized sulfur as ring member(s), said ring is optionally substituted with at least one R^{8e};
R^{8d} and R^{8e} are each independently hydrogen, F, Cl, Br, methyl, ethyl, propyl, butyl, pentyl, oxo (═O), azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl, pyrazolyl, —CN, —OR^{8h} or —NR^{8h}R⁸ⁱ, wherein each of said methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl, pyrazolyl is optionally substituted with at least one F, Cl, Br, methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl or pyrazolyl; or
two R^{8e} together with the atom(s) to which they are attached, form a 3-, 4-, 5-, 6-, 7- or 8-membered ring, said ring comprising 0, 1 or 2 additional heteroatom(s) independently selected from the group consisting of nitrogen, oxygen and optionally oxidized sulfur as ring member(s), said ring is optionally substituted with at least one F, Cl, Br, methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl, pyrazolyl or —CN;
R^{8f}, R^{8g}, R^{8h} and R⁸ⁱ are each independently hydrogen, methyl, ethyl, propyl, butyl, pentyl, azacyclopropyl, azacyclobutyl, tetrahydropyrrolyl, piperidinyl, morpholinyl, piperazinyl, pyrrolyl, imidazolyl, oxazolyl, thiazolyl, pyridinyl, pyrimidinyl, pyrazinyl, pyridazinyl, phenyl or pyrazolyl.

21. The compound of claim 1, wherein R⁸ is

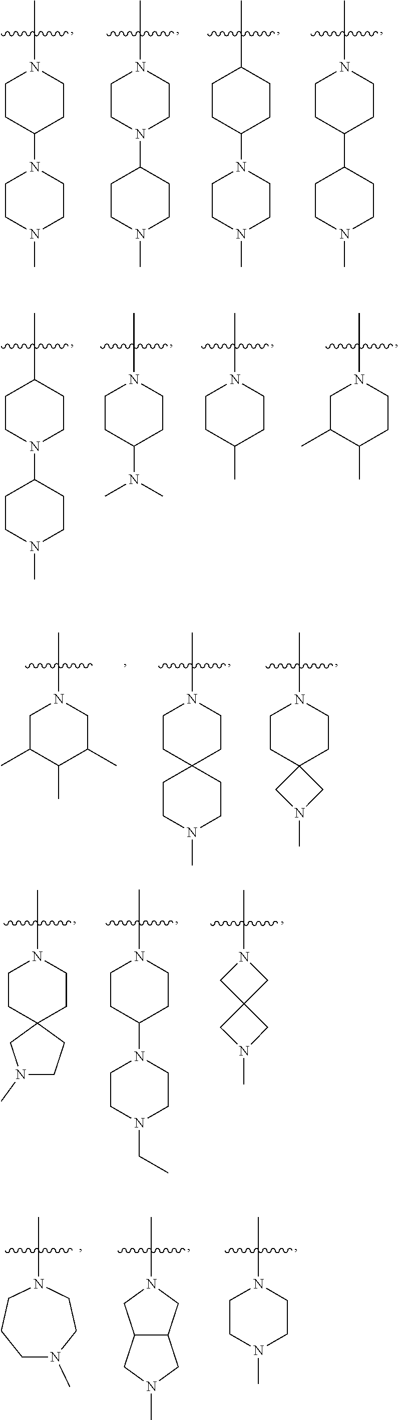

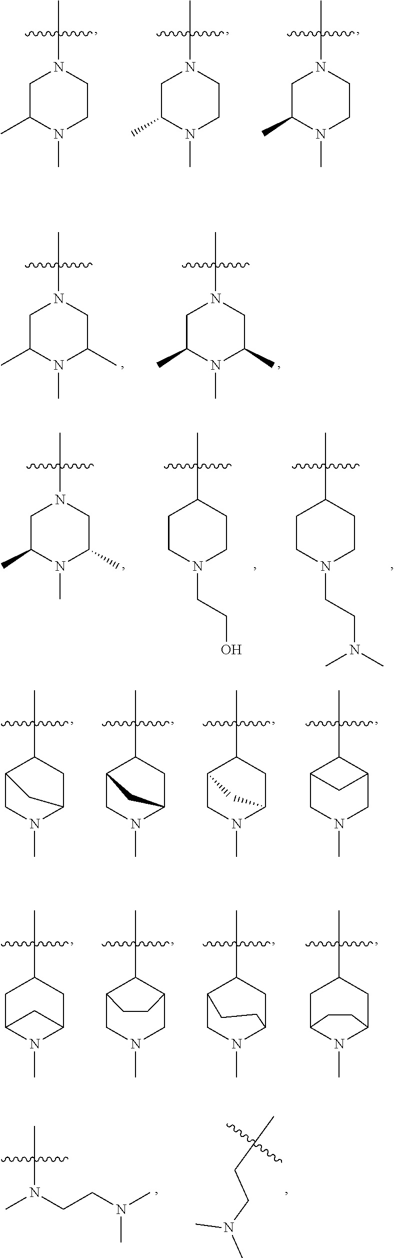

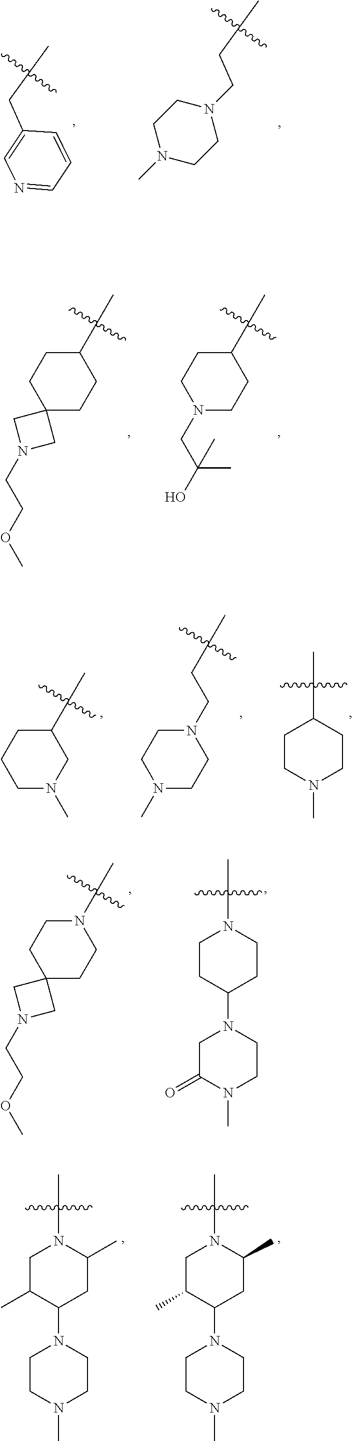

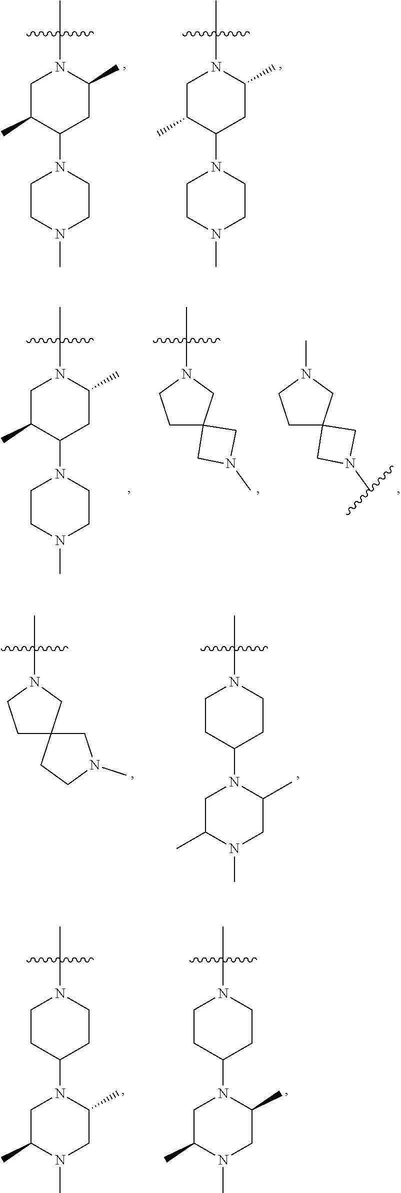

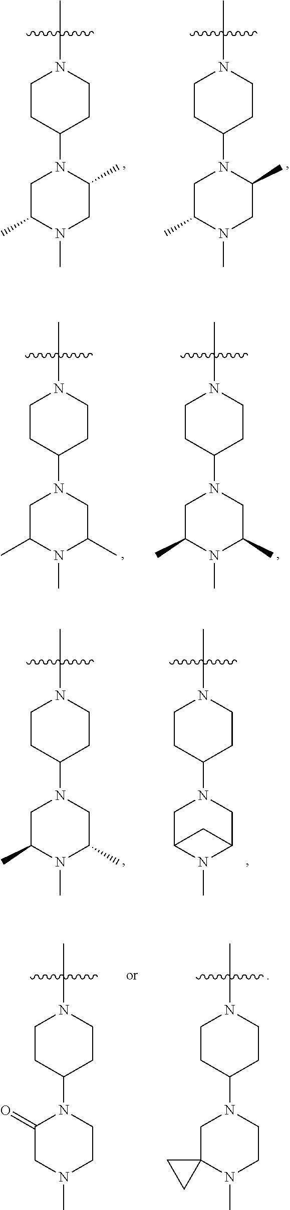

22. A compound selected from the group consisting of: 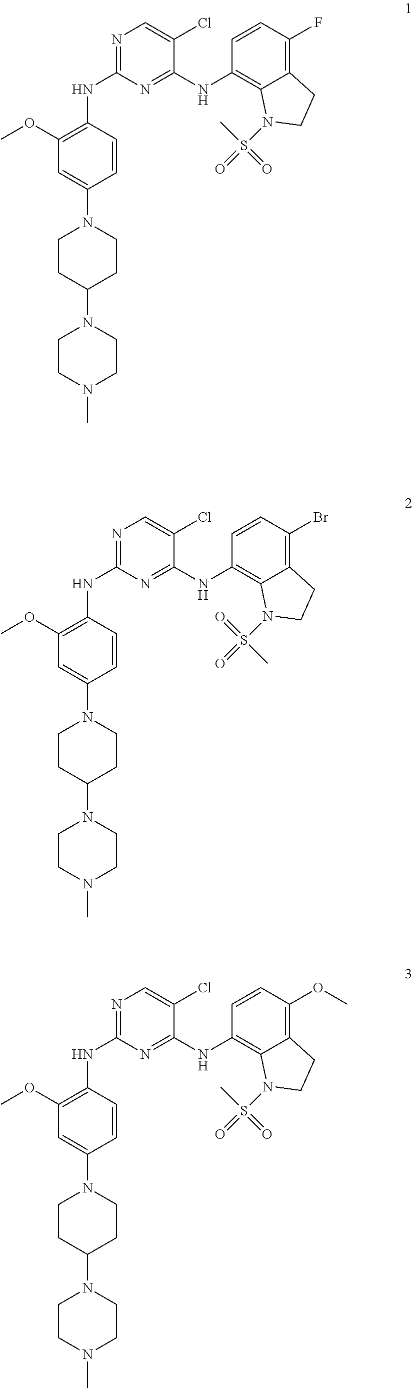 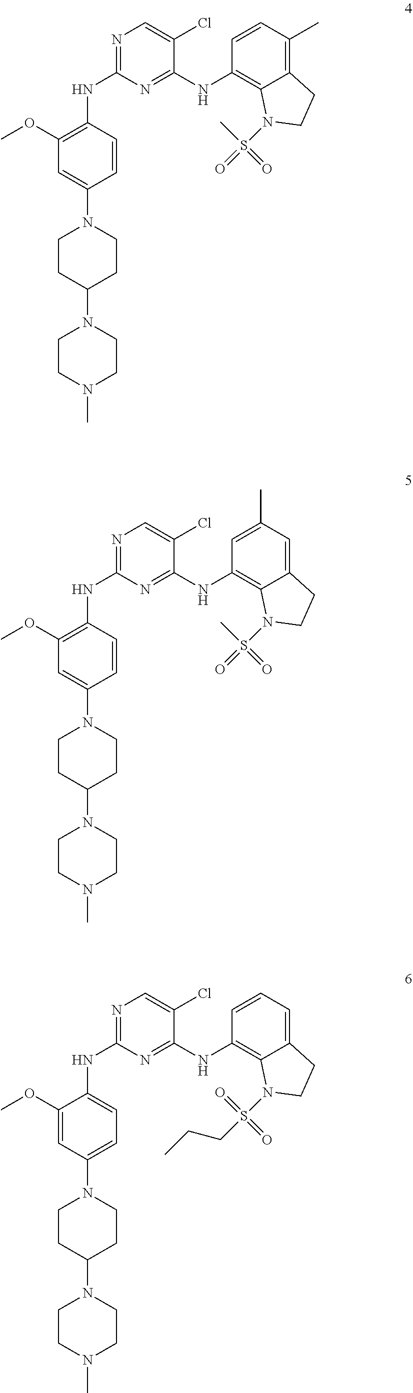 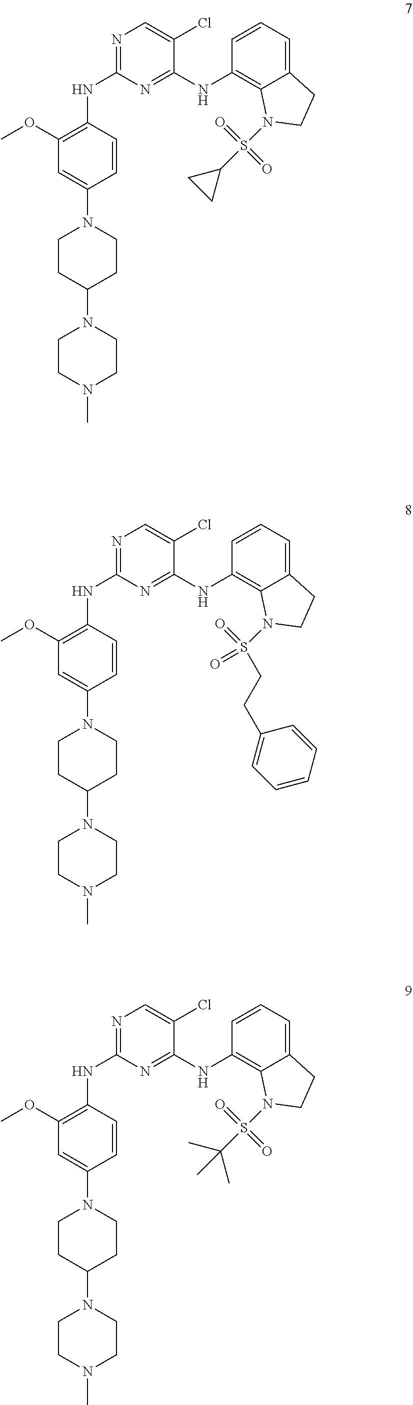 [SMILES: COc1cc(N2CCC(N3CCN(C)CC3)CC2)ccc1Nc1ncc(Cl)c(Nc2cccc3c2N(S(=O)(=O)C2CC2)CC3)n1 ; COc1cc(N2CCC(N3CCN(C)CC3)CC2)ccc1Nc1ncc(Cl)c(Nc2cccc3c2N(S(=O)(=O)CCc2ccccc2)CC3)n1 ; COc1cc(N2CCC(N3CCN(C)CC3)CC2)ccc1Nc1ncc(Cl)c(Nc2cccc3c2N(S(=O)(=O)C(C)(C)C)CC3)n1] 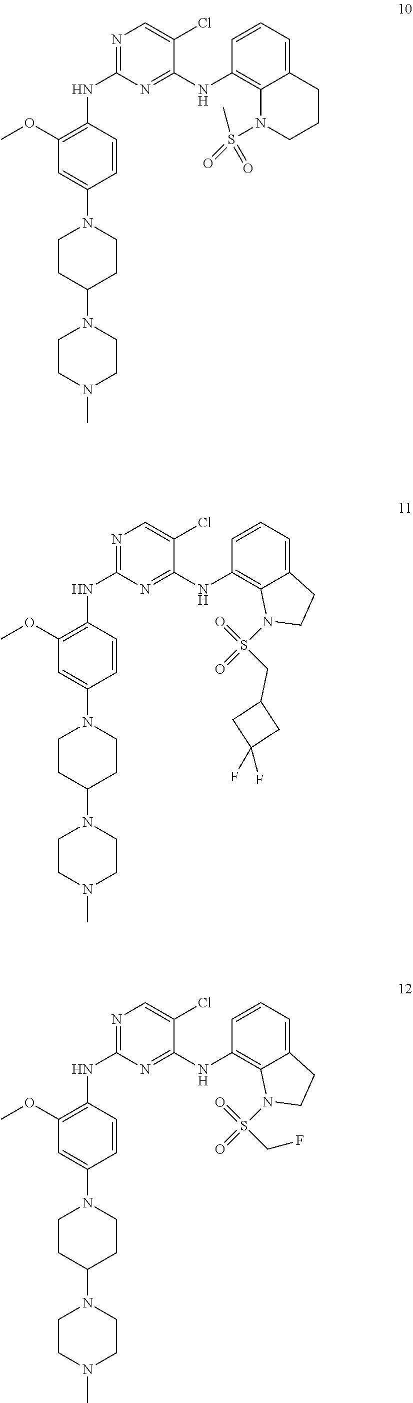 [SMILES: COc1cc(N2CCC(N3CCN(C)CC3)CC2)ccc1Nc1ncc(Cl)c(Nc2cccc3c2N(S(C)(=O)=O)CCC3)n1 ; COc1cc(N2CCC(N3CCN(C)CC3)CC2)ccc1Nc1ncc(Cl)c(Nc2cccc3c2N(S(=O)(=O)CC2CC(F)(F)C2)CC3)n1 ; COc1cc(N2CCC(N3CCN(C)CC3)CC2)ccc1Nc1ncc(Cl)c(Nc2cccc3c2N(S(=O)(=O)CF)CC3)n1] 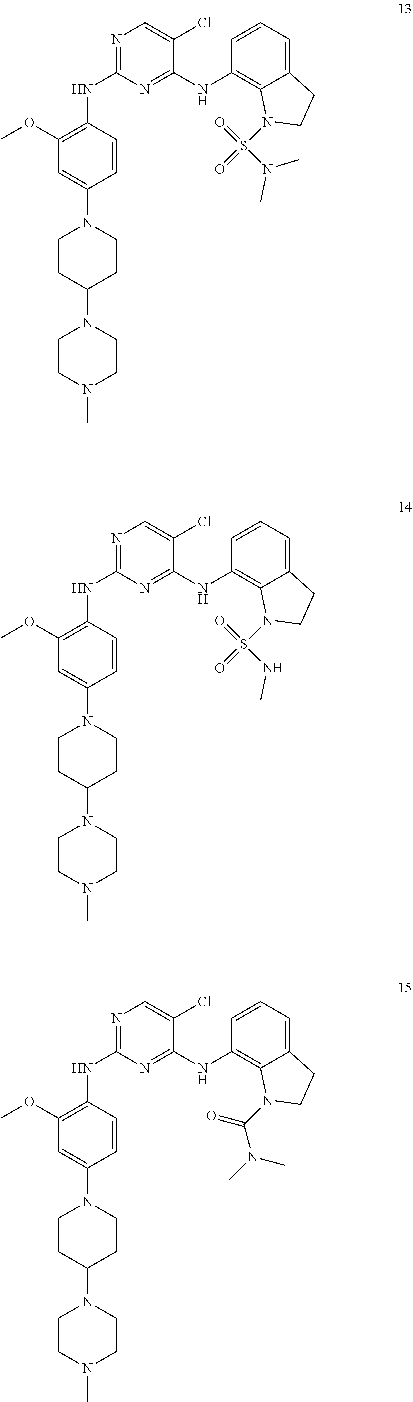 [SMILES: COc1cc(N2CCC(N3CCN(C)CC3)CC2)ccc1Nc1ncc(Cl)c(Nc2cccc3c2N(S(=O)(=O)N(C)C)CC3)n1 ; CNS(=O)(=O)N1CCc2cccc(Nc3nc(Nc4ccc(N5CCC(N6CCN(C)CC6)CC5)cc4OC)ncc3Cl)c21 ; COc1cc(N2CCC(N3CCN(C)CC3)CC2)ccc1Nc1ncc(Cl)c(Nc2cccc3c2N(C(=O)N(C)C)CC3)n1] 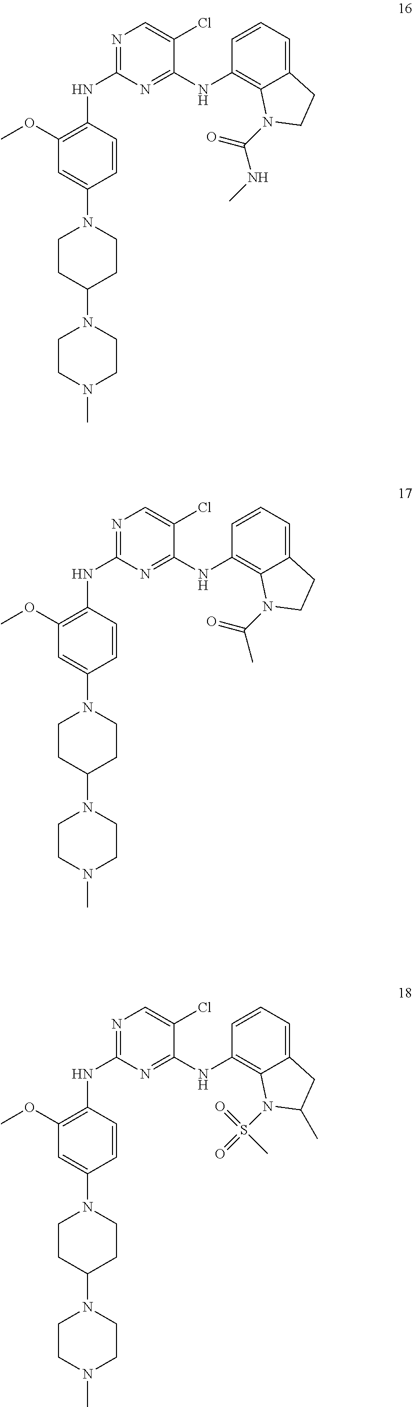 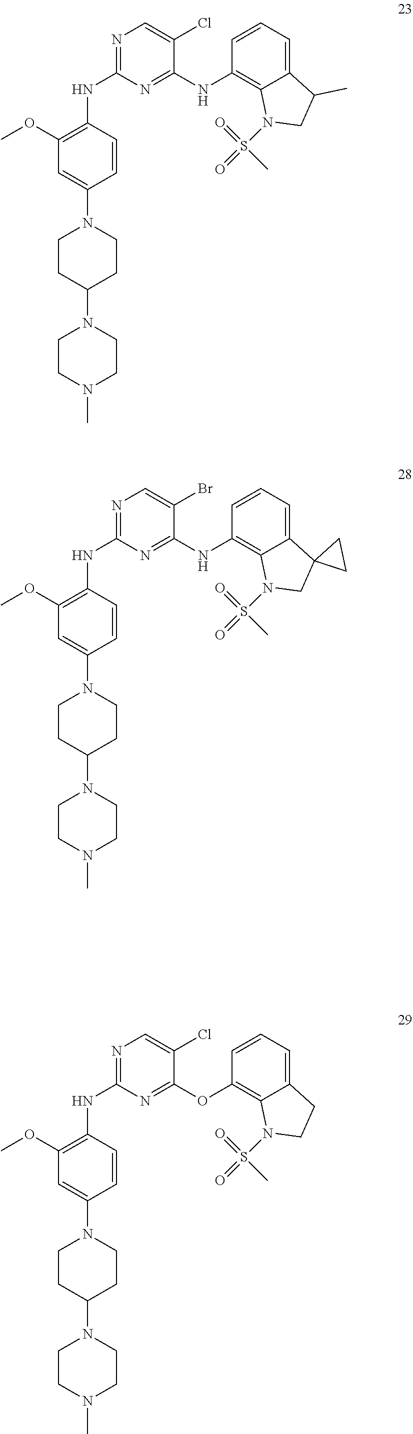 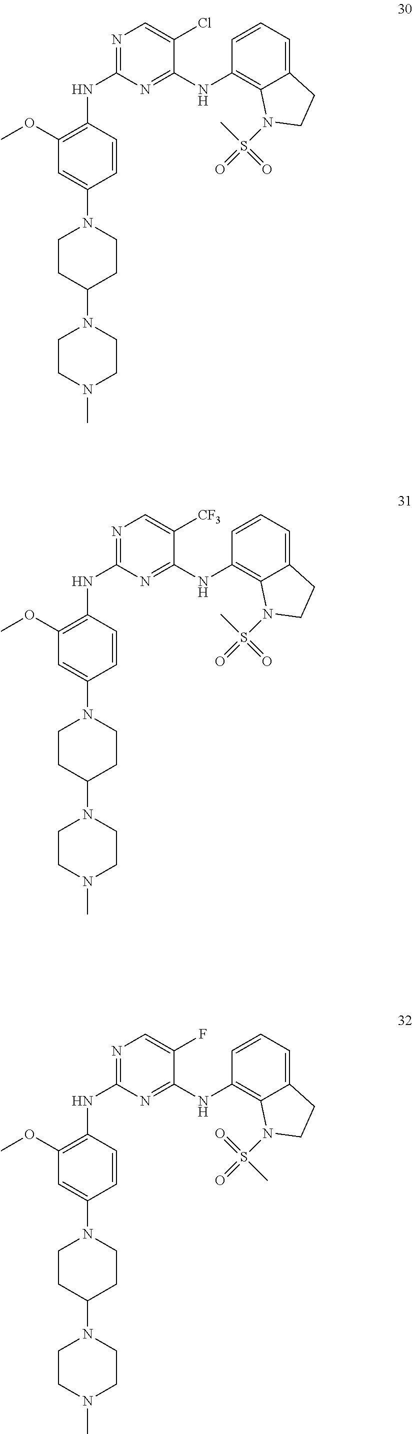 [SMILES: COc1cc(N2CCC(N3CCN(C)CC3)CC2)ccc1Nc1ncc(Cl)c(Nc2cccc3c2N(S(C)(=O)=O)CC3)n1 ; COc1cc(N2CCC(N3CCN(C)CC3)CC2)ccc1Nc1ncc(C(F)(F)F)c(Nc2cccc3c2N(S(C)(=O)=O)CC3)n1 ; COc1cc(N2CCC(N3CCN(C)CC3)CC2)ccc1Nc1ncc(F)c(Nc2cccc3c2N(S(C)(=O)=O)CC3)n1] 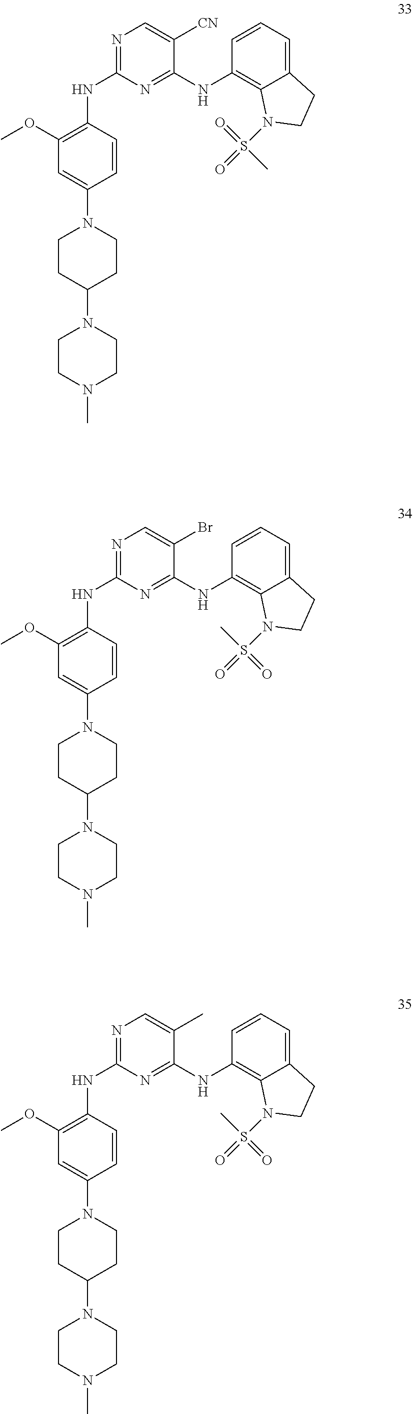 [SMILES: COc1cc(N2CCC(N3CCN(C)CC3)CC2)ccc1Nc1ncc(C#N)c(Nc2cccc3c2N(S(C)(=O)=O)CC3)n1 ; COc1cc(N2CCC(N3CCN(C)CC3)CC2)ccc1Nc1ncc(Br)c(Nc2cccc3c2N(S(C)(=O)=O)CC3)n1 ; COc1cc(N2CCC(N3CCN(C)CC3)CC2)ccc1Nc1ncc(C)c(Nc2cccc3c2N(S(C)(=O)=O)CC3)n1] 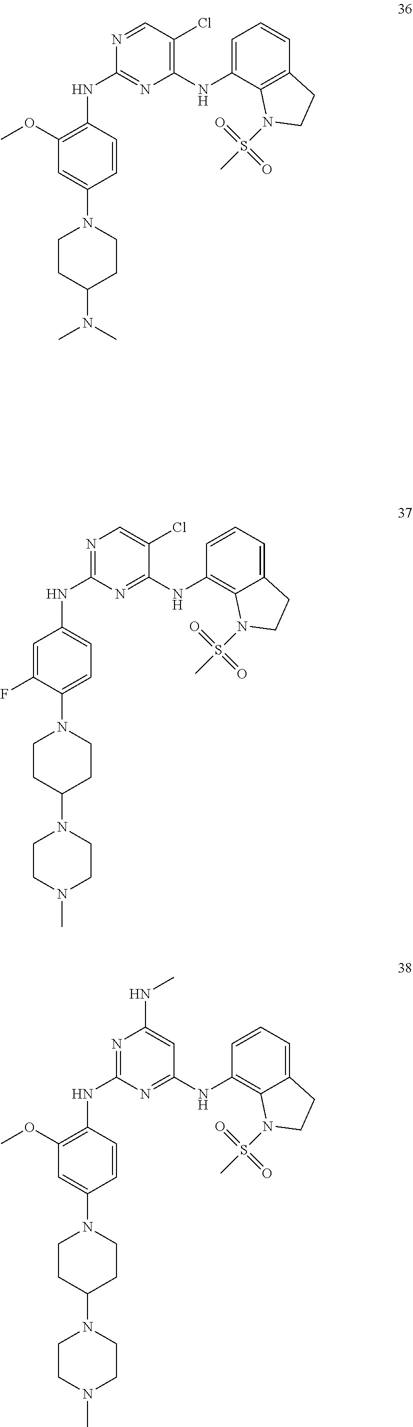 [SMILES: COc1cc(N2CCC(N(C)C)CC2)ccc1Nc1ncc(Cl)c(Nc2cccc3c2N(S(C)(=O)=O)CC3)n1 ; CN1CCN(C2CCN(c3ccc(Nc4ncc(Cl)c(Nc5cccc6c5N(S(C)(=O)=O)CC6)n4)cc3F)CC2)CC1 ; CNc1cc(Nc2cccc3c2N(S(C)(=O)=O)CC3)nc(Nc2ccc(N3CCC(N4CCN(C)CC4)CC3)cc2OC)n1] 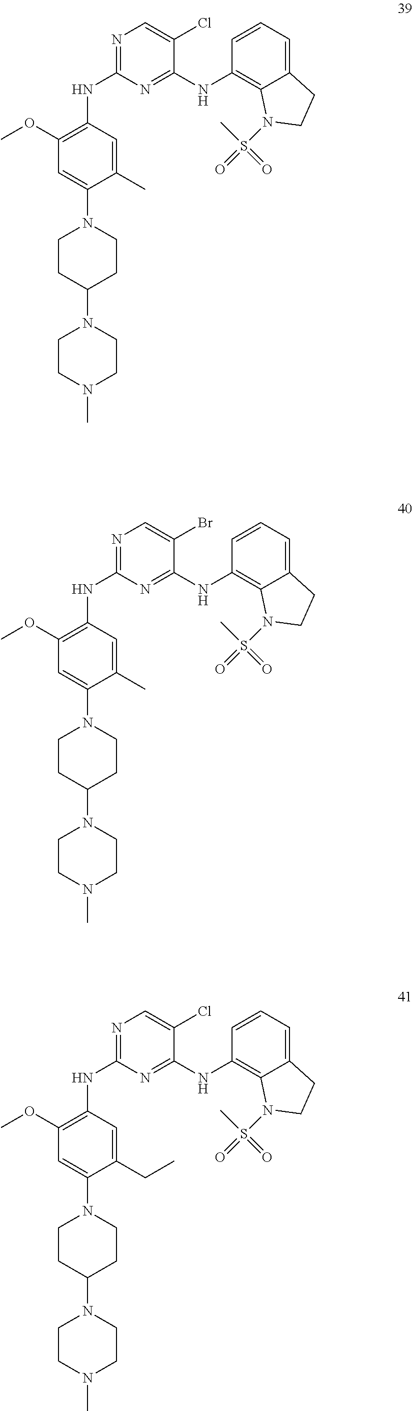 [SMILES: COc1cc(N2CCC(N3CCN(C)CC3)CC2)c(C)cc1Nc1ncc(Cl)c(Nc2cccc3c2N(S(C)(=O)=O)CC3)n1 ; COc1cc(N2CCC(N3CCN(C)CC3)CC2)c(C)cc1Nc1ncc(Br)c(Nc2cccc3c2N(S(C)(=O)=O)CC3)n1 ; CCc1cc(Nc2ncc(Cl)c(Nc3cccc4c3N(S(C)(=O)=O)CC4)n2)c(OC)cc1N1CCC(N2CCN(C)CC2)CC1] 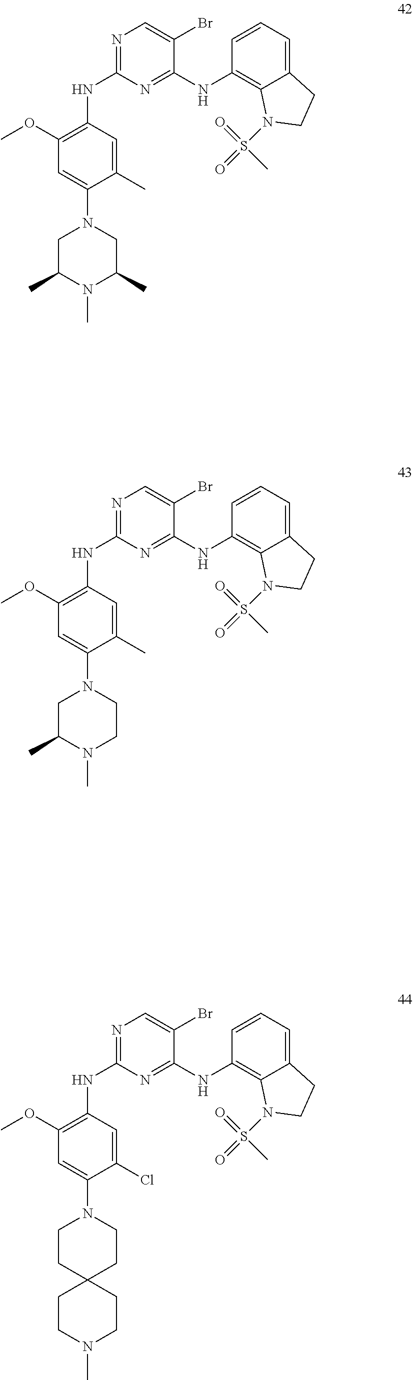 [SMILES: COc1cc(N2C[C@@H](C)N(C)[C@@H](C)C2)c(C)cc1Nc1ncc(Br)c(Nc2cccc3c2N(S(C)(=O)=O)CC3)n1 ; COc1cc(N2CCN(C)[C@@H](C)C2)c(C)cc1Nc1ncc(Br)c(Nc2cccc3c2N(S(C)(=O)=O)CC3)n1 ; COc1cc(N2CCC3(CCN(C)CC3)CC2)c(Cl)cc1Nc1ncc(Br)c(Nc2cccc3c2N(S(C)(=O)=O)CC3)n1] 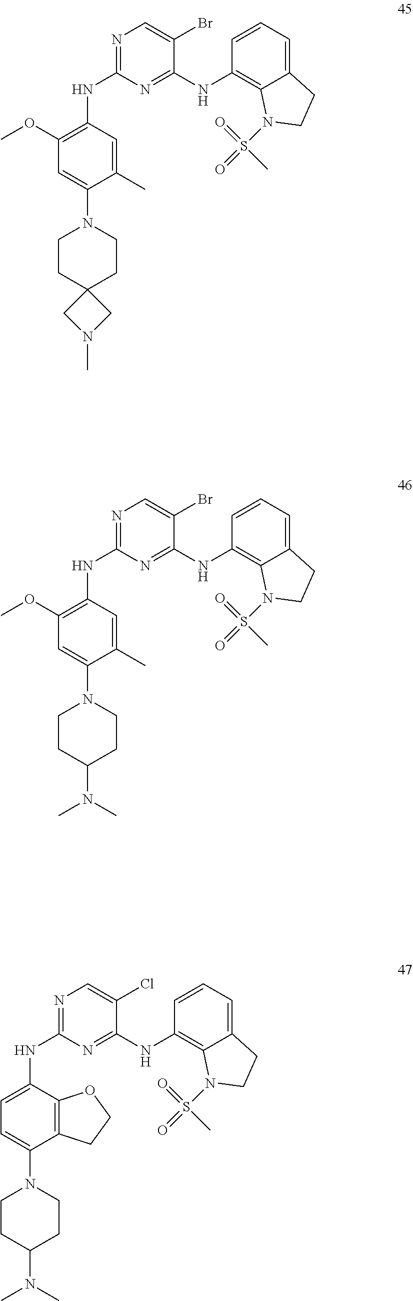 [SMILES: COc1cc(N2CCC3(CC2)CN(C)C3)c(C)cc1Nc1ncc(Br)c(Nc2cccc3c2N(S(C)(=O)=O)CC3)n1 ; COc1cc(N2CCC(N(C)C)CC2)c(C)cc1Nc1ncc(Br)c(Nc2cccc3c2N(S(C)(=O)=O)CC3)n1 ; CN(C)C1CCN(c2ccc(Nc3ncc(Cl)c(Nc4cccc5c4N(S(C)(=O)=O)CC5)n3)c3c2CCO3)CC1] 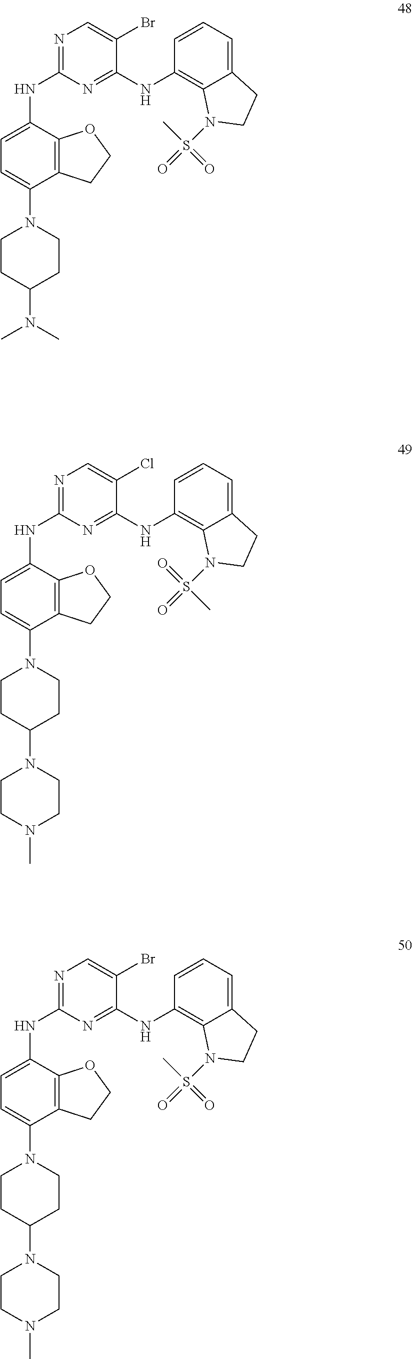 [SMILES: CN(C)C1CCN(c2ccc(Nc3ncc(Br)c(Nc4cccc5c4N(S(C)(=O)=O)CC5)n3)c3c2CCO3)CC1 ; CN1CCN(C2CCN(c3ccc(Nc4ncc(Cl)c(Nc5cccc6c5N(S(C)(=O)=O)CC6)n4)c4c3CCO4)CC2)CC1 ; CN1CCN(C2CCN(c3ccc(Nc4ncc(Br)c(Nc5cccc6c5N(S(C)(=O)=O)CC6)n4)c4c3CCO4)CC2)CC1] 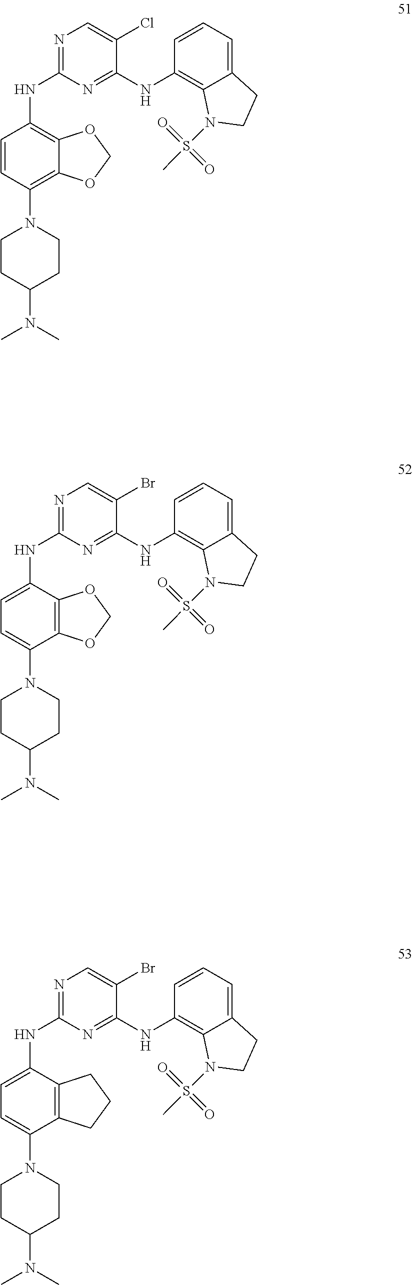 [SMILES: CN(C)C1CCN(c2ccc(Nc3ncc(Cl)c(Nc4cccc5c4N(S(C)(=O)=O)CC5)n3)c3c2OCO3)CC1 ; CN(C)C1CCN(c2ccc(Nc3ncc(Br)c(Nc4cccc5c4N(S(C)(=O)=O)CC5)n3)c3c2OCO3)CC1 ; CN(C)C1CCN(c2ccc(Nc3ncc(Br)c(Nc4cccc5c4N(S(C)(=O)=O)CC5)n3)c3c2CCC3)CC1] 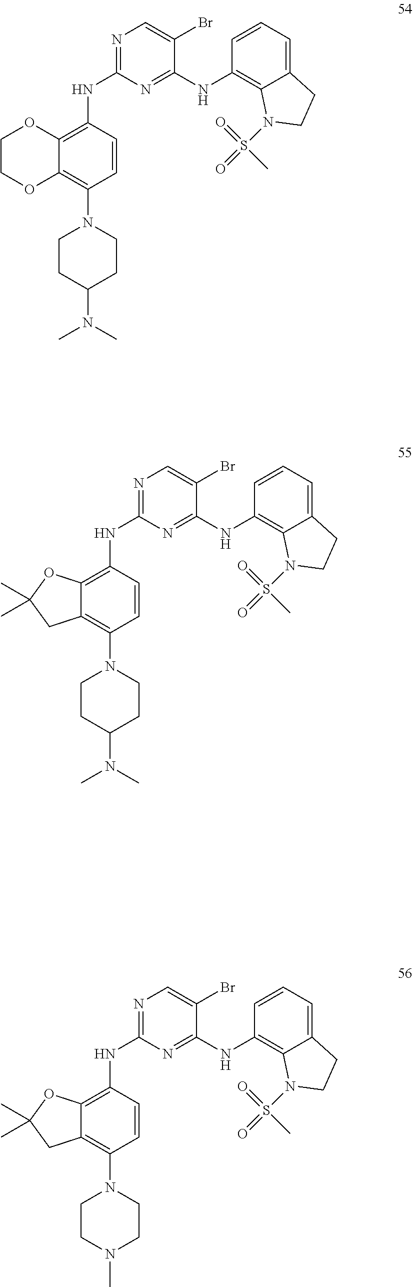 [SMILES: CN(C)C1CCN(c2ccc(Nc3ncc(Br)c(Nc4cccc5c4N(S(C)(=O)=O)CC5)n3)c3c2OCCO3)CC1 ; CN(C)C1CCN(c2ccc(Nc3ncc(Br)c(Nc4cccc5c4N(S(C)(=O)=O)CC5)n3)c3c2CC(C)(C)O3)CC1 ; CN1CCN(c2ccc(Nc3ncc(Br)c(Nc4cccc5c4N(S(C)(=O)=O)CC5)n3)c3c2CC(C)(C)O3)CC1] 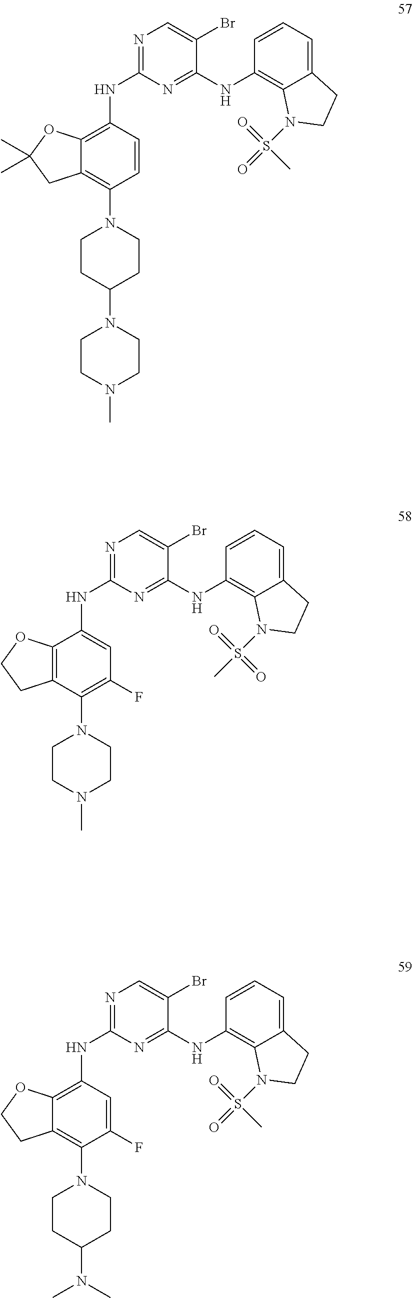 [SMILES: CN1CCN(C2CCN(c3ccc(Nc4ncc(Br)c(Nc5cccc6c5N(S(C)(=O)=O)CC6)n4)c4c3CC(C)(C)O4)CC2)CC1 ; CN1CCN(c2c(F)cc(Nc3ncc(Br)c(Nc4cccc5c4N(S(C)(=O)=O)CC5)n3)c3c2CCO3)CC1 ; CN(C)C1CCN(c2c(F)cc(Nc3ncc(Br)c(Nc4cccc5c4N(S(C)(=O)=O)CC5)n3)c3c2CCO3)CC1] 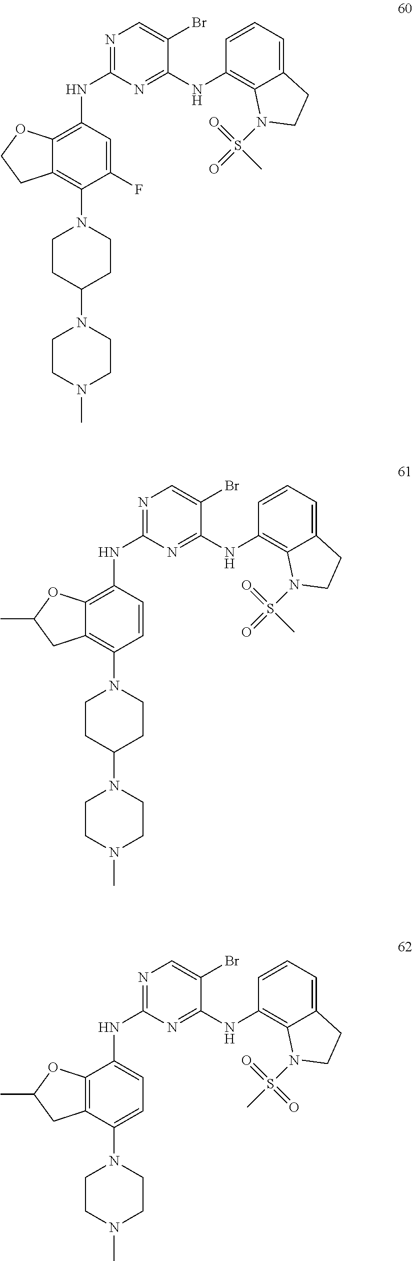 [SMILES: CN1CCN(C2CCN(c3c(F)cc(Nc4ncc(Br)c(Nc5cccc6c5N(S(C)(=O)=O)CC6)n4)c4c3CCO4)CC2)CC1 ; CC1Cc2c(N3CCC(N4CCN(C)CC4)CC3)ccc(Nc3ncc(Br)c(Nc4cccc5c4N(S(C)(=O)=O)CC5)n3)c2O1 ; CC1Cc2c(N3CCN(C)CC3)ccc(Nc3ncc(Br)c(Nc4cccc5c4N(S(C)(=O)=O)CC5)n3)c2O1] 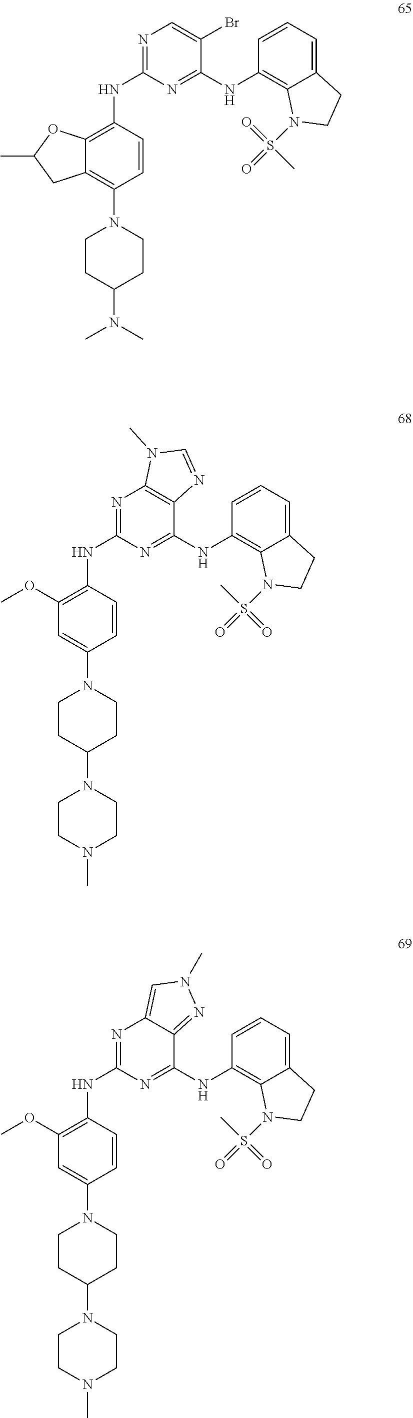 [SMILES: CC1Cc2c(N3CCC(N(C)C)CC3)ccc(Nc3ncc(Br)c(Nc4cccc5c4N(S(C)(=O)=O)CC5)n3)c2O1 ; COc1cc(N2CCC(N3CCN(C)CC3)CC2)ccc1Nc1nc(Nc2cccc3c2N(S(C)(=O)=O)CC3)c2ncn(C)c2n1 ; COc1cc(N2CCC(N3CCN(C)CC3)CC2)ccc1Nc1nc(Nc2cccc3c2N(S(C)(=O)=O)CC3)c2nn(C)cc2n1] 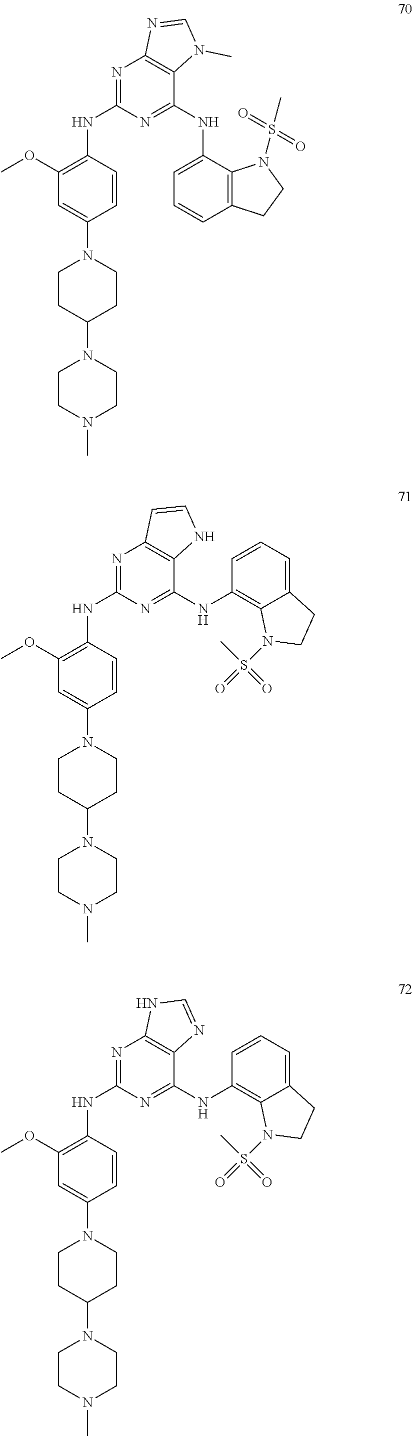 [SMILES: COc1cc(N2CCC(N3CCN(C)CC3)CC2)ccc1Nc1nc(Nc2cccc3c2N(S(C)(=O)=O)CC3)c2c(ncn2C)n1 ; COc1cc(N2CCC(N3CCN(C)CC3)CC2)ccc1Nc1nc(Nc2cccc3c2N(S(C)(=O)=O)CC3)c2[nH]ccc2n1 ; COc1cc(N2CCC(N3CCN(C)CC3)CC2)ccc1Nc1nc(Nc2cccc3c2N(S(C)(=O)=O)CC3)c2nc[nH]c2n1] 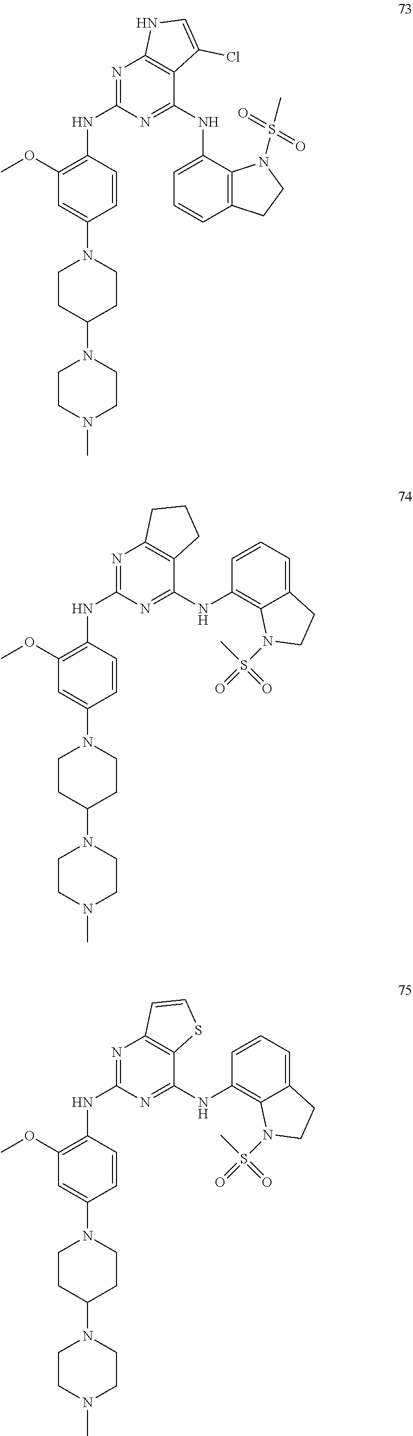 [SMILES: COc1cc(N2CCC(N3CCN(C)CC3)CC2)ccc1Nc1nc(Nc2cccc3c2N(S(C)(=O)=O)CC3)c2c(Cl)c[nH]c2n1 ; COc1cc(N2CCC(N3CCN(C)CC3)CC2)ccc1Nc1nc2c(c(Nc3cccc4c3N(S(C)(=O)=O)CC4)n1)CCC2 ; COc1cc(N2CCC(N3CCN(C)CC3)CC2)ccc1Nc1nc(Nc2cccc3c2N(S(C)(=O)=O)CC3)c2sccc2n1] 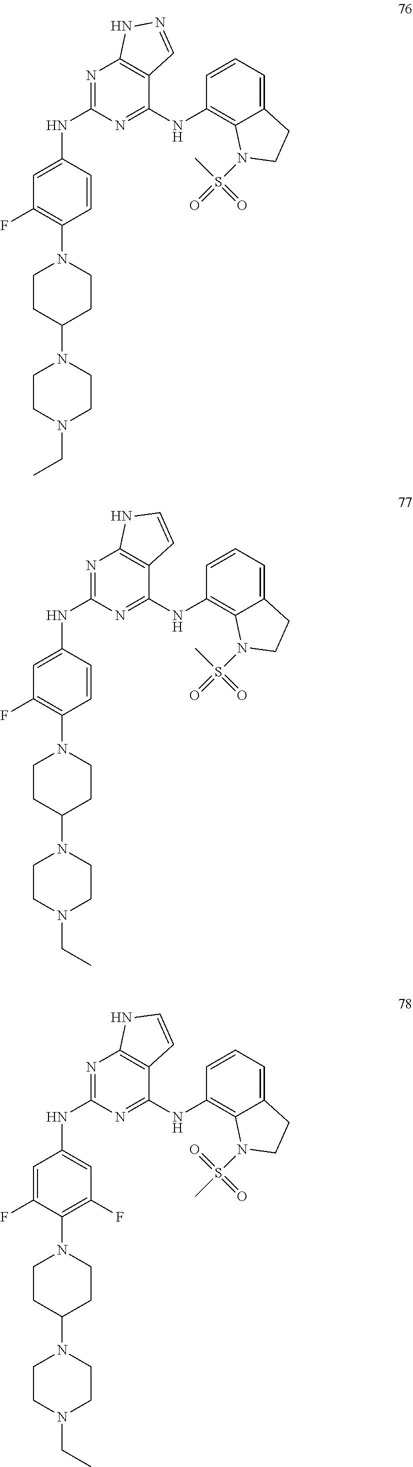 [SMILES: CCN1CCN(C2CCN(c3ccc(Nc4nc(Nc5cccc6c5N(S(C)(=O)=O)CC6)c5cn[nH]c5n4)cc3F)CC2)CC1 ; CCN1CCN(C2CCN(c3ccc(Nc4nc(Nc5cccc6c5N(S(C)(=O)=O)CC6)c5cc[nH]c5n4)cc3F)CC2)CC1 ; CCN1CCN(C2CCN(c3c(F)cc(Nc4nc(Nc5cccc6c5N(S(C)(=O)=O)CC6)c5cc[nH]c5n4)cc3F)CC2)CC1] 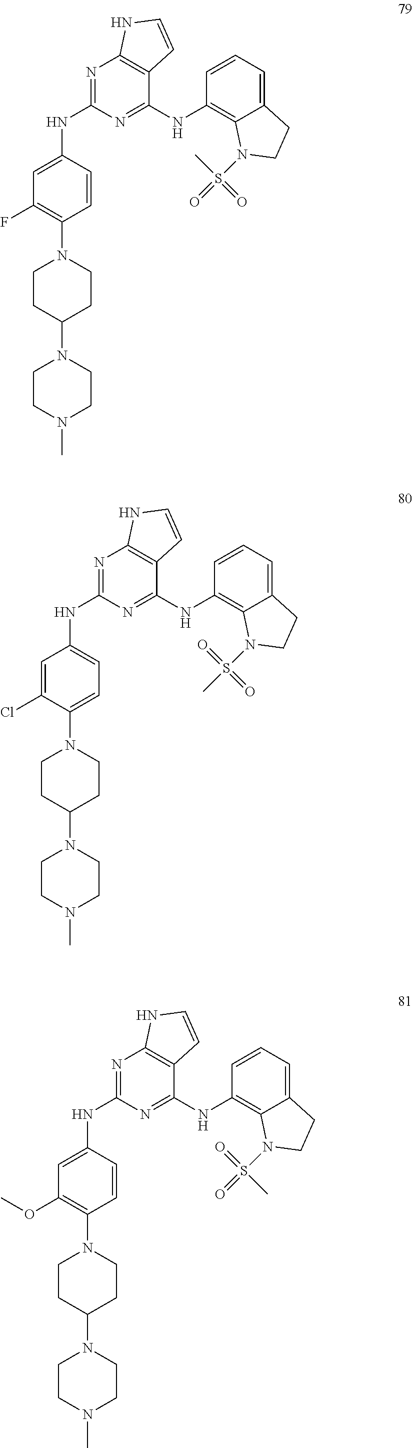 [SMILES: CN1CCN(C2CCN(c3ccc(Nc4nc(Nc5cccc6c5N(S(C)(=O)=O)CC6)c5cc[nH]c5n4)cc3F)CC2)CC1 ; CN1CCN(C2CCN(c3ccc(Nc4nc(Nc5cccc6c5N(S(C)(=O)=O)CC6)c5cc[nH]c5n4)cc3Cl)CC2)CC1 ; COc1cc(Nc2nc(Nc3cccc4c3N(S(C)(=O)=O)CC4)c3cc[nH]c3n2)ccc1N1CCC(N2CCN(C)CC2)CC1] 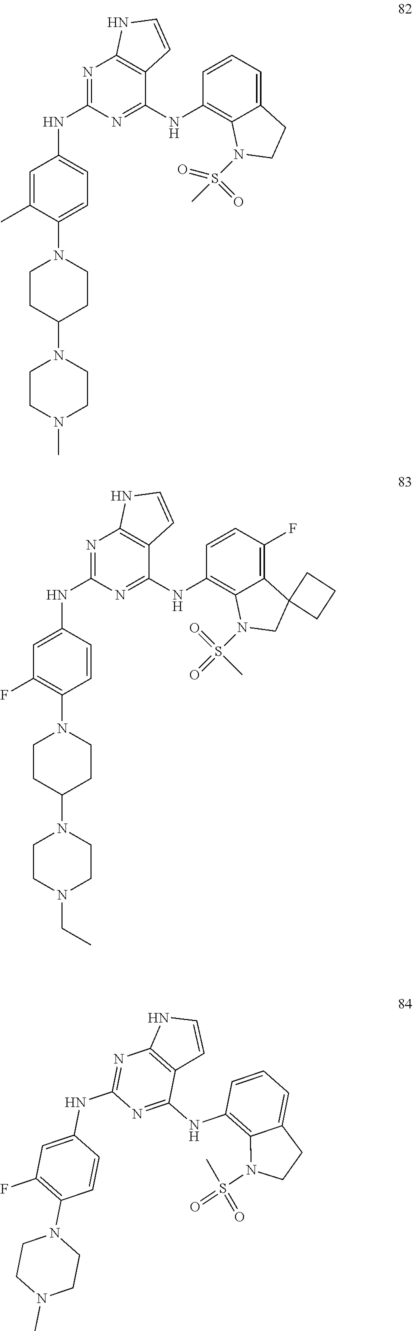 [SMILES: Cc1cc(Nc2nc(Nc3cccc4c3N(S(C)(=O)=O)CC4)c3cc[nH]c3n2)ccc1N1CCC(N2CCN(C)CC2)CC1 ; CCN1CCN(C2CCN(c3ccc(Nc4nc(Nc5ccc(F)c6c5N(S(C)(=O)=O)CC65CCC5)c5cc[nH]c5n4)cc3F)CC2)CC1 ; CN1CCN(c2ccc(Nc3nc(Nc4cccc5c4N(S(C)(=O)=O)CC5)c4cc[nH]c4n3)cc2F)CC1] 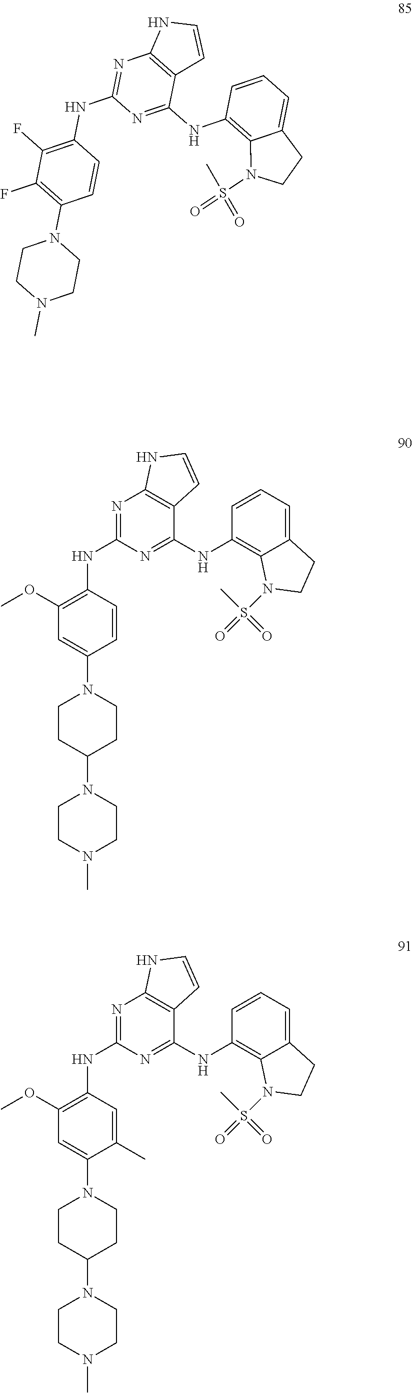 [SMILES: CN1CCN(c2ccc(Nc3nc(Nc4cccc5c4N(S(C)(=O)=O)CC5)c4cc[nH]c4n3)c(F)c2F)CC1 ; COc1cc(N2CCC(N3CCN(C)CC3)CC2)ccc1Nc1nc(Nc2cccc3c2N(S(C)(=O)=O)CC3)c2cc[nH]c2n1 ; COc1cc(N2CCC(N3CCN(C)CC3)CC2)c(C)cc1Nc1nc(Nc2cccc3c2N(S(C)(=O)=O)CC3)c2cc[nH]c2n1] 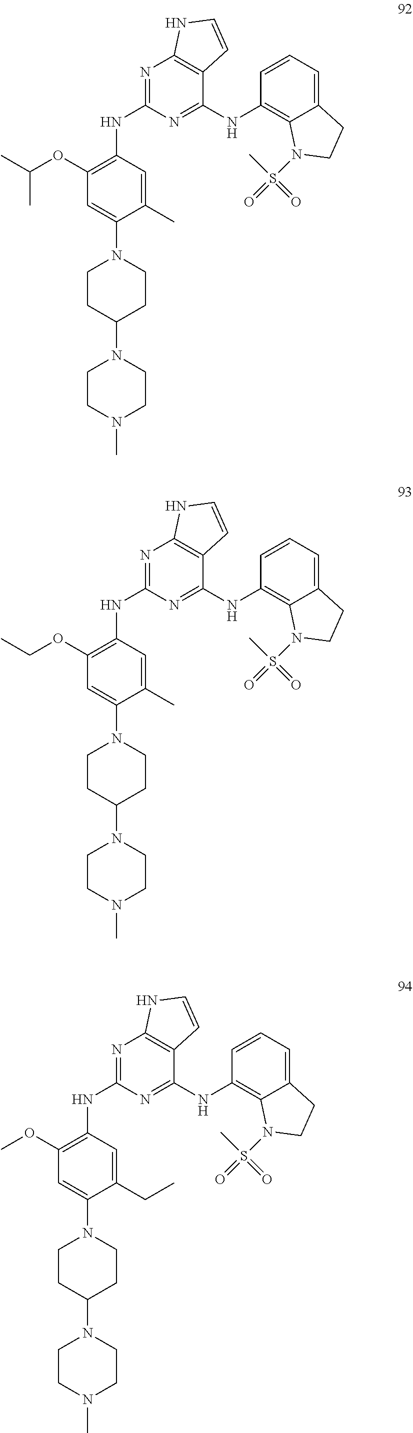 [SMILES: Cc1cc(Nc2nc(Nc3cccc4c3N(S(C)(=O)=O)CC4)c3cc[nH]c3n2)c(OC(C)C)cc1N1CCC(N2CCN(C)CC2)CC1 ; CCOc1cc(N2CCC(N3CCN(C)CC3)CC2)c(C)cc1Nc1nc(Nc2cccc3c2N(S(C)(=O)=O)CC3)c2cc[nH]c2n1 ; CCc1cc(Nc2nc(Nc3cccc4c3N(S(C)(=O)=O)CC4)c3cc[nH]c3n2)c(OC)cc1N1CCC(N2CCN(C)CC2)CC1] 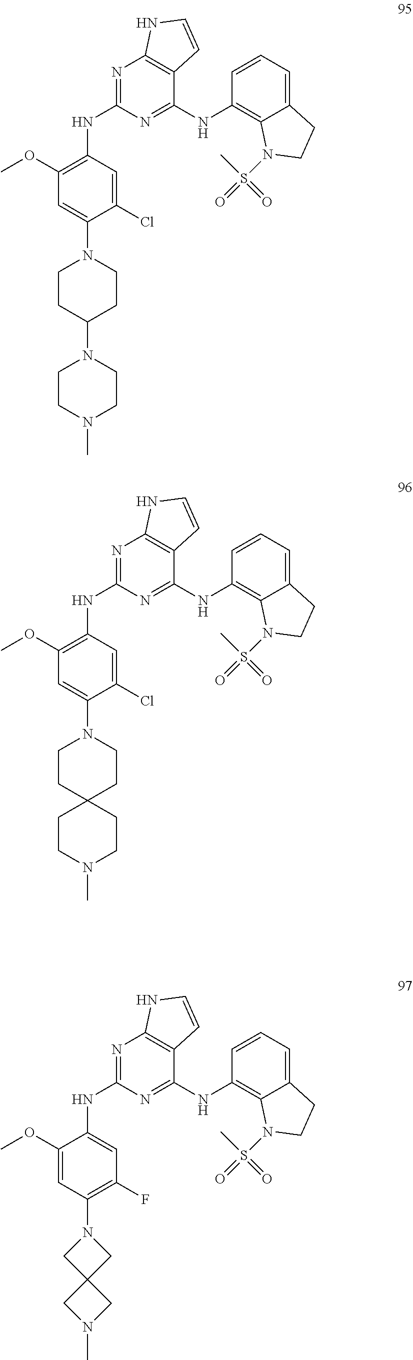 [SMILES: COc1cc(N2CCC(N3CCN(C)CC3)CC2)c(Cl)cc1Nc1nc(Nc2cccc3c2N(S(C)(=O)=O)CC3)c2cc[nH]c2n1 ; COc1cc(N2CCC3(CCN(C)CC3)CC2)c(Cl)cc1Nc1nc(Nc2cccc3c2N(S(C)(=O)=O)CC3)c2cc[nH]c2n1 ; COc1cc(N2CC3(CN(C)C3)C2)c(F)cc1Nc1nc(Nc2cccc3c2N(S(C)(=O)=O)CC3)c2cc[nH]c2n1] 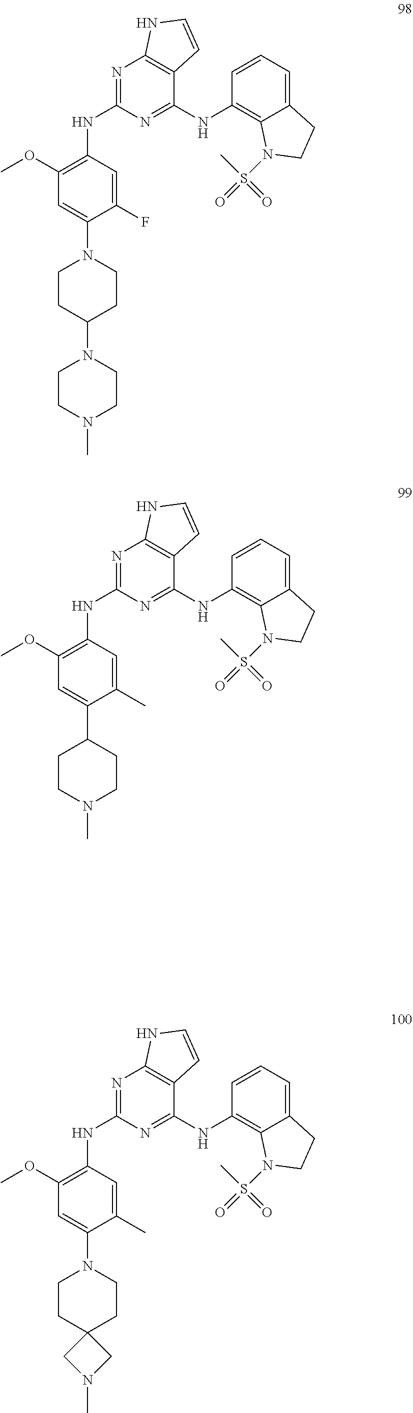 [SMILES: COc1cc(N2CCC(N3CCN(C)CC3)CC2)c(F)cc1Nc1nc(Nc2cccc3c2N(S(C)(=O)=O)CC3)c2cc[nH]c2n1 ; COc1cc(C2CCN(C)CC2)c(C)cc1Nc1nc(Nc2cccc3c2N(S(C)(=O)=O)CC3)c2cc[nH]c2n1 ; COc1cc(N2CCC3(CC2)CN(C)C3)c(C)cc1Nc1nc(Nc2cccc3c2N(S(C)(=O)=O)CC3)c2cc[nH]c2n1] 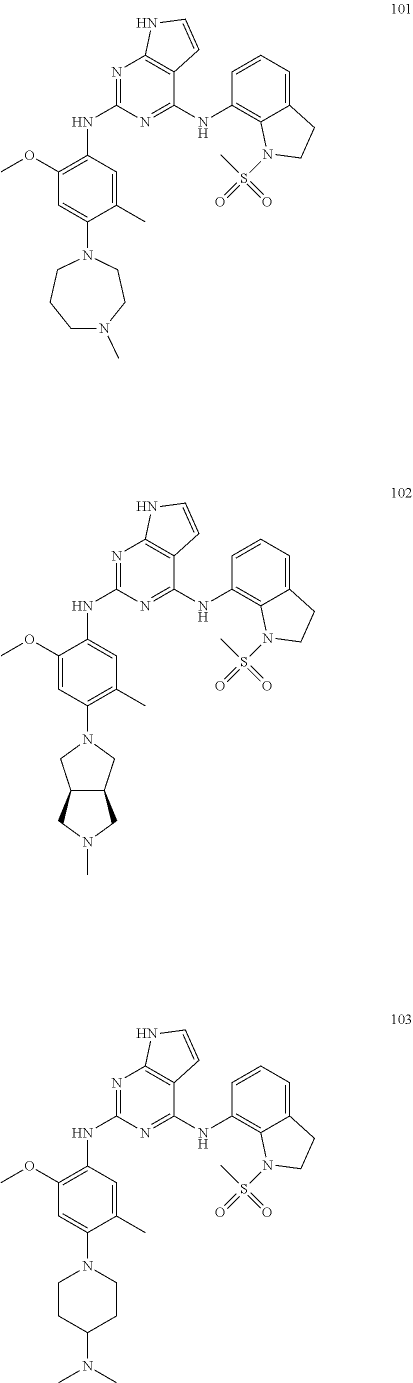 [SMILES: COc1cc(N2CCCN(C)CC2)c(C)cc1Nc1nc(Nc2cccc3c2N(S(C)(=O)=O)CC3)c2cc[nH]c2n1 ; COc1cc(N2C[C@H]3CN(C)C[C@H]3C2)c(C)cc1Nc1nc(Nc2cccc3c2N(S(C)(=O)=O)CC3)c2cc[nH]c2n1 ; COc1cc(N2CCC(N(C)C)CC2)c(C)cc1Nc1nc(Nc2cccc3c2N(S(C)(=O)=O)CC3)c2cc[nH]c2n1] 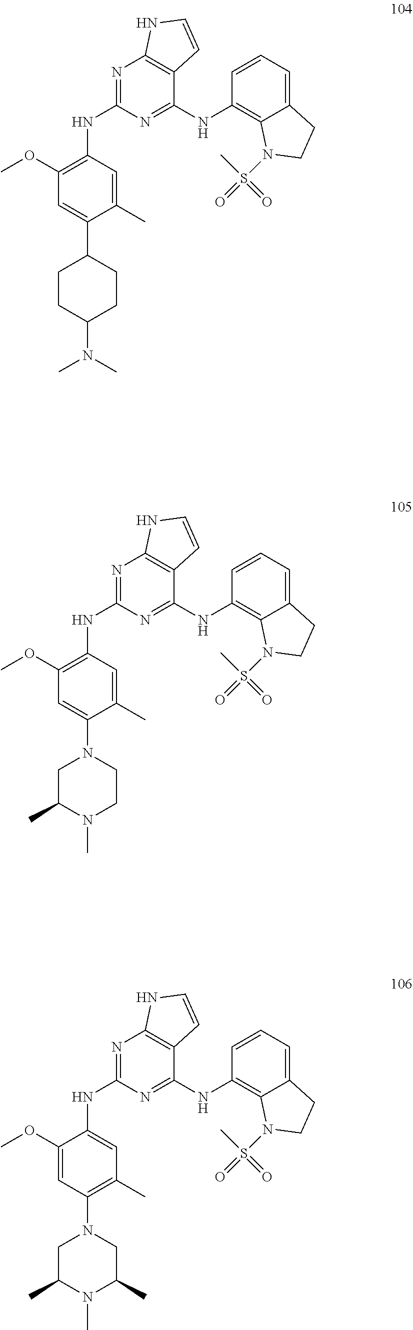 [SMILES: COc1cc(C2CCC(N(C)C)CC2)c(C)cc1Nc1nc(Nc2cccc3c2N(S(C)(=O)=O)CC3)c2cc[nH]c2n1 ; COc1cc(N2CCN(C)[C@@H](C)C2)c(C)cc1Nc1nc(Nc2cccc3c2N(S(C)(=O)=O)CC3)c2cc[nH]c2n1 ; COc1cc(N2C[C@@H](C)N(C)[C@@H](C)C2)c(C)cc1Nc1nc(Nc2cccc3c2N(S(C)(=O)=O)CC3)c2cc[nH]c2n1] 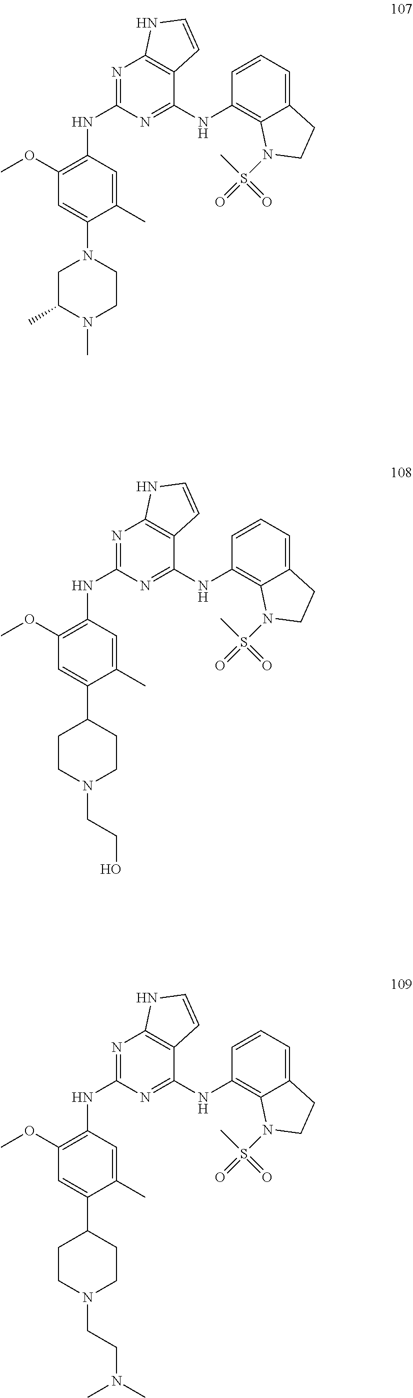 [SMILES: COc1cc(N2CCN(C)[C@H](C)C2)c(C)cc1Nc1nc(Nc2cccc3c2N(S(C)(=O)=O)CC3)c2cc[nH]c2n1 ; COc1cc(C2CCN(CCO)CC2)c(C)cc1Nc1nc(Nc2cccc3c2N(S(C)(=O)=O)CC3)c2cc[nH]c2n1 ; COc1cc(C2CCN(CCN(C)C)CC2)c(C)cc1Nc1nc(Nc2cccc3c2N(S(C)(=O)=O)CC3)c2cc[nH]c2n1] 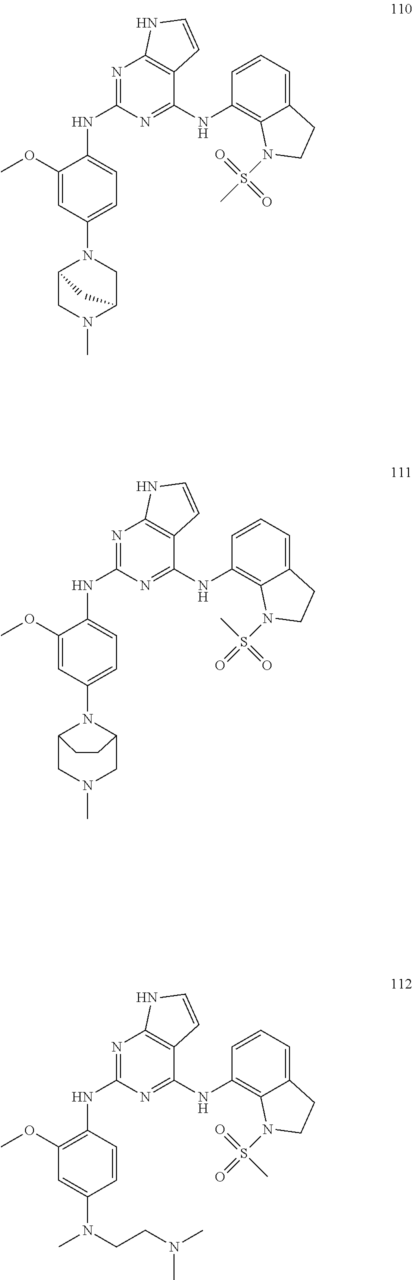 [SMILES: COc1cc(N2C[C@@H]3C[C@H]2CN3C)ccc1Nc1nc(Nc2cccc3c2N(S(C)(=O)=O)CC3)c2cc[nH]c2n1 ; COc1cc(N2C3CCC2CN(C)C3)ccc1Nc1nc(Nc2cccc3c2N(S(C)(=O)=O)CC3)c2cc[nH]c2n1 ; COc1cc(N(C)CCN(C)C)ccc1Nc1nc(Nc2cccc3c2N(S(C)(=O)=O)CC3)c2cc[nH]c2n1] 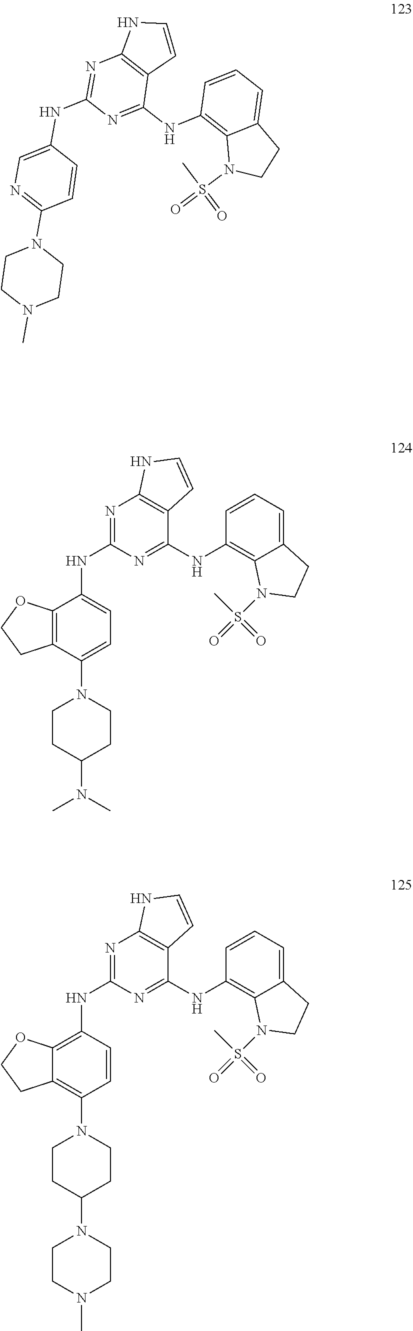 [SMILES: CN1CCN(c2ccc(Nc3nc(Nc4cccc5c4N(S(C)(=O)=O)CC5)c4cc[nH]c4n3)cn2)CC1 ; CN(C)C1CCN(c2ccc(Nc3nc(Nc4cccc5c4N(S(C)(=O)=O)CC5)c4cc[nH]c4n3)c3c2CCO3)CC1 ; CN1CCN(C2CCN(c3ccc(Nc4nc(Nc5cccc6c5N(S(C)(=O)=O)CC6)c5cc[nH]c5n4)c4c3CCO4)CC2)CC1] 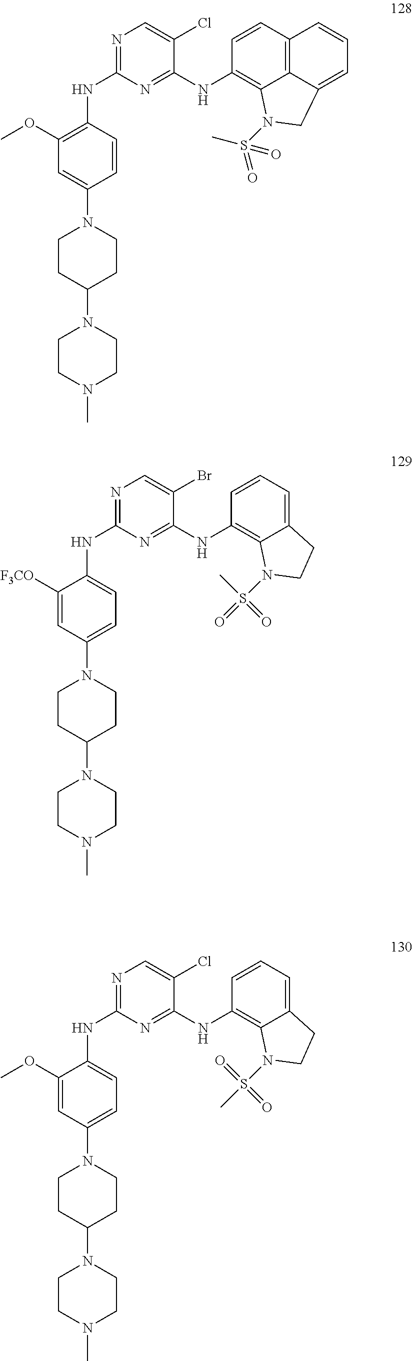 [SMILES: COc1cc(N2CCC(N3CCN(C)CC3)CC2)ccc1Nc1ncc(Cl)c(Nc2ccc3cccc4c3c2N(S(C)(=O)=O)C4)n1 ; CN1CCN(C2CCN(c3ccc(Nc4ncc(Br)c(Nc5cccc6c5N(S(C)(=O)=O)CC6)n4)c(OC(F)(F)F)c3)CC2)CC1 ; COc1cc(N2CCC(N3CCN(C)CC3)CC2)ccc1Nc1ncc(Cl)c(Nc2cccc3c2N(S(C)(=O)=O)CC3)n1] 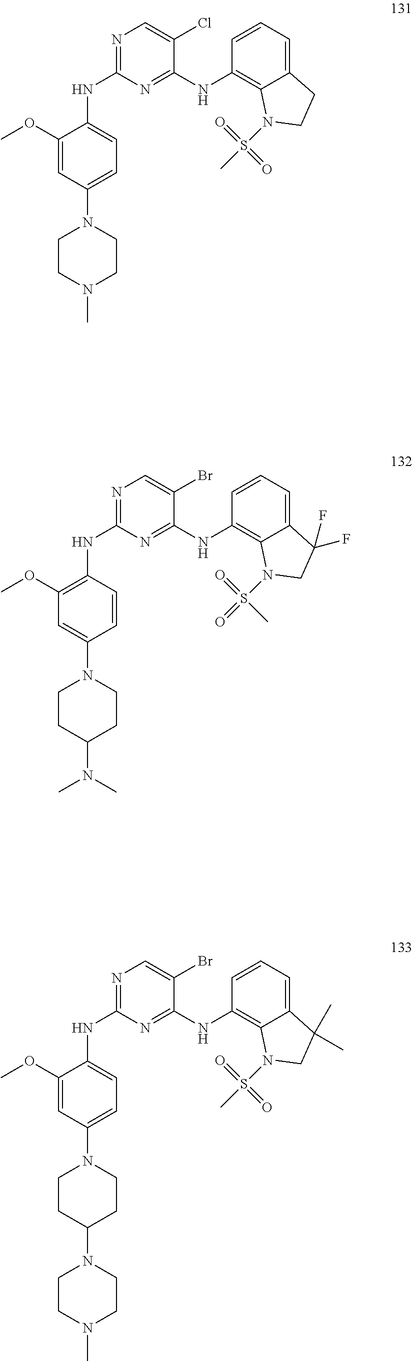 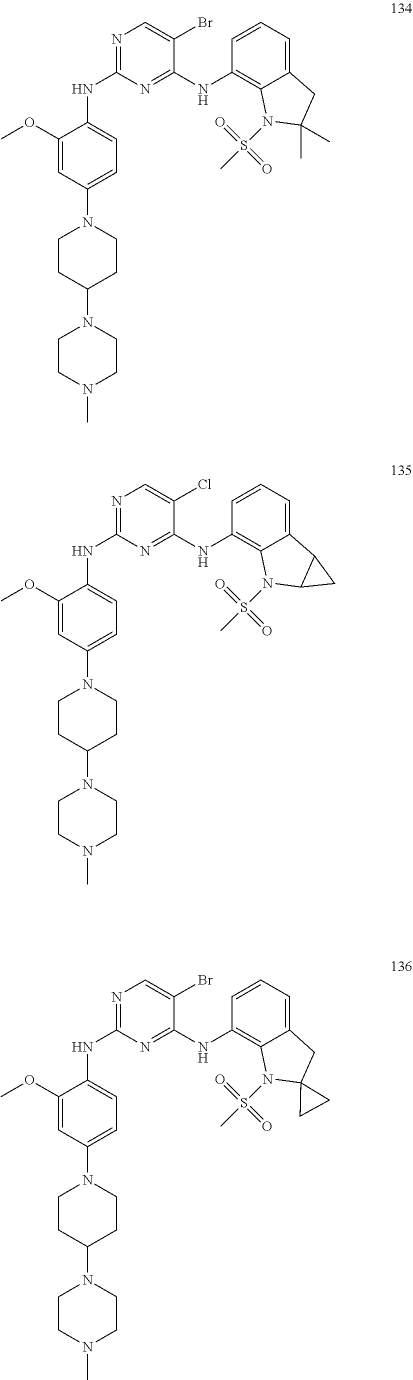 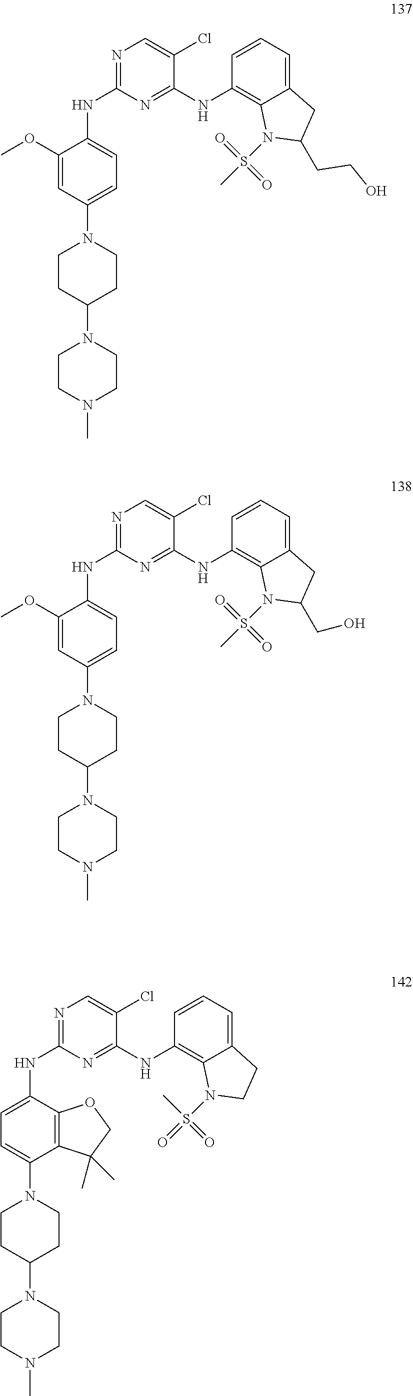 [SMILES: COc1cc(N2CCC(N3CCN(C)CC3)CC2)ccc1Nc1ncc(Cl)c(Nc2cccc3c2N(S(C)(=O)=O)C(CCO)C3)n1 ; COc1cc(N2CCC(N3CCN(C)CC3)CC2)ccc1Nc1ncc(Cl)c(Nc2cccc3c2N(S(C)(=O)=O)C(CO)C3)n1 ; CN1CCN(C2CCN(c3ccc(Nc4ncc(Cl)c(Nc5cccc6c5N(S(C)(=O)=O)CC6)n4)c4c3C(C)(C)CO4)CC2)CC1] 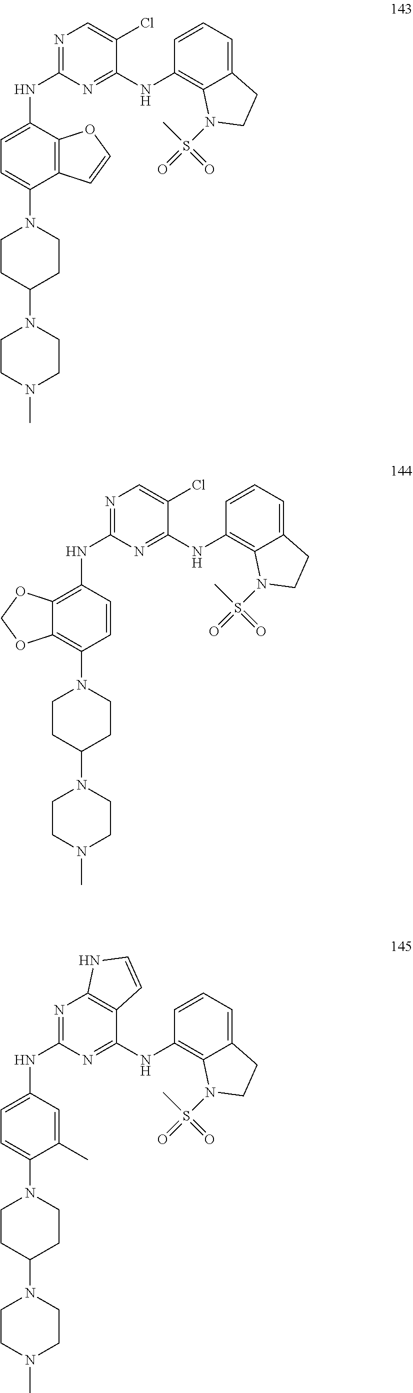 [SMILES: CN1CCN(C2CCN(c3ccc(Nc4ncc(Cl)c(Nc5cccc6c5N(S(C)(=O)=O)CC6)n4)c4occc34)CC2)CC1 ; CN1CCN(C2CCN(c3ccc(Nc4ncc(Cl)c(Nc5cccc6c5N(S(C)(=O)=O)CC6)n4)c4c3OCO4)CC2)CC1 ; Cc1cc(Nc2nc(Nc3cccc4c3N(S(C)(=O)=O)CC4)c3cc[nH]c3n2)ccc1N1CCC(N2CCN(C)CC2)CC1] 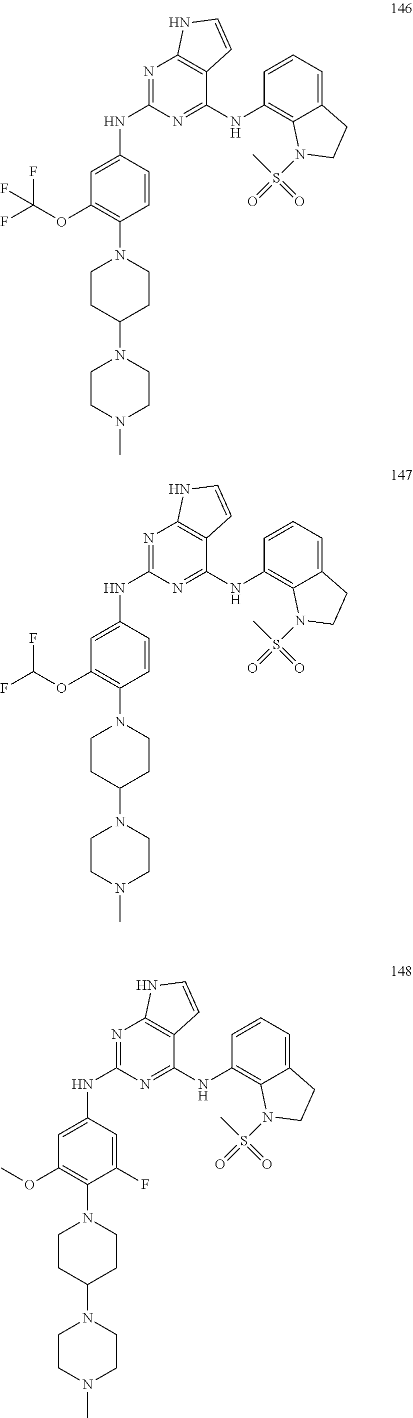 [SMILES: CN1CCN(C2CCN(c3ccc(Nc4nc(Nc5cccc6c5N(S(C)(=O)=O)CC6)c5cc[nH]c5n4)cc3OC(F)(F)F)CC2)CC1 ; CN1CCN(C2CCN(c3ccc(Nc4nc(Nc5cccc6c5N(S(C)(=O)=O)CC6)c5cc[nH]c5n4)cc3OC(F)F)CC2)CC1 ; COc1cc(Nc2nc(Nc3cccc4c3N(S(C)(=O)=O)CC4)c3cc[nH]c3n2)cc(F)c1N1CCC(N2CCN(C)CC2)CC1] 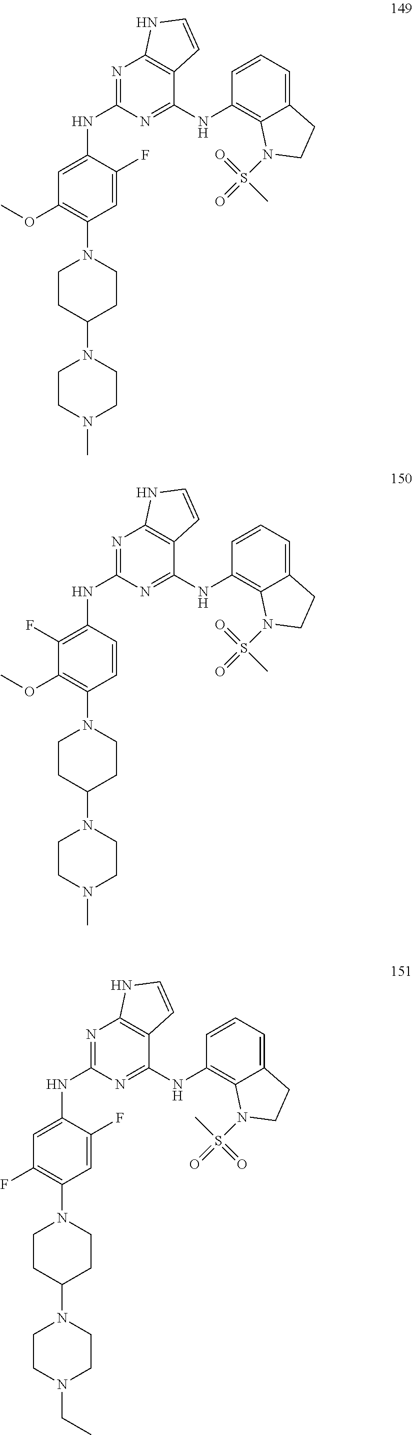 [SMILES: COc1cc(Nc2nc(Nc3cccc4c3N(S(C)(=O)=O)CC4)c3cc[nH]c3n2)c(F)cc1N1CCC(N2CCN(C)CC2)CC1 ; COc1c(N2CCC(N3CCN(C)CC3)CC2)ccc(Nc2nc(Nc3cccc4c3N(S(C)(=O)=O)CC4)c3cc[nH]c3n2)c1F ; CCN1CCN(C2CCN(c3cc(F)c(Nc4nc(Nc5cccc6c5N(S(C)(=O)=O)CC6)c5cc[nH]c5n4)cc3F)CC2)CC1] 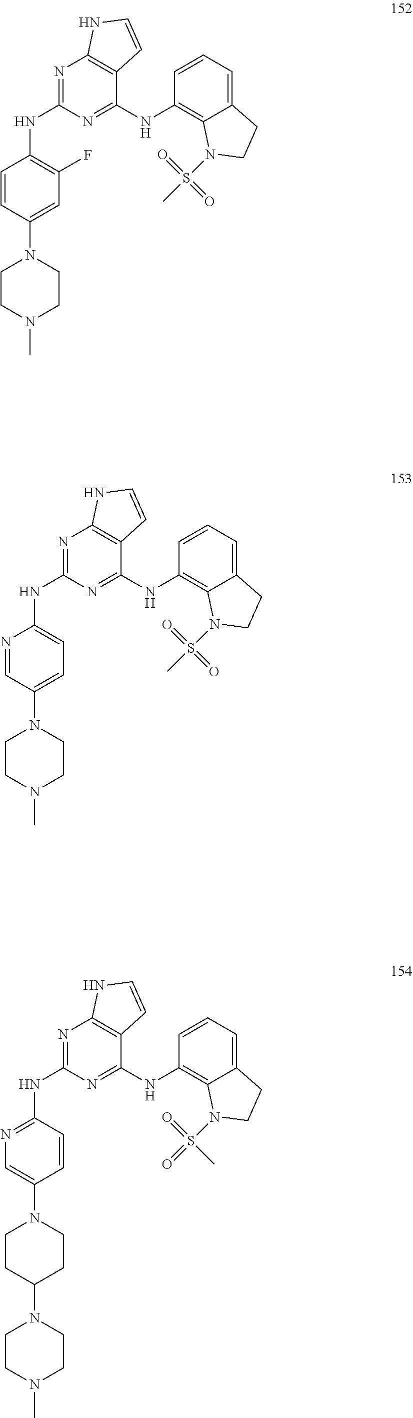 [SMILES: CN1CCN(c2ccc(Nc3nc(Nc4cccc5c4N(S(C)(=O)=O)CC5)c4cc[nH]c4n3)c(F)c2)CC1 ; CN1CCN(c2ccc(Nc3nc(Nc4cccc5c4N(S(C)(=O)=O)CC5)c4cc[nH]c4n3)nc2)CC1 ; CN1CCN(C2CCN(c3ccc(Nc4nc(Nc5cccc6c5N(S(C)(=O)=O)CC6)c5cc[nH]c5n4)nc3)CC2)CC1] 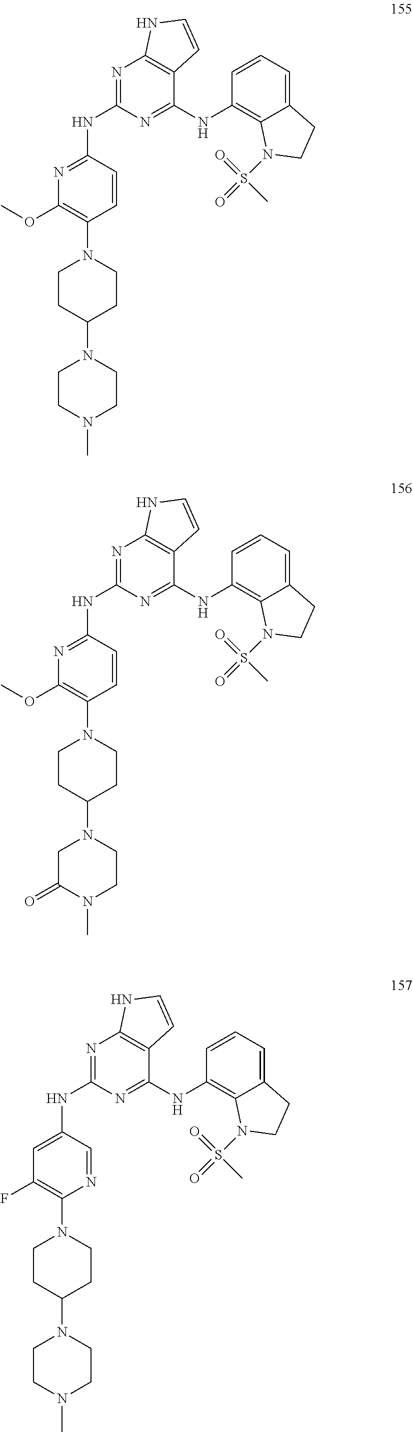 [SMILES: COc1nc(Nc2nc(Nc3cccc4c3N(S(C)(=O)=O)CC4)c3cc[nH]c3n2)ccc1N1CCC(N2CCN(C)CC2)CC1 ; COc1nc(Nc2nc(Nc3cccc4c3N(S(C)(=O)=O)CC4)c3cc[nH]c3n2)ccc1N1CCC(N2CCN(C)C(=O)C2)CC1 ; CN1CCN(C2CCN(c3ncc(Nc4nc(Nc5cccc6c5N(S(C)(=O)=O)CC6)c5cc[nH]c5n4)cc3F)CC2)CC1] 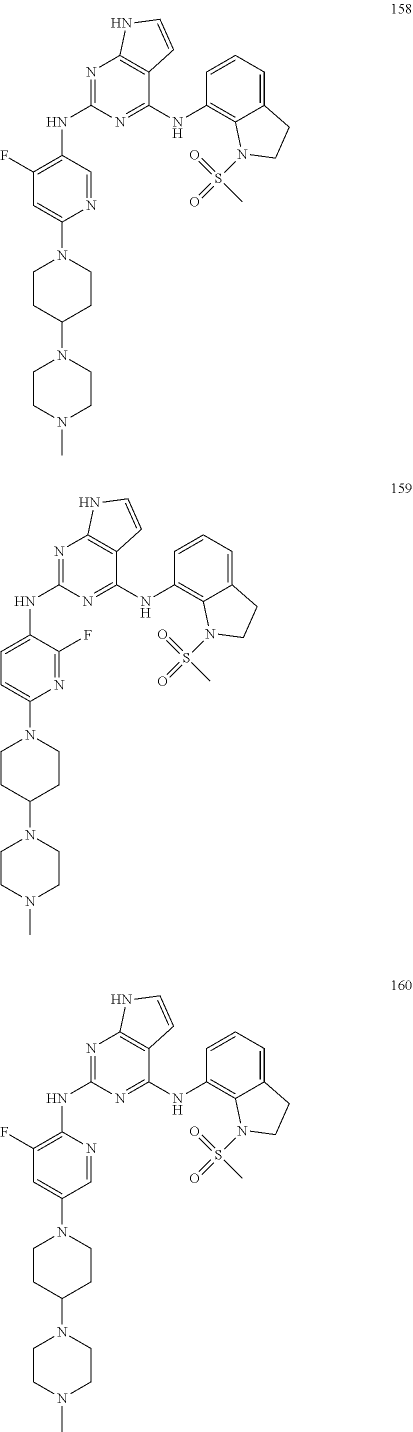 [SMILES: CN1CCN(C2CCN(c3cc(F)c(Nc4nc(Nc5cccc6c5N(S(C)(=O)=O)CC6)c5cc[nH]c5n4)cn3)CC2)CC1 ; CN1CCN(C2CCN(c3ccc(Nc4nc(Nc5cccc6c5N(S(C)(=O)=O)CC6)c5cc[nH]c5n4)c(F)n3)CC2)CC1 ; CN1CCN(C2CCN(c3cnc(Nc4nc(Nc5cccc6c5N(S(C)(=O)=O)CC6)c5cc[nH]c5n4)c(F)c3)CC2)CC1] 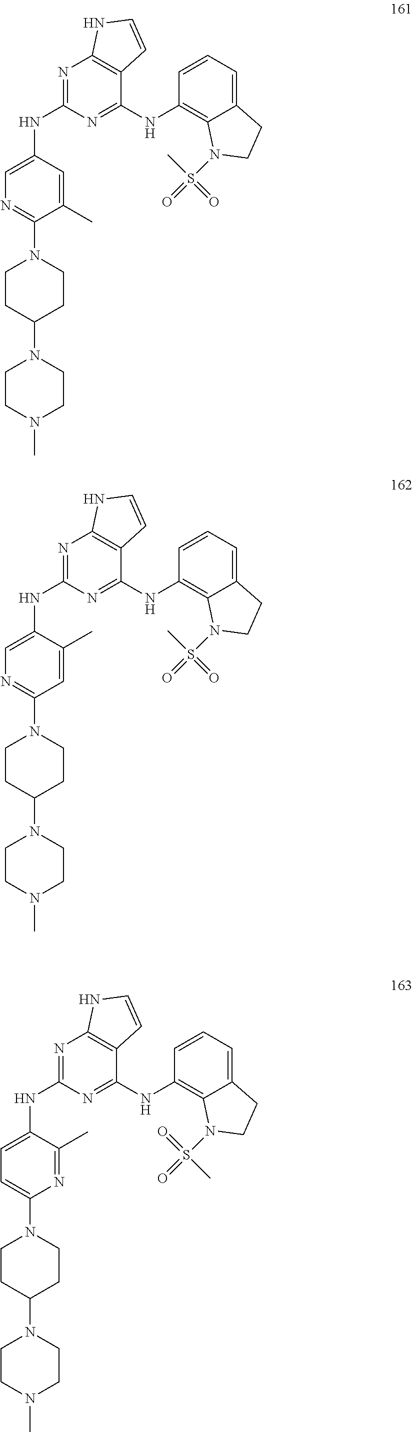 [SMILES: Cc1cc(Nc2nc(Nc3cccc4c3N(S(C)(=O)=O)CC4)c3cc[nH]c3n2)cnc1N1CCC(N2CCN(C)CC2)CC1 ; Cc1cc(N2CCC(N3CCN(C)CC3)CC2)ncc1Nc1nc(Nc2cccc3c2N(S(C)(=O)=O)CC3)c2cc[nH]c2n1 ; Cc1nc(N2CCC(N3CCN(C)CC3)CC2)ccc1Nc1nc(Nc2cccc3c2N(S(C)(=O)=O)CC3)c2cc[nH]c2n1] 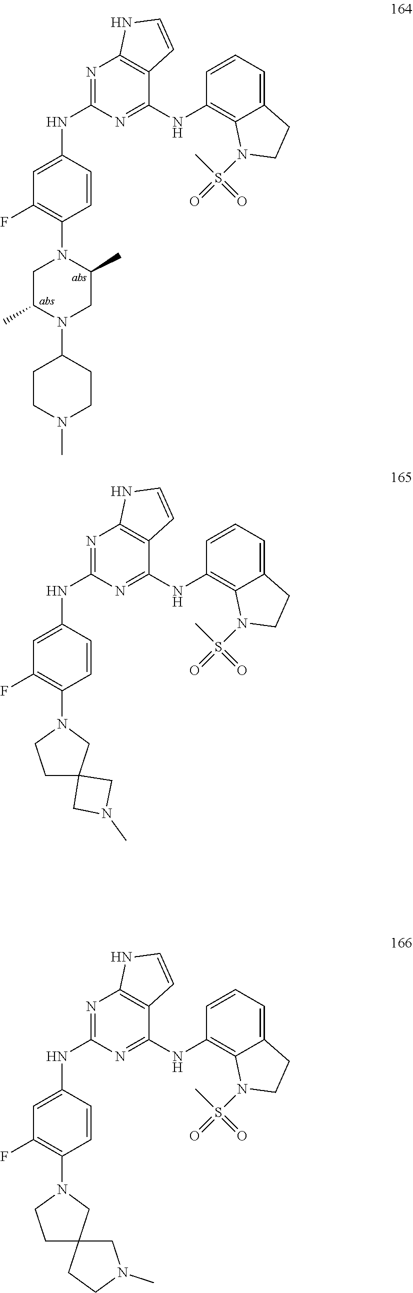 [SMILES: C[C@@H]1CN(c2ccc(Nc3nc(Nc4cccc5c4N(S(C)(=O)=O)CC5)c4cc[nH]c4n3)cc2F)[C@@H](C)CN1C1CCN(C)CC1 ; CN1CC2(CCN(c3ccc(Nc4nc(Nc5cccc6c5N(S(C)(=O)=O)CC6)c5cc[nH]c5n4)cc3F)C2)C1 ; CN1CCC2(CCN(c3ccc(Nc4nc(Nc5cccc6c5N(S(C)(=O)=O)CC6)c5cc[nH]c5n4)cc3F)C2)C1] 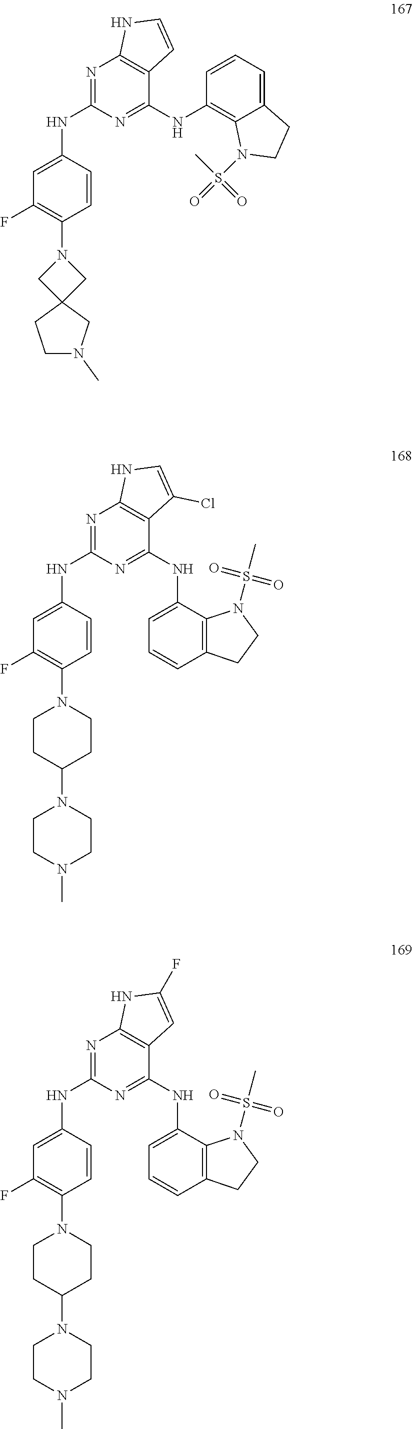 [SMILES: CN1CCC2(C1)CN(c1ccc(Nc3nc(Nc4cccc5c4N(S(C)(=O)=O)CC5)c4cc[nH]c4n3)cc1F)C2 ; CN1CCN(C2CCN(c3ccc(Nc4nc(Nc5cccc6c5N(S(C)(=O)=O)CC6)c5c(Cl)c[nH]c5n4)cc3F)CC2)CC1 ; CN1CCN(C2CCN(c3ccc(Nc4nc(Nc5cccc6c5N(S(C)(=O)=O)CC6)c5cc(F)[nH]c5n4)cc3F)CC2)CC1] 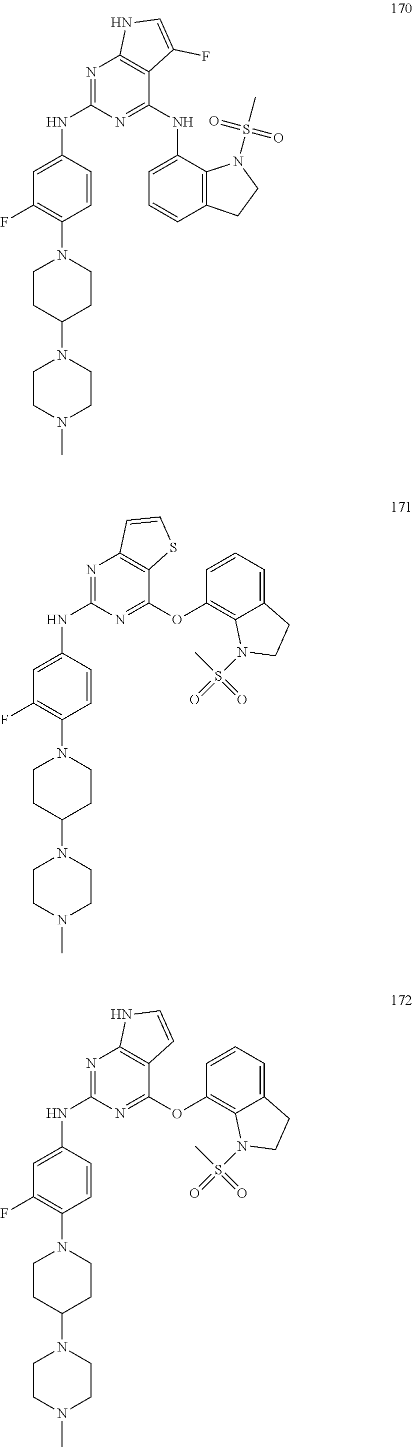 [SMILES: CN1CCN(C2CCN(c3ccc(Nc4nc(Nc5cccc6c5N(S(C)(=O)=O)CC6)c5c(F)c[nH]c5n4)cc3F)CC2)CC1 ; CN1CCN(C2CCN(c3ccc(Nc4nc(Oc5cccc6c5N(S(C)(=O)=O)CC6)c5sccc5n4)cc3F)CC2)CC1 ; CN1CCN(C2CCN(c3ccc(Nc4nc(Oc5cccc6c5N(S(C)(=O)=O)CC6)c5cc[nH]c5n4)cc3F)CC2)CC1] 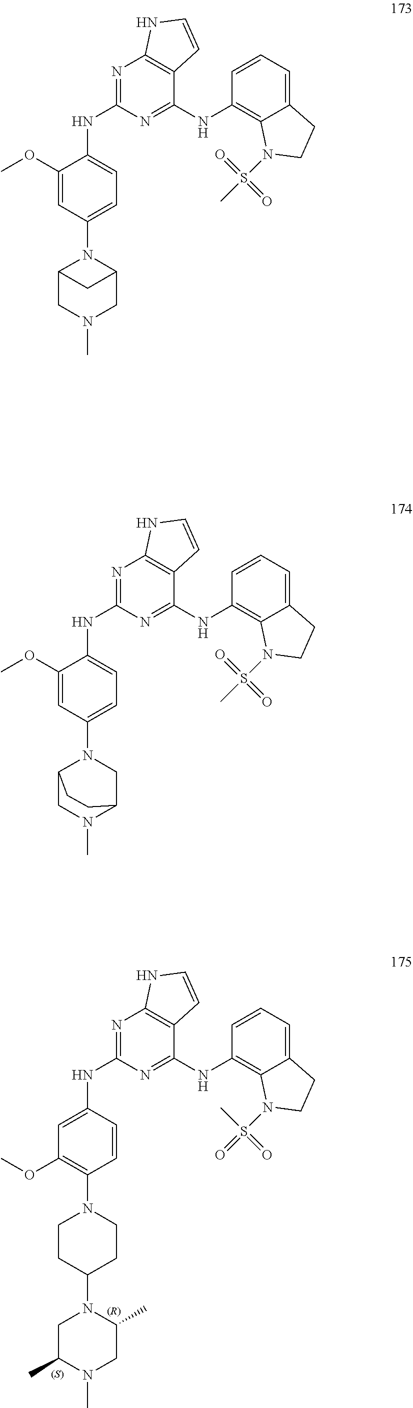 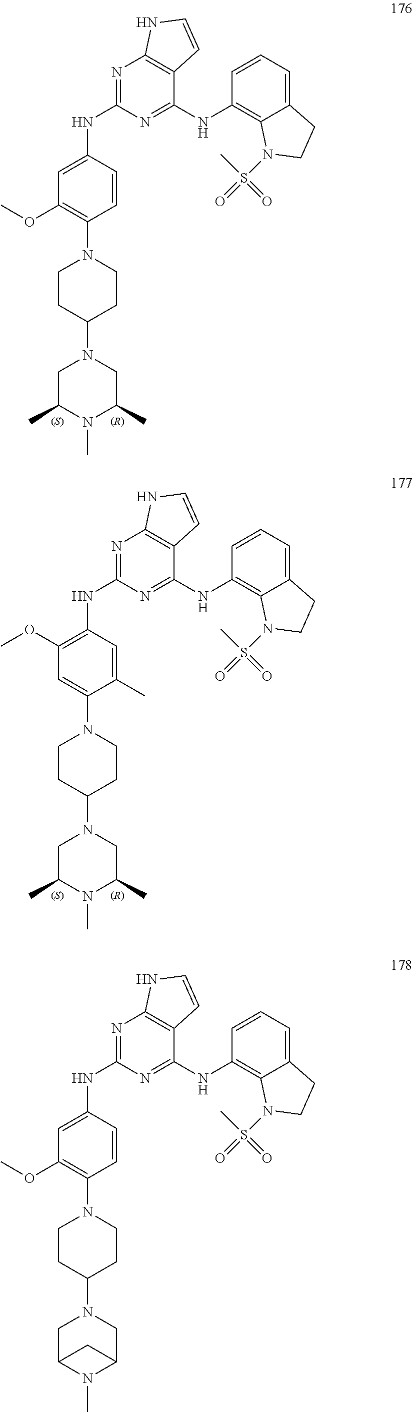 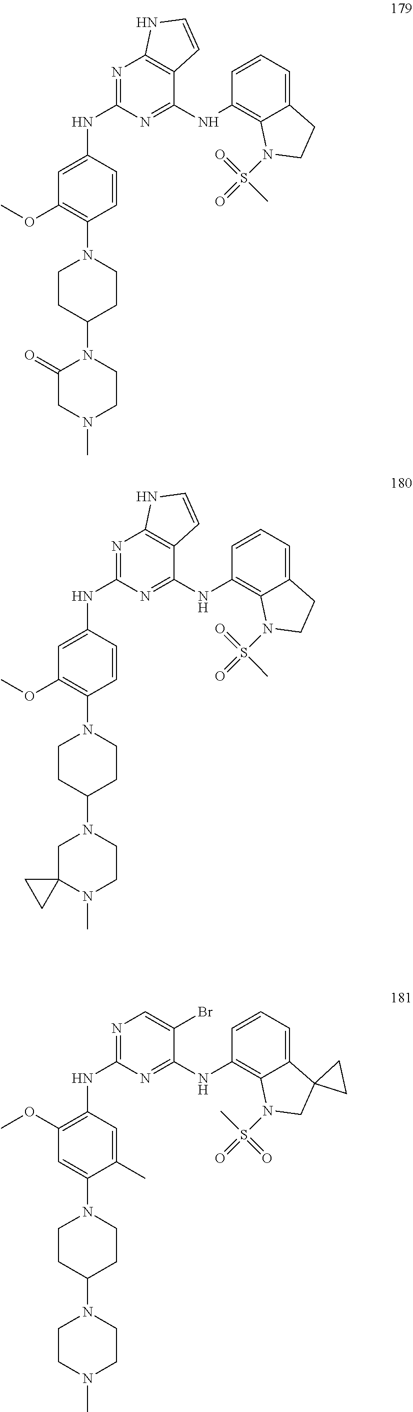 [SMILES: COc1cc(Nc2nc(Nc3cccc4c3N(S(C)(=O)=O)CC4)c3cc[nH]c3n2)ccc1N1CCC(N2CCN(C)CC2=O)CC1 ; COc1cc(Nc2nc(Nc3cccc4c3N(S(C)(=O)=O)CC4)c3cc[nH]c3n2)ccc1N1CCC(N2CCN(C)C3(CC3)C2)CC1 ; COc1cc(N2CCC(N3CCN(C)CC3)CC2)c(C)cc1Nc1ncc(Br)c(Nc2cccc3c2N(S(C)(=O)=O)CC32CC2)n1] 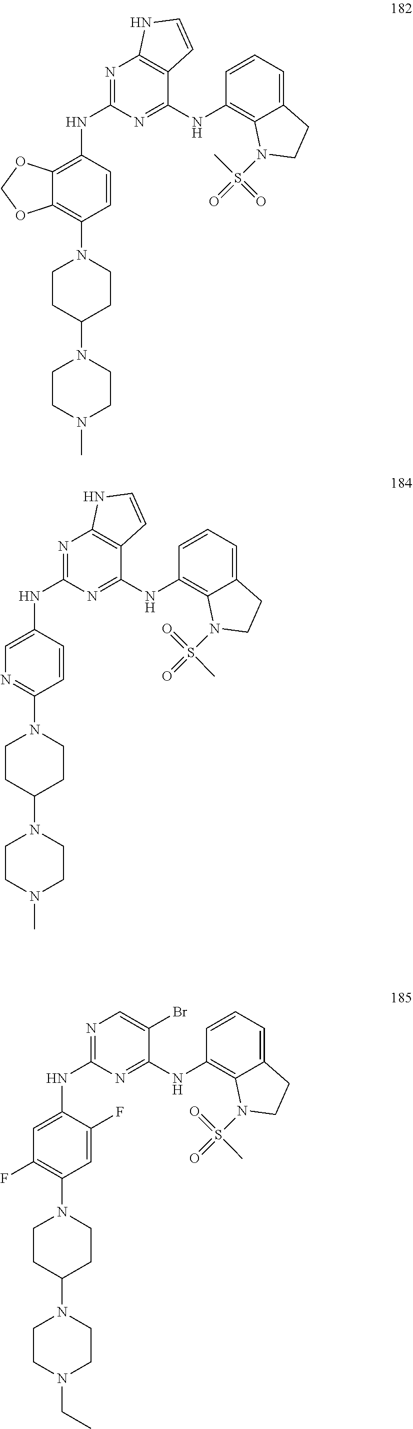 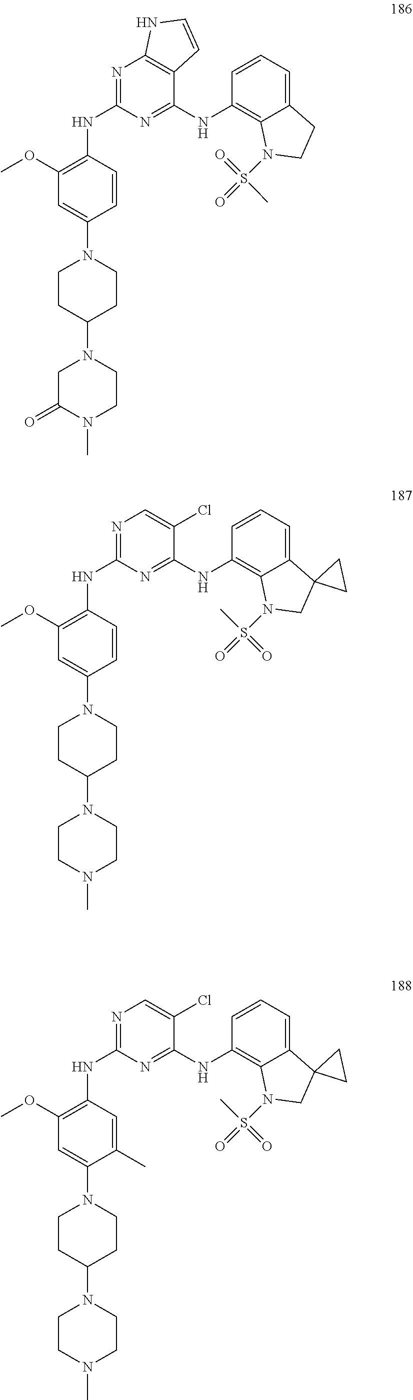 [SMILES: COc1cc(N2CCC(N3CCN(C)C(=O)C3)CC2)ccc1Nc1nc(Nc2cccc3c2N(S(C)(=O)=O)CC3)c2cc[nH]c2n1 ; COc1cc(N2CCC(N3CCN(C)CC3)CC2)ccc1Nc1ncc(Cl)c(Nc2cccc3c2N(S(C)(=O)=O)CC32CC2)n1 ; COc1cc(N2CCC(N3CCN(C)CC3)CC2)c(C)cc1Nc1ncc(Cl)c(Nc2cccc3c2N(S(C)(=O)=O)CC32CC2)n1] 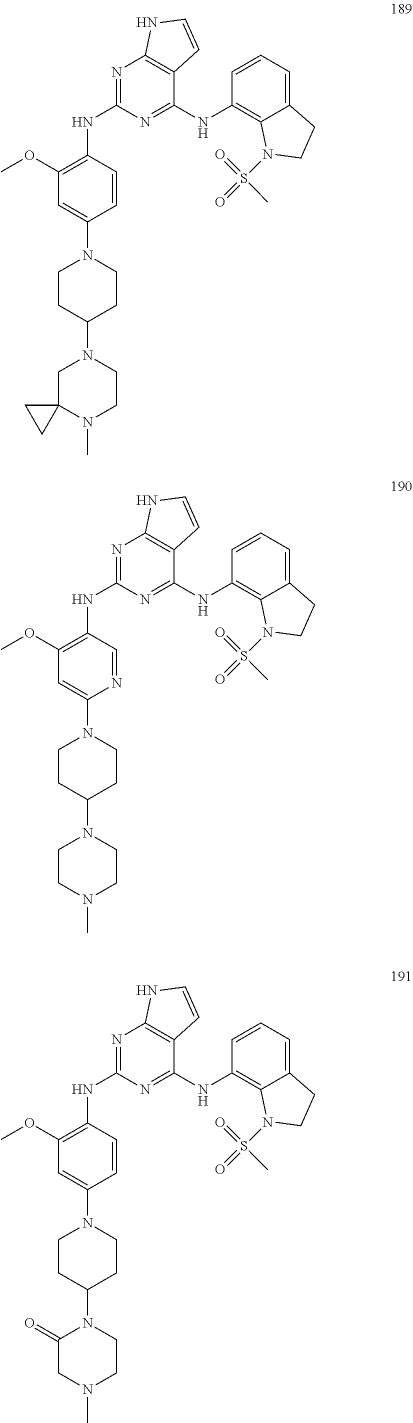 [SMILES: COc1cc(N2CCC(N3CCN(C)C4(CC4)C3)CC2)ccc1Nc1nc(Nc2cccc3c2N(S(C)(=O)=O)CC3)c2cc[nH]c2n1 ; COc1cc(N2CCC(N3CCN(C)CC3)CC2)ncc1Nc1nc(Nc2cccc3c2N(S(C)(=O)=O)CC3)c2cc[nH]c2n1 ; COc1cc(N2CCC(N3CCN(C)CC3=O)CC2)ccc1Nc1nc(Nc2cccc3c2N(S(C)(=O)=O)CC3)c2cc[nH]c2n1] 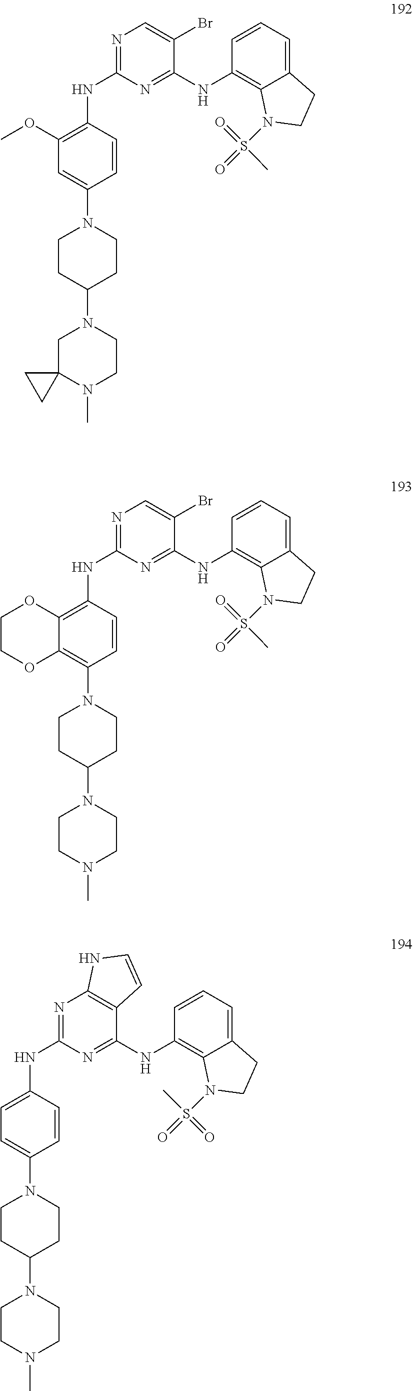 [SMILES: COc1cc(N2CCC(N3CCN(C)C4(CC4)C3)CC2)ccc1Nc1ncc(Br)c(Nc2cccc3c2N(S(C)(=O)=O)CC3)n1 ; CN1CCN(C2CCN(c3ccc(Nc4ncc(Br)c(Nc5cccc6c5N(S(C)(=O)=O)CC6)n4)c4c3OCCO4)CC2)CC1 ; CN1CCN(C2CCN(c3ccc(Nc4nc(Nc5cccc6c5N(S(C)(=O)=O)CC6)c5cc[nH]c5n4)cc3)CC2)CC1] 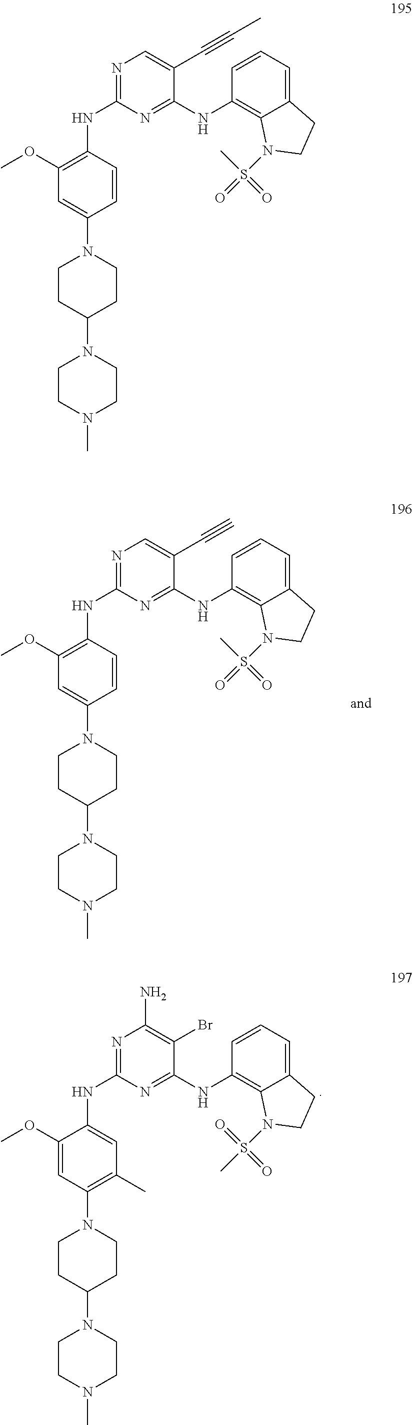 [SMILES: CC#Cc1cnc(Nc2ccc(N3CCC(N4CCN(C)CC4)CC3)cc2OC)nc1Nc1cccc2c1N(S(C)(=O)=O)CC2 ; C#Cc1cnc(Nc2ccc(N3CCC(N4CCN(C)CC4)CC3)cc2OC)nc1Nc1cccc2c1N(S(C)(=O)=O)CC2 ; COc1cc(N2CCC(N3CCN(C)CC3)CC2)c(C)cc1Nc1nc(N)c(Br)c(Nc2cccc3c2N(S(C)(=O)=O)CC3)n1]
or an N-oxide thereof or a pharmaceutically acceptable salt thereof or a stereoisomer thereof or prodrug thereof.

23. A pharmaceutical composition comprising a compound of claim 1 or a pharmaceutically acceptable salt, stereoisomer, tautomer or prodrug thereof, together with a pharmaceutically acceptable excipient.

24. A method of treating a disease in which EGFR modulation is involved, comprising administrating a subject in need thereof an effective amount of theft compound of claim 1 or an N-oxide thereof or a pharmaceutically acceptable salt thereof or a stereoisomer thereof or prodrug thereof.

25. The method of claim 24, wherein the disease is cancer.

26.-28. (canceled)
